(19) United States
(12) Patent Application Publication
(10) Pub. No.: US 2021/0340238 A1
(43) Pub. Date: Nov. 4, 2021

(54) TGFß1 INHIBITORS AND USE THEREOF

(71) Applicant: Scholar Rock, Inc., Cambridge, MA (US)

(72) Inventors: Abhishek Datta, Boston, MA (US); Thomas Schurpf, Cambridge, MA (US); Allan Capili, Somerville, MA (US); Stefan Wawersik, Westborough, MA (US); Christopher Chapron, Watertown, MA (US); Christopher Littlefield, Marblehead, MA (US); Gregory J. Carven, Maynard, MA (US); Kevin B. Dagbay, Brighton, MA (US); Susan Lin, Boston, MA (US); Justin W. Jackson, Cambridge, MA (US); Caitlin Stein, Lebanon, NH (US)

(21) Appl. No.: 17/258,908

(22) PCT Filed: Jul. 11, 2019
(86) PCT No.: PCT/US2019/041390

§ 371 Date: Jan. 8, 2021

Related U.S. Application Data
(60) Provisional application No. 62/757,917, filed on Nov. 9, 2018.
(60) Provisional application No. 62/722,081, filed on Aug. 23, 2018.
(60) Provisional application No. 62/696,774, filed on Jul. 11, 2018.

(51) Int. Cl. C07K 16/22; A61P 13/12

(52) U.S. Cl. CPC C07K 16/22; A61P 13/12; A61K 2039/507; C07K 2317/565; C07K 2317/94

(57) ABSTRACT

Disclosed herein are monoclonal antibodies and antigen-binding fragments thereof capable of selectively inhibiting TGFβ1. Related compositions, methods and therapeutic use are also disclosed.

### RELATED APPLICATIONS

This application is a U.S. national stage filing, under 35 U.S.C. § 371(c), of International Application No. PCT/US2019/041390, filed Jul. 11, 2019, which claims the benefit of and priority to U.S. Provisional Application No. 62/696,774 filed Jul. 11, 2018, 62/722,081 filed Aug. 23, 2018, and 62/757,917 filed Nov. 9, 2018, the contents of each of which are expressly incorporated herein by reference in entirety.

### SEQUENCE LISTING

The instant application contains a Sequence Listing which has been submitted electronically in ASCII format and is hereby incorporated by reference in its entirety. Said ASCII copy, created on Jul. 8, 2019, is named 127036-02320_SL.txt and is 244,219 bytes in size.

### BACKGROUND OF THE INVENTION

Transforming growth factor beta 1 (TGFβ1) is a member of the TGFβ superfamily of growth factors, along with two other structurally related isoforms, namely, TGFβ2 and TGFβ3, each of which is encoded by a separate gene. TGFβs function as pleiotropic cytokines that regulate cell proliferation, differentiation, immunomodulation (e.g., adaptive immune response), and other diverse biological processes both in homeostasis and in disease contexts. The three TGFβ isoforms signal through the same cell-surface receptors and trigger similar canonical downstream signal transduction events that include the SMAD2/3 pathway. However, gene knockout studies in mice show diverse phenotypes, suggesting that each isoform plays a discrete role in vivo. This may be achieved in part by differential expression patterns of the three isoforms.

Biological function of TGFβ1 is diverse. For example, TGFβ1 and has been implicated in a variety of biological processes including inhibition of cell growth, tissue homeostasis, extracellular matrix (ECM) remodeling, endothelial to mesenchymal transition, cell migration and invasion, and immune modulation/suppression, as well as epithelial-to-mesenchymal transition. In relation to ECM remodeling, TGFβ signaling may increase fibroblast populations and ECM deposition (e.g., collagen). In the immune system, TGFβ ligand modulates T regulatory cell function and maintenance of immune precursor cell growth and homeostasis. TGFβ dysregulation has been associated with a number of disease conditions, such as cancer, fibrosis and immune disorders.

For these and other reasons, TGFβ has been considered as an attractive therapeutic target for the treatment of fibrotic conditions, immune disorders, and various proliferative disorders. However, observations from preclinical studies, including in rats and dogs, have revealed serious toxicities associated with systemic inhibition of TGFβs in vivo. Moreover, although several TGFβ inhibitors have been developed to date, most clinical programs targeting TGFβ have been discontinued due to risk of serious side effects (summarized, for example, in WO 2017/156500). Thus, despite lines of direct and indirect evidence pointing to the involvement of TGFβ signaling in the progression of diseases such as fibrosis and cancer, there are no commercially available TGFβ inhibitors to date which are safe and efficacious.

Previously, Applicant described a class of monoclonal antibodies that functions with a novel mechanism of action to modulate growth factor signaling (see, for example, WO 2014/182676). These antibodies were designed to exploit the fact that TGFβ superfamily of growth factors are deposited as inactive (or “latent”) pro-protein complex comprised of prodomain and growth factor, which requires an activation step that triggers release of the mature growth factor from the latent complex. By taking advantage of this activation mechanism, Applicant sought to target the latent complex anchored in an extracellular niche. In comparison with the traditional approach of directly targeting the mature growth factor itself post-activation (e.g., neutralizing antibodies) or alternatively blocking its downstream receptors, the novel class of inhibitory antibodies specifically binds the pro-proprotein complex thereby preemptively blocking the activation step, upstream of ligand-receptor interaction. It was reasoned that this unique mechanism of action should provide advantages for achieving both spatial and temporal benefits in that they act at the source, that is, by targeting the latent proTGFβ1 complex within a disease microenvironment before activation takes place. Indeed, advantages of locally targeting tissue/cell-tethered complex at the source, as opposed to soluble active species (i.e., mature growth factors after being released from the source), are further supported by a recent study. Ishihara et al. (Sci. Transl. Med. 11, eaau3259 (2019) “Targeted antibody and cytokine cancer immunotherapies through collagen affinity”) reported that when systemically administered drugs are targeted to the affected tissue by conjugating with a collagen-binding moiety, they were able to enhance anti-tumor immunity and reduce treatment-related toxicities, as compared to non-targeted counterparts.

Accordingly, monoclonal antibodies that specifically bind and inhibit the activation step of TGFβ1 (that is, release of mature growth factor from the latent complex) in an isoform-selective manner were generated (see, WO 2017/156500). WO 2018/129329 further describes isoform-selective inhibitors capable of inhibiting the activation of TGFβ1 associated with multiple biological contexts. Indeed, the antibodies described therein were shown to be capable of targeting both the extracellular matrix (ECM)-associated TGFβ1 and immune cell-associated TGFβ1, thereby blocking the release of TGFβ1 from multiple sources, while maintaining the isoform-specificity. Data from a number of in vivo models showing efficacy and safety of isoform-selective TGFβ1 activation inhibitors were disclosed, demonstrating that such inhibitors are useful for the treatment of diseases that involve dysregulation of both ECM-associated TGFβ1 and immune cell-associated TGFβ1 in vivo. Data presented therein confirm the notion recognized in WO 2017/156500 that isoform-specific inhibition (as opposed to pan-inhibition) of TGFβ effectuated by the activation inhibitors can improve safety profiles in vivo.

While the earlier work described above demonstrated utility of antibodies capable of binding each of known proTGFβ1-presenting molecule complexes and inhibitory activities both in vitro and in vivo, the inventors of the present application setout to generate improved inhibitors of TGFβ1 activation having increased affinity, potency, and therapeutic efficacy.

### SUMMARY OF THE INVENTION

Disclosed herein are high-affinity, highly potent, isoform-selective inhibitors of TGFβ1. The TGFβ1-selective inhibitors according to the present disclosure are monoclonal antibodies (including immunoglobulins and antigen-binding fragments or portions thereof) which are capable of selectively inhibiting TGFβ1 signaling and meet the antibody criteria of one or more of the categories 1-5 in accordance with Table 1 herein. In some embodiments, the antibodies may be defined by amino acid sequences, such as CDRs and variable regions. In some embodiments, the antibodies may be defined by binding profiles, as determined by solution-equilibrium titration-based assays.

The invention includes compositions, such as pharmaceutical compositions (e.g., formulations, medicament) that are suitable for administration to human patients, comprising at least one of the antibodies or fragment thereof in accordance with the present disclosure, and an excipient. Thus, the antibodies or fragment thereof in accordance with the present disclosure can be used in the manufacture of such medicament.

The invention further provides therapeutic use of such antibodies. Thus, the TGFβ1-selective inhibitors (e.g., monoclonal antibodies or antigen-binding fragments thereof) of the present disclosure may be used in the treatment of TGFβ1-related indications in a subject. The TGFβ1-selective inhibitors may be particularly advantageous for treating such disease or disorders involving dysregulation of the extracellular matrix, including, for example, fibrotic disorders (such as organ fibrosis, and fibrosis involving chronic inflammation), proliferative disorders (such as cancer, e.g., solid tumors and myelofibrosis), disease involving endothelial-to-mesenchymal transition (EndMT), disease involving epithelial-to-mesenchymal transition (EMT), disease involving proteases, disease with aberrant gene expression of certain markers described herein. The TGFβ1-selective inhibitors may be used in conjunction with another therapy as combination therapies (e.g., add-on therapies). Methods for treating such disease or disorders comprising administration of the TGFβ1-selective inhibitor in a subject, either as monotherapy or combination therapy, are encompassed by the invention.

The present invention includes selection of subjects or patients who are likely to respond to or benefit from a TGFβ1 inhibition therapy. Related diagnostic methods, as well as methods for monitoring or determining therapeutic response to the TGFβ1 inhibition therapy, are encompassed herein.

Processes and methods for identifying or selecting TGFβ1-selective inhibitors suitable for therapeutic use are encompassed by the invention. An antibody or a plurality of antibodies that meets the criteria of one or more of the categories of Table 1 are selected. The selected antibody or the plurality of antibodies are evaluated in preclinical studies comprising an efficacy study and a toxcology/safety study, employing suitable preclinical models. Effective amounts of the antibody or the antibodies determined in the in efficacy study are below the level that results in undesirable toxicities determined in the toxcology/safety study. Preferably, the antibody or antibodies are selected which has/have at least 3-fold, 6-fold, and more preferably 10-fold therapeutic window. Effective amounts of the antibodies according to the present disclosure may be between about 0.1 mg/kg and about 30 mg/kg when administered weekly. In preferred embodiments, the maximally tolerated dose (MTD) of the antibodies according to the present disclosure is >100 mg/kg when dosed weekly for at least 4 weeks.

### BRIEF DESCRIPTION OF THE FIGURES

FIG. 1 is a graph that shows inhibition of LTBP1-proTGFβ activation in an LN229 assay.

FIG. 2 is a graph that shows inhibition of LTBP3-proTGFβ1 complex activation in LN229 cells.

FIG. 3 is a graph that shows inhibition of GARP pro-TGFβ1 activation in an SW480β6 assay.

FIG. 4 is a graph that shows inhibition of LRRC33-pro-TGFβ1 activation in an SW480β6 assay.

FIG. 5A is a graph that shows effect of Ab2 or Ab3 on expression of collagen genes (Col1a1 and Col3a1) in UUO mice. Mice were treated with 3, 10, or 30 mg/kg/wk of Ab3 or 3 or 10 mg/kg/week of Ab2. IgG alone was used as control.

FIG. 5B is a graph that shows effect of Ab3 or Ab2 on expression of Fn1 and Loxl2 genes in UUO mice. Mice were treated with 3, 10, or 30 mg/kg/wk of Ab3 or 3 or 10 mg/kg/week of Ab2. IgG alone was used as control.

FIG. 6 summarizes the statistical significance of the changes in gene expression (vs. UUO+IgG) after treatment in the UUO model.

FIG. 7A and FIG. 7B are graphs showing relative ratios of phosphorylated vs. total (phosphorylated and unphospohrylated) Smad2/3 in kidneys from a genetic model of Alport syndrome treated with and without antibodies Ab2 and Ab3. FIG. 7C is a graph showing the effect of Ab3 and Ab2 on gene expression in kidneys from a genetic model of Alport syndrome.

FIG. 8A is a graph that shows the serum exposure of Ab2 in the CDHFD mouse model at 6, 8, 10, and 12 weeks. FIG. 8B is a graph that shows the effect of Ab2 on Smad2/3 phosphorylation in liver tissue from CDHFD-treated mice. FIG. 8C is a graph that shows a correlation between reduced phosphorylated Smad2/3 and Ab2 exposure. FIG. 8D is a graph that shows a comparison of the effect of Ab3 and Ab2 on Smad2/3 phosphorylation in liver tissue from CDHFD-treated mice. FIG. 8E is a graph that shows the effect of Ab3 and Ab2 on liver fibrosis as measured by hydroxyproline levels. FIG. 8F is a graph that shows the effect of Ab2 on α-Col1 by IHC in CDHFD-treated mice. FIG. 8G is a graph that shows a correlation between Ab2 exposure levels and reduced α-Col1 levels.

FIG. 9 is a graph that shows the effect of Ab3 and Ab2 on picrosirius red staining (PRS) in a CCL4 mouse model of liver fibrosis.

FIG. 10A provides a HDX-MS heatmap for Ab3 Fab-LTBP3:ProTGFβ1 complex. FIG. 10B shows the protected regions by Ab3 on surface and ribbon structures of proTGFβ1. FIG. 10C provides an HDX-MS heatmap for Ab2 Fab-proTGFβ1 C4S complex. FIG. 10D shows the protected regions by Ab2 on surface and ribbon structures of proTGFβ1. Region 1 overlaps with so-called “Latency Lasso” within the prodomain of proTGFβ1, while Region 3 is within the growth factor domain. Sequence alignment among the three isoforms is also provided.

FIG. 11 provides the crystal structure of Ab2 Fab bound to proTGFβ1 and shows contact residues on proTGFβ1 and Ab2.

FIG. 12A depicts microscopic heart findings from a pan-TGFβ antibody from a 1-week rat toxcology study. FIG. 12B depicts microscopic heart findings from Ab3 as compared to an ALK5 inhibitor or pan-TGFβ antibody from a 4-week rat toxcology study. FIGS. 12C and 12D depict microscopic heart, bone, and lung, findings from Ab3 and Ab2 as compared to an ALK5 inhibitor or pan-TGFβ antibody from a 4-week rat toxicology study.

FIGS. 13A-13D provide relative expression of TGFβ isoforms. FIG. 13A shows TGFβ isoform expression vs. normal comparator (by cancer type). FIG. 13B shows frequency of TGFβ Isoform Expression by Human Cancer Type. FIG. 13C shows TGFβ isoform expression in individual tumor samples, by cancer type. FIG. 13D shows TGFβ isoform expression in mouse syngeneic cancer cell model lines.

FIG. 14 provides a set of graphs that shows the change in tumor growth (tumor volume mm3) measured over time (days) after administration of Ab3 at 30 mg/kg or 10 mg/kg or Ab2 at 3 mg/kg or 10 mg/kg, in combination with anti-PD1 (P<0.05, Mann-Whitney U test) in MBT-2 tumor model. Anti-PD1 alone was used as a control.

FIG. 15 is a graph that shows the median tumor volume (mm³) at day 15 in mice administered Ab3 at 30 mg/kg or 10 mg/kg or Ab2 at 3 mg/kg or 10 mg/kg, in combination with anti-PD1 (P<0.05, Mann-Whitney U test) in MBT-2 tumor model.

FIG. 16 provides a graph showing the S91 median tumor volumes as a function of time. The combination arms represent two different isoform-selective, TGFβ1 inhibitors (Ab3 and Ab2) at two dose levels, each in combination with anti-PD-1 treatment.

FIGS. 17A and 17B provide representative immunohistochemistry sections of S91 tumor model, stained with a CD8+ cell marker. FIG. 17A is a tumor section from an animal treated with anti-PD-1 alone. FIG. 17B is a tumor section from an animal treated with both anti-PD-1 and a representative context-independent TGFβ1 inhibitor.

FIGS. 18A and 18B provide representative immunohistochemistry sections of S91 tumor, stained with a macrophage marker. FIG. 18A is a tumor section from an animal treated with anti-PD-1 alone. FIG. 18B is a tumor section from an animal treated with both anti-PD-1 and a representative context-independent TGFβ1 inhibitor.

FIG. 19 provides a graph showing the association and dissociation of Ab2 to TGFβ1 C4S at different pHs.

### DETAILED DESCRIPTION OF CERTAIN EMBODIMENTS

Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

Advanced cancer, advanced malignancy: The term “advanced cancer” or “advanced malignancy” as used herein has the meaning understood in the pertinent art, e.g., as understood by oncologists in the context of diagnosing or treating subjects/patients with cancer. Advanced malignancy with a solid tumor can be locally advanced or metastatic. The term “locally advanced cancer” is used to describe a cancer (e.g., tumor) that has grown outside the organ it started in but has not yet spread to distant parts of the body. Thus, the term includes cancer that has spread from where it started to nearby tissue or lymph nodes. By contrast, “metastatic cancer” is a cancer that has spread from the part of the body where it started (the primary site) to other parts (e.g., distant parts) of the body.

Affinity: Affinity is the strength of binding of a molecule (such as an antibody) to its ligand (such as an antigen). It is typically measured and reported by the equilibrium dissociation constant (KD). In the context of antibody-antigen interactions, KD is the ratio of the antibody dissociation rate (“off rate” or Koff or Kdis), how quickly it dissociates from its antigen, to the antibody association rate (“on rate” or Kon) of the antibody, how quickly it binds to its antigen. For example, an antibody with an affinity of ≤5 nM has a KD value that is 5 nM or lower (i.e., 5 nM or higher affinity) determined by a suitable in vitro binding assay. Suitable in vitro assays can be used to measure KD values of an antibody for its antigen, such as Biolayer Interferometry (BLI) and Solution Equilibrium Titration (e.g., MSD-SET).

Antibody: The term “antibody” encompasses any naturally-occurring, recombinant, modified or engineered immunoglobulin or immunoglobulin-like structure or antigen-binding fragment or portion thereof, or derivative thereof, as further described elsewhere herein. Unless specified to the contrary, the term “antigen” as used herein shall encompass antigen-binding fragments and functional variants thereof. Thus, the term refers to an immunoglobuIin molecule that specifically binds to a target antigen, and includes, for instance, chimeric, humanized, fully human, and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen-binding portions thereof, can be produced in hybridomas, by recombinant DNA techniques, or by enzymatic or chemical cleavage of intact antibodies. The term antibodies, as used herein, includes monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), respectively. In some embodiments, the term also encompasses peptibodies.

Antigen: The term “antigen” broadly includes any molecules comprising an antigenic determinant within a binding region(s) to which an antibody or a binding-fragment specifically binds. An antigen can be a single-unit molecule (such as a protein monomer or a fragment) or a complex comprised of multiple components. An antigen provides an epitope, e.g., a molecule or a portion of a molecule, or a complex of molecules or portions of molecules, capable of being bound by a selective binding agent, such as an antigen-binding protein (including, e.g., an antibody). Thus, a selective binding agent may specifically bind to an antigen that is formed by two or more components in a complex. In some embodiments, the antigen is capable of being used in an animal to produce antibodies capable of binding to that antigen. An antigen can possess one or more epitopes that are capable of interacting with different antigen-binding proteins, e.g., antibodies. In the context of the present disclosure, a suitable antigen is a complex (e.g., multimeric complex comprised of multiple components in association) containing a proTGF dimerin association with a presenting molecule. Each monomer of the proTGF dimer comprises a prodomain and a growth factor domain, separated by a furin cleavage sequence. Two such monomers form the proTGF dimer complex. This in turn is covalently associated with a presenting molecule via disulfide bonds, which involve a cysteine residue present near the N-terminus of each of the proTGF monomer. This multi-complex formed by a proTGF dimer bound to a presenting molecule is generally referred to as a large latent complex. An antigen complex suitable for screening antibodies or antigen-binding fragments, for example, includes a presenting molecule component of a large latent complex Such presenting molecule component may be a full-length presenting molecule or a fragment(s) thereof. Minimum required portions of the presenting molecule typically contain at least 50 amino acids, but more preferably at least 100 amino acids of the presenting molecule polypeptide, which comprises two cysteine residues capable of forming covalent bonds with the proTGFβ1 dimer.

Antigen-binding portion/fragment: The terms “antigen-binding portion” or “antigen-binding fragment” of an antibody, as used herein, refers to one or more fragments of an antibody that retain the ability to specifically bind to an antigen (e.g., TGFβ1). Antigen-binding portions include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments, a monovalent fragment consisting of the VL, VH, CL and CH1 domains; (ii) F(ab′)2 fragments, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv (scFv) molecules (see, e.g., Bird et al. (1988) SCIENCE 242:423-426; and Huston et al. (1988) PROC. NAT'L. ACAD. SCI. USA 85:5879-5883); (vi) dAb fragments (see, e.g., Ward et al. (1989) NATURE 341: 544-546); and (vii) minimal recognition units consisting of the amino acid residues that mimic the hypervariable region of an antibody (e.g., an isolated complementarity determining region (CDR)). Other forms of single chain antibodies, such as diabodies are also encompassed. The term antigen-binding portion of an antibody includes a “s ingle chain Fab fragment” otherwise known as an “scFab,” comprising an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids.

Bias: In the context of the present disclosure, the term “bias” refers to skewed or uneven affinity towards or against a subset of antigens to which an antibody is capable of specifically binding. For example, an antibody is said to have bias when the affinity for one antigen complex and the affinity for another antigen complex are not equivalent (e.g., more than five-fold difference in affinity). Preferred antibodies of the present disclosure include “matrix-biased” (or “LTBP-biased”) antibodies, which preferentially bind EMC-associated complexes (LTBP1-proTGFβ1 and LTBP3-proTGFβ), such that relative affinities between at least one of the matrix-associated complexes and at least one of the cell-associated complexes (GARP-proTGFβ1 and/or LRRC33-proTGFβ1 complexes) is greater than five-fold. By comparison, antibodies characterized as “unbiased” have approximately equivalent affinities towards such antigen complexes (e.g., less than five-fold difference in affinity).

Binding region: As used herein, a “binding region” is a portion of an antigen (e.g., an antigen complex) that, when bound to an antibody or a fragment thereof, can form an interface of the antibody-antigen interaction. Upon antibody binding, a binding region becomes “protected” from surface exposure, which can be detected by suitable techniques, such as HDX-MS. Antibody-antigen interaction may be mediated via multiple (e.g., two or more) binding regions. A binding region can comprise an antigenic determinant, or epitope.

Cancer: The term “cancer” as used herein refers to the physiological condition in multicellular eukaryotes that is typically characterized by unregulated cell proliferation and malignancy. The term broadly encompasses, solid and liquid malignancies, including tumors, blood cancers (e.g., leukemias, lymphomas and myelomas), as well as myelofibrosis.

Cell-associated TGFβ1/proTGFβ1: The term refers to TGFβ1 or its signaling complex (e.g., pro/latentTGFβ1) that is membrane-bound (e.g., tethered to cell surface). Typically, such cell is an immune cell. TGFβ1 that is presented by GARP or LRRC33 is a cell-associated TGFβ1. GARP and LRRC33 are transmembrane presenting molecules that are expressed on cell surface of certain cells. GARP-proTGFβ1 and LRRC33-proTGFβ1 may be collectively referred to as “cell-associated” (or “cell-surface”) proTGFβ1 complexes, that mediate cell-associated (e.g., immune cell-associated) TGFβ1 activation/signaling. The term also includes recombinant, purified GARP-proTGFβ1 and LRRC33-proTGFβ1 complexes in solution (e.g., in vitro assays) which are not physically attached to cell membranes. Average KD values of an antibody (or its fragment) to a GARP-proTGFβ1 complex and an LRRC33-proTGFβ1 complex may be calculated to collectively represent affinities for cell-associated (e.g., immune cell-associated) proTGFβ1 complexes. See, for example, Table, column (G). Human counterpart of a presenting molecule or presenting molecule complex may be indicated by an “h” preceding the protein or protein complex, e.g., “hGARP,” “hGARP-proTGFβ1,” hLRRC33” and “hLRRC33-proTGFβ1.”

Checkpoint inhibitor: In the context of this disclosure, checkpoint inhibitors refer to immune checkpoint inhibitors and carries the meaning as understood in the art. Typically, target is a receptor molecule on T cells or N K cells, or corresponding cell surface ligand on antigen-presenting cells (APCs) or tumor cells. Immune checkpoints are activated in immune cells to prevent inflammatory immunity developing against the “self”. Therefore, changing the balance of the immune system via checkpoint inhibition should allow it to be fully activated to detect and eliminate the cancer. The best known inhibitory receptors implicated in control of the immune response are cytotoxic T-lymphocyte antigen-4 (CTLA-4), programmed cell death protein 1 (PD-1), PD-L1, T-cell immunoglobulin domain and mucin domain-3 (TIM3), lymphocyte-activation gene 3 (LAG3), killer cell immunoglobulin-like receptor (KIR), glucocorticoid-induced tumor necrosis factor receptor (GITR) and V-domain immunoglobulin (Ig)-containing suppressor of T-cell activation (VISTA). Non-limiting examples of checkpoint inhibitors include: Nivolumab, Pembrolizumab, BMS-936559, Atezolizumab, Avelumab, Durvalumab, Ipilimumab, Tremelimumab, IMP-321, BMS-986016, and Lirilumab. Keytruda® is one example of PD-1 inhibitors. Therapies or therapeutic regimens that employ one or more of immune checkpoint inhibitors may be referred to as checkpoint blockade therapy (CBT).

Clinical benefit: As used herein, the term “clinical benefits” is intended to include both efficacy and safety of a therapy. Thus, therapeutic treatment that achieves a desirable clinical benefit is both efficacious and safe (e.g., with tolerable or acceptable toxicities or adverse events).

Combination therapy: “Combination therapy” refers to treatment regimens for a clinical indication that comprise two or more therapeutic agents. Thus, the term refers to a therapeutic regimen in which a first therapy comprising a first composition (e.g., active ingredient) is administered in conjunction with a second therapy comprising a second composition (active ingredient) to a patient, intended to treat the same or overlapping disease or clinical condition. The first and second compositions may both act on the same cellular target, or discrete cellular targets. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies. When a subject who has been treated with a first therapy for the treatment of a disease is administered with a second therapy to treat the same disease, the second therapy may be referred to as an “add-on therapy” or “adjunct therapy.”

Combinatory or combinatorial epitope: A combinatorial epitope is an epitope that is recognized and bound by a combinatorial antibody at a site (i.e., antigenic determinant) formed by non-contiguous portions of a component or components of an antigen, which, in a three-dimensional structure, come together in close proxmity to form the epitope. Thus, antibodies of the invention may bind an epitope formed by two or more components (e.g., portions or segments) of a pro/latentTGFβ1 complex. A combinatory epitope may comprise amino acid residue(s) from a first component of the complex, and amino acid residue(s) from a second component of the complex, and so on. Each component may be of a single protein or of two or more proteins of an antigenic complex. A combinatory epitope is formed with structural contributions from two or more components (e.g., portions or segments, such as amino acid residues) of an antigen or antigen complex.

Compete or cross-compete: The term “compete” when used in the context of antigen-binding proteins (e.g., an antibody or antigen-binding portion thereof) that compete for the same epitope means competition between antigen-binding proteins as determined by an assay in which the antigen-binding protein being tested prevents or inhibits (e.g., reduces) specific binding of a reference antigen-binding protein to a common antigen (e.g., TGFβ1 or a fragment thereof). Numerous types of competitive binding assays can be used to determine if one antigen-binding protein competes with another, for example: solid phase director indirect radioimmunoassay (RIA), solid phase direct or indirect enzyme immunoassay (EIA), sandwich competition assay; solid phase direct biotin-avidin EIA; solid phase direct labeled assay, and solid phase direct labeled sandwich assay. Usually, when a competing antigen-binding protein is present in excess, it will inhibit (e.g., reduce) specific binding of a reference antigen-binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more. In some embodiments, a first antibody or antigen-binding portion thereof and a second antibody or antigen-binding portion thereof cross-block with each other with respect to the same antigen, for example, as assayed by BLI (such as Biacoror Octet®), using standard test conditions, e.g., according to the manufacturer's instructions (e.g., binding assayed at room temperature, ˜20-25° C.). In some embodiments, the first antibody or fragment thereof and the second antibody or fragment thereof may have the same epitope. In other embodiments, the first antibody or fragment thereof and the second antibody or fragment thereof may have non-identical but overlapping epitopes. In yet further embodiments, the first antibody or fragment thereof and the second antibody or fragment thereof may have separate (different) epitopes which are in close proximity in a three-dimensional space, such that antibody binding is cross-blocked via steric hindrance. “Cross-block” means that binding of the first antibody to an antigen prevents binding of the second antibody to the same antigen, and similarly, binding of the second antibody to an antigen prevents binding of the first antibody to the same antigen.

Complementary determining region (CDR): As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al. (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L-CDR1, L-CDR2 and L-CDR3 or H-CDR1, H-CDR2 and H-CDR3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen-binding (see, for example: Lu X et al., MAbs. 2019 January; 11(1):45-57). The methods used herein may utilize CDRs defined according to any of these systems, although certain embodiments use Kabat- or Chothia-defined CDRs.

Conformational epitope: A conformational epitope is an epitope that is recognized and bound by a conformational antibody in a three-dimensional conformation, but not in an unfolded peptide of the same amino acid sequence. A conformational epitope may be referred to as a conformation-specific epitope, conformation-dependent epitope, or conformation-sensitive epitope. A corresponding antibody or fragment thereof that specifically binds such an epitope may be referred to as conformation-specific antibody, conformation-selective antibody, or conformation-dependent antibody. Binding of an antigen to a conformational epitope depends on the three-dimensional structure (conformation) of the antigen or antigen complex.

Constant region/domain: An immunoglobulin constant domain refers to a heavy or light chain constant domain. Human IgG heavy chain and light chain constant domain amino acid sequences are known in the art.

Context-biased: As used herein, “context-biased antibodies” refer to a type of conformational antibodies that binds an antigen with differential affinities when the antigen is associated with (i.e., bound to or attached to) an interacting protein or a fragment thereof. Thus, a context-biased antibody that specifically binds an epitope within proTGFβ1 may bind LTBP1-proTGFβ1, LTBP3-proTGFβ1, GARP-proTGFβ1 and LRRC33-proTGFβ1 with different affinities. For example, an antibody is said to be “matrix-biased” if it has higher affinities for matrix-associated proTGFβ1 complexes (e.g., LTBP1-proTGFβ1 and LTBP3-proTGFβ1) than for cell-associated proTGFβ1 complexes (e.g., GARP-proTGFβ1 and LRRC33-proTGFβ1). Relative affinities of [matrix-associated complexes]: [cell-associated complexes] may be obtained by taking average KD values of the former, taking average KD values of the latter, and calculating the ratio of the two, as exemplified herein. A context-biased antibody may also be biased for or against one presenting molecule-proTGFβ1 complex relative to the other presenting molecule-proTGFβ1 complexes, such that the affinity (as measured by KD) for the former is more than 10-fold weaker or greater than the average of the latter, respectively.

Context-independent: According to the present disclosure, “a context-independent antibody” that binds proTGFβ1 has equivalent affinities across the four known presenting molecule-proTGFβ1 complexes, namely, LTBP1-proTGFβ1, LTBP3-proTGFβ1, GARP-proTGFβ1 and LRRC33-proTGFβ1. Context-independent antibodies may also be characterized as “unbiased” or “balanced.” Typically, context-independent antibodies show no more than five-fold bias in affinities, such that relative ratios of measured KD values between matrix-associated complexes and cell-associated complexes are no greater than 5 as measured by a suitable in vitro binding assay, such as surface plasmon resonance, Biolayer Interferometry (BLI), and/or solution equilibrium titration (e.g., MSD-SET).

ECM-associated TGFβ1/proTGFβ1: The term refers to TGFβ1 or its signaling complex (e.g., pro/latent TGFβ1) that is a component of (e.g., deposited into) the extracellular matrix. TGFβ1 that is presented by LTBP1 or LTBP3 is an ECM-associated TGFβ1. LTBPs are critical for correct deposition and subsequent bioavailability of TGFβ in the ECM, where fibrillin (Fbn) and fibronectin (FN) are believed to be the main matrix proteins responsible for the association of LTBPs with the ECM. Average KD values of an antibody (or its fragment) to an LTBP1-proTGFβ1 complex and an LTBP3-proTGFβ1 complex may be calculated to collectively represent affinities for ECM-associated (or matrix-associated) proTGFβ1 complexes. See, for example, Table, column (D). Human counterpart of a presenting molecule or presenting molecule complex may be indicated by an “h” preceding the protein or protein complex e.g., “hLTBP1,” “hLTBP1-proTGFβ1,” hLTBP3” and “hLTBP3-proTGFβ1.”

Effective amount: An “effective amount” (or therapeutically effective amount, or therapeutic dose) is a dosage, concentration, or dosing regimen that achieves statistically significant clinical benefits (e.g., efficacy) in a patient population. For example, Ab2 has been shown to be efficacious at doses as low as 3 mg/kg or less and as high as 30 mg/kg in preclinical models. Thus, an effective amount for Ab2 can be said to be between about 3-30 mg/kg.

Effective tumor control: The term “effective tumor control” may be used to refer to a degree of tumor regression achieved in response to treatment, where, for example, the tumor is regressed by a defined fraction (such as <25%) of an endpoint tumor volume. For instance, in a particular model, if the endpoint tumor volume is set at 2,000 mm³, effective tumor control is achieved if the tumor is reduced to less than 500 mm³ assuming the threshold of <25%. Therefore, effective tumor control encompasses complete regression. Clinically, effective tumor control includes partial response (PR) and complete response (CR) based on art-recognized criteria, such as RECIST 1.1 and corresponding iRECIST. In some embodiments, effective tumor control in clinical settings also includes stable disease, where tumors that are typically expected to grow at certain rates are prevented from such growth by the treatment, even though shrinkage is not achieved.

Effector T cells: Effector T cells, as used herein, are T lymphocytes that actively respond immediately to a stimulus, such as co-stimulation and include, but are not limited to, CD4+ T cells (also referred to as T helper or Th cells) and CD8+ T cells (also referred to as cytotoxic T cells). Th cells assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including Th1, Th2, Th3, Th17, Th9, or TFh, which secrete different cytokines to facilitate different types of immune responses. Signaling from the APC directs T cells into particular subtypes. Cytotoxc (Killer). Cytotoxc T cells (TC cells, CTLs, T-killer cells, killer T cells), on the other hand, destroy virus-infected cells and cancer cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Cytotoxic effector cell (e.g., CD8+ cells) include, e.g., perforin and granzyme B.

Epitope: The term “epitope” may be also referred to as an antigenic determinant, is a molecular determinant (e.g., polypeptide determinant) that can be specifically bound by a binding agent, immunoglobulin or T-cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody specifically binds an antigen when it recognizes its target antigen in a complex mixture of proteins and/or macromolecules.

Fibrosis: The term “fibrosis” or “fibrotic condition/disorder” refers to the process or manifestation characterized by the pathological accumulation of extracellular matrix (ECM) components, such as collagens, within a tissue or organ.

Fibrotic microenvironment: The term “fibrotic microenvironment” refers to a local disease niche within a tissue, in which fibrosis occurs in vivo. The fibrotic microenvironment may comprise disease-associated molecular signature (a set of chemokines, cytokines, etc.), disease-associated cell populations (such as activated macrophages, MDSCs, etc.) as well as disease-associated ECM environments (alterations in ECM components and/or structure). Fibrotic microenvironment is thought to support the transition of fibroblast to α-smooth muscle actin-positive myofibroblastin a TGFβ-dependent manner. Fibrotic microenvironment may be further characterized by the infiltration of certain immune cells (such as macrophages and MDSCs).

GARP-TGFβ1 complex: As used herein, the term “GARP-TGFβ1 complex” (or “GARP-proTGFβ1 complex”) refers to a protein complex comprising a pro-protein form or latent form of a transforming growth factor-β1 (TGFβ1) protein and a glycoprotein-A repetitions predominant protein (GARP) or fragment or variant thereof. In some embodiments, a pro-protein form or latent form of TGFβ1 protein may be referred to as “pro/latent TGFβ1 protein”. In some embodiments, a GARP-TGFβ1 complex comprises GARP covalently linked with pro/latentTGFβ1 via one or more disulfide bonds. In nature, such covalent bonds are formed with cysteine residues present near the N-terminus (e.g., amino acid position 4) of a proTGFβ1 dimer complex. In other embodiments, a GARP-TGFβ1 complex comprises GARP non-covalently linked with pro/latentTGFβ1. In some embodiments, a GARP-TGFβ1 complex is a naturally-occurring complex, for example a GARP-TGFβ1 complex in a cell. The term “hGARP” denotes human GARP.

Human antibody: The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Humanized antibody: The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment a humanized antibody also comprises at least a portion of an immunoglobulin Fc region, typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

Hydrogen/deuterium exchange mass spectrometry (HDX-MS): HDX-MS is a well-known technique employed to interrogate protein confirmation and protein-protein interactions in solution by measuring the degree of solvent accessibility. See, for example, Wei et al., (2014) Drug DiscovToday 19(1): 95-102. “Hydrogen/deuterium exchange mass spectrometry for probing higher order structure of protein therapeutics: methodology and applications.” The HDX-MS technique may be employed to determine a region or regions of an antigen bound by an antibody (i.e., “binding region(s)”). Thus, such binding region(s) may contain or form an epitope.

Immunosuppression, immunosuppressive: The terms refer to the ability to suppress immune cells, such as T cells, NK cells and B cells. The gold standard for evaluating immunosuppressive function is the inhibition of T cell activity, which may include antigen-specific suppression and non-specific suppression. Regulatory T cells (Tregs) and MDSCs may be considered immunosuppressive cells. M2-polarized macrophages (e.g., TAMs) may also be characterized as immunosuppressive.

Isoform-specific/selective: The term “isoform specificity” or “isoform selectivity” refers to an agent's ability to discriminate one isoform over other structurally related isoforms (i.e., selectivity). An isoform-specific TGFβ inhibitor exerts its inhibitory activity towards one isoform of TGFβ but not the other isoforms of TGFβ at a given concentration. For example, an isoform-specific TGFβ1 antibody selectively binds TGFβ1. A TGFβ1-specific inhibitor (antibody) preferentially targets (binds thereby inhibits) the TGFβ1 isoform over TGFβ2 or TGFβ3 with substantially greater affinity. For example, the selectivity in this context may refer to at least a 500-1000-fold difference in respective affinities as measured by an in vitro binding assay such as Octet® and Biacor®. In some embodiments, the selectivity is such that the inhibitor when used at a dosage effective to inhibit TGFβ1 in vivo does not inhibit TGFβ2 and TGFβ3. For instance, an antibody may preferentially bind TGFβ1 at affinity of ˜1 pM, while the same antibody may bind TGFβ2 and/or TGFβ3 at ˜0.5-50 nM. For such an inhibitor to be useful as a therapeutic, dosage to achieve desirable effects (e.g., therapeutically effective amounts) must fall within the window within which the inhibitor can effectively inhibit the TGFβ1 isoform without inhibiting TGFβ2 or TGFβ3.

Isolated: An “isolated” antibody as used herein, refers to an antibody that is substantially free of other antibodies having different antigenic specificities. In some embodiments, an isolated antibody is substantially free of other unintended cellular material and/or chemicals.

Localized: In the context of the present disclosure, the term “localized” (as in “localized tumor”) refers to anatomically isolated or isolatable abnormalities, such as solid malignancies, as opposed to systemic disease. Certain leukemia, for example, may have both a localized component (for instance the bone marrow) and a systemic component (for instance circulating blood cells) to the disease.

LRRC33-TGFβ1 complex: As used herein, the term “LRRC33-TGFβ1 complex” (or “LRRC33-proTGFβ1 complex”) refers to a complex between a pro-protein form or latent form of transforming growth factor-β1 (TGFβ1) protein and a Leucine-Rich Repeat-Containing Protein 33 (LRRC33; also known as Negative Regulator Of Reactive Oxygen Species or NRROS) or fragment or variant thereof. In some embodiments, a LRRC33-TGFβ1 complex comprises LRRC33 covalently linked with pro/latentTGFβ1 via one or more disulfide bonds. In nature, such covalent bonds are formed with cysteine residues present near the N-terminus (e.g., amino acid position 4) of a proTGFβ1 dimer complex. In other embodiments, a LRRC33-TGFβ1 complex comprises LRRC33 non-covalently linked with pro/latentTGFβ1. In some embodiments, a LRRC33-TGFβ1 complex is a naturally-occurring complex, for example a LRRC33-TGFβ1 complex in a cell. The term “hLRRC33” denotes human LRRC33.

LTBP1-TGFβ1 complex: As used herein, the term “LTBP1-TGFβ1 complex” (or “LTBP1-proTGFβ1 complex”) refers to a protein complex comprising a pro-protein form or latent form of transforming growth factor-β1 (TGFβ1) protein and a latent TGF-beta binding protein 1 (LTBP1) or fragment or variant thereof. In some embodiments, a LTBP1-TGFβ1 complex comprises LTBP1 covalently linked with pro/latentTGFβ1 via one or more disulfide bonds. In nature, such covalent bonds are formed with cysteine residues present near the N-terminus (e.g., amino acid position 4) of a proTGFβ1 dimer complex. In other embodiments, a LTBP1-TGFβ1 complex comprises LTBP1 non-covalently linked with pro/latent TGFβ1. In some embodiments, a LTBP1-TGFβ1 complex is a naturally-occurring complex for example a LTBP1-TGFβ1 complex in a cell. The term “hLTBP1” denotes human LTBP1.

LTBP3-TGFβ1 complex: As used herein, the term “LTBP3-TGFβ1 complex” (or “LTBP3-proTGFβ1 complex”) refers to a protein complex comprising a pro-protein form or latent form of transforming growth factor-β1 (TGFβ1) protein and a latent TGF-beta binding protein 3 (LTBP3) or fragment or variant thereof. In some embodiments, a LTBP3-TGFβ1 complex comprises LTBP3 covalently linked with pro/latentTGFβ1 via one or more disulfide bonds. In nature, such covalent bonds are formed with cysteine residues present near the N-terminus (e.g., amino acid position 4) of a proTGFβ1 dimer complex. In other embodiments, a LTBP3-TGFβ1 complex comprises LTBP1 non-covalently linked with pro/latent TGFβ1. In some embodiments, a LTBP3-TGFβ1 complex is a naturally-occurring complex for example a LTBP3-TGFβ1 complex in a cell. The term “hLTBP3” denotes human LTBP3.

M2 or M2-like macrophage: M2 macrophages represent a subset of activated or polarized macrophages and include disease-associated macrophages in both fibrotic and tumor microenvironments. Cell-surface markers for M2-polarized macrophages typically include CD206 and CD163 (i.e., CD206+/CD163+). Applicant recently discovered that the M2-polarized macrophages may also express cell-surface LRRC33. The activation of M2 macrophages is promoted mainly by IL-4, IL-13, IL-10 and TGFβ; they secrete the same cytokines that activate them (IL-4, IL-13, IL-10 and TGFβ). These cells have high phagocytic capacity and produce ECM components, angiogenic and chemotactic factors. The release of TGFβ by macrophages may perpetuate the myofibroblast activation, EMT and EndMT induction in the fibrotic tissue. For example, M2 macrophages are essential for TGFβ-driven lung fibrosis and are also enriched in a number of tumors.

Matrix-associated proTGFβ1: LTBP1 and LTBP3 are presenting molecules that are components of the extracellular matrix (ECM). LTBP1-proTGFβ1 and LTBP3-proTGFβ1 may be collectively referred to as “ECM-associated” (or “matrix-associated”) proTGFβ1 complexes, which mediate ECM-associated TGFβ1 activation/signaling. The term also includes recombinant, purified LTBP1-proTGFβ1 and LTBP3-proTGFβ1 complexes in solution (e.g., in vitro assays) which are not physically attached to a matrix or substrate.

Maximally tolerated dose (MTD): The term MTD generally refers to, in the context of safety/toxcology considerations, the highest amount of a test article (such as a TGFβ1 inhibitor) evaluated with no observed adverse effect level (NOAEL). For example, the NOAEL for Ab2 in rats was the highest dose evaluated (100 mg/kg), suggesting that the MTD for Ab2 is >100 mg/kg, based on a four-week toxicology study.

Meso-Scale Discovery: “Meso-Scale Discovery” or “MSD” is a type of immunoassay that employs high binding carbon electrodes to capture proteins (e.g., antibodies). The antibodies can be incubated with particular antigens, which binding can be detected with secondary antibodies that are conjugated to electrochemiluminescent labels. Upon an electrical signal, light intensity can be measured to quantify analytes in the sample.

Myelofibrosis: “Myelofibrosis,” also known as osteomyelofibrosis, is a relatively rare bone marrow proliferative disorder (e.g., cancer), which belongs to a group of diseases called myeloproliferative disorders and includes primary myelofibrosis and secondary myelofibrosis. Myelofibrosis characterized by the proliferation of an abnormal clone of hematopoieticstem cells in the bone marrow and other sites results in fibrosis, or the replacement of the marrow with scar tissue. Myelofibrosis is characterized by mutations that cause upregulation or overactivation of the downstream JAK pathway.

Myeloid-derived suppressor cell: Myeloid-derived suppressor cells (MDSCs) are a heterogeneous population of cells generated during various pathologic conditions and thought to represent a pathologic state of activation of monocytes and relatively immature neutrophils. MDSCs include at least two categories of cells termed i) “granulocytic” (G-MDSC) or polymorphonuclear (PMN-MDSC), which are phenotypically and morphologically similar to neutrophils; and ii) monocytic (M-MDSC) which are phenotypically and morphologically similar to monocytes. MDSCs are characterized by a distinct set of genomic and biochemical features, and can be distinguished by specific surface molecules. For example, human G-MDSCs/PMN-MDSCs typically express the cell-surface markers CD11b, CD33, CD15 and CD66. In addition, human G-MDSCs/PMN-MDSCs may also express HLA-DR and/or Arginase. By comparison, human M-MDSCs typically express the cell surface markers CD11b, CD33 and CD14. The MDSCs may also express CD39 and CD73 to mediate adenosine signaling involved in organ fibrosis (such as liver fibrosis, and lung fibrosis), cancer and myelofibrosis). In addition, human M-MDSCs may also express HLA-DR. In addition to such cell-surface markers, MDSCs are characterized by the ability to suppress immune cells, such as T cells, NK cells and B cells. Immune suppressive functions of MDSCs may include inhibition of antigen-non-specific function and inhibition of antigen-specific function. MDSCs can express cell surface LRRC33 and/or LRRC33-proTGFβ1.

Myofibroblast: Myofibroblasts are cells with certain phenotypes of fibroblasts and smooth muscle cells and generally express vimentin, alpha-smooth muscle actin (α-SMA; human gene ACTA2) and paladin. In many disease conditions involving extracellular matrix dysregulations (such as increased matrix stiffness), normal fibroblast cells become de-differentiated into myofibroblasts in a TGFβ-dependent manner. Aberrant overexpression of TGFβ is common among myofibroblast-driven pathologies. TGFβ is known to promote myofibroblast differentiation, cell proliferation, and matrix production. Myofibroblasts or myofibroblast-like cells within the fibrotic microenvironment may be referred to as fibrosis-associated fibroblasts (or “FAFs”), and myofibroblasts or myofibroblast-like cells within the tumor microenvironment may be referred to as cancer-associated fibroblasts (or “CAFs”).

Pan-TGFβ inhibitor/pan-inhibition of TGFβ: The term “pan-TGFβ inhibitor” or “pan inhibitor of TGFβ” refers to any agent that is capable of inhibiting or antagonizing all three isoforms of TGFβ. Such an inhibitor may be a small molecule inhibitor of TGFβ isoforms. The term includes pan-TGFβ antibody which refers to any antibody capable of binding to each of TGFβ isoforms, i.e., TGFβ1, TGFβ2, and TGFβ3. In some embodiments, a pan-TGFβ antibody binds and neutralizes activities of all three isoforms, i.e., TGFβ1, TGFβ2, and TGFβ3 activities. The antibody 1 D11 (or the human analog Fresolimumab (GC1008)) is a well-known example of a pan-TGFβ antibody that neutralizes all three isoforms of TGFβ. Examples of small molecule pan-TGFβ inhibitors include galunisertib (LY2157299 monohydrate), which is an antagonist for the TGFβ receptor I kinase/ALK5 that mediates signaling of all three TGFβ isoforms.

Potency: The term “potency” as used herein refers to activity of a drug, such as an inhibitory antibody (or fragment) having inhibitory activity, with respect to concentration or amount of the drug to produce a defined effect. For example, an antibody capable of producing certain effects at a given dosage is more potent than another antibody that requires twice the amount (dosage) to produce equivalent effects. Potency may be measured in cell-based assays, such as TGFβ activation/inhibition assays, whereby the degree of TGFβ activation, such as activation triggered by integrin binding, can be measured in the presence or absence of test article (e.g., inhibitory antibodies) in a cell-based system. Typically, among those capable of binding to the same or overlapping binding regions of an antigen (e.g., cross-blocking antibodies), antibodies with higher affinities (lower KD values) tend to show higher potency than antibodies with lower affinities (greater KD values).

Presenting molecule: Presenting molecules in the context of the present disclosure refer to anchoring proteins that can form covalent bonds with latent pro-proteins (e.g., proTGFβ1) and “present” the inactive complex in an extracellular niche (such as ECM or immune cell surface) thereby maintaining its latency until an activation event occurs. Known presenting molecules for proTGFβ1 include: LTBP1, LTBP3, GARP (also known as LRRC32) and LRRC33, which can form presenting molecule-proTGFβ1 complexes (LLCs), namely, LTBP1-proTGFβ1, LTBP3-proTGFβ1, GARP-proTGFβ1 and LRRC33-proTGFβ1, respectively. In nature, LTBP1 and LTBP3 are components of the extracellular matrix (ECM); therefore, LTBP1-proTGFβ1 and LTBP3-proTGFβ1 may be collectively referred to as “ECM-associated” (or “matrix-associated”) proTGFβ1 complexes that mediate ECM-associated TGFβ1 signaling/activities. GARP and LRRC33, on the other hand, are trans membrane proteins expressed on cell surface of certain cells; therefore, GARP-proTGFβ1 and LRRC33-proTGFβ1 may be collectively referred to as “cell-associated”(or “cell-surface”) proTGFβ1 complexes, that mediate cell-associated (e.g., immune cell-associated) TGFβ1 signaling/activities.

Protection (from solvent exposure): In the context of HDX-MS-based assessment of protein-protein interactions, such as antibody-antigen binding, the degree by which a protein (e.g., a region of a protein containing an epitope) is exposed to a solvent, thereby allowing proton exchange to occur, inversely correlates with the degree of binding/interaction. Therefore, when an antibody described herein binds to a region of an antigen, the binding region is “protected” from being exposed to the solvent because the protein-protein interaction precludes the binding region from being accessible by the surrounding solvent. Thus, the protected region is indicative of a site of interaction. Typically, suitable solvents are physiological buffers.

ProTGFβ1: The term “proTGFβ1” as used herein is intended to encompass precursor forms of inactive TGFβ1 dimer complex that comprises a prodomain sequence of TGFβ1 within the complex. Thus, the term can include the pro-, as well as the latent-forms of TGFβ1. The expression “pro/latent TGFβ1” may be used interchangeably. The “pro” form of TGFβ1 exists prior to proteolytic cleavage at the furin site. Once cleaved, the resulting form is said to be the “latent” form of TGFβ1. The “latent” complex remains associated until further activation trigger, such as integrin-driven activation event. The proTGFβ1 complex is comprised of dimeric TGFβ1 pro-protein polypeptides, linked with disulfide bonds. The latent dimer complex is covalently linked to a single presenting molecule via the cysteine residue at position 4 (Cys4) of each of the proTGFβ1 polypeptides. The adjective “latent” may be used generally to describe the “inactive” state of TGFβ1, prior to integrin-mediated or other activation events. The proTGFβ1 polypeptide contains a prodomain (LAP) and a growth factor domain (SEQ ID NO: 24).

Regression: Regression of tumor or tumor growth can be used as an in vivo efficacy measure. In preclinical settings, median tumor volume (MTV) and Criteria for Regression Responses Treatment efficacy may be determined from the tumor volumes of animals remaining in the study on the last day. Treatment efficacy may also be determined from the incidence and magnitude of regression responses observed during the study. Treatment may cause partial regression (PR) or complete regression (CR) of the tumor in an animal. Complete regression achieved in response to therapy (e.g., administration of a drug) may be referred to as “complete response” and the subject that achieves complete response may be referred to as a “complete responder”. In some embodiments of preclinical tumor models, a PR response is defined as the tumor volume that is 50% or less of its Day 1 volume for three consecutive measurements during the course of the study, and equal to or greater than 13.5 mm3 for one or more of these three measurements. In some embodiments, a CR response is defined as the tumor volume that is less than 13.5 mm3 for three consecutive measurements during the course of the study. In preclinical model, an animal with a CR response at the termination of a study may be additionally classified as a tumor-free survivor (TFS). The term “effective tumor control” may be used to refer to a degree of tumor regression achieved in response to treatment, where, for example, the tumor volume is reduced to <25% of the endpoint tumor volume. For instance, in a particular model, if the endpoint tumor volume is 2,000 mm3, effective tumor control is achieved if the tumor is reduced to less than 500 mm3. Therefore, effective tumor control encompasses complete regression, as well as partial regression that reaches the threshold reduction. Similarly, regression of fibrosis can be used as an in vivo efficacy measure of a therapy such as a TGFβ1 inhibitor. The regression of fibrotic conditions may be determined based on the standard criteria to assess the severity of fibrotic manifestation by disease stage.

Regulatory T cells: “Regulatory T cells,” or Tregs, are a type of immune cells characterized by the expression of the biomarkers CD4, FOXP3, and CD25. Tregs are sometimes referred to as suppressor T cells and represent a subpopulation of T cells that modulate the immune system, maintain tolerance to self-antigens, and prevent autoimmune disease. Tregs are immunosuppressive and generally suppress or downregulate induction and proliferation of effector T (Teff) cells. Tregs can develop in the thymus (so-called CD4+ Foxp3+ “natural” Tregs) or differentiate from naïve CD4+ T cells in the periphery, for example, following exposure to TGFβ or retinoic acid. Tregs can express cell surface GARP-proTGFβ1.

Resistance (to therapy): Resistance to a particular therapy (such as CBT) may be due to the innate characteristics of the disease such as cancer (“primary resistance”), or due to acquired phenotypes that develop over time following the treatment (“acquired resistance”). Patients who do not show therapeutic response to a therapy (e.g., those who are non-responders or poorly responsive to the therapy) are said to have primary resistance to the therapy and may be characterized as primary non-responders. Patients who initially show therapeutic response to a therapy but later lose effects (e.g., progression or recurrence despite continued therapy) are said to have acquired resistance to the therapy.

Response Evaluation Criteria in Solid Tumors (RECIST) and iRECIST: RECIST is a set of published rules that define when tumors in cancer patients improve (“respond”), stay the same (“stabilize”), or worsen (“progress”) during treatment. The criteria were published in February 2000 by an international collaboration including the European Organisation for Research and Treatment of Cancer (EORTC), National Cancer Institute of the United States, and the National Cancer Institute of Canada Clinical Trials Group. Subsequently, a revised version of the RECIST guideline (RECIST v 1.1) has been widely adapted (see: Eisenhauera et al. (2009), “New response evaluation criteria in solid tumours: Revised RECIST guideline (version 1.1)” Eur J Cancer 45: 228-247, incorporated herein). Response criteria are as follows: Complete response (CR): Disappearance of all target lesions; Partial response (PR): At least a 30% decrease in the sum of the LD of target lesions, taking as reference the baseline s u m LD; Stable disease (SD): Neither sufficient shrinkage to qualify for PR nor sufficient increase to qualify for PD, taking as reference the smallest sum LD since the treatment started; Progressive disease (PD): At least a 20% increase in the sum of the LD of target lesions, taking as reference the smallest sum LD recorded since the treatment started or the appearance of one or more new lesions. On the other hand, iRECIST provides a modified set of criteria that takes into account immune-related response. See: www.ncbi.nlm.nih.gov/pmc/articles/PMC5648544/. The RECIST and iRECIST criteria are standardized, may be revised from time to time as more data become available, and are well understood in the art.

Solid tumor: The term “solid tumor” refers to proliferative disorders resulting in an abnormal growth or mass of tissue that usually does not contain cysts or liquid areas. Solid tumors may be benign (non-cancerous), or malignant (cancerous). Solid tumors are typically comprised of multiple cell types, including, without limitation, cancerous (malignant) cells, stromal cells such as CAFs, and infiltrating leukocytes, such as macrophages and lymphocytes. Solid tumors to be treated with an isoform-selective inhibitor of TGFβ1, such as those described herein, are typically TGFβ1-positive (TGFβ1+) tumors.

Solution Equilibrium Titration (SET): The SET is an assay whereby binding between two molecules (such as an antigen and an antibody that binds the antigen) can be measured at equilibrium in a solution. For example, Meso-Scale Discovery (“MSD”)-based SET, or MSD-SET, is a useful mode of determining dissociation constants for particularly high-affinity protein-protein interactions at equilibrium, such as picomolar-affinity antibodies binding to their antigens (see, for example: Ducata et al. (2015) J Biomolecular Screening 20(10):1256-1267). The SET-based assays are particularly useful for determining KD values of antibodies with sub-nanomolar (e.g., picomolar) affinities.

Specific binding: As used herein, the term “specific binding” or“specifically binds” means that the interaction of the antibody, or antigen-binding portion thereof, with an antigen or amino acid residue is dependent upon the presence of a particular structure (e.g., an antigenic determinant or epitope). For example, the antibody, or antigen-binding portion thereof, binds to a specific protein rather than to proteins generally. In some embodiments, an antibody, or antigen-binding portion thereof, specifically binds to a target, e.g., TGFβ1, if the antibody has a KD for the target of at least about 10⁻⁸ M, 10⁻⁹ M, 10⁻¹⁰ M, 10⁻¹¹ M, 10⁻¹² M, or less. In some embodiments, the term “specific binding to an epitope of proTGFβ1”, “specifically binds to an epitope of proTGFβ1”, “specific binding to proTGFβ1”, or “specifically binds to proTGFβ1” as used herein, refers to an antibody, or antigen-binding portion thereof, that binds to proTGFβ1 and has a dissociation constant (KD) of 1.0×10⁻⁸ M or less, as determined by suitable in vitro binding assays. In one embodiment, an antibody, or antigen-binding portion thereof, can specifically bind to both human and a non-human (e.g., mouse) orthologues of proTGFβ1.

Subject: The term “subject” in the context of therapeutic applications refers to an individual who receives clinical care or intervention, such as treatment, diagnosis, etc. Suitable subjects include vertebrates, including but not limited to mammals (e.g., human and non-human mammals). Where the subject is a human subject, the term “patient” may be used interchangeably. In a clinical context, the term “a patient population” or“patient subpopulation” is used to refer to a group of individuals that falls within a set of criteria, such as clinical criteria (e.g., disease presentations, disease stages, susceptibility to certain conditions, responsiveness to therapy, etc.), medical history, health status, gender, age group, genetic criteria (e.g., carrier of certain mutation, polymorphism, gene duplications, DNA sequence repeats, etc.) and lifestyle factors (e.g., diet, smoking, alcohol consumption, exercise, etc.).

TGFβ1-related indication: A “TGFβ1-related indication” means any disease, disorder and/or condition related to expression, activity and/or metabolism of a TGFβ1 or any disease, disorder and/or condition that may benefit from inhibition of the activity and/or levels TGFβ1. Certain TGFβ1-related indications are driven predominantly by the TGFβ1 isoform. TGFβ1-related indications include, but are not limited to: fibrotic conditions (such as organ fibrosis, and fibrosis of tissues involving chronic inflammation), proliferative disorders (such as cancer, e.g., solid tumors and myelofibrosis), disease associated with ECM dysregulation (such as conditions involving matrix stiffening and remodeling), disease involving endothelial-to-mesenchymal transition (EndMT), disease involving epithelial-to-mesenchymal transition (EMT), disease involving proteases, disease with aberrant gene expression of certain markers described herein. These disease categories are not intended to be mutually exclusive.

TGFβ inhibitor: The term “TGFβ inhibitor” refers broadly to any agent capable of antagonizing biological activities, signaling or function of TGFβ growth factor (e.g., TGFβ1, TGFβ2 and/or TGFβ3). The term is not intended to limit its mechanism of action and includes, for example, neutralizing antibodies, receptor antagonists, soluble ligand traps, and activation inhibitors of TGFβ. Non-selective TGFβ inhibitors are commonly referred to as “pan-inhibitors” of TGFβ. TGFβ inhibitors also include antibodies that are capable of reducing the availability of latent proTGFβ which can be activated in the niche, for example, by inducing antibody-dependent cell mediated cytotoxicity (ADCC), and/or antibody-dependent cellular phagocytosis (ADPC), as well as antibodies that result in internalization of cell-surface complex comprising latent proTGFβ, thereby removing the precursor from the plasma membrane without depleting the cells themselves. Internalization may be a suitable mechanism of action for LRRC33-containing protein complexes (such as human LRRC33-proTGFβ1) which results in reduced levels of cells expressing LRRC33-containing protein complexes on cell surface.

The “TGF/3 family” is a class within the TGFβ superfamily and contains three members in human: TGFβ1, TGFβ2, and TGFβ3, which are structurally similar and are encoded by separate genes. The three growth factors are known to signal via the same receptors.

Therapeutic window: The term “therapeutic window” refers to a range of doses/concentrations that produces therapeutic response without causing significant/observable/unacceptable adverse effect (e.g., within adverse effects that are acceptable or tolerable) in subjects. Therapeutic window may be calculated as a ratio between minimum effective concentrations (MEC) to the minimum toxic concentrations (MTC). To illustrate, a TGFβ1 inhibitor that achieves in vivo efficacy at 10 mg/kg and shows tolerability or acceptable toxicities at 100 mg/kg provides at least a 10-fold (e.g., 1 Ox) therapeutic window. By contrast, a pan-inhibitor of TGFβ that is efficacious at 10 mg/kg but causes adverse effects at 5 mg/kg is said to have “dose-limiting toxicities.” For example, Ab2 has been shown to be efficacious at dosage ranging between about <3 and 30 mg/kg/week and was also shown to be free of observable toxicities associated with pan-inhibition of TGFβ at least 100 mg/kg/week for 4 weeks in preclinical models such as rats. Based on this, Ab2 shows at minimum a 3.3-fold and up to 33-fold therapeutic window.

Toxicity: As used herein, the term “toxcity” or “toxicities” refers to unwanted in vivo effects in subjects (e.g., patients) associated with a therapy administered to the subjects (e.g., patients), such as undesirable side effects and adverse events. “Tolerability” refers to a level of toxicities associated with a therapy or therapeutic regimen, which can be reasonably tolerated by patients, without discontinuing the therapy due to the toxicities. Typically, toxcity/toxcology studies are carried out in one or more preclinical models prior to clinical development to assess safety profiles of a drug candidate (e.g., monoclonal antibody therapy). Toxcity/toxcology studies may help determine the “no observed adverse effect level (NOAEL)” and the “maximally tolerated dose (MTD)” of a test article, based on which a therapeutic window may be deduced. Preferably, a species that is shown to be sensitive to the particular intervention should be chosen as a preclinical animal model in which safety/toxcity study is to be carried out. In case of TGFβ inhibition, suitable species include rats, dogs, and cynos. Mice are reported to be less sensitive to pharmacological inhibition of TGFβ and may not reveal toxicities that are potentially dangerous in other species, including human, although certain studies reportoicities observed with pan-inhibition of TGFβ in mice. To illustrate in the context of the present disclosure, the NOAEL for Ab2 in rats was the highest dose evaluated (100 mg/kg), suggesting that the MTD is >100 mg/kg, based on a four-week toxicology study.

Treat/treatment: The term “treat” or “treatment” includes therapeutic treatments, prophylactic treatments, and applications in which one reduces the risk that a subject will develop a disorder or other risk factor. Thus the term is intended to broadly mean: causing therapeutic benefits in a patient by, for example, slowing disease progression, reversing certain disease features, normalizing gene expression, enhancing or boosting the body's immunity; reducing or reversing immune suppression; reducing, removing or eradicating harmful cells or substances from the body; reducing disease burden (e.g., fibrosis and tumor burden); preventing recurrence or relapse; prolonging a refractory period, and/or otherwise improving survival. The term includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In the context of combination therapy, the term may also refer to: i) the ability of a second therapeutic to reduce the effective dosage of a first therapeutic so as to reduce side effects and increase tolerability; ii) the ability of a second therapy to render the patient more responsive to a first therapy; and/or iii) the ability to effectuate additive or synergistic clinical benefits.

Tumor-associated macrophage (TAM): TAMs are polarized/activated macrophages with pro-tumor phenotypes (M2-like macrophages). TAMs can be either marrow-originated monocytes/macrophages recruited to the tumor site or tissue-resident macrophages which are derived from erythro-myeloid progenitors. Differentiation of monocytes/macrophages into TAMs is influenced by a number of factors, including local chemical signals such as cytokines, chemokines, growth factors and other molecules that act as ligands, as well as cell-cell interactions between the monocytes/macrophages that are present in the niche (tumor microenvironment). Generally, monocytes/macrophages can be polarized into so-called “M1” or “M2” subtypes, the latter being associated with more pro-tumor phenotype. In a solid tumor, up to 50% of the tumor mass may correspond to macrophages, which are preferentially M2-polarized. Among tumor-associated monocytes and myeloid cell populations, M1 macrophages typically express cell surface HLA-DR, CD68 and CD86, while M2 macrophages typically express cell surface HLA-DR, CD68, CD163 and CD206. Tumor-associated, M2-like macrophages (such as M2c and M2d subtypes) can express cell surface LRRC33 and/or LRRC33-proTGFβ1. M2-like macrophages may be also enriched in fibrotic microenvironment.

Tumor microenvironment: The term “tumor microenvironment (TME)” refers to a local disease niche, in which a tumor (e.g., solid tumor) resides in vivo. The TME may comprise disease-associated molecular signature (a set of chemokines, cytokines, etc.), disease-associated cell populations (such as TAMs, CAFs, MDSCs, etc.) as well as disease-associated ECM environments (alterations in ECM components and/or structure).

Variable region: The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

Other than in the operating examples, or where otherwise indicated, all numbers expressing quantities of ingredients or reaction conditions used herein should be understood as modified in all instances by the term “about.” The term “about” when used in connection with percentages can mean±1%.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the phrase “at least one,” in reference to a list of one or more elements, should be understood to mean at least one element selected from anyone or more of the elements in the list of elements, but not necessarily including at least one of each and every element specifically listed with in the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Ranges provided herein are understood to be short hand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, e.g., 10-20, 1-10, 30-40, etc.

### Transforming Growth Factor-Beta (TGFβ)

The Transforming Growth Factor-beta (TGFβ) activities and subsequent partial purification of the soluble growth factors were first described in the late 1970's to early 1980's, with which the TGFβ field began some 40 years ago. To date, 33 gene products have been identified that make up the large TGFβ superfamily. The TGFβ superfamily can be categorized into at least three subclasses by structural similarities: TGFβs, Growth-Differentiation Factors (GDFs) and Bone-Morphogenetic Proteins (BMPs). The TGFβ subclass is comprised of three highly conserved isoforms, namely, TGFβ1, TGFβ2 and TGFβ3, which are encoded by three separate genes in human.

The TGFβs are thought to play key roles in diverse processes, such as inhibition of cell proliferation, extracellular matrix (ECM) remodeling, and immune homeostasis. The importance of TGFβ1 for T cell homeostasis is demonstrated by the observation that TGFβ1−/− mice survive only 3-4 weeks, succumbing to multiorgan failure du e to massive immune activation (Kulkarni, A. B., et al., Proc Natl Acad Sci USA, 1993. 90(2): p. 770-4; Shull, M. M., et al., Nature, 1992. 359(6397): p. 693-9). The roles of TGFβ2 and TGFβ3 are less clear. Whilst the three TGFβ isoforms have distinct temporal and spatial expression patterns, they signal through the same receptors, TGFβRI and TGFβRII, although in some cases, for example for TGFβ2 signaling, type III receptors such as betaglycan are also required (Feng, X. H. and R. Derynck, Annu Rev Cell Dev Biol, 2005. 21: p. 659-93; Massague, J., Annu Rev Biochem, 1998. 67: p. 753-91). Ligand-induced oligomerization of TGFβRI/II triggers the phosphorylation of SMAD transcription factors, resulting in the transcription of target genes, such as Col1a1, Col3a1, ACTA2, and SERPINE1 (Massague, J., J. Seoane, and D. Wotton, Genes Dev, 2005. 19(23): p. 2783-810). SMAD-independent TGFβ signaling pathways have also been described, for example in cancer or in the aortic lesions of Marfan mice (Derynck, R. and Y. E. Zhang, Nature, 2003. 425(6958): p. 577-84; Holm, T. M., et al., Science, 2011.332(6027): p. 358-61).

The biological importance of the TGFβ pathway in humans has been validated by genetic diseases. Camurati-Engelman disease results in bone dysplasia due to an autosomal dominant mutation in the TGFB1 gene, leading to constitutive activation of TGFβ1 signaling (Janssens, K., et al., J Med Genet, 2006. 43(1): p. 1-11). Patients with Loeys/Dietz syndrome carry autosomal dominant mutations in components of the TGFβ signaling pathway, which cause aortic aneurism, hypertelorism, and bifid uvula (Van Laer, L., H. Dietz, and B. Loeys, AdvExp Med Biol, 2014. 802: p. 95-105). As TGFβ pathway dysregulation has been implicated in multiple diseases, several drugs that target the TGFβ pathway have been developed and tested in patients, but with limited success.

Dysregulation of the TGFβ signaling has been associated with a wide range of human diseases. Indeed, in a number of disease conditions, such dysregulation may involve multiple facets of TGFβ function. Diseased tissue, such as fibrotic and/or inflamed tissues and tumors, may create a local environment in which TGFβ activation can cause exacerbation or progression of the disease, which may be at least in part mediated by interactions between multiple TGFβ-responsive cells, which are activated in an autocrine and/or paracrine fashion, together with a number of other cytokines, chemokines and growth factors that playa role in a particular disease setting.

### Novel TGFβ1-Selective Inhibitors of the Invention

Applicant's previous work described antibodies capable of specifically binding a proTGFβ1 complex when the complex is in association with any one of the presenting molecules, namely, LTBP1, LTBP3, GARP and LRRC33. Such antibodies were referred to as “context-permissive” or “context-independent” antibodies of TGFβ1 for their ability to target and inhibit the release of TGFβ1 growth factor from any one of the presenting molecule-proTGFβ1 complexes. Notably, the binding and inhibitory activities of these antibodies were specific to the TGFβ1 isoform only. In fact, neither significant binding nor inhibition was detected towards the TGFβ2 and TGFβ3 counterparts.

Traditional approaches to antagonizing TGFβ signaling have been to i) directly neutralize the mature growth factor after it has already become active so as to deplete free ligands (e.g., released from its latent precursor complex that are available for receptor binding; ii) employ soluble receptor fragments capable of sequestering free ligands (e.g., so-called ligand traps); or, iii) target its cell-surface receptor(s) to block ligand-receptor interactions. Each of these conventional approaches requires the antagonist to compete against endogenous counterparts. Moreover, the first two approaches (i and ii) above target the active, soluble ligand, which is a transient species. Therefore, such antagonist must be capable of kinetically outcompeting the endogenous receptor during the brief temporal window. The third approach may provide a more durable effect in comparison but inadvertently results in unwanted inhibitory effects (hence possible toxicities) because many growth factors (e.g., up to ˜20) signal via the same receptor(s).

Indeed, the notion of isoform selectivity is important also for safety considerations. Conventional inhibitors of TGFβ are typically not selective for one isoform; rather, most are known as “pan-inhibitors”; that is, they antagonize TGFβ1, TGFβ2 and TGFβ3 and are associated with potentially severe toxicities when administered to animals such as rats, dogs, non-human primates, mice and human. Based on these observations, Applicant of the present disclosure previously hypothesized that the observed toxicities might result from the lack of isoform selectivity of the prior art inhibitors. Subsequently, Applicant described antibodies capable of selectively binding and inhibiting TGFβ1 activation and demonstrated that such antibodies showed significantly improved safety profiles. For example, no test article-related toxicities were observed up to the highest dose tested, which was 100 mg/kg per week in four-week rat toxicology studies. Applicant further sought to identify TGFβ1-selective inhibitory antibodies with at least an equivalent level of safety/toxcology profiles as the previously identified antibodies, but with even greater potency.

Accordingly, for the work presented herein, it was envisaged that improved antibodies should embody the following features: 1) selectivity towards TGFβ1 should be maintained to minimize unwanted toxicities associated with pan-inhibition (“isoform-selectivity”) (see, for example, WO 2017/156500); 2) should exhibit broad binding activities to accommodate various biological contexts, or, both matrix-associated and cell-associated categories (WO 2018/129329); 3) should have robust inhibitory activities (“potency”); 4) the preferred mechanism of action remains blocking the activation step so the inhibitor can target a tissue-tethered, latentTGFβ1 complex, so as to preemptively prevent downstream activation events to achieve durable effects, rather than to directly target soluble/free growth factors (“durability”); and, 5) preferably, the improved antibodies should exert enhanced bias (e.g., preferential binding affinity) towards matrix-associated TGFβ1, which is preferably determined at equilibrium, in order to more accurately reflect the mechanism of action of these antibodies.

For the last feature (5) above, it was contemplated that enhanced context bias may be exploited as a feature that offers a therapeutic advantage in certain disease conditions, in which dysregulation of the extracellular matrix plays a role (e.g., fibrotic disorders). Accordingly, the improved antibodies should embody preferential (e.g., biased) binding activities towards matrix-associated TGFβ1 (e.g., LTBP1-proTGFβ1 and LTBP3-proTGFβ1), preferably with five-fold or greater affinities towards LTBP1-proTGFβ1 and/or LTBP3-proTGFβ1 complexes over GARP-proTGFβ1, as measured by suitable in vitro binding assays.

In some embodiments, such antibodies are biased against a GARP-proTGFβ1 complex such that binding affinity for a GARP-presented latent TGFβ1 is weaker than binding affinities for latent TGFβ1 presented by one of the other three known presenting molecules. In other embodiments, such antibodies are biased against so-called cell-associated proTGFβ1 complexes (GARP or LRRC33-associated) over matrix-associated complexes.

Accordingly, disclosed herein are improved inhibitors of TGFβ1, characterized in that, as compared to TGFβ1-selective inhibitors of earlier disclosures, these antibodies have enhanced biding properties, increased inhibitory potency, and maintain the desirable safety profiles. The TGFβ1-selective inhibitors of the present disclosure are monoclonal antibodies (e.g., immunoglobulins, engineered immunoglobulin-like molecules, antigen-binding fragments or portions thereof) that specifically bind at least a portion of the prodomain (sometimes referred to as “LAP”) of a proTGFβ1 complex and have isoform-selective inhibitory activity towards TGFβ1 (see “Core Properties” of Table 1). In addition to these features, the antibodies disclosed herein further meet the Antibody Criteria of one or more of Categories 1-5 as set forth in Table 1 herein.

TABLE 1
Improved TGFβ1-selective inhibitors of the present disclosure

| Category | Antibody Criteria | Exemplary Antibodies |
|---|---|---|
| (All) | Core Properties: | Present disclosure |
| | Selectively inhibits TGFβ1 signaling, over TGFβ2 and TGFβ3 | WO 2018/129329 (Ab3) |
| | signaling | WO 2017/156500 |
| | Specifically binds each of: LTBP1-proTGFβ1, LTBP3-proTGFβ1, | |
| | GARP-proTGFβ1 and LRRC33-proTGFβ1 complexes (human | |
| | and/or murine) | |
| | Binding involves at least a portion of the prodomain of proTGFβ1 | |
| | Improved safety/toxicology profiles, as compared to pan-TGFβ | |
| | inhibitors | |

Additional required criteria defined by CDR sequences

| | | |
|---|---|---|
| 1 | i) the H-CDR1 has an amino acid sequence represented by | Ab7 |
| | FTF(X1)(X2)(X3)AM(X4), wherein, X1 is A or S; X2 is N, D, S, or A; | Ab8 |
| | X3 is Y or F; and/or X4 is S, T, or V (SEQ ID NO: 252); | Ab9 |
| | ii) the H-CDR2 has an amino acid sequence represented by | Ab12 |
| | (X1)IS(X2)(X3)(X4)(X5)(X6)(X7)Y(X8)ADSVKG, wherein optionally, | Ab13 |
| | X1 is S or A; X2 is G or S; X3 is S, T, or F; X4 is G or A; X5 is G, A, | Ab14 |
| | F, or S; X6 is A, H, T, S, or V; X7 is T or I; and/or, X8 is Y or F (SEQ | Ab15 |
| | ID NO: 253); | Ab2 |
| | iii) the H-CDR3 has an amino acid sequence represented by | Ab16 |
| | A(X1)VSS(X2)(X3)WD(X4)D(X5), wherein optionally, X1 is R or T; | Ab17 |
| | X2 is G or Y; X3 is H or L; X4 is F, Y, or L; and/or X5 is Y or E (SEQ | Ab18 |
| | ID NO: 254); | Ab19 |
| | iv) the L-CDR1 has an amino acid sequence represented by | Ab20 |
| | (X1)ASQ(X2)IS(X3)(X4)LN, wherein optionally, X1 is R or Q; X2 is S | |
| | or D; X3 is S or N; and/or X4 is F, Y or S (SEQ ID NO: 255); | |
| | v) the L-CDR2 has an amino acid sequence represented by | |
| | (X1)AS(X2)L(X3)(X4), wherein optionally, X1 is D or A; X2 is S or N; | |
| | X3 is Q or E; and/or X4 is S or T (SEQ ID NO: 256); and, | |
| | vi) the L-CDR3 has an amino acid sequence represented by | |
| | QQ(X1)(X2)(X3)(X4)P(X5)T, wherein optionally, X1 is S, A, T, or V; | |
| | X2 is F, Y or P; X3 is S, N, T, or D; X4 is A, L, V, or P; and/or X5 is F | |
| | or L (SEQ ID NO: 257). | |
| 2 | Comprises at least two of the following CDRs | Ab7 |
| | i) H-CDR1 = FTFSDYAMT (SEQ ID NO: 250) | Ab8 |
| | ii) H-CDR2 = AISGSGAATYYADSVKG (SEQ ID NO: 251) | Ab9 |
| | iii) H-CDR3 = ARVSSGHWDFDY (SEQ ID NO: 110) | Ab11 |
| | iv) L-CDR1 = RASQSISSYLN (SEQ ID NO: 111) | Ab12 |
| | v) L-CDR2 = AASNLQS (SEQ ID NO: 136) | Ab13 |
| | vi) L-CDR3 = QQTYTVPLT (SEQ ID NO: 12) | Ab14 |
| | wherein optionally each ofthe CDRs contains one amino acid | Ab15 |
| | change, | Ab2 |
| | with the proviso that the antibody comprises at least one CDR that is | Ab16 |
| | selected from the group consisting of the H-CDR1, H-CDR2, L-CDR2 | Ab17 |
| | and L-CDR3 listed above. | Ab18 |
| | | Ab19 |
| | | Ab20 |

Additional required criteria defined by binding profiles (as determined by solution equilibrium titration)

| | | |
|---|---|---|
| 3 | binds human LTBP1-proTGFβ1 and LTBP3-proTGFβ1 complexes | At least: |
| | each with KD of ≤1 nM; and, human GARP-proTGFβ1 and LRRC33- | Ab12 |
| | proTGFβ1 complexes each with KD > 1 nM | Ab2 |
| | | Ab20 |
| | | Ab35 |
| 4 | binds human LTBP1-proTGFβ1 and LTBP3-proTGFβ1 complexes | At least: |
| | each with KD of ≤1 nM; and, average affinity for human LTBP1- | Ab2 |
| | proTGFβ1 and LTBP3-proTGFβ1 complexes is at least five-fold | Ab20 |
| | greater than that for human GARP-proTGFβ1 and LRRC33- | |
| | proTGFβ1 complexes | |
| 5 | binds human LTBP1-proTGFβ1, human LTBP3-proTGFβ1, human | At least: |
| | GARP-proTGFβ1 and human LRRC33-proTGFβ1 complexes each | Ab11 |
| | with an affinity of ≤10 nM (preferably ≤5 nM); and, the antibody has | Ab2 |
| | more than five-fold matrix/LTBP-bias in relative affinities between at | Ab20 |
| | least one of the matrix-associated complexes and at least one of the | Ab35 |
| | cell-associated complexes | |

### Category 1 Antibodies

In one aspect, the present disclosure provides antibodies (e.g., immunoglobulins, modified or engineered immunoglobulin-like molecules, and antigen-binding fragments thereof) having the Core Properties of Table 1 and further meet the additional required criteria defined by CDR sequences of Category 1. Thus, such antibodies or fragments thereof are capable of specifically binding each of the following antigen complexes: hLTBP1-proTGFβ1, hLTBP3-proTGFβ1, hGARP-pro TGFβ1 and hLRRC33-proTGFβ1, and, the antibodies or the fragments comprise the six CDR consensus sequences summarized in Table 2 below.

TABLE 2
CDR sequence requirements for Category 1 antibodies

| CDR | Consensus sequences |
|---|---|
| H-CDR1 | FTF(X₁)(X₂)(X₃)AM(X₄), wherein: X₁ = A or S; X₂ = N, D, S, or A; X₃ = Y or |
| | F; and/or X₄ = S, T, or V (SEQ ID NO: 252). |
| H-CDR2 | (X₁)IS(X₂)(X₃)(X₄)(X₅)(X₆)(X₇)Y(X₈)ADSVKG, wherein optionally: X₁ = S or |
| | A; X₂ = G or S; X₃ = S, T, or F; X₄ = G or A; X₅ = G, A, F, or S; X₆ =A, H, T, |
| | S, or V; X₇ = T or I; and/or, X₈ = Y or F (SEQ ID NO: 253). |
| H-CDR3 | A(X₁)VSS(X₂)(X₃)WD(X₄)D(X₅), wherein optionally: X₁ = R or T; X₂ = G or |
| | Y; X₃ = H or L; X₄ = F, Y, or L; and/or X₅ = Y or E (SEQ ID NO: 254). |
| L-CDR1 | (X₁)ASQ(X₂)IS(X₃)(X₄)LN, wherein optionally: X₁ = R or Q; X₂ = S or D; X₃ = |
| | S or N; and/or X₄ = F, Y or S (SEQ ID NO: 255). |
| L-CDR2 | (X₁)AS(X₂)L(X₃)(X₄), wherein optionally: X₁ = D or A; X₂ = S or N; X₃ = Q or |
| | E; and/or X₄ = S or T (SEQ ID NO: 256). |
| L-CDR3 | QQ(X₁)(X₂)(X₃)(X₄)P(X₅)T, wherein optionally: X₁ = S, A, T, or V; X₂ = F, Y |
| | or P; X₃ = S, N, T, or D; X₄ = A, L, V, or P; and/or X₅ = F or L (SEQ ID NO: |
| | 257). |

Thus, the invention provides an isolated monoclonal antibody or antigen-binding fragment thereof comprising an H-CDR1, an H-CDR-2, an H-CDR3, an L-CDR1, an L-CDR2 and an L-CDR3, wherein:
i) the H-CDR1 has an amino acid sequence represented by FTF (X₁)(X₂)(X₃)AM(X₄), wherein, X₁ is A or S; X₂ is N, D, S, or A; X₃ is Y or F; and/or X₄ is S, T, or V(SEQ ID NO: 252);
ii) the H-CDR2 has an amino acid sequence represented by (X₁)IS(X₂)(X₃)(X₄)(X₅)(X₆)(X₇)Y(X₈)ADSVKG, wherein optionally, X₁ is S or A; X₂ is G or S; X₃ is S, T, or F; X₄ is G or A; X₅ is G, A, F, or S; X₆ is A, H, T, S, or V; X₇ is T or I; and/or, X₈ is Y or F(SEQ ID NO: 253);
iii) the H-CDR3 has an amino acid sequence represented by A(X₁)VSS(X₂)(X₃)WD(X₄)D(X₅), wherein optionally, X₁ is R or T; X₂ is G or Y; X₃ is H or L; X₄ is F, Y, or L; and/or X₅ is Y or E (SEQ ID NO: 254);
iv) the L-CDR1 has an amino acid sequence represented by (X₁)ASQ(X₂)IS(X₃)(X₄)LN, wherein optionally, X₁ is R or Q; X₂ is S or D; X₃ is S or N; and/or X₄ is F, Y or S (SEQ ID NO: 255);
v) the L-CDR2 has an amino acid sequence represented by (X₁)AS(X₂)L(X₃)(X₄), wherein optionally, X₁ is D or A; X₂ is S or N; X₃ is Q or E; and/or X₄ is S or T (SEQ ID NO: 256); and,
vi) the L-CDR3 has an amino acid sequence represented by QQ(X₁)(X₂)(X₃)(X₄)P(X₅)T, wherein optionally, X₁ is S, A, T, or V; X₂ is F, Y or P; X₃ is S, N, T, or D; X₄ is A, L, V, or P; and/or X₅ is F or L (SEQ ID NO: 257).

In some embodiments, the H-CDR1 comprises a D at position X₂.

In some embodiments, the H-CDR2 comprises an S at position X₃.

In some embodiments, the H-CDR3 comprises a G at position X₂.

In some embodiments, the H-CDR3 comprises an H at position X₃.

In some embodiments, the L-CDR1 comprises a Y at position X₄.

In some embodiments, the L-CDR3 comprises a T at position X₁.

In some embodiments, the L-CDR3 comprises a Y at position X₂.

In some embodiments, the antibody or the fragment thereof is characterized in that: the X₂ of the H-CDR1 is D; the X₃ of the H-CDR2 is S; the X₂ and the X₃ of the H-CDR3 are G and H, respectively; the L-CDR2 immediately follows a Y residue; and/or, the X₁ and the X₂ of the L-CDR3 are T and Y, respectively.

In some embodiments, the antibody or the fragment comprises an H-CDR1, an H-CDR-2, an H-CDR3, an L-CDR1, an L-CDR2 and an L-CDR3, wherein:
i) the H-CDR1 has an amino acid sequence represented by FTF(X₁)D(X₃)AM(X₄), wherein X₁ is A or S; X₃ is Y or F; and/or X₄ is S, T, or V (SEQ ID NO: 276);
ii) the H-CDR2 has an amino acid sequence represented by (X₁)IS(X₂)S(X₄)(X₅)(X₆)(X₇)Y(X₈)ADSVKG, wherein optionally, X₁ is S or A; X₂ is G or S; X₄ is G or A; X₅ is G, A, F, or S; X₆ is A, H, T, S, or V; X₇ is T or I; and/or, X₈ is Y or F (SEQ ID NO: 277);
iii) the H-CDR3 has an amino acid sequence represented by A(X₁)VSSGHWD(X₄)D(X₅), wherein optionally, X₁ is R or T; X₄ is F, Y, or L; and/or X₅ is Y or E (SEQ ID NO: 278);
iv) the L-CDR1 has an amino acid sequence represented by (X₁)ASQ(X₂)IS(X₃)(X₄)LN, wherein optionally, X₁ is R or Q; X₂ is S or D; X₃ is S or N; and/or X₄ is F, Y or S (SEQ ID NO: 255);
v) the L-CDR2 has an amino acid sequence represented by Y(X₁)AS(X₂)L(X₃)(X₄), wherein optionally, X₁ is D or A; X₂ is S or N; X₃ is Q or E; and/or X₄ is S or T (SEQ ID NO: 279); and,
vi) the L-CDR3 has an amino acid sequence represented by QQTY(X₃)(X₄)P(X₅)T, wherein optionally, X₃ is S, N, T, or D; X₄ is A, L, V, or P; and/or X₅ is F or L (SEQ ID NO: 280).

Exemplary antibodies and their respective CDR sequences are provided in Table 3 below.

TABLE 3
Exemplary antibodies and CDRs based on the numbering scheme
described in Lu X et al., MAbs. 2019 Jan;11(1):45-57

| Antibody | H-CDR1 | H-CDR2 | H-CDR3 | L-CDR1 | L-CDR2 | L-CDR3 |
|---|---|---|---|---|---|---|
| Ab7 | FTFADYAMT | AISGTGAHTY | ARVSSGHWD | RASQSISSYL | DASSLQS | QQSYSAPFT |
| | (SEQ ID NO: | YADSVKG | FDY | N | (SEQ ID NO: | (SEQ ID NO: |
| | 108) | (SEQ ID NO: | (SEQ ID NO: | (SEQ ID NO: | 112) | 113) |
| | | 109) | 110) | 111) | | |
| Ab8 | FTFSDYAMV | AISGSGFTTY | ARVSSGHWD | RASQSISSYL | DASSLQS | QQSYSAPFT |
| | (SEQ ID NO: | YADSVKG | FDY | N | (SEQ ID NO: | (SEQ ID NO: |
| | 114) | (SEQ ID NO: | (SEQ ID NO: | (SEQ ID NO: | 112) | 113) |
| | | 115) | 110) | 111) | | |
| Ab9 | FTFSSFAMT | AISGSGAATY | ARVSSGHWD | RASQSISSYL | DASSLQS | QQSYSAPFT |
| | (SEQ ID NO: | FADSVKG | FDY | N | (SEQ ID NO: | (SEQ ID NO: |
| | 120) | (SEQ ID NO: | (SEQ ID NO: | (SEQ ID NO: | 112) | 113) |
| | | 121) | 110) | 111) | | |
| Ab10 | FTFRNYAMS | SISGSGGATY | ARVSSGHWD | QASQDISNSL | DASNLET | QQAPNLPFT |
| | (SEQ ID NO: | YADSVKG | FDY | N | (SEQ ID NO: | (SEQ ID NO: |
| | 126) | (SEQ ID NO: | (SEQ ID NO: | (SEQ ID NO: | 130) | 131) |
| | | 127) | 110) | 129) | | |
| Ab1l | FTFRNYAMS | SISGSGGATY | ARVSSGHWD | RASQSISSYL | AASNLQS | QQTYTVPLT |
| | (SEQ ID NO: | YADSVKG | FDY | N | (SEQ ID NO: | (SEQ ID NO: |
| | 126) | (SEQ ID NO: | (SEQ ID NO: | (SEQ ID NO: | 136) | 12) |
| | | 127) | 110) | 111) | | |
| Ab12 | FTFSDYAMV | AISGSGFTTY | ARVSSGHWD | RASQSISSYL | AASNLQS | QQTYTVPLT |
| | (SEQ ID NO: | YADSVKG | FDY | N | (SEQ ID NO: | (SEQ ID NO: |
| | 114) | (SEQ ID NO: | (SEQ ID NO: | (SEQ ID NO: | 136) | 12) |
| | | 115) | 110) | 111) | | |
| Ab13 | FTFADYAMT | AISSFASAIYY | ARVSSGHWD | RASQSISSYL | AASSLQS | QQVYDPPLT |
| | (SEQ ID NO: | ADSVKG | FDY | N | (SEQ ID NO: | (SEQ ID NO: |
| | 108) | (SEQ ID NO: | (SEQ ID NO: | (SEQ ID NO: | 148) | 149) |
| | | 145) | 110) | 111) | | |
| Ab14 | FTFSAYAMT | AISGTGGSTY | ARVSSGHWD | RASQSISSYL | AASSLQS | QQVYDPPLT |
| | (SEQ ID NO: | YADSVKG | FDY | N | (SEQ ID NO: | (SEQ ID NO: |
| | 150) | (SEQ ID NO: | (SEQ ID NO: | (SEQ ID NO: | 148) | 149) |
| | | 151) | 110) | 111) | | |
| Ab15 | FTFSDYAMV | AISGSGFTTY | ARVSSGHWD | RASQSISSYL | AASNLQS | QQTYTVPLT |
| | (SEQ ID NO: | YADSVKG | FDY | N | (SEQ ID NO: | (SEQ ID NO: |
| | 114) | (SEQ ID NO: | (SEQ ID NO: | (SEQ ID NO: | 136) | 12) |
| | | 115) | 110) | 111) | | |
| Ab2 | FTFADYAMT | AISGSGAATY | ARVSSGHWD | RASQSISSYL | AASNLQS | QQTYTVPLT |
| | (SEQ ID NO: | FADSVKG | FDY | N | (SEQ ID NO: | (SEQ ID NO: |
| | 108) | (SEQ ID NO: | (SEQ ID NO: | (SEQ ID NO: | 136) | 12) |
| | | 121) | 110) | 111) | | |
| Ab16 | FTFSNYAMS | AISGSGFTTY | ARVSSGHWD | RASQSISSYL | AASNLQS | QQTYTVPLT |
| | (SEQ ID NO: | YADSVKG | FDY | N | (SEQ ID NO: | (SEQ ID NO: |
| | 168) | (SEQ ID NO: | (SEQ ID NO: | (SEQ ID NO: | 136) | 12) |
| | | 115) | 110) | 111) | | |
| Ab17 | FTFSSFAMT | AISGSGAATY | ARVSSGHWD | RASQSISSYL | AASNLQS | QQTYTVPLT |
| | (SEQ ID NO: | FADSVKG | FDY | N | (SEQ ID NO: | (SEQ ID NO: |
| | 120) | (SEQ ID NO: | (SEQ ID NO: | (SEQ ID NO: | 136) | 12) |
| | | 121) | 110) | 111) | | |
| Ab18 | FTFSDYAMV | AISGSGAATY | ATVSSGHWD | RASQSISSYL | AASNLQS | QQTYTVPLT |
| | (SEQ ID NO: | FADSVKG | FDY | N | (SEQ ID NO: | (SEQ ID NO: |
| | 114) | (SEQ ID NO: | (SEQ ID NO: | (SEQ ID NO: | 136) | 12) |
| | | 121) | 182) | 111) | | |
| Ab19 | FTFSSFAMT | AISGSGAATY | ARVSSGHWD | RASQSISSYL | AASNLQS | QQTYTVPLT |
| | (SEQ ID NO: | FADSVKG | FDE | N | (SEQ ID NO: | (SEQ ID NO: |
| | 120) | (SEQ ID NO: | (SEQ ID NO: | (SEQ ID NO: | 136) | 12) |
| | | 121) | 188) | 111) | | |
| Ab20 | FTFSAYAMT | AISGSGGVTY | ARVSSGHWD | RASQSISSYL | AASNLQS | QQTYTVPLT |
| | (SEQ ID NO: | YADSVKG | YDY | N | (SEQ ID NO: | (SEQ ID NO: |
| | 150) | (SEQ ID NO: | (SEQ ID NO: | (SEQ ID NO: | 136) | 12) |
| | | 193) | 194) | 111) | | |
| Ab35 | FTFRNYAMS | SISGSGGATY | ARVSSYLWD | RASQSISSYL | DASSLQS | QQSYSAPFT |
| | (SEQ ID NO: | YADSVKG | FDY | N | (SEQ ID NO: | (SEQ ID NO: |
| | 126) | (SEQ ID NO: | (SEQ ID NO: | (SEQ ID NO: | 112) | 113) |
| | | 127) | 200) | 111) | | |
| Ab36 | FTFRNYAMS | SISGSGGATY | ARVSSGHWD | RASQSISSYL | DASSLQS | QQSYSAPFT |
| | (SEQ ID NO: | YADSVKG | LDY | N | (SEQ ID NO: | (SEQ ID NO: |
| | 126) | (SEQ ID NO: | (SEQ ID NO: | (SEQ ID NO: | 112) | 113) |
| | | 127) | 206) | 111) | | |

In related embodiments, the Category 1 antibody may comprise six CDRs, each of which has at least 85% sequence identify with the corresponding CDR of any one of the exemplary antibodies provided in Table 3, except Ab36.

In preferred embodiments, by the Kabat numbering scheme, the antibody comprises an H-CDR1 comprising GFTFADYAM (SEQ ID NO: 2), an H-CDR2 comprising ISGSGAA (SEQ ID NO: 4), and H-CDR3 comprising CARVSSGHWDFDY (SEQ ID NO: 6), an L-CDR1 comprising QSISSY(SEQ ID NO: 8), and L-CDR2 comprising AAS (SEQ ID NO: 10) and an L-CDR3 comprising QQTYTVPLT (SEQ ID NO: 12). Alternatively, by the numbering system of Lu et al., the antibody comprises an H-CDR1 comprising FTFADYAMT (SEQ ID NO: 108), an H-CDR2 comprising AISGSGAATYFADSVKG (SEQ ID NO: 121), and H-CDR3 comprising ARVSSGHWDFDY (SEQ ID NO: 110), an L-CDR1 comprising RASQSISSYLN (SEQ ID NO: 111), and L-CDR2 comprising AASNLQS (SEQ ID NO: 136) and an L-CDR3 comprising QQTYTVPLT (SEQ ID NO: 12).

In some embodiments, the antibody or the antigen-binding fragment comprises a heavy chain variable region (V_{H}) that is at least 95% identical to the amino acid sequence set for in SEQ ID NO: 13, and a light chain variable region (V_{L}) that is at least 95% identical to the amino acid sequence set forth in SEQ ID NO: 15. Within the heavy chain variable region in accordance with SEQ ID NO: 13, the residue at position 31 may be D, the residue at position 33 may be A, the residue at position 54 may be S, residue at position 59 may be Y, the residue at position 101 may be S, the residue at position 102 may be G, the residue at position 103 may be H, the residue at position 104 may be W, or, any combinations thereof. Within the light chain variable region in accordance with SEQ ID NO: 15, the residue at position 32 may be Y, the residue at position 49 may be Y, the residue at position 91 may be T, the residue at position 92 may be Y, or any combinations thereof.

In some embodiments, the antibody or the antigen-binding fragment comprises a heavy chain variable region (V_{H}) that is at least 90% identical to the amino acid sequence set for in SEQ ID NO: 13 and comprises amino acid residues D31, A33, S54, Y59, S101, G102, H103, and W104 based on the amino acid sequence set forth in SEQ ID NO: 13, and a light chain variable region (V_{L}) that is at least 90% identical to the amino acid sequence set forth in SEQ ID NO: 15 and comprises amino acid residues Y32, Y49, T91, and Y92, based on the amino acid sequence s et forth in SEQ ID NO: 15.

The heavy chain variable region and light chain variable region sequences of exemplary Category 1 antibodies are provided in Table 4 below:

TABLE 4
Exemplary antibodies and their heavy and light chain variable domain sequences

| Antibody | Heavy Chain Variable Domain (V_{H}) | Light Chain Variable Domain (V_{L}) |
|---|---|---|
| Ab7 | EVQLLESGGGLVQPGGSLRLSCAASGFTFADY | DIMITQSPSSLSASVGDRVTITCRASQSISSYLN |
| | AMTWVRQAPGKGLEWVSAISGTGAHTYYADSV | WYQQKPGKAPKLLIYDASSLQSGVPSRFSGSG |
| | KGRFTISRDNSKNTLYLQMNSLRAEDTAVYYCA | SGTDFTLTISSLQPEDFATYYCQQSYSAPFTFG |
| | RVSSGHWDFDYWGQGTLVTVSS (SEQ ID NO: | QGTKVEIK (SEQ ID NO: 211) |
| | 210) | |
| Ab8 | QVQLVESGGGVVQPGRSLRLSCAASGFTFSDY | DIQMTQSPSSLSASVGDRVTITCRASQSISSYLN |
| | AMTWVRQAPGKGLEWVSAISGSGFTTYYADSV | WYQQKPGKAPKLLIYDASSLQSGVPSRFSGSG |
| | KGRFTISRDNSKNTLYLQMNSLRAEDTAVYYCA | SGTDFTLTISSLQPEDFATYYCQQSYSAPFTFG |
| | RVSSGHWDFDYWGQGTLVTVSS (SEQ ID NO: | QGTKVEIK (SEQ ID NO: 211) |
| | 212) | |
| Ab9 | QVQLVESGGGVVQPGGSLRLSCAASGFTFSSF | DIQMTQSPSSLSASVGDRVTITCRASQSISSYLN |
| | AMTWVRQAPGKGLEWVSAISGSGAATYFADSV | WYQQKPGKAPKLLIYDASSLQSGVPSRFSGSG |
| | KGRFTISRDNSKNTLYLQMNSLRAEDTAVYYCA | SGTDFTLTISSLQPEDFATYYCQQSYSAPFTFG |
| | RVSSGHWDFDYWGQGTLVTVSS (SEQ ID NO: | QGTKVEIK (SEQ ID NO: 211) |
| | 214) | |
| Ab10 | EVQLLESGGGLVQPGGSLRLSCAASGFTFRNY | DIQMTQSPSSLSASVGDRVTITCQASQDISNSLN |
| | AMSWVRQAPGKGLEWVSSISGSGGATYYADSV | WYQQKPGKAPKLLIYDASNLETGVPSRFSGSRS |
| | KGRFTISRDNSKNTLYLQMNSLRAEDTAVYYCA | GTDFTFTISSLQPEDIATYYCQQAPNLPFTFGGG |
| | RVSSGHWDFDYWGQGTLVTVSS (SEQ ID NO: | TKVEIK (SEQ ID NO: 217) |
| | 216) | |
| Ab11 | EVQLLESGGGLVQPGGSLRLSCAASGFTFRNY | DIQLTQSPSSLSASVGDRVTITCRASQSISSYLN |
| | AMSWVRQAPGKGLEWVSSISGSGGATYYADSV | WYQQKPGKAPKLLIYAASNLQSGVPSRFSGSG |
| | KGRFTISRDNSKNTLYLQMNSLRAEDTAVYYCA | SGTDFTLTISSLQPEDFATYYCQQTYTVPLTFGG |
| | RVSSGHWDFDYWGQGTLVTVSS (SEQ ID NO: | GTKVEIK (SEQ ID NO: 15) |
| | 216) | |
| Ab12 | QVQLVESGGGVVQPGRSLRLSCAASGFTFSDY | DIQLTQSPSSLSASVGDRVTITCRASQSISSYLN |
| | AMVWVRQAPGKGLEWVSAISGSGFTTYYADSV | WYQQKPGKAPKLLIYAASNLQSGVPSRFSGSG |
| | KGRFTISRDNSKNTLYLQMNSLRAEDTAVYYCA | SGTDFTLTISSLQPEDFATYYCQQTYTVPLTFGG |
| | RVSSGHWDFDYWGQGTLVTVSS (SEQ ID NO: | GTKVEIK (SEQ ID NO: 15) |
| | 212) | |
| Ab13 | EVQLLESGGGLVQPGGSLRLSCAASGFTFADY | DIQMTQSPSSLSASVGDRVTITCRASQSISSYLN |
| | AMTWVRQAPGKGLEWVSAISSFASAIYYADSVK | WYQQKPGKAPKLLIYAASSLQSGVPSRFSGSG |
| | GRFTISRDNSKNTLYLQMNSLRAEDTAVYYCAR | SGTDFTLTISSLQPEDFATYYCQQVYDPPLTFG |
| | VSSGHWDFDYWGQGTLVTVSS (SEQ ID NO: | GGTKVEIK (SEQ ID NO: 223) |
| | 222) | |
| Ab14 | EVQLLESGGGLVQPGGSLRLSCAASGFTFSAYA | DIQMTQSPSSLSASVGDRVTITCRASQSISSYLN |
| | MTWVRQAPGKGLEWVSAISGTGGSTYYADSVK | WYQQKPGKAPKLLIYAASSLQSGVPSRFSGSG |
| | GRFTISRDNSKNTLYLQMNSLRAEDTAVYYCAR | SGTDFTLTISSLQPEDFATYYCQQVYDPPLTFG |
| | VSSGHWDFDYWGQGTLVTVSS (SEQ ID NO: | GGTKVEIK (SEQ ID NO: 223) |
| | 224) | |
| Ab15 | EVQLVESGGGVVQPGRSLRLSCAASGFTFSDY | DIQLTQSPSSLSASVGDRVTITCRASQSISSYLN |
| | AMVWVRQAPGKGLEWVSAISGSGFTTYYADSV | WYQQKPGKAPKLLIYAASNLQSGVPSRFSGSG |
| | KGRFTISRDNSKNTLYLQMNSLRAEDTAVYYCA | SGTDFTLTISSLQPEDFATYYCQQTYTVPLTFGG |
| | RVSSGHWDFDYWGQGTLVTVSS (SEQ ID NO: | GTKVEIK (SEQ ID NO: 15) |
| | 226) | |
| Ab2 | EVQLLESGGGLVQPGGSLRLSCAASGFTFADY | DIQLTQSPSSLSASVGDRVTITCRASQSISSYLN |
| | AMTWVRQAPGKGLEWVSAISGSGAATYFADSV | WYQQKPGKAPKLLIYAASNLQSGVPSRFSGSG |
| | KGRFTISRDNSKNTLYLQMNSLRAEDTAVYYCA | SGTDFTLTISSLQPEDFATYYCQQTYTVPLTFGG |
| | RVSSGHWDFDYWGQGTLVTVSS (SEQ ID NO: | GTKVEIK (SEQ ID NO: 15) |
| | 13) | |
| Ab16 | EVQLLESGGGLVQPGGSLRLSCAASGFTFSNY | DIQLTQSPSSLSASVGDRVTITCRASQSISSYLN |
| | AMSWVRQAPGKGLEWVSAISGSGFTTYYADSV | WYQQKPGKAPKLLIYAASNLQSGVPSRFS GSG |
| | KGRFTISRDNSKNTLYLQMNSLRAEDTAVYYCA | SGTDFTLTISSLQPEDFATYYCQQTYTVPLTFGG |
| | RVSSGHWDFDYWGQGTLVTVSS (SEQ ID NO: | GTKVEIK (SEQ ID NO: 15) |
| | 228) | |
| Ab17 | EVQLLESGGGVVQPGGSLRLSCAASGFTFSSF | DIQLTQSPSSLSASVGDRVTITCRASQSISSYLN |
| | AMTWVRQAPGKGLEWVSAISGSGAATYFADSV | WYQQKPGKAPKLLIYAASNLQSGVPSRFS GSG |
| | KGRFTISRDNSKNTLYLQMNSLRAEDTAVYYCA | SGTDFTLTISSLQPEDFATYYCQQTYTVPLTFGG |
| | RVSSGHWDFDYWGQGTLVTVSS (SEQ ID NO: | GTKVEIK (SEQ ID NO: 15) |
| | 230) | |
| Ab18 | QVQLVESGGGVVQPGRSLRLSCAASGFTFSDY | DIQLTQSPSSLSASVGDRVTITCRASQSISSYLN |
| | AMVWVRQAPGKGLEWVSAISGSGAATYFADSV | WYQQKPGKAPKLLIYAASNLQSGVPSRFSGSG |
| | KGRFTISRDNSKNTLYLQMNSLRAEDTAVYYCA | SGTDFTLTISSLQPEDFATYYCQQTYTVPLTFGG |
| | TVSSGHWDFDYWGQGTLVTVSS (SEQ ID NO: | GTKVEIK (SEQ ID NO: 15) |
| | 232) | |
| Ab19 | QVQLVESGGGVVQPGRSLRLSCAASGFTFSSF | DIQLTQSPSSLSASVGDRVTITCRASQSISSYLN |
| | AMTWVRQAPGKGLEWVSAISGSGAATYFADSV | WYQQKPGKAPKLLIYAASNLQSGVPSRFSGSG |
| | KGRFTISRDNSKNTLYLQMNSLRAEDTAVYYCA | SGTDFTLTISSLQPEDFATYYCQQTYTVPLTFGG |
| | RVSSGHWDFDEWGQGTLVTVSS (SEQ ID NO: | GTKVEIK (SEQ ID NO: 15) |
| | 234) | |
| Ab20 | EVQLLESGGGLVQPGGSLRLSCAASGFTFSAYA | DIQLTQSPSSLSASVGDRVTITCRASQSISSYLN |
| | MTWVRQAPGKGLEWVSAISGSGGVTYYADSVK | WYQQKPGKAPKLLIYAASNLQSGVPSRFSGSG |
| | GRFTISRDNSKNTLYLQMNSLRAEDTAVYYCAR | SGTDFTLTISSLQPEDFATYYCQQTYTVPLTFGG |
| | VSSGHWDYDYWGQGTLVTVSS (SEQ ID NO: | GTKVEIK (SEQ ID NO: 15) |
| | 236) | |
| Ab35 | EVQLLESGGGLVQPGGSLRLSCAASGFTFRNY | DIQMTQSPSSLSASVGDRVTITCRASQSISSYLN |
| | AMSWVRQAPGKGLEWVSSISGSGGATYYADSV | WYQQKPGKAPKLLIYDASSLQSGVPSRFSGSG |
| | KGRFTISRDNSKNTLYLQMNSLRAEDTAVYYCA | SGTDFTLTISSLQPEDFATYYCQQSYSAPFTFG |
| | RVSSYLWDFDYWGQGTLVTVSS (SEQ ID NO: | QGTKVEIK (SEQ ID NO: 211) |
| | 238) | |
| Ab36 | EVQLLESGGGLVQPGGSLRLSCAASGFTFRNY | DIQMTQSPSSLSASVGDRVTITCRASQSISSYLN |
| | AMSWVRQAPGKGLEWVSSISGSGGATYYADSV | WYQQKPGKAPKLLIYDASSLQSGVPSRFSGSG |
| | KGRFTISRDNSKNTLYLQMNSLRAEDTAVYYCA | SGTDFTLTISSLQPEDFATYYCQQSYSAPFTFG |
| | RVSSGHWDLDYWGQGTLVTVSS (SEQ ID NO: | QGTKVEIK (SEQ ID NO: 211) |
| | 240) | |
| Ab1 | EVQLLESGGGLVQPGGSLRLSCAASGFTFRNY | DIQLTQSPSSLSASVGDRVTITCQASQDISNFLN |
| | AMSWVRQAPGKGLEWVSSISGSGGATYYADSV | WYQQKPGKAPKLLIYDASNLETGVPSRFSGSGS |
| | KGRFTISRDNSKNTLYLQMNSLRAEDTAVYYCA | GTDFTFTISSLQPEDIATYYCQQVFNPPLTFGGG |
| | RVSSGHWDFDYWGQGTLVTVSS (SEQ ID NO: | TKVEIK (SEQ ID NO: 243) |
| | 216) | |

In related embodiments, the antibody or the fragment thereof may comprise a heavy chain variable domain having at least 95% sequence identity with anyone of the heavy chain variable domain sequences provided in Table 4 (e.g., selected from SEQ ID NOs: 210, 212, 214, 216, 222, 224, 226, 13, 228, 230, 232, 234, and 236) and a corresponding light chain variable domain having at least 95%, 96%, 97%, 98% or 99% sequence identity with any one of the light chain variable domain sequences provided in Table 4 (e.g., selected from SEQ ID NOs: 211, 217, 223, 15, and 243). For example, the antibody may comprise a heavy chain variable domain having at least 95%, 96%, 97%, 98% or 99% sequence identity with the heavy chain variable domain of Ab2 (SEQ ID NO: 13) and a light chain variable domain having at least 95%, 96%, 97%, 98% or 99% sequence identity with the light chain variable domain of Ab2 (SEQ ID NO: 15).

In preferred embodiments, the antibody cross-reacts with a murine counterpart. In particularly preferred embodiments, the antibody shows cross-reactivity to human, cynomolgus monkey, mouse and rat antigens.

In the context of antibody-antigen interactions, the area of the antigen where the binding of the antibody forms an interface may be referred to as a “binding region.” In some embodiments, the binding region(s) of a Category 1 antibody to the proTGFβ1 complexes may comprise at least a portion of the prodomain of the proTGFβ1. In preferred embodiments, the portion of the prodomain to which the antibody binds comprises one or more amino acid residues of Latency Lasso. In particularly preferred embodiments, the antibody binds a combinatorial epitope that comprises one or more amino acid residues of the Latency Lasso (within the prodomain) and additionally one or more amino acid residues of the growth factor domain of the proTGFβ1. In some embodiments of the Category 1 antibodies, the antibody or the antigen-binding fragment makes contact with one or more of the following amino acid residues of human proTGFβ1: S35, G37, E38, V39, P40, P41, G42, P43, R274, K280, and H283, based on the amino acid sequence set forth as SEQ ID NO: 24.

In some embodiments, the Category 1 antibodies have inhibitory potency against TGFβ1 such that, when measured by a suitable cell-based assay(such as CAGA assays described herein), the antibody has an IC₅₀ of ≤5 nM (e.g., ≤5 nM, ≤4 nM, ≤3 nM, ≤52 nM, ≤1 nM) against a hLTBP1-TGFβ1 complex and a hLTBP3-TGFβ1 complex.

In preferred embodiments, the antibody comprises a heavy chain comprising an amino acid

(SEQ ID NO: 17)
EVQLLESGGGLVQPGGSLRLSCAASGFTFADYAMTWVRQAPGKGLEWVSA
ISGSGAATYFADSVKGRFTISRDNSKNTLYLQMNSLRAEDTAVYYCARVS
SGHWDFDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG,

and a light chain comprising an amino acid sequence

(SEQ ID NO: 19)
DIQLTQSPSSLSASVGDRVTITCRASQSISSYLNWYQQKPGKAPKLLIYAA
SNLQSGVPSRFSGSGSGTDFTLTISSLQPEDFATYYCQQTYTVPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

### Category 2 Antibodies

In another aspect, the present disclosure provides antibodies (e.g., immunoglobulins, modified or engineered immunoglobulin-like molecules, and antigen-binding fragments thereof) having the Core Properties of Table 1 and further meet the additional required criteria defined by CDR sequences of Category 2. Thus, such antibodies or fragments thereof specifically bind each of hLTBP1-proTGFβ1, hLTBP3-proTGFβ1, hGARP-proTGFβ1 and hLRRC33-proTGFβ1 complexes, wherein the antibodies or the fragments comprise two or more of the following CDRs, provided in Table 5 below, with the proviso that the antibody comprises at least one CDR that is selected from the group consisting of: H-CDR1, H-CDR2, L-CDR2, and L-CDR3, provided in the Table 5 below.

TABLE 5
CDR sequence requirements for Category 2 antibodies

| CDR | Sequences |
|---|---|
| H-CDR1 | FTFSDYAMT (SEQ ID NO: 250), optionally with 1 amino acid change |
| H-CDR2 | AISGSGAATYYADSVKG (SEQ ID NO: 251), optionally with 1 amino acid |
| | change |
| H-CDR3 | ARVSSGHWDFDY (SEQ ID NO: 110), optionally with 1 amino acid change |
| L-CDR1 | RASQSISSYLN (SEQ ID NO: 111), optionally with 1 amino acid change |
| L-CDR2 | AASNLQS (SEQ ID NO: 136), optionally with 1 amino acid change |
| L-CDR3 | QQTYTVPLT (SEQ ID NO: 12), optionally with 1 amino acid change |

Thus, the invention provides an isolated monoclonal antibody or antigen-binding fragment thereof comprising an H-CDR1, an H-CDR-2, an H-CDR3, an L-CDR1, an L-CDR2 and an L-CDR3, wherein:
the H-CDR1 comprises the amino acid sequence FTFSDYAMT (SEQ ID NO: 250), optionally with 1 amino acid change;
the H-CDR2 comprises the amino acid sequence AISGSGAATYYADSVKG (SEQ ID NO: 251), optionally with 1 amino acid change;
the H-CDR3 comprises the amino acid sequence ARVSSGHWDFDY (SEQ ID NO: 110), optionally with 1 amino acid change;
the L-CDR1 comprises the amino acid sequence RASQSISSYLN (SEQ ID NO: 111), optionally with 1 amino acid change;
the L-CDR2 comprises the amino acid sequence AASNLQS (SEQ ID NO: 136), optionally with 1 amino acid change; and,
the L-CDR3 comprises the amino acid sequence QQTYTVPLT (SEQ ID NO: 12), optionally with 1 amino acid change.

As used herein, the phrase “amino acid changes” or“changes in amino acid residues” includes amino acid substitutions and/or deletions.

In some embodiments, the antibody comprises three or more (e.g., 3, 4, 5 or all 6) of the following CDR sequences:

(SEQ ID NO: 250)
the H-CDR1 is FTFSDYAMT;
(SEQ ID NO: 251)
the H-CDR2 is AISGSGAATYYADSVKG;
(SEQ ID NO: 110)
the H-CDR3 is ARVSSGHWDFDY;
(SEQ ID NO: 111)
the L-CDR1 is RASQSISSYLN;
(SEQ ID NO: 136)
the L-CDR2 is AASNLQS;
and,
(SEQ ID NO: 12)
the L-CDR3 is QQTYTVPLT.

In some embodiments, the antibody comprises all six of the CDR sequences: FTFSDYAMT (SEQ ID NO: 250), AISGSGAATYYADSVKG (SEQ ID NO: 251), ARVSSGHWDFDY (SEQ ID NO: 110), RASQSISSYLN (SEQ ID NO: 111), AASNLQS (SEQ ID NO: 136) and QQTYTVPLT (SEQ ID NO: 12).

In some embodiments of the Category 2 antibodies, the antibody or the antigen-binding fragment comprises a heavy chain variable region (V_{H}) that is at least 95% identical to the amino acid sequence set for in SEQ ID NO: 13, and a light chain variable region (V_{L}) that is at least 95% identical to the amino acid sequence set forth in SEQ ID NO: 15. Within the heavy chain variable region in accordance with SEQ ID NO: 13, the residue at position 31 may be D, the residue at position 33 may be A, the residue at position 54 may be S, residue at position 59 may be Y, the residue at position 101 may be S, the residue at position 102 may be G, the residue at position 103 may be H, the residue at position 104 may be W, or, any combinations thereof. Within the light chain variable region in accordance with SEQ ID NO: 15, the residue at position 32 may be Y, the residue at position 49 may be Y, the residue at position 91 may be T, the residue at position 92 may be Y, or any combinations thereof.

In some embodiments, the antibody or the antigen-binding fragment comprises a heavy chain variable region (V_{H}) that is at least 90% identical to the amino acid sequence set for in SEQ ID NO: 13 and comprises amino acid residues D31, A33, S54, Y59, S101, G102, H103, and W104 based on the amino acid sequence set forth in SEQ ID NO: 13, and a light chain variable region (V_{L}) that is at least 90% identical to the amino acid sequence set forth in SEQ ID NO: 15 and comprises amino acid residues Y32, Y49, T91, and Y92, based on the amino acid sequence set forth in SEQ ID NO: 15.

In some embodiments, the antibody cross-reacts with a murine counterpart. In particularly preferred embodiments, the antibody shows cross-reactivity to human, cynomolgus monkey, mouse and rat antigens.

In some embodiments, the binding of a Category 2 antibody to the proTGFβ1 complexes involves at least a portion of the prodomain of the proTGFβ1. In preferred embodiments, the portion of the prodomain to which the antibody binds comprises one or more amino acid residues of Latency Lasso. In particularly preferred embodiments, the antibody binds a combinatorial epitope that comprises one or more amino acid residues of the Latency Lasso (within the prodomain) and one or more amino acid residues of the growth factor domain of the proTGFβ1. In some embodiments of the Category 2 antibodies, the antibody or the antigen-binding fragment makes contact with one or more of the following amino acid residues of human proTGFβ1: S35, G37, E38, V39, P40, P41, G42, P43, R274, K280, and H283, based on the amino acid sequence set forth as SEQ ID NO: 24.

In some embodiments, the Category 2 antibodies have inhibitory potency against TGFβ1 such that, when measured by a suitable cell-based assay(such as CAGA assays described herein), the antibody has an IC₅₀ of ≤5 nM (e.g., ≤5 nM, ≤4 nM, ≤3 nM, ≤2 nM, ≤1 nM) against a hLTBP1-TGFβ1 complex and a hLTBP3-TGFβ1 complex.

### Category 3 Antibodies

In a further aspect, the present disclosure provides monoclonal antibodies or antigen-binding fragments thereof that specifically bind each of: hLTBP1-proTGFβ1, hLTBP3-proTGFβ1, hGARP-proTGFβ1 and hLRRC33-proTGFβ1 complexes and are capable of selectively inhibiting TGFβ1 signaling, wherein the antibodies or the fragments bind each of the hLTBP1-proTGFβ1 and hLTBP3-proTGFβ1 complexes with KD of ≤1 nM and bind each of the hGARP-proTGFβ1 and hLRRC33-proTGFβ1 complexes with KD >1 nM, as measured by solution equilibrium titration.

In some embodiments, the antibody cross-reacts with a murine counterpart. In particularly preferred embodiments, the antibody shows cross-reactivity to human, cyno, mouse and rat antigens.

In some embodiments, binding region(s) of the proTGFβ1 complex bound by a Category 3 antibody may include at least a portion of the prodomain of the proTGFβ1. In preferred embodiments, the portion of the prodomain to which the antibody binds comprises one or more amino acid residues of Latency Lasso. In particularly preferred embodiments, the antibody binds a combinatorial epitope that comprises one or more amino acid residues of the Latency Lasso (within the prodomain) and one or more amino acid residues of the growth factor domain of the proTGFβ1. In some embodiments, the Category 3 antibody or the antigen-binding fragment makes contact with one or more of the following amino acid residues of human proTGFβ1: S35, G37, E38, V39, P40, P41, G42, P43, R274, K280, and H283, based on the amino acid sequence set forth in SEQ ID NO: 24.

In some embodiments, the Category 3 antibodies have inhibitory potency against TGFβ1 such that, when measured by a suitable cell-based assay(such as CAGA assays described herein), the antibody has an IC₅₀ of ≤5 nM (e.g., ≤5 nM, ≤4 nM, ≤3 nM, ≤2 nM, ≤1 nM) against a hLTBP1-TGFβ1 complex and a hLTBP3-TGFβ1 complex.

### Category 4 Antibodies

In a yet further aspect, the present disclosure provides monoclonal antibodies or antigen-binding fragments thereof that specifically bind each of: hLTBP1-proTGFβ1, hLTBP3-proTGFβ1, hGARP-proTGFβ1 and hLRRC33-proTGFβ1 complexes and are capable of selectively inhibiting TGFβ1 signaling, wherein the antibodies or the fragments bind each of the hLTBP1-proTGFβ1 and hLTBP3-proTGFβ1 complexes with KD of ≤1 nM as measured by solution equilibrium titration, and, wherein the average affinity for hLTBP1-proTGFβ1 and hLTBP3-proTGFβ1 complexes is at least five-fold greater than the average affinity for hGARP-proTGFβ1 and hLRRC33-proTGFβ1 complexes.

In some embodiments, the antibody cross-reacts with a murine counterpart. In particularly preferred embodiments, the antibody shows cross-reactivity to human, cyno, mouse and rat antigens.

In some embodiments, binding region(s) of the proTGFβ1 complex bound by a Category 4 antibody may include at least a portion of the prodomain of the proTGFβ1. In preferred embodiments, the portion of the prodomain to which the antibody binds comprises one or more amino acid residues of Latency Lasso. In particularly preferred embodiments, the antibody binds a combinatorial epitope that comprises one or more amino acid residues of the Latency Lasso (within the prodomain) and one or more amino acid residues of the growth factor domain of the proTGFβ1. In some embodiments, the Category 4 antibody or the antigen-binding fragment makes contact with one or more of the following amino acid residues of human proTGFβ1: S35, G37, E38, V39, P40, P41, G42, P43, R274, K280, and H283, based on the amino acid sequence set forth in SEQ ID NO: 24.

In some embodiments, the Category 4 antibodies have inhibitory potency against TGFβ1 such that, when measured by a suitable cell-based assay(such as CAGA assays described herein), the antibody has an IC₅₀ of ≤5 nM (e.g., ≤5 nM, ≤4 nM, ≤3 nM, ≤2 nM, ≤1 nM) against a hLTBP1-TGFβ1 complex and a hLTBP3-TGFβ1 complex.

### Category 5 Antibodies

In another aspect, the present disclosure provides monoclonal antibodies or antigen-binding fragments thereof that specifically bind each of: hLTBP1-proTGFβ1, hLTBP3-proTGFβ1, hGARP-proTGFβ1 and hLRRC33-proTGFβ1 complexes with KD of ≤10 nM (preferably ≤5 nM); and, wherein the antibody has greater than five-fold matrix/LTBP-bias in relative affinities between at least one of the matrix-associated complexes (e.g., hLTBP1-proTGFβ1 and/or hLTBP3-proTGFβ1) and at least one of the cell-associated complexes (e.g., hGARP-proTGFβ1 and/or hLRRC33-proTGFβ1).

In some embodiments, the antibody cross-reacts with a murine counterpart. In particularly preferred embodiments, the antibody shows cross-reactivity to human, cyno, mouse and rat antigens.

In some embodiments, binding region(s) of the proTGFβ1 complex bound by a Category 5 antibody may include at least a portion of the prodomain of the proTGFβ1. In preferred embodiments, the portion of the prodomain to which the antibody binds comprises one or more amino acid residues of Latency Lasso. In particularly preferred embodiments, the antibody binds a combinatorial epitope that comprises one or more amino acid residues of the Latency Lasso (within the prodomain) and one or more amino acid residues of the growth factor domain of the proTGFβ1. In some embodiments, the Category 5 antibody or the antigen-binding fragment makes contact with one or more of the following amino acid residues of human proTGFβ1: S35, G37, E38, V39, P40, P41, G42, P43, R274, K280, and H283, based on the amino acid sequence set forth in SEQ ID NO: 24.

In some embodiments, the Category 5 antibodies have inhibitory potency against TGFβ1 such that, when measured by a suitable cell-based assay(such as CAGA assays described herein), the antibody has an IC₅₀ of ≤5 nM (e.g., ≤5 nM, ≤4 nM, ≤3 nM, ≤2 nM, ≤1 nM) against a hLTBP1-TGFβ1 complex and a hLTBP3-TGFβ1 complex.

### Characterization of the Novel Improved Antibodies of the Invention

### Binding Profiles

Antibodies disclosed herein have enhanced binding activities. The antibodies are capable of specifically binding to each of the presenting molecule-proTGFβ1 complexes (sometimes referred to as “Large Latency Complex” which is a ternary complex comprised of a proTGFβ1 dimer coupled to a single presenting molecule), namely, LTBP1-proTGFβ1, LTBP3-proTGFβ1, GARP-proTGFβ1 and LRRC33-proTGFβ1. Typically, recombinantly produced, purified protein complexes are used as antigens (e.g., antigen complexes) to evaluate or confirm the ability of an antibody to bind the antigen complexes in suitable in vitro binding assays. Such assays are well known in the art and include, but are not limited to Bio-Layer Interferometry (BLI)-based assays (such as Octet®) and solution equilibrium titration-based assays (such as MSD-SET).

BLI-based binding assays are widely used in the art for measuring affinities and kinetics of antibodies to antigens. It is a label-free technology in which biomolecular interactions are analyzed on the basis of optical interference. One of the proteins, for example, an antibody being tested, can be immobilized on the biosensor tip. When the other protein in solution, for example, an antigen, becomes bound to the immobilized antibody, it causes a shift in the interference pattern, which can be measured in real-time. This allows the monitoring of binding specificity, rates of association and dissociation, as well as concentration dependency. Thus, BLI is a kinetic measure that reveals the dynamics of the system. Due to its ease of use and fast results, BLI-based assays such as the Octet® system (available from ForteBio/Molecular Devices, Fremont Calif.), are particularly convenient when used as an initial screening method to identify and separate a pool of “binders” from a pool of “non-binders” or “weak binders” in the screening process.

BLI-based binding assays revealed two classes of binders. The first class is referred to as “context-balanced/context-independent” antibodies. Ab11, Ab2, and Ab20 fall within this class when binding affinity is measured by Octet® (highlighted in Table 6). As can be seen in the Table summarizing BLI-based binding profiles of non-limiting examples of antibodies, these antibodies show relatively uniform KD values in a sub-nanomolar range across the four target complexes, with relatively low matrix-to-cell differentials (no greater than five-fold bias) (see column (H)). This can be contrasted against the previously identified antibody Ab3, which shows significantly higher relative affinities towards matrix-associated complexes (27+ fold bias) over cell-associated complexes. The bias correction achieved here was done so without detrimentally affecting the affinities towards LTBP1/3 complexes. In fact, while significantly reducing the degree of context-bias across the four complexes, overall enhancement of affinities was also achieved.

The second class represents a majority of the antibodies exemplified in Table below and is referred to as “context-biased” antibodies. As compared to the reference antibody Ab3, overall affinities are improved in these antibodies, while the preference/bias for the matrix complexes over cell-associated complexes is maintained or enhanced when measured by Octet®. In some embodiments, context-biased antibodies show bias against a GARP-proTGFβ1 complex (e.g., human GARP-proTGFβ1) over the other three target complexes. As used herein, the expression “bias against a GARP-proTGFβ1 complex” means that the affinity of the antibody to the GARP complex is weaker(i.e., greater KD values) than affinities to the other target complexes. Such context-biased antibodies may be advantageously used to treat conditions where it is undesirable to stimulate the subject's immune system.

Note that the term “context-independent” is used herein with a greater degree of stringency as compared to previous more general usage. According to the present disclosure, the term confers a level of uniformity in relative affinities (i.e., unbias) that the antibody can exert towards different antigen complexes. Thus, the context-independent antibody disclosed herein is capable of targeting multiple types of TGFβ1 precursor complexes (e.g., presenting molecule-proTGFβ1 complexes) and of binding to each such complex with equivalent affinities (i.e., without bias) with KD values of at least 5 nM as measured by Octet®. As presented below, many antibodies encompassed by the invention have KD values in a sub-nanomolar range.

Table 6 below provides non-limiting examples of high-affinity, context-independent and context-biased proTGFβ1 antibodies encompassed by the present invention. The table provides representative results from in vitro binding assays, as measured by Octet®. Similar results are also obtained by an SPR-based technique (Biacore® System).

Column (A) of the table provides monoclonal antibodies with discrete amino acid sequences. Ab3 (shown in bold) is a reference antibody identified previously, which was shown to be potent in cell-based assays; efficacious in various animal models; and, with a clean toxicology profile (disclosed in: PCT/US2018/012601). Columns (B), (C), (E) and (F) provide affinities of each of the listed antibodies, measured in KD, towards the antigen complex as indicated, respectively. More specifically, column (B) shows the affinity to a recombinant human LTBP1-proTGFβ1 complex; column (C) shows the affinity to a recombinant human LTBP3-proTGFβ1 complex; (E) shows the affinity to a recombinant human GARP-proTGFβ1 complex; and (F) shows the affinity to a recombinant human LRRC33-proTGFβ1 complex, of each of the antibodies. Average KD values calculated from (B) and (C) are shown in the corresponding column (D), which collectively represents affinities of the antibodies to ECM- or matrix-associated proTGFβ1 complexes. Similarly, Average KD values calculated from (E) and (F) are shown in the corresponding column (G), which collectively represents affinities of the antibodies to cell-surface or cell-associated proTGF complexes. Finally, relative ratios between the average KD values from columns (D) and (G) are expressed as fold bias” in column (H). Thus, the greater the numerical of column (H) is, the greater bias exists for the particular antibody, when comparing binding preferences of the antibody for matrix-associated complexes to cell-surface complexes. This is one way of quantitatively representing and comparing inherent bias of antibodies to their target complexes. Such analyses may be useful in guiding the selection process for a candidate antibody for particular therapeutic use.

TABLE 6
In vitro kinetic binding profiles of representative antibodies by Octet ®

| | Matrix-associated proTGFβ1 | | | Cell-associated proTGFβ1 | | | |
|---|---|---|---|---|---|---|---|
| (A) | | | (D) | | | (G) | (H) |
| Ab | (B) | (C) | ECM AVRG | (E) | (F) | Cell AVRG | G/D (fold |
| Ref | hLTBP1 | hLTBP3 | (nM) | hGARP | hLRRC33 | (nM) | bias) |
| Ab3 | 4.70E−10 | 4.59E−10 | 0.4645 | 1.73E−08 | 8.52E−09 | 12.91 | 27.79 |
| Ab7 | 4.87E−10 | 4.17E−10 | 0.452 | 1.80E−08 | 1.03E−08 | 14.15 | 31.3 |
| Ab8 | 3.29E−10 | 3.02E−10 | 0.3155 | 1.54E−08 | 1.00E−08 | 12.7 | 40.25 |
| Ab9 | 3.48E−10 | 2.84E−10 | 0.316 | 1.55E−08 | 1.30E−09 | 8.4 | 26.58 |
| Ab10 | 2.62E−10 | 3.19E−10 | 0.2905 | 1.02E−08 | 1.06E−09 | 5.63 | 19.38 |
| Ab11 | 1.67E−10 | 1.54E−10 | 0.1605 | 8.42E−10 | 5.55E−10 | 0.6985 | 4.352 |
| Ab12 | 1.57E−10 | 1.83E−10 | 0.17 | 7.01E−09 | 7.75E−10 | 3.8925 | 22.897 |
| Ab13 | 2.76E−10 | 2.49E−10 | 0.2625 | 1.00E−08 | 1.40E−09 | 5.7 | 21.71 |
| Ab14 | 3.36E−10 | 3.46E−10 | 0.341 | 1.05E−08 | 1.34E−09 | 5.92 | 17.36 |
| Ab15 | 1.50E−10 | 1.68E−10 | 0.159 | 7.01E−09 | 7.02E−10 | 3.856 | 24.25 |
| Ab2 | 1.65E−10 | 1.48E−10 | 0.1565 | 7.78E−10 | 5.08E−10 | 0.643 | 4.1086 |
| Ab16 | 1.81E−10 | 1.32E−10 | 0.1565 | 4.16E−09 | 6.31E−10 | 2.3955 | 15.307 |
| Ab17 | 1.74E−10 | 2.08E−10 | 0.191 | 8.62E−09 | 6.58E−10 | 4.639 | 24.288 |
| Ab18 | 3.12E−10 | 2.57E−10 | 0.2845 | 1.15E−08 | 1.02E−09 | 6.26 | 22.00 |
| Ab19 | 1.89E−10 | 1.56E−10 | 0.1725 | 1.12E−08 | 6.92E−10 | 5.946 | 34.47 |
| Ab20 | 1.92E−10 | 1.31E−10 | 0.1615 | 8.84E−10 | 5.45E−10 | 0.7145 | 4.424 |
| Ab35 | 1.59E−10 | 1.69E−10 | 0.164 | 7.37E−09 | 5.45E−10 | 3.9575 | 24.13 |
| Ab36 | 3.53E−10 | 4.15E−10 | 0.384 | 1.28E−08 | 1.42E−09 | 7.11 | 18.5156 |

Whilst the kinetics of binding profiles (e.g., “on” and “off” rates) obtainable from BLI-based assays provide useful information, Applicant of the present disclosure contemplated that, based on the mechanism of action of the activation inhibitors disclosed herein, that is, antibodies that work by binding to a tethered (e.g., tissue-localized) inactive (e.g., latent) target thereby preventing it from getting activated, binding properties measured at equilibrium might more accurately reflect their in vivo potency. For example, antibodies with fast “on” rate (“Kₒₙ”) which would be reflected in binding measurements obtained by BLI, may provide useful parameters for evaluating neutralizing antibodies (e.g., antibodies that directly target and sequester the active, soluble growth factor itself). However, the same may not necessarily apply for antibodies that function as activation inhibitors, such as those disclosed herein. Taking this difference in the mechanism of action into consideration, further evaluation of binding properties was carried out by the use of another mode of in vitro binding assays that allows the determination of affinity at equilibrium.

Solution equilibrium titration (“SET”) is an assay whereby binding between two molecules (such as an antigen and an antibody that binds the antigen) can be measured at equilibrium in a solution. For example, Meso-Scale Discovery (“MSD”)-based SET, or MSD-SET, is a useful mode of determining dissociation constants for particularly high-affinity protein-protein interactions at equilibrium (see, for example: Ducata et al. (2015) J Biomolecular Screening 20(10): 1256-1267). The SET-based assays are particularly useful for determining KD values of antibodies with sub-nanomolar(e.g., picomolar) affinities.

Select antibodies of the present disclosure, as measured by MSD-SET, show context-bias (i.e., greater than five-fold bias) or unbias (i.e., less than 5-fold bias) toward matrix-associated complexes over cell-associated complexes. For example, as shown in Table 7, Ab2 shows context-bias toward matrix-associated complexes over cell-associated complexes, when measured by MSD-SET (i.e., 8.66-fold bias). Alternatively, Ab3 and Ab19 show lesser degrees of such bias (i.e., only 2.16-fold and 1.675-fold bias, respectively) when measured by MSD-SET. Without being bound by a particular theory, the differences in the kinetics of the assays and/or differences in the nature of recombinant proteins may account for the noticeable differences seen in Ab19 binding to hGARP complexes observed in the above Octet assay (see Table 6) compared to the below MSD-SET assay (see Table 7 below). For example, the Octet assay measures kinetic on and off rates, while the MSD-SET assay measures binding at equilibrium. In addition, the recombinant antibodies used in the Octet assay were expressed as IgG1 antibodies in yeast, while the antibodies used in the MSD assay were expressed as IgG4 antibodies in mammalian cells.

TABLE 7
In vitro binding profiles of select antibodies measured at equilibrium by MSD-SET

| | Matrix-associated proTGFβ1 | | | Cell-associated proTGFβ1 | | | |
|---|---|---|---|---|---|---|---|
| (A) | | | (D) | | | (G) | (H) |
| Ab | (B) | (C) | ECM AVRG | (E) | (F) | Cell AVRG | G/D (fold |
| Ref | hLTBP1 | hLTBP3 | (nM) | hGARP | hLRRC33 | (nM) | bias) |
| C1 | 3.30E−08 | 1.40E−08 | 23.2 | 5.10E−09 | 2.20E−09 | 3.65 | 0.16 |
| C2 | 2.10E−08 | 1.20E−08 | 16.5 | 8.80E−09 | 6.10E−09 | 7.45 | 0.48 |
| Ab3 | 1.30E−08 | 1.62E−08 | 14.6 | 2.80E−08 | 3.50E−08 | 31.5 | 2.16 |
| Ab11 | 1.20E−09 | 1.20E−09 | 1.2 | 3.00E−09 | 6.30E−09 | 4.65 | 3.87 |
| AB12 | 7.80E−10 | 6.60E−10 | 0.72 | 1.70E−09 | 3.20E−09 | 2.45 | 3.40 |
| Ab2 | 3.60E−10 | 1.71E−10 | 0.266 | 1.40E−09 | 3.20E−09 | 2.30 | 8.66 |
| Ab19 | 2.10E−09 | 2.70E−09 | 2.835 | 3.80E−09 | 5.70E−09 | 4.75 | 1.68 |
| Ab20 | 2.20E−10 | 1.80E−10 | 0.20 | 1.10E−09 | 1.60E−09 | 1.35 | 6.75 |
| Ab35 | 2.70E−10 | 2.60E−10 | 0.265 | 1.10E−09 | 1.10E−09 | 1.1 | 4.15 |

Table 7 also includes three previously described TGFβ1-selective antibodies (C1, C2 and Ab3) as reference antibodies. C1 and C2 were first disclosed in PCT/US2017/021972 published as WO 2017/156500, and Ab3 was described in PCT/US2018/012601 published as WO2018/129329.

As can be seen in Table 7, binding activities of the novel antibodies towards matrix-associated complexes (hLTBP1-proTGFβ1 and hLTBP3-proTGFβ1) measured at equilibriums how significant improvement as compared to the earlier TGFβ31-selective antibodies. Many of the novel antibodies disclosed herein have high affinities for the hLTBP1-proTGFβ1 and hLTBP3-proTGFβ1 complexes with KD values in a sub-nanomolar range (e.g., less than 1 nM), while maintaining moderate affinities (e.g., single-digit nanomolar range, e.g., between 1 and 5 nM) for cell-associated complexes (hGARP-proTGFβ1 and hLRRC33-proTGFβ1), rendering these novel antibodies particularly advantageous for use in the treatment of disease that involves dysregulation of the extracellular matrix. Extracellular matrix dysregulation may include remodeling and/or increased stiffness of the matrix caused by, inter alia, aberrant expression of ECM scaffold proteins. Increased frequency of myofibroblasts and myofibroblast-like cells in the affected tissue (such as fibrosis and tumor) may be observed, which may be normalized by the TGFβ1-selective inhibitors of the present invention.

In a related aspect, the invention provides an antibody or antigen-binding fragment thereof that i) cross-competes with anyone of the Category 1 or Category 2 antibodies and ii) satisfies the criteria set forth in Category 3, Category 4 and/or Category 5.

### Potency

Antibodies disclosed herein may be broadly characterized as “functional antibodies” for their ability to inhibit TGFβ1 signaling. As used herein, “a functional antibody” confers one or more biological activities by virtue of its ability to bind an antigen (e.g., antigen complexes). Functional antibodies therefore broadly include those capable of modulating the activity/function of target molecules (i.e., antigen). Such modulating antibodies include inhibiting antibodies (or inhibitory antibodies) and activating antibodies. The present disclosure is drawn to antibodies which can inhibit a biological process mediated by TGFβ1 signaling associated with multiple contexts of TGFβ1. Inhibitory agents used to carry out the present invention, such as the antibodies described herein, are intended to be TGFβ 1-selective and not to target or interfere with TGFβ2 and TGFβ3 when administered at a therapeutically effective dose (dose at which sufficient efficacy is achieved within acceptable toxicity levels). The novel antibodies of the present disclosure have enhanced inhibitory activities (potency) as compared to previously identified activation inhibitors of TGFβ1.

In some embodiments, potency of an inhibitory antibody may be measured in suitable cell-based assays, such as CAGA reporter assays described herein. Generally, cultured cells, such as heterologous cells and primary cells, may be used for carrying out cell-based potency assays. Cells that express endogenous TGFβ1 and/or a presenting molecule of interest, such as LTBP1, LTBP3, GARP and LRRC33, may be used. Alternatively, exogenous nucleic acids encoding protein(s) of interest, such as TGFβ1 and/or a presenting molecule of interest, such as LTBP1, LTBP3, GARP and LRRC33, may be introduced into such cells, for example by transfection (e.g., stable transfection or transient transfection) or by viral vector-based infection. In some embodiments, LN229 cells are employed for such assays. The cells expressing TGFβ1 and a presenting molecule of interest (e.g., LTBP1, LTBP3, GARP or LRRC33) are grown in culture, which “present” the large latent complex either on cell surface (when associated with GARP or LRRC33) or deposit into the ECM (when associated with an LTBP). Activation of TGFβ1 may be triggered by integrin, expressed on another cell surface. The integrin-expressing cells may be the same cells co-expressing the large latent complex or a separate cell type. Reporter cells are added to the assay system, which incorporates a TGFβ-responsive element. In this way, the degree of TGFβ activation may be measured by detecting the signal from the reporter cells (e.g., TGFβ-responsive reporter genes, such as luciferase coupled to a TGFβ-responsive promoter element) upon TGFβ activation. Using such cell-based assay systems, inhibitory activities of the antibodies can be determined by measuring the change (reduction) or difference in the reporter signal (e.g., luciferase activities as measured by fluorescence readouts) either in the presence or absence of test antibodies.

In some embodiments, the inhibitory potency (IC50) of the novel antibodies of the present disclosure calculated based on cell-based assays (such as LN229 cell assays described elsewhere herein) may be less than 10 nM measured against each of the hLTBP1-proTGFβ1 and hLTBP3-proTGFβ1 complexes. In some embodiments, the antibodies have an IC50 of 5 nM or less (i.e., ≤5 nM) measured against each of the hLTBP1-proTGFβ1 and hLTBP3-proTGFβ1 complexes. In preferred embodiments, the IC50 of the antibody measured against at least one of the hLTBP1-proTGFβ1 and hLTBP3-proTGFβ1 complexes is less than 1 nM. In some embodiments, the antibody has an IC50 of less than 1 nM against at least one of the hLTBP1-proTGFβ1 and hLTBP3-proTGFβ1 complexes and further at least one of the mLTBP1-proTGFβ1 and mLTBP3-proTGFβ1 complexes. In some embodiments, the antibody of the present disclosure has an IC50 of 10 nM or less (i.e., ≤10 nM) for each of the hLTBP1-proTGFβ1, hLTBP3-proTGFβ1, hGARP-proTGFβ1 and hLRRC33-proTGFβ1 complexes. In some embodiments, the antibody of the present disclosure has an IC50 of 5 nM or less (i.e., ≤5 nM) for each of the hLTBP1-proTGFβ1, hLTBP3-proTGFβ1, hGARP-proTGFβ1 and hLRRC33-proTGFβ1 complexes.

In some embodiments, potency may be evaluated in suitable in vivo models as a measure of efficacy and/or pharmacodynamics effects. For example, if the first antibody is efficacious in an in vivo model at a certain concentration, and the second antibody is equally efficacious at a lower concentration than the first in the same in vivo model, then, the second antibody can be said to me more potent than the first antibody. Any suitable disease models known in the art may be used to assess relative potencies of TGFβ1 inhibitors, depending on the particular indication of interest, e.g., cancer models and fibrosis models. Preferably, multiple doses or concentrations of each test antibody are included in such studies.

Similarly, pharmacodynamics (PD) effects may be measured to determine relative potencies of inhibitory antibodies. Commonly used PD measures for the TGFβ signaling pathway include, without limitation, phosphorylation of SMAD2/3 and expression of downstream effector genes, the transcription of which is sensitive to TGFβ activation, such as those with a TGFβ-responsive promoter element(e.g., Smad-binding elements). In some embodiments, the antibodies of the present disclosure are capable of completely blocking disease-induced SMAD2/3 phosphorylation in preclinical fibrosis models when the animals are administered at a dose of 3 mg/kg or less. In some embodiments, the antibodies of the present disclosure are capable of significantly suppressing fibrosis-induced expression of a panel of marker genes including Acta2, Col1a1, Col3a1, Fn1, Itga11, Lox, Lox12, when the animals are administered at a dose of 10 mg/kg or less in the UUO model of kidney fibrosis.

### Safety/Toxicology

As mentioned above, known pan-inhibitors that antagonize all TGFβ isoforms, namely, TGFβ1, TGFβ2 and TGFβ3, have been documented to cause various toxicities across multiple mammalian species. Most notable known toxicities include cardiovascular toxicities (such as valvulopathy) and epithelial hyperplasia, inflammation and bleeding. More specifically, some of the observed toxicities associated with pan-TGFβ inhibitors (e.g., small molecule antagonists of the TGFβR and non-selective neutralizing antibodies) reported in the literature include the following.

Cardiovascular toxicities associated with TGFβ inhibition include, hyperplasisa in aortic valve, right AV valve, and left AV valve; inflammation in aortic valve, left AV valve, and ascending aorta; hemorrhage in ascending aorta, aortic valve and left AV valve; connective tissue degeneration in ascending aorta (see for example, Strauber et al. (2014) “Nonclinical safety evaluation of a Transforming Growth Factor β receptor I kinase inhibitor in Fischer 344 rats and beagle dogs” J. Clin. Pract 4(3): 1000196).

In addition, neutralizing antibodies that bind all three TGFβ isoforms have been associated with certain epithelial toxicities, which are summarized in the table below.

TABLE 8
Epithelial and other toxicities across species for 1D11 and GC1008

| | Mice | Cyno | Human |
|---|---|---|---|
| Toxicities | Hyperplasia and | Hyperplasia of gingiva, | Gigival bleeding |
| | inflammation of tongue, | nasal epithelium, and | Epistaxis |
| | gingiva, and esophagus. | bladder | Headache |
| | Findings not reversible | Anemia lead to cessation | Fatigue |
| | (12 wk recovery) | of treatment | Various skin disorders, |
| | | Changes were reversible | including |
| | | (except bladder) | keratoacanthomas (KA), |
| | | | hyperkeratosis, |
| | | | cutaneous SCC, and |
| | | | basal cell carcinoma |
| Drug/Dose/ | 1D11 | GC1008 | GC1008 |
| Duration | Dosing: 50 mg/kg(3x/week) | Dosing: 10 and 50 mg/kg | Dose: 0.1, 0.3, 1, 3, 10, 15 mg/kg |
| | Duration: 9-12 weeks | Duration: 6 months | Duration: 4 monthlydoses |
| Exposure | Serum conc. = 1-2 mg/mL | Not disclosed | Half-life: 21.7 d |
| | (over 4-12 weeks) | | DN Cmax~(350 ng/mL)mg |

*Vitsky et. Al. Am. J Pathology vol. 174, 2009; and Lonning et. al. Current Pharmaceutical Biotech, 2011

Applicant of the present disclosure previously demonstrated the improved safety profiles of monoclonal antibodies that selectively block the activation step of TGFβ1 by targeting latent proTGFβ1 complex (see, for example, WO 2017/156500 and WO 2018/129329). In rat toxicology studies described therein, there were no observable test article-related toxicities when the animals were dosed with the inhibitors up to 100 mg/kg per week for 4 weeks.

Building upon the earlier recognition by the applicant of the present disclosure (see PCT/US2017/021972) that lack of isoform-specificity of conventional TGFβ antagonists may underlie the source of toxicities associated with TGFβ inhibition, the present inventors sought to further achieve broad-spectrum TGFβ1 inhibition for treating various diseases that manifest multifaceted TGFβ1 dysregulation, while maintaining the safety/tolerability aspect of isoform-selective inhibitors. One of the aims of the work presented herein was therefore to maintain at least the same or equivalent level of safety profiles while increasing potency of such inhibitors. Indeed, data presented herein show that the objective has been achieved.

Thus, in some embodiments, the novel antibody according to the present disclosure has the maximally tolerated dose (MTD) of >100 mg/kg when dosed weekly for at least 4 weeks. In some embodiments, the novel antibody according to the present disclosure has the no observed adverse effect level (NOAEL) of up to 100 mg/kg when dosed weekly for at least 4 weeks. Suitable animal models to be used for conducting safety/toxcology studies for TGFβ inhibitors and TGFβ1 inhibitors include, but are not limited to: rats, dogs, cynos, and mice. In preferred embodiments, the minimum effective amount of the antibody based on a suitable preclinical efficacy study is below the NOAEL. More preferably, the minimum effective amount of the antibody is about one-third or less of the NOAEL. In particularly preferred embodiments, the minimum effective amount of the antibody is about one-sixth or less of the NOAEL. In some embodiments, the minimum effective amount of the antibody is about one-tenth or less of the NOAEL.

In some embodiments, the invention encompasses an isoform-selective antibody capable of inhibiting TGFβ1 signaling, which, when administered to a subject, does not cause cardiovascular or known epithelial toxicities at a dose effective to treat a TGFβ1-related indication. In some embodiments, the antibody has a minimum effective amount of about 3-10 mg/kg administered weekly, biweekly or monthly. Preferably, the antibody causes no to minimum toxicities at a dose that is at least six-times the minimum effective amount (e.g., a six-fold therapeutic window). More preferably, the antibody causes no to minimum toxicities at a dose that is at least ten-times the minimum effective amount (e.g., a ten-fold therapeutic window). Even more preferably, the antibody causes no to minimum toxicities at a dose that is at least fifteen-times the minimum effective amount(e.g., a fifteen-fold therapeutic window).

Thus, selection of an antibody or an antigen-binding fragment thereof for therapeutic use may include: selecting an antibody or antigen-binding fragment that meets the criteria of one or more of Categories 1-5 described herein; carrying out an in vivo efficacy study in a suitable preclinical model to determine an effective amount of the antibody or the fragment; carrying out an in vivo safety/toxicology study in a suitable model to determine an amount of the antibody that is safe or toxic (e.g., MTD, NOAEL, or any art-recognized parameters for evaluating safety/toxcity); and, selecting the antibody or the fragment that provides at least a three-fold therapeutic window (preferably 6-fold, more preferably a 10-fold therapeutic window, even more preferably a 15-fold therapeutic window). The selected antibody or the fragment may be used in the manufacture of a pharmaceutical composition comprising the antibody or the fragment. Such pharmaceutical composition may be used in the treatment of a TGFβ1 indication in a subject as described herein. For example, the TGFβ1 indication may be a fibrotic disorder and/or a proliferative disorder.

### Binding Regions

In the context of the present disclosure, “binding region(s)” of an antigen provides a structural basis for the antibody-antigen interaction. As used herein, a “binding region” refers to the areas of interface between the antibody and the antigen, such that, when bound to the proTGFβ1 complex (“antigen”) in a physiological solution, the antibody or the fragment protects the binding region from solvent exposure, as determined by suitable techniques, such as hydrogen-deuterium exchange mass spectrometry (HDX-MS).

The art is familiar with HDX-MS, which is a widely used technique for exploring protein conformation or protein-protein interactions in solution. This method relies on the exchange of hydrogens in the protein backbone amide with deuterium present in the solution. By measuring hydrogen-deuterium exchange rates, one can obtain information on protein dynamics and conformation (reviewed in: Wei et al. (2014) “Hydrogen/deuterium exchange mass spectrometry for probing higher order structure of protein therapeutics: methodology and applications.” Drug Disco Today. 19(1): 95-102; incorporated by reference). The application of this technique is based on the premise that when an antibody-antigen complex forms, the interface between the binding partners may occlude solvent, thereby reducing or preventing the exchange rate due to steric exclusion of solvent.

Using this technique, binding (hence the protected) regions of proTGFβ1 can be determined. In some embodiments, a portion on proTGFβ1 identified to be important in binding of the antibody or fragment that satisfies the criteria set forth herein (the criteria required by one or more of Antibody Categories 1-5 of Table 1) includes at least a portion of the amino acid stretch SPPSQGEVPPGPLPEAVLALYNST (SEQ ID NO: 261) (“first binding region”), which largely overlaps with the protein domain within the LAP commonly referred to as latency Lasso. In some embodiments, the antibody binds (hence protects) at least portions of the amino acid sequence LREAVPE (SEQ ID NO: 259) (“second binding region”) within the Arm domain of the LAP. In some embodiments, antibody binds (hence protects) at least portion of the amino acid sequence WKWIHEPKGYHANFCLG (SEQ ID NO: 262) (“third binding region”), which largely overlaps with so-called Finger-1 within the growth factor domain. In some embodiments, the antibody binds to an epitope of a proTGFβ1 complex comprising one or more amino acid residues of SPPSQGEVPPGPLPEAVLALYNST (SEQ ID NO: 261) (“first binding region”), LREAVPE (SEQ ID NO: 259) (“second binding region”), and/or one or more amino acid residues of WKWIHEPKGYHANFCLG (SEQ ID NO: 262) (“third binding region”).

In some embodiments, the first binding region and/or the second binding region confers the isoform selectivity of the antibody or the fragment.

Advantageously, preferred inhibitory antibodies of the present disclosure are capable of inhibiting the release of mature growth factor from a latent complex, thereby reducing growth factor signaling. Such antibodies may target any epitope that results in a reduction of growth factor release or activity when associated with such antibodies. In some embodiments, the antibodies of the present disclosure specifically bind a combinatorial epitope, i.e., an epitope formed by two or more components/portions of an antigen or antigen complex. For example, a combinatorial epitope may be formed by contributions from multiple portions of a single protein, i.e., amino acid residues from more than one non-contiguous segments of the same protein. Alternatively, a combinatorial epitope may be formed by contributions from multiple protein components of an antigen complex. In some embodiments, the antibodies of the present disclosure specifically bind a conformational epitope (or conformation-specific epitope), e.g., an epitope that is sensitive to the three-dimensional structure (i.e., conformation) of an antigen or antigen complex. In preferred embodiments, the combinatorial epitope comprises an amino acid residue within Latency Lasso and an amino acid residue within the growth factor domain.

TABLE 9
Select protein domains/modules of human TGFβ1 polypeptide

| Human TGFβ1 | | |
|---|---|---|
| domain/module | Amino Acid Sequence | SEQ ID NO |
| Latency Associated | LSTCKTIDMELVKRKRIEAIRGQILSKLRLASPPSQGEVPPGPLPEAVLA | 263 |
| Peptide (LAP) | LYNSTRDRVAGESAEPEPEPEADYYAKEVTRVLMVETHNEIYDKFKQS | |
| (prodomain) | THSIYMFFNTSELREAVPEPVLLSRAELRLLRLKLKVEQHVELYQKYSN | |
| | NSWRYLSNRLLAPSDSPEWLSFDVTGVVRQWLSRGGEIEGFRLSAHC | |
| | SCDSRDNTLQVDINGFTTGRRGDLATIHGMNRPFLLLMATPLERAQHL | |
| | QSSRHRR | |
| | (“First binding region” is underlined) | |
| Straight Jacket | LSTCKTIDMELVKRKRIEAIRGQILSKLRLASPPSQGEVPPGPLP | 264 |
| Growth Factor | ALDTNYCFSSTEKNCCVRQLYIDFRKDLGWKWIHEPKGYHANFCLGP | 265 |
| Domain | CPYIWSLDTQYSKVLALYNQHNPGASAAPCCVPQALEPLPIVYYVGRK | |
| | PKVEQLSNMIVRSCKCS | |
| Fastener | residues 74-76, YYA | n/a |
| Furin cleavage site | RHRR | 266 |
| Arm | EAVLALYNSTRDRVAGESAEPEPEPEADYYAKEVTRVLMVETHNEIYD | 267 |
| | KFKQSTHSIYMFFNTSELREAVPEPVLLSRAELRLLRLKLKVEQHVELY | |
| | QKYSNNSWRYLSNRLLAPSDSPEWLSFDVTGWRQWLSRGGEIEGFR | |
| | LSAHCSCDSRDNTLQVDINGFTTGRRGDLATIHGMNRPFLLLMATPLE | |
| | RAQHLQSSRHRR | |
| | (“Second binding region” is underlined) | |
| Finger-1 | CVRQLYIDFRKDLGWKWIHEPKGYHANFC | 268 |
| | (“Third binding region” is underlined) | |
| Finger-2 | CVPQALEPLPIVYYVGRKPKVEQLSNMIVRSCKCS | 269 |
| Residue for | Cys 4 | n/a |
| presenting molecule | | |
| association | | |
| Latency Lasso | LASPPSQGEVPPGPL | 270 |
| Extended Latency | LASPPSQGEVPPGPLPEAVLALYNSTR | 271 |
| Lasso | | |
| Alpha-1 Helix | LSTCKTIDMELVKRKRIEAIRGQILSKLR | 272 |
| Alpha-2 Helix | AVLALYNSTR | 273 |
| Trigger Loop | NGFTTGRRGDLATIHGMNRP | 274 |
| Integrin binding | residue 215-217, RGD | n/a |
| Bowtie | CSCDSRDNTLQVD | 275 |

### Antigen Complexes and Components

The novel antibodies of the present disclosure specifically binds each of the four known human large latency complexes (e.g., hLTBP1-proTGFβ1, hLTBP3-proTGFβ1, hGARP-proTGFβ1 and hLRRC33-proTGFβ1), selectively inhibits TGFβ1 activation, and, satisfy the criteria of one or more of Categories 1-5 set forth in Table 1. Screening (e.g., identification and selection) of such antibodies involves the use of suitable antigen complexes, which are typically recombinantly produced. Useful protein components that may comprise such antigen complexes are provided, including TGFβ isoforms and related polypeptides, fragments and variants, presenting molecules (e.g., LTBPs, GARP, LRRC33) and related polypeptides, fragments and variants. These components may be expressed, purified, and allowed to form a protein complex (such as large latent complexes), which can be used in the process of antibody screening. The screening may include positive selection, in which desirable binders are selected from a pool or library of binders and non-binders, and negative selection, in which undesirable binders are removed from the pool.

In some embodiments, the TGFβ1 comprises a naturally occurring mammalian amino acid sequence. In some embodiment, the TGFβ1 comprises a naturally occurring human amino acid sequence. In some embodiments, the TGFβ1 comprises a human, a monkey, a rat or a mouse amino acid sequence. In some embodiments, an antibody, or antigen-binding portion thereof, described herein does not specifically bind to TGFβ2. In some embodiments, an antibody, or antigen-binding portion thereof, described herein does not specifically bind to TGFβ3. In some embodiments, an antibody, or antigen-binding portion thereof, described herein does not specifically bind to TGFβ2 or TGFβ3. In some embodiments, an antibody, or antigen-binding portion thereof, described herein specifically binds to a TGFβ1 comprising the amino acid sequence set forth in SEQ ID NO: 24. The amino acid sequences of TGFβ2, and TGFβ3 amino acid sequence are set forth in SEQ ID NOs: 28 and 32, respectively. In some embodiments, an antibody, or antigen-binding portion thereof, described herein specifically binds to a TGFβ1 comprising a non-naturally-occurring amino acid sequence (otherwise referred to herein as a non-naturally-occurring TGFβ1). For example, a non-naturally-occurring TGFβ1 may comprise one or more recombinantly generated mutations relative to a naturally-occurring TGFβ1 amino acid sequence. In some embodiments, a TGFβ1, TGFβ2, or TGFβ3 amino acid sequence comprises the amino acid sequence as set forth in SEQ ID NOs: 24-35, as shown in Table 10. In some embodiments, a TGFβ1, TGFβ2, or TGFβ3 amino acid sequence comprises the amino acid sequence as set forth in SEQ ID NOs: 36-43, as shown in Table 11.

TGFβ1 (prodomain + growth factor domain)
(SEQ ID NO: 24)
LSTCKTIDMELVKRKRIEAIRGQILSKLRLASPPSQGEVPPGPLPEAVLA
LYNSTRDRVAGESAEPEPEPEADYYAKEVTRVLMVETHNEIYDKFKQSTH
SIYMFFNTSELREAVPEPVLLSRAELRLLRLKLKVEQHVELYQKYSNNSW
RYLSNRLLAPSDSPEWLSFDVTGWRQWLSRGGEIEGFRLSAHCSCDSRDN
TLQVDINGFTTGRRGDLATIHGMNRPFLLLMATPLERAQHLQSSRHRRAL
DTNYCFSSTEKNCCVRQLYIDFRKDLGWKWIHEPKGYHANFCLGPCPYIW
SLDTQYSKVLALYNQHNPGASAAPCCVPQALEPLPIVYYVGRKPKVEQLS
NMIVRSCKCS
TGFβ2 (prodomain + growth factor domain)
(SEQ ID NO: 28)
SLSTCSTLDMDQFMRKRIEAIRGQILSKLKLTSPPEDYPEPEEVPPEVIS
IYNSTRDLLQEKASRRAAACERERSDEEYYAKEVYKIDMPPFFPSENAIP
PTFYRPYFRIVRFDVSAMEKNASNLVKAEFRVFRLQNPKARVPEQRIELY
QILKSKDLTSPTQRYIDSKVVKTRAEGEWLSFDVTDAVHEWLHHKDRNLG
FKISLHCPCCTFVPSNNYIIPNKSEELEARFAGIDGTSTYTSGDQKTIKS
TRKKNSGKTPHLLLMLLPSYRLESQQTNRRKKRALDAAYCFRNVQDNCCL
RPLYIDFKRDLGWKWIHEPKGYNANFCAGACPYLWSSDTQHSRVLSLYNT
INPEASASPCCVSQDLEPLTILYYIGKTPKIEQLSNMIVKSCKCS
TGFβ3 (prodomain + growth factor domain)
(SEQ ID NO: 32)
SLSLSTCTTLDFGHIKKKRVEAIRGQILSKLRLTSPPEPTVMTHVPYQVL
ALYNSTRELLEEMHGEREEGCTQENTESEYYAKEIHKFDMIQGLAEHNEL
AVCPKGITSKVFRFNVSSVEKNRTNLFRAEFRVLRVPNPSSKRNEQRIEL
FQILRPDEHIAKQRYIGGKNLPTRGTAEWLSFDVTDTVREWLLRRESNLG
LEISIHCPCHTFQPNGDILENIHEVMEIKFKGVDNEDDHGRGDLGRLKKQ
KDHHNPHLILMMIPPHRLDNPGQGGQRKKRALDTNYCFRNLEENCCVRPL
YIDFRQDLGWKWVHEPKGYYANFCSGPCPYLRSADTTHSTVLGLYNTLNP
EASASPCCVPQDLEPLTILYYVGRTPKVEQLSNMVVKSCKCS

TABLE 10
Exemplary TGFβ1, TGFβ2, and TGFβ3 amino acid sequences

| | | SEQ ID |
|---|---|---|
| Protein | Sequence | NO |
| proTGFβ1 | LSTCKTIDMELVKRKRIEAIRGQILSKLRLASPPSQGEVPPGPLPEAVLAL | 24 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHRRALDTNYCFSSTEKNCCVRQLYIDFRKDLGWKWIHEPKGYHANFC | |
| | LGPCPYIWSLDTQYSKVLALYNQHNPGASAAPCCVPQALEPLPIVYYVG | |
| | RKPKVEQLSNMIVRSCKCS | |
| proTGFβ1 C4S | LSTSKTIDMELVKRKRIEAIRGQILSKLRLASPPSQGEVPPGPLPEAVLAL | 25 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHRRALDTNYCFSSTEKNCCVRQLYIDFRKDLGWKWIHEPKGYHANFC | |
| | LGPCPYIWSLDTQYSKVLALYNQHNPGASAAPCCVPQALEPLPIVYYVG | |
| | RKPKVEQLSNMIVRSCKCS | |
| proTGFβ1 D2G | LSTCKTIDMELVKRKRIEAIRGQILSKLRLASPPSQGEVPPGPLPEAVLAL | 26 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHGALDTNYCFSSTEKNCCVRQLYIDFRKDLGWKWIHEPKGYHANFCL | |
| | GPCPYIWSLDTQYSKVLALYNQHNPGASAAPCCVPQALEPLPIVYYVGR | |
| | KPKVEQLSNMIVRSCKCS | |
| proTGFβ1 C4S D2G | LSTSKTIDMELVKRKRIEAIRGQILSKLRLASPPSQGEVPPGPLPEAVLAL | 27 |
| | YNSTRDRVAGESAEPEPEPEADYYAKEVTRVLMVETHNEIYDKFKQST | |
| | HSIYMFFNTSELREAVPEPVLLSRAELRLLRLKLKVEQHVELYQKYSNNS | |
| | WRYLSNRLLAPSDSPEWLSFDVTGVVRQWLSRGGEIEGFRLSAHCSC | |
| | DSRDNTLQVDINGFTTGRRGDLATIHGMNRPFLLLMATPLERAQHLQSS | |
| | RHGALDTNYCFSSTEKNCCVRQLYIDFRKDLGWKWIHEPKGYHANFCL | |
| | GPCPYIWSLDTQYSKVLALYNQHNPGASAAPCCVPQALEPLPIVYYVGR | |
| | KPKVEQLSNMIVRSCKCS | |
| proTGFβ2 | SLSTCSTLDMDQFMRKRIEAIRGQILSKLKLTSPPEDYPEPEEVPPEVISI | 28 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKKRALDAAYCFRNVQDN | |
| | CCLRPLYIDFKRDLGWKWIHEPKGYNANFCAGACPYLWSSDTQHSRVL | |
| | SLYNTINPEASASPCCVSQDLEPLTILYYIGKTPKIEQLSNMIVKSCKCS | |
| proTGFβ2 C5S | SLSTSSTLDMDQFMRKRIEAIRGQILSKLKLTSPPEDYPEPEEVPPEVISI | 29 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKKRALDAAYCFRNVQDN | |
| | CCLRPLYIDFKRDLGWKWIHEPKGYNANFCAGACPYLWSSDTQHSRVL | |
| | SLYNTINPEASASPCCVSQDLEPLTILYYIGKTPKIEQLSNMIVKSCKCS | |
| proTGFβ2 C5S D2G | SLSTSSTLDMDQFMRKRIEAIRGQILSKLKLTSPPEDYPEPEEVPPEVISI | 30 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKGALDAAYCFRNVQDNC | |
| | CLRPLYIDFKRDLGWKWIHEPKGYNANFCAGACPYLWSSDTQHSRVLS | |
| | LYNTINPEASASPCCVSQDLEPLTILYYIGKTPKIEQLSNMIVKSCKCS | |
| proTGFβ2 D2G | SLSTCSTLDMDQFMRKRIEAIRGQILSKLKLTSPPEDYPEPEEVPPEVISI | 31 |
| | YNSTRDLLQEKASRRAAACERERSDEEYYAKEVYKIDMPPFFPSENAIP | |
| | PTFYRPYFRIVRFDVSAMEKNASNLVKAEFRVFRLQNPKARVPEQRIEL | |
| | YQILKSKDLTSPTQRYIDSKVVKTRAEGEWLSFDVTDAVHEWLHHKDRN | |
| | LGFKISLHCPCCTFVPSNNYIIPNKSEELEARFAGIDGTSTYTSGDQKTIK | |
| | STRKKNSGKTPHLLLMLLPSYRLESQQTNRRKGALDAAYCFRNVQDNC | |
| | CLRPLYIDFKRDLGWKWIHEPKGYNANFCAGACPYLWSSDTQHSRVLS | |
| | LYNTINPEASASPCCVSQDLEPLTILYYIGKTPKIEQLSNMIVKSCKCS | |
| proTGFβ3 | SLSLSTCTTLDFGHIKKKRVEAIRGQILSKLRLTSPPEPTVMTHVPYQVLA | 32 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKKRALDTNYCFRNLEENCC | |
| | VRPLYIDFRQDLGWKWVHEPKGYYANFCSGPCPYLRSADTTHSTVLGL | |
| | YNTLNPEASASPCCVPQDLEPLTILYYVGRTPKVEQLSNMVVKSCKCS | |
| proTGFβ3 C7S | SLSLSTSTTLDFGHIKKKRVEAIRGQILSKLRLTSPPEPTVMTHVPYQVLA | 33 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKKRALDTNYCFRNLEENCC | |
| | VRPLYIDFRQDLGWKWVHEPKGYYANFCSGPCPYLRSADTTHSTVLGL | |
| | YNTLNPEASASPCCVPQDLEPLTILYYVGRTPKVEQLSNMVVKSCKCS | |
| proTGFβ3 C7S D2G | SLSLSTSTTLDFGHIKKKRVEAIRGQILSKLRLTSPPEPTVMTHVPYQVLA | 34 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKGALDTNYCFRNLEENCCV | |
| | RPLYIDFRQDLGWKWVH EPKGYYANFCSGPCPYLRSADTTHSTVLGLY | |
| | NTLNPEASASPCCVPQDLEPLTILYYVGRTPKVEQLSNMVVKSCKCS | |
| proTGFβ3 D2G | SLSLSTCTTLDFGHIKKKRVEAIRGQILSKLRLTSPPEPTVMTHVPYQVLA | 35 |
| | LYNSTRELLEEMHGEREEGCTQENTESEYYAKEIHKFDMIQGLAEHNEL | |
| | AVCPKGITSKVFRFNVSSVEKNRTNLFRAEFRVLRVPNPSSKRNEQRIE | |
| | LFQILRPDEHIAKQRYIGGKNLPTRGTAEWLSFDVTDTVREWLLRRESN | |
| | LGLEISIHCPCHTFQPNGDILENIHEVMEIKFKGVDNEDDHGRGDLGRLK | |
| | KQKDHHNPHLILMMIPPHRLDNPGQGGQRKGALDTNYCFRNLEENCCV | |
| | RPLYIDFRQDLGWKWVHEPKGYYANFCSGPCPYLRSADTTHSTVLGLY | |
| | NTLNPEASASPCCVPQDLEPLTILYYVGRTPKVEQLSNMVVKSCKCS | |

TABLE 11
Exemplary non-human amino acid sequences

| | | | SEQ ID |
|---|---|---|---|
| Protein | Species | Sequence | NO |
| proTGFβ1 | Mouse | LSTCKTIDMELVKRKRIEAIRGQILSKLRLASPPSQGEVPPGPLPEAVL | 36 |
| | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRRALDTNYCFSSTEKNCCVRQLYIDFRKDLGWK | |
| | | WIHEPKGYHANFCLGPCPYIWSLDTQYSKVLALYNQHNPGASASPC | |
| | | CVPQALEPLPIVYYVGRKPKVEQLSNMIVRSCKCS | |
| proTGFβ1 | Cyno | LSTCKTIDMELVKRKRIEAIRGQILSKLRLASPPSQGEVPPGPLPEAVL | 37 |
| | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRRALDTNYCFSSTEKNCCVRQLYIDFRKDLGWKWI | |
| | | HEPKGYHANFCLGPCPYIWSLDTQYSKVLALYNQHNPGASAAPCCV | |
| | | PQALEPLPIVYYVGRKPKVEQLSNMIVRSCKCS | |
| TGFβ1 LAP | Mouse | LSTSKTIDMELVKRKRIEAIRGQILSKLRLASPPSQGEVPPGPLPEAVL | 38 |
| C4S | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRR | |
| TGFβ1 LAP | Cyno | LSTSKTIDMELVKRKRIEAIRGQILSKLRLASPPSQGEVPPGPLPEAVL | 39 |
| C4S | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRR | |
| proTGFβ1 | Mouse | LSTSKTIDMELVKRKRIEAIRGQILSKLRLASPPSQGEVPPGPLPEAVL | 40 |
| C4S D2G | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHGALDTNYCFSSTEKNCCVRQLYIDFRKDLGWKWI | |
| | | HEPKGYHANFCLGPCPYIWSLDTQYSKVLALYNQHNPGASASPCCV | |
| | | PQALEPLPIVYYVGRKPKVEQLSNMIVRSCKCS | |
| proTGFβ1 | Mouse | LSTSKTIDMELVKRKRIEAIRGQILSKLRLASPPSQGEVPPGPLPEAVL | 41 |
| C4S | | ALYNSTRDRVAGESADPEPEPEADYYAKEVTRVLMVDRNNAIYEKT | |
| | | KDISHSIYMFFNTSDIREAVPEPPLLSRAELRLQRLKSSVEQHVELYQ | |
| | | KYSNNSWRYLGNRLLTPTDTPEWLSFDVTGVVRQWLNQGDGIQGF | |
| | | RFSAHCSCDSKDNKLHVEINGISPKRRGDLGTIHDMNRPFLLLMATP | |
| | | LERAQHLHSSRHRRALDTNYCFSSTEKNCCVRQLYIDFRKDLGWK | |
| | | WIHEPKGYHANFCLGPCPYIWSLDTQYSKVLALYNQHNPGASASPC | |
| | | CVPQALEPLPIVYYVGRKPKVEQLSNMIVRSCKCS | |
| proTGFβ1 | Cyno | LSTSKTIDMELVKRKRIEAIRGQILSKLRLASPPSQGEVPPGPLPEAVL | 42 |
| C4S | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHRRALDTNYCFSSTEKNCCVRQLYIDFRKDLGWKWI | |
| | | HEPKGYHANFCLGPCPYIWSLDTQYSKVLALYNQHNPGASAAPCCV | |
| | | PQALEPLPIVYYVGRKPKVEQLSNMIVRSCKCS | |
| proTGFβ1 | Cyno | LSTSKTIDMELVKRKRIEAIRGQILSKLRLASPPSQGEVPPGPLPEAVL | 43 |
| C4S D2G | | ALYNSTRDRVAGESAEPEPEPEADYYAKEVTRVLMVETHNEIYDKFK | |
| | | QSTHSIYMFFNTSELREAVPEPVLLSRAELRLLRLKLKVEQHVELYQK | |
| | | YSNNSWRYLSNRLLAPSDSPEWLSFDVTGVVRQWLSRGGEIEGFR | |
| | | LSAHCSCDSKDNTLQVDINGFTTGRRGDLATIHGMNRPFLLLMATPL | |
| | | ERAQHLQSSRHGALDTNYCFSSTEKNCCVRQLYIDFRKDLGWKWIH | |
| | | EPKGYHANFCLGPCPYIWSLDTQYSKVLALYNQHNPGASAAPCCVP | |
| | | QALEPLPIVYYVGRKPKVEQLSNMIVRSCKCS | |
| LTBP3 | CYNO | GPAGERGAGGGGALARERFKVVFAPVICKRTCLKGQCRDSCQQGS | 44 |
| | | NNFLIGENGHSTDTLTGSGFRVVVCPLPCMNGGQCSSRNQCLCPP | |
| | | DFTGRFCQVPAGGAGGGTGGSGPGLSRAGALSTGALPPLAPEGDS | |
| | | VASKHAIYAVQVIADPPGPGEGPPAQHAAFLVPLGPGQISAEVQAPP | |
| | | PVVNVRVHHPPEASVQVHRIESSNAEGAAPSQHLLPHPKPSHPRPP | |
| | | TQKPLGRCFQDTLPKQPCGSNPLPGLTKQEDCCGSIGTAWGQSKC | |
| | | HKCPQLQYTGVQKPGPVRGEVGADCPQGYKRLNSTHCQDINECAM | |
| | | PGVCRHGDCLNNPGSYRCVCPPGHSLGPSRTQCIADKPEEKSLCF | |
| | | RLVSPEHQCQHPLTTRLTRQLCCCSVGKAWGARCQRCPADGTAAF | |
| | | KEICPAGKGYHILTSHQTLTIQGESDFSLFLHPDGPPKPQQLPESPS | |
| | | QAPPPEDTEEERGVTTDSPVSEERSVQQSHPTATTSPARPYPELIS | |
| | | RPSPPTMRWFLPDLPPSRSAVEIAPTQVTETDECRLNQNICGHGEC | |
| | | VPGPPDYSCHCNPGYRSHPQHRYCVDVNECEAEPCGPGRGICMN | |
| | | TGGSYNCHCNRGYRLHVGAGGRSCVDLNECAKPHLCGDGGFCINF | |
| | | PGHYKCNCYPGYRLKASRPPVCEDIDECRDPSSCPDGKCENKPGS | |
| | | FKCIACQPGYRSQGGGACRDVNECAEGSPCSPGWCENLPGSFRC | |
| | | TCAQGYAPAPDGRSCVDVDECEAGDVCDNGICTNTPGSFQCQCLS | |
| | | GYHLSRDRSHCEDIDECDFPAACIGGDCINTNGSYRCLCPQGHRLV | |
| | | GGRKCQDIDECTQDPGLCLPHGACKNLQGSYVCVCDEGFTPTQDQ | |
| | | HGCEEVEQPHHKKECYLNFDDTVFCDSVLATNVTQQECCCSLGAG | |
| | | WGDHCEIYPCPVYSSAEFHSLCPDGKGYTQDNNIVNYGIPAHRDIDE | |
| | | CMLFGAEICKEGKCVNTQPGYECYCKQGFYYDGNLLECVDVDECL | |
| | | DESNCRNGVCENTRGGYRCACTPPAEYSPAQRQCLSPEEMDVDE | |
| | | CQDPAACRPGRCVNLPGSYRCECRPPWVPGPSGRDCQLPESPAE | |
| | | RAPERRDVCWSQRGEDGMCAGPQAGPALTFDDCCCRQGRGWGA | |
| | | QCRPCPPRGAGSQCPTSQSESNSFWDTSPLLLGKPRRDEDSSEED | |
| | | SDECRCVSGRCVPRPGGAVCECPGGFQLDASRARCVDIDECRELN | |
| | | QRGLLCKSERCVNTSGSFRCVCKAGFARSRPHGACVPQRRR | |
| LTBP3 | Mouse | GPAGERGTGGGGALARERFKVVFAPVICKRTCLKGQCRDSCQQGS | 45 |
| | | NNFLIGENGHSTDTLTGSAFRVVVCPLPCMNGGQCSSRNQCLCPP | |
| | | DFTGRFCQVPAAGTGAGTGSSGPGLARTGAMSTGPLPPLAPEGES | |
| | | VASKHAIYAVQVIADPPGPGEGPPAQHAAFLVPLGPGQISAEVQAPP | |
| | | PVVNVRVHHPPEASVQVHRIEGPNAEGPASSQHLLPHPKPPHPRPP | |
| | | TQKPLGRCFQDTLPKQPCGSNPLPGLTKQEDCCGSIGTAWGQSKC | |
| | | HKCPQLQYTGVQKPVPVRGEVGADCPQGYKRLNSTHCQDINECAM | |
| | | PGNVCHGDCLNNPGSYRCVCPPGHSLGPLAAQCIADKPEEKSLCFR | |
| | | LVSTEHQCQHPLTTRLTRQLCCCSVGKAWGARCQRCPADGTAAFK | |
| | | EICPGKGYHILTSHQTLTIQGESDFSLFLHPDGPPKPQQLPESPSRAP | |
| | | PLEDTEEERGVTMDPPVSEERSVQQSHPTTTTSPPRPYPELISRPSP | |
| | | PTFHRFLPDLPPSRSAVEIAPTQVTETDECRLNQNICGHGQCVPGPS | |
| | | DYSCHCNAGYRSHPQHRYCVDVNECEAEPCGPGKGICMNTGGSY | |
| | | NCHCNRGYRLHVGAGGRSCVDLNECAKPHLCGDGGFCINFPGHYK | |
| | | CNCYPGYRLKASRPPICEDIDECRDPSTCPDGKCENKPGSFKCIAC | |
| | | QPGYRSQGGGACRDVNECSEGTPCSPGWCENLPGSYRCTCAQYE | |
| | | PAQDGLSCIDVDECEAGKVCQDGICTNTPGSFQCQCLSGYHLSRDR | |
| | | SRCEDIDECDFPAACIGGDCINTNGSYRCLCPLGHRLVGGRKCKKDI | |
| | | DECSQDPGLCLPHACENLQGSYVCVCDEGFTLTQDQHGCEEVEQP | |
| | | HHKKECYLNFDDTVFCDSVLATNVTQQECCCSLGAGWGDHCEIYP | |
| | | CPVYSSAEFHSLVPDGKRLHSGQQHCELCIPAHRDIDECILFGAEICK | |
| | | EGKCVNTQPGYECYCKQGFYYDGNLLECVDVDECLDESNCRNGVC | |
| | | ENTRGGYRCACTPPAEYSPAQAQCLIPERWSTPQRDVKCAGASEE | |
| | | RTACVWGPWAGPALTFDDCCCRQPRLGTQCRPCPPRGTGSQCPT | |
| | | SQSESNSFWDTSPLLLGKSPRDEDSSEEDSDECRCVSGRCVPRPG | |
| | | GAVCECPGGFQLDASRARCVDIDECRELNQRGLLCKSERCVNTSG | |
| | | SFRCVCKAGFTRSRPHGPACLSAAADDAAIAHTSVIDHRGYFH | |
| LTBP1S | Cyno | NHTGRIKVVFTPSICKVTCTKGSCQNSCEKGNTTTLISENGHAADTLT | 46 |
| | | ATNFRVVLCHLPCMNGGQCSSRDKCQCPPNFTGKLCQIPVHGASV | |
| | | PKLYQHSQQPGKALGTHVIHSTHTLPLTVTSQQGVKVKFPPNIVNIH | |
| | | VKHPPEASVQIHQVSRIDGPTGQKTKEAQPGQSQVSYQGLPVQKTQ | |
| | | TIHSTYSHQQVIPHVYPVAAKTQLGRCFQETIGSQCGKALPGLSKQE | |
| | | DCCGTVGTSWGFNKCQKCPKKPSYHGYNQMMECLPGYKRVNNTF | |
| | | CQDINECQLQGVCPNGECLNTMGSYRCTCKIGFGPDPTFSSCVPDP | |
| | | PVISEEKGPCYRLVSSGRQCMHPLSVHLTKQLCCCSVGKAWGPHC | |
| | | EKCPLPGTAAFKEICPGGMGYTVSGVHRRRPIHHHVGKGPVFVKPK | |
| | | NTQPVAKSTHPPPLPAKEEPVEALTFSREHGPGVAEPEVATAPPEK | |
| | | EIPSLDQEKTKLEPGQPQLSPGISTIHLHPQFPWIEKTSPPVPVEVAP | |
| | | EASTSSASQVIAPTQVTEINECTVNPDICGAGHCINLPVRYTCICYEG | |
| | | YKFSEQQRKCVDIDECTQVQHLCSQGRCENTEGSFLCICPAGFMAS | |
| | | EEGTNCIDVDECLRPDVCGEGHCVNTVGAFRCEYCDSGYRNITQRG | |
| | | RCEDIDECLNPSTCPDEQCVNSPGSYQCVPCTEGFRGWNGQCLDV | |
| | | DECLEPNVCTNGDCSNLEGSYMCSCHKGYTRTPDHKHCKDIDECQ | |
| | | QGNLCVNGQCKNTEGSFRCTCGQGYQLSAAKDQCEDIDECQHHHL | |
| | | CAHGQCRNTEGSFQCVCDQGYRASGLGDHCEDINECLEDKSVCQR | |
| | | GDCINTAGSYDCTCPDGFQLDDNKTCQDINECEHPGLCGPQGECL | |
| | | NTEGSFHCVCQQGFSISADGRTCEDIDECVNNTVCDSHGFCDNTAG | |
| | | SFRCLCYQGFQAPQDGQGCVDVNECELLSGVCGEAFCENVEGSFL | |
| | | CVCADENQEYSPMTGQCRSRTSTDLDVEQPKEEKKECYYNLNDAS | |
| | | LCDNVLAPNVTKQECCCTSGAGWGDNCEIFPCPVLGTAEFTEMCPK | |
| | | GKGFVPAGESSSEAGGENYKDADECLLFGQEICKNGFCLNTRPGYE | |
| | | CYCKQGTYYDPVKLQCFDMDECQDPSSCIDGQCVNTEGSYNCFCT | |
| | | HPMVLDASEKRCIRPAESNEQIEETDVYQDLCWEHLSDEYVCSRPL | |
| | | VGKQTTYTECCCLYGEAWGMQCALCPMKDSDDYAQLCNIPVTGRR | |
| | | QPYGRDALVDFSEQYAPEADPYFIQDRFLNSFEELQAEECGILNGCE | |
| | | NGRCVRVQEGYTCDCFDGYHLDTAKMTCVDVNECDELNNRMSLCK | |
| | | NAKCINTEGSYKCLCLPGYVPSDKPNYCTPLNTALNLEKDSDLE | |
| LTBP1S | mouse | NHTGRIKVVFTPSICKVTCTKGNCQNSCQKGNTTTLISENGHAADTL | 47 |
| | | TATNFRVVICHLPCMNGGQCSSRDKCQCPPNFTGKLCQIPVLGASM | |
| | | PKLYQHAQQQGKALGSHVIHSTHTLPLTMTSQQGVKVKFPPNIVNIH | |
| | | VKHPPEASVQIHQVSRIDSPGGQKVKEAQPGQSQVSYQGLPVQKT | |
| | | QTVHSTYSHQQLIPHVYPVAAKTQLGRCFQETIGSQCGKALPGLSK | |
| | | QEDCCGTVGTSWGFNKCQKCPKKQSYHGYTQMMECLQGYKRVN | |
| | | NTFCQDINECQLQGVCPNGECLNTMGSYRCSCKMGFGPDPTFSSC | |
| | | VPDPPVISEEKGPCYRLVSPGRHCMHPLSVHLTKQICCCSVGKAWG | |
| | | PHCEKCPLPGTAAFKEICPGGMGYTVSGVHRRRPIHQHIGKEAVYV | |
| | | KPKNTQPVAKSTHPPPLPAKEEPVEALTSSWEHGPRGAEPEVVTAP | |
| | | PEKEIPSLDQEKTRLEPGQPQLSPGVSTIHLHPQFPWVEKTSPPVP | |
| | | VEVAPEASTSSASQVIAPTQVTEINECTVNPDICGAGHCINLPVRYTC | |
| | | ICYEGYKFSEQLRKCVDIDECAQVRHLCSQGRCENTEGSFLCVCPA | |
| | | GFMASEEGTNCIDVDECLRPDMCRDGRCINTAGAFRCEYCDSGYR | |
| | | MSRRGYCEDIDECLKPSTCPEEQCVNTPGSYQCVPCTEGFRGWNG | |
| | | QCLDVDECLQPKVCTNGSCTNLEGSYMCSCHRGYSPTPDHRHCQ | |
| | | DIDECQQGNLCMNGQCRNTDGSFRCTCGQGYQLSAAKDQCEDIDE | |
| | | CEHHHLCSHGQCRNTEGSFQCVCNQGYRASVLGDHCEDINECLED | |
| | | SSVCQGGDCINTAGSYDCTCPDGFQLNDNKGCQDINECAQPGLCG | |
| | | SHGECLNTQGSFHCVCEQGFSISADGRTCEDIDECVNNTVCDSHGF | |
| | | CDNTAGSFRCLCYQGFQAPQDGQGCVDVNECELLSGVCGEAFCE | |
| | | NVEGSFLCVCADENQEYSIDNITGQCRSRVTEDSGVDRQPREEKKEC | |
| | | YYNLNDASLCDNVLAPNVTKQECCCTSGAGWGDNCEIFPCPVQGT | |
| | | AEFTEMCPRGKGLVPAGESSYDTGGENYKDADECLLFGEEICKNGY | |
| | | CLNTQPGYECYCKQGTYYDPVKLQCFDMDECQDPNSCIDGQCVNT | |
| | | EGSYNCFCTHPMVLDASEKRCVQPTESNEQIEETDVYQDLCWEHLS | |
| | | EEYVCSRPLVGKQTTYTECCCLYGEAWGMQCALCPMKDSDDYAQL | |
| | | CNIPVTGRRRPYGRDALVDFSEQYGPETDPYFIQDRFLNSFEELQAE | |
| | | ECGILNGCENGRCVRVQEGYTCDCFDGYHLDMAKMTCVDVNECSE | |
| | | LNNRMSLCKNAKCINTEGSYKCLCLPGYIPSDKPNYCTPLNSALNLD | |
| | | KESDLE | |
| GARP | mouse | ISQRREQVPCRTVNKEALCHGLGLLQVPSVLSLDIQALYLSGNQLQSI | 48 |
| | | LVSPLGFYTALRHLDLSDNQISFLQAGVFQALPYLEHLNLAHNRLAT | |
| | | GMALNSGGLGRLPLLVSLDLSGNSLHGNLVERLLGETPRLRTLSLAE | |
| | | NSLTRLARHTFWGMPAVEQLDLHSNVLMDIEDGAFEALPHLTHLNLS | |
| | | RNSLTCISDFSLQQLQVLDLSCNSIEAFQTAPEPQAQFQLAWLDLRE | |
| | | NKLLHFPDLAVFPRLIYLNVSNNLIQLPAGLPRGSEDLHAPSEGWSA | |
| | | SPLSNPSRNASTHPLSQLLNLDLSYNEIELVPASFLEHLTSLRFLNLS | |
| | | RNCLRSFEARQVDSLPCLVLLDLSHNVLEALELGTKVLGSLQTLLLQ | |
| | | DNALQELPPYTFASLASLQRLNLQGNQVSPCGGPAEPGPPGCVDFS | |
| | | GIPTLHVLNMAGNSMGMLRAGSFLHTPLTELDLSTNPGLDVATGALV | |
| | | GLEASLEVLELQGNGLTVLRVDLPCFLRLKRLNLAENQLSHLPAWTR | |
| | | AVSLEVLDLRNNSFSLLPGNAMGGLETSLRRLYLQGNPLSCCGNGW | |
| | | LAAQLHQGRVDVDATQDLICRFGSQEELSLSLVRPEDCEKGGLKNV | |
| | | NLILLLSFTLVSAIVLTTLATICFLRRQKLSQQYKA | |
| s GARP | mouse | ISQRREQVPCRTVNKEALCHGLGLLQVPSVLSLDIQALYLSGNQLQSI | 49 |
| | | LVSPLGFYTALRHLDLSDNQISFLQAGVFQALPYLEHLNLAHNRLAT | |
| | | GMALNSGGLGRLPLLVSLDLSGNSLHGNLVERLLGETPRLRTLSLAE | |
| | | NSLTRLARHTFWGMPAVEQLDLHSNVLMDIEDGAFEALPHLTHLNLS | |
| | | RNSLTCISDFSLQQLQVLDLSCNSIEAFQTAPEPQAQFQLAWLDLRE | |
| | | NKLLHFPDLAVFPRLIYLNVSNNLIQLPAGLPRGSEDLHAPSEGWSA | |
| | | SPLSNPSRNASTHPLSQLLNLDLSYNEIELVPASFLEHLTSLRFLNLS | |
| | | RNCLRSFEARQVDSLPCLVLLDLSHNVLEALELGTKVLGSLQTLLLQ | |
| | | DNALQELPPYTFASLASLQRLNLQGNQVSPCGGPAEPGPPGCVDFS | |
| | | GIPTLHVLNMAGNSMGMLRAGSFLHTPLTELDLSTNPGLDVATGALV | |
| | | GLEASLEVLELQGNGLTVLRVDLPCFLRLKRLNLAENQLSHLPAWTR | |
| | | AVSLEVLDLRNNSFSLLPGNAMGGLETSLRRLYLQGNPLSCCGNGW | |
| | | LAAQLHQGRVDVDATQDLICRFGSQEELSLSLVRPEDCEKGGLKNV | |
| | | N | |

In some embodiments, antigenic protein complexes (e.g., a LTBP-TGFβ1 complex) may comprise one or more presenting molecules such as LTBP proteins (e.g., LTBP1, LTBP2, LTBP3, and LTBP4), GARP proteins, LRRC33 proteins, or fragment(s) thereof. Typically, a minimum required fragment suitable for carrying out the embodiments disclosed herein includes at least 50 amino acids, preferably at least 100 amino acids, of a presenting molecule protein, comprising at least two cysteine residues capable of forming disulfide bonds with a proTGFβ1 complex Specifically, these Cys residues form covalent bonds with Cysteine resides present near the N-terminus of each monomer of the proTGFβ1 complex.

An antibody, or antigen-binding portion thereof, as described herein, is capable of binding to a LTBP1-TGFβ 1 complex. In some embodiments, the LTBP1 protein is a naturally-occurring protein or fragment thereof. In some embodiments, the LTBP1 protein is a non-naturally occurring protein or fragment thereof. In some embodiments, the LTBP1 protein is a recombinant protein. Such recombinant LTBP1 protein may comprise LTBP1, alternatively spliced variants thereof and/or fragments thereof. Recombinant LTBP1 proteins may also be modified to comprise one or more detectable labels. In some embodiments, the LTBP1 protein comprises a leader sequence (e.g., a native or non-native leader sequence). In some embodiments, the LTBP1 protein does not comprise a leader sequence (i.e., the leader sequence has been processed or cleaved). Such detectable labels may include, but are not limited to biotin labels, polyhistidine tags, myctags, HA tags and/or fluorescent tags. In some embodiments, the LTBP1 protein is a mammalian LTBP1 protein. In some embodiments, the LTBP1 protein is a human, a m on key, a mouse, or a rat LTBP1 protein. In some embodiments, the LTBP1 protein comprises an amino acid sequence as set forth in SEQ ID NOs: 46 and 47 in Table 11. In some embodiments, the LTBP1 protein comprises an amino acid sequence as set forth in SEQ ID NO: 50 in Table 12.

The antibody, or antigen-binding portion thereof, as described herein, is capable of binding to a LTBP3-TGFβ1 complex. In some embodiments, the LTBP3 protein is a naturally-occurring protein or fragment thereof. In some embodiments, the LTBP3 protein is a non-naturally occurring protein or fragment thereof. In some embodiments, the LTBP3 protein is a recombinant protein. Such recombinant LTBP3 protein may comprise LTBP3, alternatively spliced variants thereof and/or fragments thereof. In some embodiments, the LTBP3 protein comprises a leader sequence (e.g., a native or non-native leader sequence). In some embodiments, the LTBP3 protein does not comprise a leader sequence (i.e., the leader sequence has been processed or cleaved). Recombinant LTBP3 proteins may also be modified to comprise one or more detectable labels. Such detectable labels may include, but are not limited to biotin labels, polyhistidine tags, myctags, HA tags and/or fluorescent tags. In some embodiments, the LTBP3 protein is a mammalian LTBP3 protein. In some embodiments, the LTBP3 protein is a human, a monkey, a mouse, or a rat LTBP3 protein. In some embodiments, the LTBP3 protein comprises an amino acid sequence as set forth in SEQ ID NOs: 44 and 45 in Table 11. In some embodiments, the LTBP1 protein comprises an amino acid sequence as set forth in SEQ ID NO: 51 in Table 12.

The antibody, or antigen-binding portion thereof, as described herein, is capable of binding to a GARP-TGFβ1 complex. In some embodiments, the GARP protein is a naturally-occurring protein or fragment thereof. In some embodiments, the GARP protein is a non-naturally occurring protein or fragment thereof. In some embodiments, the GARP protein is a recombinant protein. Such a GARP may be recombinant, referred to herein as recombinant GARP. Some recombinant GARPs may comprise one or more modifications, truncations and/or mutations as compared to wild type GARP. Recombinant GARPs may be modified to be soluble. In some embodiments, the GARP protein comprises a leader sequence (e.g., a native or non-native leader sequence). In some embodiments, the GARP protein does not comprise a leader sequence (i.e., the leader sequence has been processed or cleaved). In other embodiments, recombinant GARPs are modified to comprise one or more detectable labels. In further embodiments, such detectable labels may include, but are not limited to biotin labels, polyhistidine tags, flag tags, myctags, HA tags and/or fluorescent tags. In some embodiments, the GARP protein is a mammalian GARP protein. In some embodiments, the GARP protein is a human, a monkey, a mouse, or a rat GARP protein. In some embodiments, the GARP protein comprises an amino acid sequence as set forth in SEQ ID NOs: 48-49 in Table 11. In some embodiments, the GARP protein comprises an amino acid sequence as set forth in SEQ ID NOs: 52 and 53 in Table 13. In some embodiments, the antibodies, or antigen-binding portions thereof, described herein do not bind to TGFβ1 in a context-dependent manner, for example binding to TGFβ1 would only occur when the TGFβ1 molecule was complexed with a specific presenting molecule, such as GARP. Instead, the antibodies, and antigen-binding portions thereof, bind to TGFβ1 in a context-independent manner. In other words, the antibodies, or antigen-binding portions thereof, bind to TGFβ1 when bound to any presenting molecule: GARP, LTBP1, LTBP3, and/or LRCC33.

The antibody, or antigen-binding portion thereof, as described herein, is capable of binding to a LRRC33-TGFβ1 complex. In some embodiments, the LRRC33 protein is a naturally-occurring protein or fragment thereof. In some embodiments, the LRRC33 protein is a non-naturally occurring protein or fragment thereof. In some embodiments, the LRRC33 protein is a recombinant protein. Such a LRRC33 may be recombinant, referred to herein as recombinant LRRC33. Some recombinant LRRC33 proteins may comprise one or more modifications, truncations and/or mutations as compared to wild type LRRC33. Recombinant LRRC33 proteins may be modified to be soluble. For example, in some embodiments, the ectodomain of LRRC33 may be expressed with a C-terminal His-tag in order to express soluble LRRC33 protein (sLRRC33; see, e.g., SEQ ID NO: 84). In some embodiments, the LRRC33 protein comprises a leader sequence (e.g., a native or non-native leader sequence). In some embodiments, the LRRC33 protein does not comprise a leader sequence (i.e., the leader sequence has been processed or cleaved). In other embodiments, recombinant LRRC33 proteins are modified to comprise one or more detectable labels. In further embodiments, such detectable labels may include, but are not limited to biotin labels, polyhistidine tags, flag tags, myc tags, HA tags and/or fluorescent tags. In some embodiments, the LRRC33 protein is a mammalian LRRC33 protein. In some embodiments, the LRRC33 protein is a human, a monkey, a mouse, or a rat LRRC33 protein. In some embodiments, the LRRC33 protein comprises an amino acid sequence as set forth in SEQ ID NOs: 83, 84, and 101 in Table 13.

TABLE 12
Exemplary LTBP amino acid sequences

| | | SEQ |
|---|---|---|
| Protein | Sequence | ID NO |
| LTBP1S | NHTGRIKVVFTPSICKVTCTKGSCQNSCEKGNTTTLISENGHAADTLT | 50 |
| | ATNFRWICHLPCMNGGQCSSRDKCQCPPNFTGKLCQIPVHGASVP | |
| | KLYQHSQQPGKALGTHVIHSTHTLPLTVTSQQGVKVKFPPNIVNIHVK | |
| | HPPEASVQIHQVSRIDGPTGQKTKEAQPGQSQVSYQGLPVQKTQTIH | |
| | STYSHQQVIPHVYPVAAKTQLGRCFQETIGSQCGKALPGLSKQEDCC | |
| | GTVGTSWGFNKCQKCPKKPSYHGYNQMMECLPGYKRVNNTFCQDI | |
| | NECQLQGVCPNGECLNTMGSYRCTCKIGFGPDPTFSSCVPDPPVISE | |
| | EKGPCYRLVSSGRQCMHPLSVHLTKQLCCCSVGKAWGPHCEKCPL | |
| | PGTAAFKEICPGGMGYTVSGVHRRRPIHHHVGKGPVFVKPKNTQPV | |
| | AKSTHPPPLPAKEEPVEALTFSREHGPGVAEPEVATAPPEKEIPSLDQ | |
| | EKTKLEPGQPQLSPGISTIHLHPQFPWIEKTSPPVPVEVAPEASTSSA | |
| | SQVIAPTQVTEINECTVNPDICGAGHCINLPVRYTCICYEGYRFSEQQ | |
| | RKCVDIDECTQVQHLCSQGRCENTEGSFLCICPAGFMASEEGTNCID | |
| | VDECLRPDVCGEGHCVNTVGAFRCEYCDSGYRMTQRGRCEDIDECL | |
| | NPSTCPDEQCVNSPGSYQCVPCTEGFRGWNGQCLDVDECLEPNVC | |
| | ANGDCSNLEGSYMCSCHKGYTRTPDHKHCRDIDECQQGNLCVNGQ | |
| | CKNTEGSFRCTCGQGYQLSAAKDQCEDIDECQHRHLCAHGQCRNT | |
| | EGSFQCVCDQGYRASGLGDHCEDINECLEDKSVCQRGDCINTAGSY | |
| | DCTCPDGFQLDDNKTCQDINECEHPGLCGPQGECLNTEGSFHCVCQ | |
| | QGFSISADGRTCEDIDECVNNTVCDSHGFCDNTAGSFRCLCYQGFQ | |
| | APQDGQGCVDVNECELLSGVCGEAFCENVEGSFLCVCADENQEYSP | |
| | MTGQCRSRTSTDLDVDVDQPKEEKKECYYNLNDASLCDNVLAPNVT | |
| | KQECCCTSGVGWGDNCEIFPCPVLGTAEFTEMCPKGKGFVPAGESS | |
| | SEAGGENYKDADECLLFGQEICKNGFCLNTRPGYECYCKQGTYYDP | |
| | VKLQCFDMDECQDPSSCIDGQCVNTEGSYNCFCTHPMVLDASEKRC | |
| | IRPAESNEQIEETDVYQDLCWEHLSDEYVCSRPLVGKQTTYTECCCL | |
| | YGEAWGMQCALCPLKDSDDYAQLCNIPVTGRRQPYGRDALVDFSEQ | |
| | YTPEADPYFIQDRFLNSFEELQAEECGILNGCENGRCVRVQEGYTCD | |
| | CFDGYHLDTAKMTCVDVNECDELNNRMSLCKNAKCINTDGSYKCLCL | |
| | PGYVPSDKPNYCTPLNTALNLEKDSDLE | |
| LTBP3 | GPAGERGAGGGGALARERFKVVFAPVICKRTCLKGQCRDSCQQGS | 51 |
| | NMTLIGENGHSTDTLTGSGFRVVVCPLPCMNGGQCSSRNQCLCPPD | |
| | FTGRFCQVPAGGAGGGTGGSGPGLSRTGALSTGALPPLAPEGDSVA | |
| | SKHAIYAVQVIADPPGPGEGPPAQHAAFLVPLGPGQISAEVQAPPPVV | |
| | NVRVHHPPEASVQVHRIESSNAESAAPSQHLLPHPKPSHPRPPTQKP | |
| | LGRCFQDTLPKQPCGSNPLPGLTKQEDCCGSIGTAWGQSKCHKCPQ | |
| | LQYTGVQKPGPVRGEVGADCPQGYKRLNSTHCQDINECAMPGVCR | |
| | HGDCLNNPGSYRCVCPPGHSLGPSRTQCIADKPEEKSLCFRLVSPEH | |
| | QCQHPLTTRLTRQLCCCSVGKAWGARCQRCPTDGTAAFKEICPAGK | |
| | GYHILTSHQTLTIQGESDFSLFLHPDGPPKPQQLPESPSQAPPPEDTE | |
| | EERGVTTDSPVSEERSVQQSHPTATTTPARPYPELISRPSPPTMRWF | |
| | LPDLPPSRSAVEIAPTQVTETDECRLNQNICGHGECVPGPPDYSCHC | |
| | NPGYRSHPQHRYCVDVNECEAEPCGPGRGICMNTGGSYNCHCNRG | |
| | YRLHVGAGGRSCVDLNECAKPHLCGDGGFCINFPGHYKCNCYPGYR | |
| | LKASRPPVCEDIDECRDPSSCPDGKCENKPGSFKCIACQPGYRSQG | |
| | GGACRDVNECAEGSPCSPGWCENLPGSFRCTCAQGYAPAPDGRSC | |
| | LDVDECEAGDVCDNGICSNTPGSFQCQCLSGYHLSRDRSHCEDIDE | |
| | CDFPAACIGGDCINTNGSYRCLCPQGHRLVGGRKCQDIDECSQDPSL | |
| | CLPHGACKNLQGSYVCVCDEGFTPTQDQHGCEEVEQPHHKKECYL | |
| | NFDDTVFCDSVLATNVTQQECCCSLGAGWGDHCEIYPCPVYSSAEF | |
| | HSLCPDGKGYTQDNNIVNYGIPAHRDIDECMLFGSEICKEGKCVNTQ | |
| | PGYECYCKQGFYYDGNLLECVDVDECLDESNCRNGVCENTRGGYR | |
| | CACTPPAEYSPAQRQCLSPEEMDVDECQDPAACRPGRCVNLPGSY | |
| | RCECRPPWVPGPSGRDCQLPESPAERAPERRDVCWSQRGEDGMC | |
| | AGPLAGPALTFDDCCCRQGRGWGAQCRPCPPRGAGSHCPTSQSES | |
| | NSFWDTSPLLLGKPPRDEDSSEEDSDECRCVSGRCVPRPGGAVCEC | |
| | PGGFQLDASRARCVDIDECRELNQRGLLCKSERCVNTSGSFRCVCK | |
| | AGFARSRPHGACVPQRRR | |

TABLE 13
Exemplary GARP and LRRC33 amino acid sequences

| | | SEQ |
|---|---|---|
| Protein | Sequence | ID NO |
| GARP | AQHQDKVPCKMVDKKVSCQVLGLLQVPSVLPPDTETLDLSGNQLRSILA | 52 |
| | SPLGFYTALRHLDLSTNEISFLQPGAFQALTHLEHLSLAHNRLAMATALS | |
| | AGGLGPLPRVTSLDLSGNSLYSGLLERLLGEAPSLHTLSLAENSLTRLTR | |
| | HTFRDMPALEQLDLHSNVLMDIEDGAFEGLPRLTHLNLSRNSLTCISDFS | |
| | LQQLRVLDLSCNSIEAFQTASQPQAEFQLTWLDLRENKLLHFPDLAALP | |
| | RLIYLNLSNNLIRLPTGPPQDSKGIHAPSEGVVSALPLSAPSGNASGRPLS | |
| | QLLNLDLSYNEIELIPDSFLEHLTSLCFLNLSRNCLRTFEARRLGSLPCLM | |
| | LLDLSHNALETLELGARALGSLRTLLLQGNALRDLPPYTFANLASLQRLN | |
| | LQGNRVSPCGGPDEPGPSGCVAFSGITSLRSLSLVDNEIELLRAGAFLH | |
| | TPLTELDLSSNPGLEVATGALGGLEASLEVLALQGNGLMVLQVDLPCFIC | |
| | LKRLNLAENRLSHLPAWTQAVSLEVLDLRNNSFSLLPGSAMGGLETSLR | |
| | RLYLQGNPLSCCGNGWLAAQLHQGRVDVDATQDLICRFSSQEEVSLSH | |
| | VRPEDCEKGGLKNINLIIILTFILVSAILLTTLAACCCVRRQKFNQQYKA | |
| s GARP | AQHQDKVPCKMVDKKVSCQVLGLLQVPSVLPPDTETLDLSGNQLRSILA | 53 |
| | SPLGFYTALRHLDLSTNEISFLQPGAFQALTHLEHLSLAHNRLAMATALS | |
| | AGGLGPLPRVTSLDLSGNSLYSGLLERLLGEAPSLHTLSLAENSLTRLTR | |
| | HTFRDMPALEQLDLHSNVLMDIEDGAFEGLPRLTHLNLSRNSLTCISDFS | |
| | LQQLRVLDLSCNSIEAFQTASQPQAEFQLTWLDLRENKLLHFPDLAALP | |
| | RLIYLNLSNNLIRLPTGPPQDSKGIHAPSEGWSALPLSAPSGNASGRPLS | |
| | QLLNLDLSYNEIELIPDSFLEHLTSLCFLNLSRNCLRTFEARRLGSLPCLM | |
| | LLDLSHNALETLELGARALGSLRTLLLQGNALRDLPPYTFANLASLQRLN | |
| | LQGNRVSPCGGPDEPGPSGCVAFSGITSLRSLSLVDNEIELLRAGAFLH | |
| | TPLTELDLSSNPGLEVATGALGGLEASLEVLALQGNGLMVLQVDLPCFIC | |
| | LKRLNLAENRLSHLPAWTQAVSLEVLDLRNNSFSLLPGSAMGGLETSLR | |
| | RLYLQGNPLSCCGNGWLAAQLHQGRVDVDATQDLICRFSSQEEVSLSH | |
| | VRPEDCEKGGLKNIN | |
| LRRC33 (also known as | MELLPLWLCLGFHFLTVGWRNRSGTATAASQGVCKLVGGAADCRGQ | 83 |
| NRROS; Uniprot | SLASVPSSLPPHARMLTLDANPLKTLWNHSLQPYPLLESLSLHSCHLERI | |
| Accession No. Q86YC3) | SRGAFQEQGHLRSLVLGDNCLSENYEETAAALHALPGLRRLDLSGNAL | |
| | TEDMAALMLQNLSSLRSVSLAGNTIMRLDDSVFEGLERLRELDLQRNYI | |
| | FEIEGGAFDGLAELRHLNLAFNNLPCIVDFGLTRLRVLNVSYNVLEWFLA | |
| | TGGEAAFELETLDLSHNQLLFFPLLPQYSKLRTLLLRDNNMGFYRDLYN | |
| | TSSPREMVAQFLLVDGNVTNITTVSLWEEFSSSDLADLRFLDMSQNQF | |
| | QYLPDGFLRKMPSLSHLNLHQNCLIVTLHIREHEPPGALTELDLSHNQLS | |
| | ELHLAPGLASCLGSLRLFNLSSNQLLGVPPGLFANARNITTLDMSHNQIS | |
| | LCPLPAASDRVGPPSCVDFRNMASLRSLSLEGCGLGALPDCPFQGTSL | |
| | TYLDLSSNWGVLNGSLAPLQDVAPMLQVLSLRNMGLHSSFMALDFSGF | |
| | GNLRDLDLSGNCLTTFPRFGGSLALETLDLRRNSLTALPQKAVSEQLSR | |
| | GLRTIYLSQNPYDCCGVDGWGALQHGQTVADWAMVTCNLSSKIIRVTE | |
| | LPGGVPRDCKWERLDLGLLYLVLILPSCLTLLVACTVIVLTFKKPLLQVIK | |
| | SRCHWSSVY | |
| | * Native signal peptide is depicted in bold font. | |
| soluble LRRC33 | MDMRVPAQLLGLLLLWFSGVLGWRNRSGTATAASQGVCKLVGGAAD | 84 |
| (sLRRC33) | CRGQSLASVPSSLPPHARMLTLDANPLKTLWNHSLQPYPLLESLSLHSC | |
| | HLERISRGAFQEQGHLRSLVLGDNCLSENYEETAAALHALPGLRRLDLS | |
| | GNALTEDMAALMLQNLSSLRSVSLAGNTIMRLDDSVFEGLERLRELDLQ | |
| | RNYIFEIEGGAFDGLAELRHLNLAFNNLPCIVDFGLTRLRVLNVSYNVLE | |
| | WFLATGGEAAFELETLDLSHNQLLFFPLLPQYSKLRTLLLRDNNMGFYR | |
| | DLYNTSSPREMVAQFLLVDGNVTNITTVSLWEEFSSSDLADLRFLDMSQ | |
| | NQFQYLPDGFLRKMPSLSHLNLHQNCLIVTLHIREHEPPGALTELDLSHN | |
| | QLSELHLAPGLASCLGSLRLFNLSSNQLLGVPPGLFANARNITTLDMSH | |
| | NQISLCPLPAASDRVGPPSCVDFRNMASLRSLSLEGCGLGALPDCPFQ | |
| | GTSLTYLDLSSNWGVLNGSLAPLQDVAPMLQVLSLRNMGLHSSFMALD | |
| | FSGFGNLRDLDLSGNCLTTFPRFGGSLALETLDLRRNSLTALPQKAVSE | |
| | QLSRGLRTIYLSQNPYDCCGVDGWGALQHGQTVADWAMVTCNLSSKII | |
| | RVTELPGGVPRDCKWERLDLGLHHHHHH | |
| | * Modified human kappa lightchain signal peptide | |
| | is depicted in bold font. | |
| | ** Histidinetag is underlined. | |
| Human LRRC33-GARP | MDMRVPAQLLGLLLLWFSGVLGWRNRSGTATAASQGVCKLVGGAAD | 101 |
| chimera | CRGQSLASVPSSLPPHARMLTLDANPLKTLWNHSLQPYPLLESLSLHSC | |
| | HLERISRGAFQEQGHLRSLVLGDNCLSENYEETAAALHALPGLRRLDLS | |
| | GNALTEDMAALMLQNLSSLRSVSLAGNTIMRLDDSVFEGLERLRELDLQ | |
| | RNYIFEIEGGAFDGLAELRHLNLAFNNLPCIVDFGLTRLRVLNVSYNVLE | |
| | WFLATGGEAAFELETLDLSHNQLLFFPLLPQYSKLRTLLLRDNNMGFYR | |
| | DLYNTSSPREMVAQFLLVDGNVTNITTVSLWEEFSSSDLADLRFLDMSQ | |
| | NQFQYLPDGFLRKMPSLSHLNLHQNCLIVTLHIREHEPPGALTELDLSHN | |
| | QLSELHLAPGLASCLGSLRLFNLSSNQLLGVPPGLFANARNITTLDMSH | |
| | NQISLCPLPAASDRVGPPSCVDFRNMASLRSLSLEGCGLGALPDCPFQ | |
| | GTSLTYLDLSSNWGVLNGSLAPLQDVAPMLQVLSLRNMGLHSSFMALD | |
| | FSGFGNLRDLDLSGNCLTTFPRFGGSLALETLDLRRNSLTALPQKAVSE | |
| | QLSRGLRTIYLSQNPYDCCGVDGWGALQHGQTVADWAMVTCNLSSKII | |
| | RVTELPGGVPRDCKWERLDLGLLIIILTFILVSAILLTTLAACCCVRRQKFN | |
| | QQYKA | |
| | * Modified human kappa lightchain signal peptide | |
| | is depicted in bold font. | |
| | ** LRRC33 ectodomain is underlined. | |
| | # GARP trans membrane domain is italicized. | |
| | ## GARP intracellulartail is double underlined. | |

### Therapeutic Targets and In Vivo Mechanism of Action

Accordingly, such pharmaceutical compositions and formulations are used to target TGFβ-containing latent complexes accessible by the inhibitors in vivo. Thus, the antibody of the invention is aimed to target the following complexes in a disease site (e.g., TME or fibrotic tissue) where it preemptively binds the latent complex thereby preventing the growth factor from being released: i) proTGFβ1 presented by GARP; ii) proTGFβ1 presented by LRRC33; iii) proTGFβ1 presented by LTBP1; and iv) proTGFβ1 presented by LTBP3. Typically, complexes (i) and (ii) above are present on cell surface because both GARP and LRRC33 are transmembrane proteins capable of presenting or tethering latent proTGFβ1 on the extracellular face of the cell expressing GARP or LRRC33, whilst complexes (iii) and (iv) are components of the extracellular matrix. In this way, the inhibitors embodied herein do away with having to complete binding with endogenous high affinity receptors for exerting inhibitory effects. Moreover, targeting upstream of the ligand/receptor interaction may enable more durable effects since the window of target accessibility is longer and more localized to relevant tissues than conventional inhibitors that target active, soluble growth factors only after it has been released from the latent complex.

A number of studies have shed light on the mechanisms of TGFβ1 activation. Three integrins, αVβ6, αVβ8, and αVβ1 have been demonstrated to be key activators of latent TGFβ1 (Reed, N. I., et al., Sci Transl Med, 2015. 7(288): p. 288ra79; Travis, M. A. and D. Sheppard, Annu Rev Immunol, 2014. 32: p. 51-82; Munger, J. S., et al., Cell, 1999. 96(3): p. 319-28). αV integrins bind the RGD sequence present in TGFβ1 and TGFβ1 LAPs with high affinity (Dong, X., et al., Nat Struct Mol Biol, 2014. 21(12): p. 1091-6). Transgenic mice with a mutation in the TGFβ1 RGD site that prevents integrin binding, but not secretion, phenocopy the TGFβ1−/− mouse (Yang, Z., et al., J Cell Biol, 2007. 176(6): p. 787-93). Mice that lack both β6 and β8 integrins recapitulate all essential phenotypes of TGFβ1 and TGFβ3 knockout mice, including multiorgan inflammation and cleft palate, confirming the essential role of these two integrins for TGFβ1 activation in development and homeostasis (Aluwihare, P., et al., J Cell Sci, 2009. 122(Pt 2): p. 227-32). Key for integrin-dependent activation of latent TGFβ1 is the covalent tether to presenting molecules; disruption of the disulfide bonds between GARP and TGFβ1 LAP by mutagenesis does not impair complex formation, but completely abolishes TGFβ1 activation by αVβ6 (Wang, R., et al., Mol Biol Cell, 2012. 23(6): p. 1129-39). The recent structure of latent TGFβ1 illuminates how integrins enable release of active TGFβ1 from the latent complex the covalent link of latentTGFβ1 to its presenting molecule anchors latentTGFβ1, either to the ECM through LTBPs, or to the cytoskeleton through GARP or LRRC33. Integrin binding to the RGD sequence results in a force-dependent change in the structure of LAP, allowing active TGFβ1 to be released and bind nearby receptors (Shi, M., et al., Nature, 2011. 474(7351): p. 343-9). The importance of integrin-dependent TGFβ1 activation in disease has also been well validated. A small molecular inhibitor of αVβ1 protects against bleomycin-induced lung fibrosis and carbon tetrachloride-induced liver fibrosis (Reed, N. I., et al., Sci Transl Med, 2015. 7(288): p. 288ra79), and αVβ6 blockade with an antibody or loss of integrin β6 expression suppresses bleomycin-induced lung fibrosis and radiation-induced fibrosis (Munger, J. S., et al., Cell, 1999. 96(3): p. 319-28); Horan, G. S., et al., Am J Respir Crit Care Med, 2008. 177(1): p. 56-65). In addition to integrins, other mechanisms of TGFβ1 activation have been implicated, including thrombospondin-1 and activation by proteases such as Plasmin, matrix metalloproteinases (MMPs, e.g., MMP2, MMP9 and MMP12), cathepsin D and kallikrein. Knockout of thrombospondin-1 recapitulates some aspects of the TGFβ1−/− phenotype in some tissues, but is not protective in bleomycin-induced lung fibrosis, known to be TGFβ-dependent (Ezzie, M. E., et al., Am J Respir Cell Mol Biol, 2011. 44(4): p. 556-61). Additionally, knockout of candidate proteases did not result in a TGFβ1 phenotype (Worthington, J. J., J. E. Klementowicz, and M. A. Travis, Trends Biochem Sci, 2011. 36(1): p. 47-54). This could be explained by redundancies or by these mechanisms being critical in specific diseases rather than development and homeostasis.

The antibodies of the present disclosure work by preventing the step of TGFβ1 activation. In some embodiments, such inhibitors can inhibit integrin-dependent(e.g., mechanical or force-driven) activation of TGFβ1. In some embodiments, such inhibitors can inhibit protease-dependent or protease-induced activation of TGFβ1. The latter includes inhibitors that inhibit the TGFβ1 activation step in an integrin-independent manner. In some embodiments, such inhibitors can inhibit TGFβ1 activation irrespective of the mode of activation, e.g., inhibit both integrin-dependent activation and protease-dependent activation of TGFβ1. Non-limiting examples of proteases which may activate TGFβ1 include serine proteases, such as Kallikreins, Chemotrypsin, Trypsin, Elastases, Plasmin, as well as zinc metalloproteases (MMP family) such as MMP-2, MMP-9, MMP-12, MMP-13 and ADAM proteases (e.g., ADAM10 and ADAM17). Kallikreins include plasma-Kallikreins and tissue Kallikreins, such as KLK1, KLK2, KLK3, KLK4, KLK5, KLK6, KLK7, KLK8, KLK9, KLK10, KLK11, KLK12, KLK13, KLK14 and KLK15. Data presented herein demonstrate examples of an isoform-specific TGFβ1 inhibitors, capable of inhibiting Kallikrein-dependent activation of TGFβ1 in vitro. In some embodiments, inhibitors of the present invention prevent release or dissociation of active (mature) TGFβ1 growth factor from the latent complex.

In some embodiments, the antibodies according to the present disclosure may induce internalization of the complex comprising proTGFβ1 bound to LRRC33 or GARP on cell surface. In some embodiments, the antibodies are inhibitors of cell-associated TGFβ1 (e.g., GARP-presented proTGFβ1 and LRRC33-presented proTGFβ1). The invention includes antibodies or fragments thereof that specifically bind such complex (e.g., GARP-pro/latent TGFβ1 and LRRC33-pro/latent TGFβ1), thereby triggering internalization of the complex (e.g., endocytosis). This mode of action causes removal or depletion of the inactive TGFβ1 complexes from the cell surface (e.g., Treg, macrophages, MDSCs, etc.), hence reducing latentTGFβ1 available for activation.

### TGFβ Inhibitors Suitable for Therapeutic Use

A body of evidence supports the notion that many diseases manifest complex perturbations of TGFβ signaling, which likely involve participation of heterogeneous cell types that confer different effects of TGFβ function, which are mediated by its interactions with so-called presenting molecules. At least four such presenting molecules have been identified, which can “present” TGFβ in various extracellular niches to enable its activation in response to local stimuli. In one category, TGFβ is deposited into the ECM in association with ECM-associated presenting molecules, such as LTBP1 and LTBP3, which mediate ECM-associated TGFβ activities. In another category, TGFβ is tethered onto the surface of cells (e.g., immune cells), via presenting molecules such as GARP and LRRC33, which mediate certain immune function. These presenting molecules show differential expression, localization and/or function in different tissues and cell types, indicating that triggering events and outcome of TGFβ activation will vary, depending on the biological or pathological microenvironment. Based on the notion that many TGFβ effects may interact and contribute to disease progression, therapeutic agents that can antagonize multiple facets of TGFβ function may provide greater efficacy.

It has been recognized that various diseases involve heterogeneous populations of cells as multiple sources of TGFβ1 that collectively contribute to the pathogenesis and/or progression of the disease. More than one type of TGFβ1-containing complexes (“contexts”) likely coexist within the same disease microenvironment. Therefore, the ability to inhibit TGFβ1 in different biological contexts may be important.

However, in certain situations, so-called context-biased antibodies that still specifically bind to all four antigen complexes but with stronger affinities for matrix-associated complexes over cell-associated complexes may be advantageous. The feature, i.e., differential binding affinities of these antibodies for the ECM complexes relative to immune cell complexes may raise the possibility that such inhibitors may be particularly suited as therapeutics to treat fibrotic conditions, such as organ fibrosis, in which affected patients receive a long-term therapeutic regimen to treat chronic conditions. In these circumstances, itis desirable to minimize unwanted inflammation triggered by immune stimulation.

In some embodiments, the antibodies of the present disclosure have greater affinities towards EMC-complexes, e.g., hLTBP1-proTGFβ1 and hLTBP3-proTGFβ1 (KD of <1 nM) over cell-associated complexes, as determined by, for example, solution equilibrium titration. It is envisaged that the EMC-biased antibodies are capable of preferentially targeting and inhibiting EMC-associated TGFβ1 in vivo. Such antibodies may be advantageous for use in the treatment of conditions with ECM dysregulation, such as abnormal remodeling and/or stiffness of the EC M. Typically, the ECM dysregulation may be accompanied by an increased number of myofibroblasts or myofibroblast-like cells in the disease environment, such as tumor microenvironment and fibrotic microenvironment. Many of the abnormal features of the ECM are often manifested in a wide range of pathological conditions, including fibrosis and proliferative disorders are at least in part driven by the TGFβ1 pathway.

In some embodiments, the context-biased TGFβ1 antibodies of the present disclosure show bias for matrix-associated complexes (LTBP1- and LTBP3-associated proTGFβ1 complexes) over cell-associated complexes (GARP- and LRRC33-associated proTGFβ1 complexes). In some embodiments, average K_{D} values for the ECM-associated complexes are in a sub-nanomolar range (e.g., ˜0.1-0.9 nM) and average K_{D} values for the cell-associated complexes are in 1 nM or greater, as determined by solution equilibrium titration.

In some embodiments, the context-biased TGFβ1 antibodies of the present disclosure show bias specifically against a GARP-associated complex over the other three complexes. In preferred embodiments, such antibodies exhibit specific but weaker binding to (biased against) human GARP-proTGFβ1 over human LRRC33-proTGFβ1, human LTBP1-proTGFβ1 and human LTBP3-proTGFβ1. In some embodiments, measured K_{D} values of such antibodies to a human GARP-proTGFβ1 complex may be about 5-20-fold greater than measured K_{D} values to human LRRC33-proTGFβ1, human LTBP1-proTGFβ1 and human LTBP3-proTGFβ1 complexes. In some embodiments, a measured K_{D} values of the context-biased antibody for a human GARP-proTGFβ1 complex is 1 nM or greater, while measured KD values for each of the other three complexes (human LRRC33-proTGFβ1, human LTBP1-proTGFβ1 and human LTBP3-proTGFβ1) is in a sub-nanomolar range (˜0.1-0.9 nM).

The context-biased antibodies with weaker binding to a GARP-associated TGFβ complex (e.g., human GARP-proTGFβ1) are used in the treatment of a condition where it is undesirable to stimulate the subject's immune response and/or in situations where the subject is expected to benefit from a long-term TGFβ inhibition therapy. Rationale for the therapeutic use of a TGFβ1 inhibitor with a weaker binding affinity for GARP-proTGFβ1 is at least threefold:

First, GARP is predominantly expressed on regulatory T cells, which playa crucial role in maintaining immune tolerance to self-antigens and in preventing autoimmune disease. Since Tregs generally suppress, dampen or downregulate induction and proliferation of effector T cells, systemic inhibition of this function may lead to overactive or exaggerated immune responses in the host by disabling the “break” that is normally provided by Treg cells. Thus, the approach taken here (e.g., TGFβ1 inhibition without fully disabling Treg function) is aimed to avoid the risk of eliciting autoimmunity. Furthermore, patients who already have a propensity for developing over-sensitive immune responses or autoimmunity may be particularly at risk of triggering or exacerbating such conditions, without the availability of functional Tregs; and therefore, the inhibitors that at least partially preserve GARP-mediated TGFβ1 function may advantageously minimize such risk.

Second, evidence suggests that an alteration in the Th17/Treg ratio leads to an imbalance in pro-fibrotic Th17 cytokines, which correlate with severity of fibrosis, such as liver fibrosis (see, for example, Shoukry et al. (2017) J Immunol 198(1 Supplement):197.12). The present inventors reasoned that disabling perturbation of the GARP arm of TGFβ1 function may directly or indirectly exacerbate fibrotic conditions.

Third, regulatory T cells are indispensable for immune homeostasis and the prevention of autoimmunity. It was reasoned that, particularly for a TGFβ1 inhibition therapy intended for a long-term or chronic administration, it would be desirable to spare at least part of GARP-mediated TGFβ1 and avoid potential side effects stemming from complete perturbation of normal Treg function in maintaining immune homeostasis (reviewed in, for example, Richert-Spuhler and Lund (2015) Prog Mol Biol Transl Sci. 136: 217-243). This strategy is at least in part aimed to preserve normal immune function, which is required, inter alia, for combatting infections.

### TGFβ1-Related Indications

The isoform-specific TGFβ1 inhibitors described herein may be used to treat a TGFβ1-related indication in subjects. Various disease conditions have been suggested to involve dysregulation of TGFβ signaling as a contributing factor. Indeed, the pathogenesis and/or progression of certain human conditions appear to be predominantly driven by or dependent on TGFβ1 activities. Moreover, it is contemplated that there is crosstalk among TGFβ1-responsive cells. In some cases, interplays between multifaceted activities of the TGFβ1 ads may trigger a cascade of events that lead to disease progression, aggravation, and/or suppression of the host's ability to combat disease. For example, certain disease microenvironments, such as tumor microenvironment (TME), may be associated with TGFβ1 presented by multiple different presenting molecules, e.g., LTBP1-proTGFβ1, LTBP3-proTGFβ1, GARP-proTGFβ1, LRRC33-proTGFβ1, and any combinations thereof. TGFβ1 activities of one context may in turn regulate or influence TGFβ1 activities of another context, raising the possibility that when dysregulated, this may result in exacerbation of disease conditions. Therefore, it is desirable to broadly inhibit across multiple modes of TGFβ1 function (i.e., multiple contexts) while selectively limiting such inhibitory effects to the TGFβ1 isoform. The aim is not to perturb homeostatic TGFβ signaling mediated by the other isoforms, including TGFβ3, which plays an important role in would healing.

The present invention extends the notion of selecting “the right TGFβ1 inhibitor” for “the right patient population” to treat a disease condition with certain criteria and/or clinical features. At least two inquiries may be made as to the identification/selection of suitable indications and/or patient populations for which the inhibitors of TGFβ1 described herein, are likely to have advantageous effects (e.g., clinical benefits): i) whether the disease is driven by or dependent predominantly on the TGFβ1 isoform over the other isoforms in human (or at least co-dominant); and, ii) whether the disease involves both matrix-associated and/or immune cell-associated TGFβ1 function.

As to the first inquiry above, differential expressions of the three known TGFβ isoforms, namely, TGFβ1, TGFβ2, and TGFβ3, have been observed under normal (healthy; homeostatic) as well as disease conditions in various tissues (note that “TGFB” is sometimes used to refer to the gene as opposed to protein). Nevertheless, the concept of isoform selectivity has neither been fully exploited nor achieved with conventional approaches that favor pan-inhibition of TGFβ across multiple isoforms. Moreover, expression patterns of the isoforms may be differentially regulated, not only in normal (homeostatic) vs, abnormal (pathologic) conditions, but also in different subpopulations of patients. Because most preclinical studies are conducted in a limited number of animal models, data obtained with the use of such models may be biased, resulting in misinterpretations of data or misleading conclusions as to the applicability to human conditions (i.e., translatability).

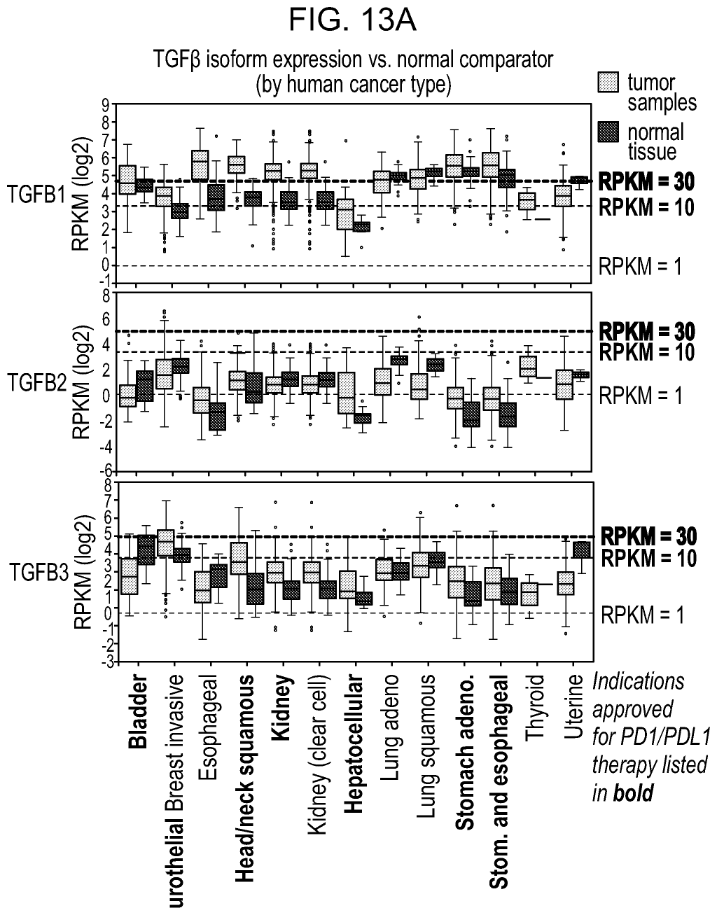

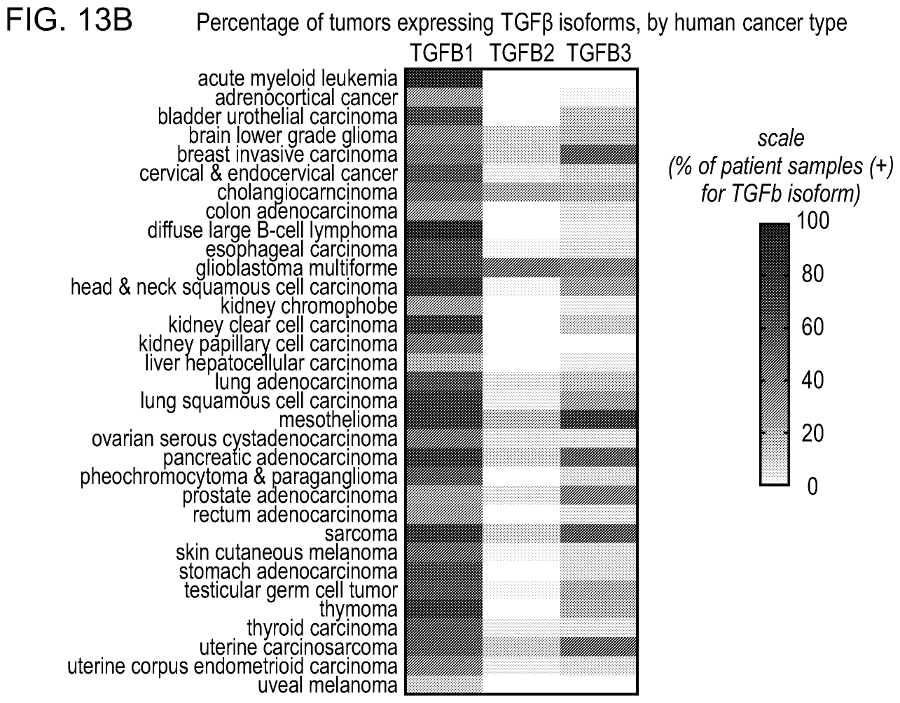

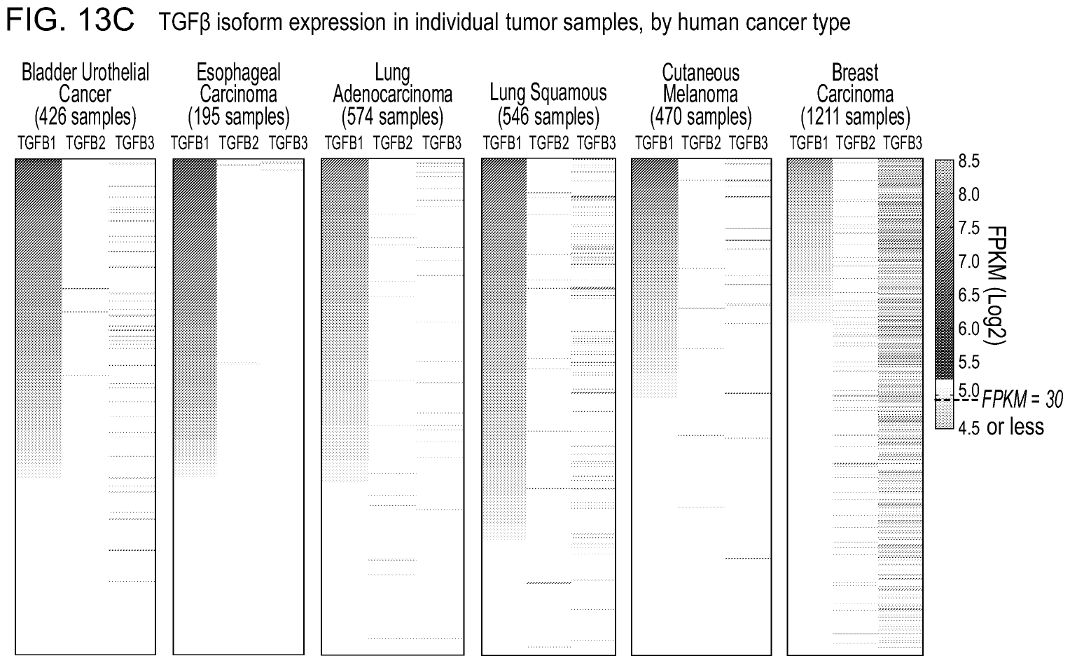

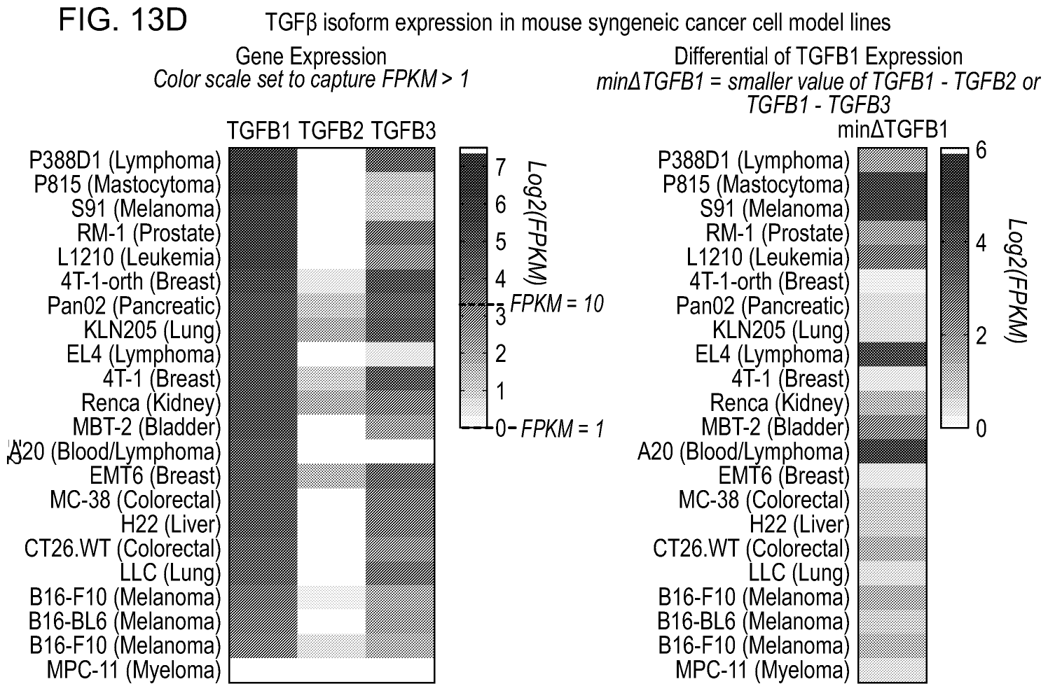

Accordingly, the present invention includes the recognition that differential expression of TGFβ isoforms in preclinical animal models should betaken into account in predicting effectiveness of particular inhibitors, as well as in the meaningful interpretation of preclinical data as to the translatability into human clinical conditions. As exemplified herein, TGFβ1 and TGFβ3 are co-dominant in certain murine syngeneic cancer models (e.g., EMT-6 and 4T1) that are widely used in preclinical studies (see FIG. 13D). By contrast, numerous other cancer models (e.g., S91, B16 and MBT-2) express almost exclusively TGFβ1, similar to that observed in many human tumors, in which TGFβ1 appears to be more frequently the dominant isoform over TGFβ2/3 (see FIGS. 13B and 13C). Furthermore, the TGFβ isoform(s) predominantly expressed under homeostatic conditions may not be the disease-associated isoform(s). For example, in normal lung tissues in healthy rats, tonic TGFβ signaling appears to be mediated mainly by TGFβ3. However, TGFβ1 appears to become markedly upregulated in disease conditions, such as lung fibrosis. Taken together, it is beneficial to test or confirm relative expression of TGFβ isoforms in clinical samples so as to select suitable therapeutics to which the patient is likely to respond.

As described herein, the isoform-selective TGFβ1 inhibitors are particularly advantageous for the treatment of diseases in which the TGFβ1 isoform is predominantly expressed relative to the other isoforms (e.g., referred to as TGFβ1-dominant). As an example, a non-limiting list of human cancer clinical samples with relative expression levels of TGFB1 (left), TGFB2 (center) and TGFB3 (right) is provided in FIG. 13C. Each horizontal lime across the three isoforms represents a single patient. As can be seen, overall TGFβ1 expression (TGFB1) is significantly higher in most of these human tumors/cancers than the other two isoforms across many tumor/cancer types, suggesting that TGFβ1-selective inhibition may be beneficial in these disease types.

Certain exceptions should be noted, however. First, such trend is not always applicable in certain individual patients within the disease type. That is, even in a type of cancer that shows almost uniformly TGFβ1-dominance over TGFβ2/3 overall, there are a few individuals that do not follow this general rule/trend. Patients that fall within the minority subpopulation therefore may not respond to a TGFβ1 isoform-specific inhibitor therapy in the way that works for a majority of patients. Second, there are a few cancer types in which TGFβ1 is co-dominant with another isoform or in which TGFβ2 and/or TGFβ3 expression is significantly greater than TGFβ1. In these situations, TGFβ1-selective inhibitors such as those described herein are not likely to be efficacious used alone. Rather, suitable additional inhibitor(s) that target other isoform(s) may be employed in conjunction (see, for example, WO 2016/201282). To manage potentially serious toxicities, however, pan-TGFβ inhibitors, as well as inhibitors that antagonize both TGFβ2 and TGFβ3, should be avoided.

For example, in diseases (such as certain types of carcinoma and sarcoma) or individual patients where TGFβ1 is co-dominant with TGFβ3 (for example as shown by biopsy analysis), suitable therapeutic regimen may include both a TGFβ1 inhibitor and a TGFβ3 inhibitor. Preferably, each of the inhibitors is an isoform-selective inhibitor, so as to avoid unwanted side effects or toxicities associated with pan-inhibition of all TGFβ isoforms. In some embodiments, one or both of the isoform-selective inhibitors inhibit(s) the activation step of the TGFβ isoform (e.g., TGFβ1 and/or TGFβ3). In preferred embodiments, the isoform-selective TGFβ1 inhibitor is an activation inhibitor such as those described herein. In some embodiments, the isoform-selective TGFβ3 inhibitor is an activation inhibitor of TGFβ3, made by the process comprising the step of selecting an antibody or antigen-binding fragment that specifically binds a proTGFβ3 complex. Typically, such process further includes selection or confirmation of antibody or fragment for the ability to bind multiple antigen complexes, e.g., LTBP1-proTGFβ3, LTBP3-proTGFβ3, GARP-proTGFβ3, and/or LRRC33-proTGFβ3. Preferably, such process further includes selection or confirmation of antibody or fragment for the ability to inhibit the release of the growth factor from the latent complex (i.e., activation inhibition).

When suitable therapeutic regimens include two isoform-selective TGFβ inhibitors, such as TGFβ1 and TGFβ3 (as in the example above), such therapy may comprise a single formulation that includes both TGFβ1 and TGFβ3 inhibitors. Such formulation may contain, for example, 10-50 mg/mL of each inhibitor and one or more pharmaceutically acceptable excipients.

Alternatively, such therapy may comprise the use of two separate formulations each comprising a single inhibitor for administration to a patient or patient population. This offers added flexibility in adjusting the ratios of the two inhibitor dosages to be administered to the patient or patient population, depending on (and tailored to) relative expression levels (above healthy levels) of the two TGFβ isoforms shown to be present in one or more biological samples collected from the patient or patient population. For example, for use in the treatment of TGFβ1-positive, TGFβ3-positive cancer/tumors (such as breast cancer), where the former is the dominant disease-associated isoform relative to the latter, TGFβ1 inhibitor may be used at higher dose and/or longer duration as part of the therapeutic regimens.

In some embodiments, the TGFβ1-selective inhibitors disclosed herein are sufficient to treat a disease (e.g., fibrosis, solid tumors, etc.) despite co-expression of TGFβ1 and TGFβ3.

Therefore, it is beneficial to test or confirm relative expression levels of the three TGFβ isoforms (i.e., TGFβ1, TGFβ2 and TGFβ3) in clinical samples collected from individual patients. Such information may provide better prediction as to the effectiveness of a particular therapy in individual patients or patient populations, which can help ensure selection of appropriate treatment regimen (e.g., individualized/personalized treatment) in order to increase the likelihood of a clinical response.

Accordingly, the invention includes a method for selecting a patient population or a subject who is likely to respond to a therapy comprising an isoform-specific TGFβ1 inhibitor according to the present disclosure. Such method comprises the steps of: providing a biological sample (e.g., clinical sample) collected from a subject, determining (e.g., measuring or assaying) relative levels of TGFβ1, TGFβ2 and TGFβ3 in the sample, and, administering to the subject a composition comprising the TGFβ1 inhibitor, if TGFβ1 is the dominant isoform over TGFβ2 and TGFβ3; and/or, if TGFβ1 is significantly overexpressed or upregulated as compared to control. In some embodiments, such method comprises the steps of: obtaining information on the relative expression levels of TGFβ1, TGFβ2 and TGFβ3 which was previously determined; identifying a subject to have TGFβ1-positive, preferably TGFβ1-dominant disease; and, administering to the subject the TGFβ1 inhibitor. In some embodiments, such subject has a disease (such as cancer) that is resistant to a therapy (such as cancer therapy). In some embodiments, such subject shows intolerance to the therapy and therefore has or is likely to discontinue the therapy. Addition of the TGFβ1 inhibitor to the therapeutic regimen may enable reducing the dosage of the first therapy and still achieve clinical benefits in combination.

Relative levels of the isoforms may be determined by RNA-based assays and/or protein-based assays, which are well-known in the art. In some embodiments, the step of administration may also include another therapy, such as immune checkpoint inhibitors, or other agents provided elsewhere herein. Such methods may optionally include a step of evaluating a therapeutic response by monitoring changes in relative levels of TGFβ1, TGFβ2 and TGFβ3 at two or more time points. In some embodiments, clinical samples (such as biopsies) are collected both prior to and following administration. In some embodiments, clinical samples (such as biopsies) are collected multiple times following treatment to assess in vivo effects over time.

In addition to the first inquiry drawn to the aspect of isoform specificity, the second inquiry interrogates the breadth of TGFβ1 function involved in a particular disease. This may be represented by the number of TGFβ1 contexts, namely, which presenting molecule(s) mediate disease-associated TGFβ1 function. TGFβ1-specific, broad-context inhibitors, such as context-independent inhibitors, are advantageous for the treatment of diseases that involve both an ECM component and an immune component of TGFβ1 function. Such disease may be associated with dysregulation in the ECM as well as perturbation in immune cell function or immune response.

Whether or not a particular condition of a patient involves or is driven by multiple aspects of TGFβ1 function may be assessed by evaluating expression profiles of the presenting molecules, in a clinical sample collected from the patient. Various assays are known in the art, including RNA-based assays and protein-based assays, which may be performed to obtain expression profiles. Relative expression levels (and/or changes/alterations thereof) of LTBP1, LTBP3, GARP, and LRRC33 in the sample(s) may indicate the source and/or context of TGFβ1 activities associated with the condition. For instance, a biopsy sample taken from a solid tumor may exhibit high expression of all four presenting molecules. For example, LTBP1 and LTBP3 may be highly expressed in CAFs within the tumor stroma, while GARP and LRRC33 may be highly expressed by disease-associated immune cells, such as Tregs, MDSCs and leukocyte infiltrate, respectively. Similarly, LTBP1 and LTBP3 may be highly expressed in FAFs (e.g., myofibroblasts) within the fibrotic microenvironment, while LRRC33 may be highly expressed by fibrosis-associated immune cells, such as M2 macrophages and MDSCs.

Accordingly, the invention includes a method for determining (e.g., testing or confirming) the involvement of TGFβ1 in the disease, relative to TGFβ2 and TGFβ3. In some embodiments, the method further comprises a step of: identifying a source (or context) of disease-associated TGFβ1. In some embodiments, the source/context is assessed by determining the expression of TGFβ presenting molecules, e.g., LTBP1, LTBP3, GARP and LRRC33 in a clinical sample collected from patients.

Isoform-selective TGFβ1 inhibitors, such as those described herein, may be used to treat a wide variety of diseases, disorders and/or conditions that are associated with TGFβ1 dysregulation (i.e., TGFβ1-related indications) in human subjects, As used herein, “disease (disorder or condition) associated with TGFβ1 dysregulation” or “TGFβ1-related indication” means any disease, disorder and/or condition related to expression, activity and/or metabolism of a TGFβ1 or any disease, disorder and/or condition that may benefit from inhibition of the activity and/or levels TGFβ1.

The present invention includes the use of such isoform-specific TGFβ1 inhibitor in a method for treating a disease associated with TGFβ1 dysregulation in a human subject. Such inhibitor is typically formulated into a pharmaceutical composition that further comprises a pharmaceutically acceptable excipient. TGFβ is a key regulator of ECM components, structure and function. Advantageously, the inhibitor targets both ECM-associated TGFβ1 and immune cell-associated TGFβ1 but does not target TGFβ2 or TGFβ3 in vivo. In some embodiments, the inhibitor preferentially binds ECM-associated proTGFβ1 complexes thereby blocking TGFβ1 signaling in the matrix niche. The disease may involve dysregulation or impairment of ECM components or function and comprises increased collagen deposition. In some embodiments, the dysregulation or impairment of ECM components or function may further comprise increased stiffness and/or ECM reorganization. In some embodiments, the dysregulation or impairment of ECM components or function includes increased myofibroblast cells within the disease site. In some embodiments, the dysregulation of the ECM includes increased stiffness of the matrix. In some embodiments, the dysregulation of the ECM involves fibronectin and/or fibrillin.

In some embodiments, the disease is characterized by dysregulation or impairment of myeloid cell proliferation or differentiation; wherein optionally the dysregulation or impairment of myeloid cells comprises monocyte recruitment to the disease site or differentiation into polarized M2 cells, and/or, abnormal macrophage function. In some embodiments, the dysregulation of myeloid cells comprises increased levels of MDSCs. Elevated MDSCs may comprise an increased number/frequency of circulating MDSCs, e.g., in peripheral blood. Elevated MDSCs may be observed at the site of the disease, such as fibrotic tissues and solid tumors.

In some embodiments, the disease is characterized by abnormal cell differentiation involving epithelial-to-mesenchymal transition (EMT) and/or endothelial-to-mesenchymal transition (EndMT). In some embodiments, these processes occurring at the disease sites (such as TME and fibrotic microenvironment) result in increased myofibroblasts or myofibroblast-like cells at the site. These include, for example, CAFs and FAFs.

In some embodiments, the disease is characterized by abnormal gene expression in one or more of marker genes selected from the group consisting of: PAI-1, ACTA2, CCL2, Col1a1, Col3a1, FN-1, CTGF, and TGFB1.

A therapeutically effective amount of such inhibitor is administered to the subject suffering from or diagnosed with the disease.

In some embodiments, the dysregulation or impairment of fibroblast differentiation comprises increased myofibroblasts or myofibroblast-like cells. In some embodiments, the myofibroblasts or myofibroblast-like cells are cancer-associated fibroblasts (CAFs). In some embodiments, the CAFs are associated with a tumor stroma and may produce CCL2/MCP-1 and/or CXCL12/SDF-1. In some embodiments, the myofibroblasts or myofibroblast-like cells are localized to a fibrotic tissue.

In some embodiments, the dysregulation or impairment of regulatory T cells comprises increased Treg activity.

In some embodiments, the dysregulation or impairment of effector T cell (Teff) proliferation or function comprises suppressed CD4+/CD8+ cell proliferation.

In some embodiments, the dysregulation or impairment of myeloid cell proliferation or differentiation comprises increased proliferation of myeloid progenitor cells. The increased proliferation of myeloid cells may occur in a bone marrow,

In some embodiments, the dysregulation or impairment of monocyte differentiation comprises increased differentiation of bone marrow-derived and/or tissue resident monocytes into macrophages at a disease site, such as a fibrotic tissue and/or a solid tumor.

In some embodiments, the dysregulation or impairment of monocyte recruitment comprises increased bone marrow-derived monocyte recruitment into a disease site such as TME, leading to increased macrophage differentiation and M2 polarization, followed by increased TAMs.

In some embodiments, the dysregulation or impairment of macrophage function comprises increased polarization of the macrophages into M2 phenotypes.

In some embodiments, the dysregulation or impairment of myeloid cell proliferation or differentiation comprises an increased number of Tregs, MDSCs and/or TANs.

TGFβ-related indications may include conditions comprising an immune-excluded disease microenvironment, such as tumor or cancerous tissue that suppresses the body's normal defense mechanism/immunity in part by excluding effector immune cells (e.g., CD4+ and/or CD8+ T cells). In some embodiments, such immune-excluding conditions are associated with poor responsiveness to treatment(e.g., cancer therapy). Non-limiting examples of the cancer therapies, to which patients are poorly responsive, include but are not limited to: checkpoint inhibitor therapy, cancer vaccines, chemotherapy, and radiation therapy. Without intending to be bound by particular theory, it is contemplated that TGFβ inhibitors, such as those described herein, may help counter the tumor's ability to evade or exclude anti-cancer immunity by restoring T cell (e.g., CD8+ cells) access by promoting T cell expansion and/or infiltration into tumor.

Thus, TGFβ inhibition may overcome treatment resistance (e.g., immune checkpoint resistance, cancer vaccine resistance, CAR-T resistance, chemotherapy resistance, radiation therapy resistance, etc.) in immune-excluded disease environment (such as TME) by unblocking and restoring effector T cell access and cytotoxic effector functions. Such effects of TGFβ inhibition may further provide long-lasting immunological memory mediated, for example, by CD8+ T cells.

Non-limiting examples of TGFβ-related indications include: fibrosis, including organ fibrosis (e.g., kidney fibrosis, liver fibrosis, cardiac/cardiovascular fibrosis, muscle fibrosis, skin fibrosis, uterine fibrosis/endometriosis and lung fibrosis), scleroderma, Alport syndrome, cancer (including, but not limited to: blood cancers such as leukemia, myelofibrosis, multiple myeloma, colon cancer, renal cancer, breast cancer, malignant melanoma, glioblastoma), fibrosis associated with solid tumors (e.g., cancer desmoplasia, such as desmoplastic melanoma, pancreatic cancer-associated desmoplasia and breast carcinoma desmoplasia), stromal fibrosis (e.g., stromal fibrosis of the breast), radiation-induced fibrosis (e.g., radiation fibrosis syndrome), facilitation of rapid hematopoiesis following chemotherapy, bone healing, wound healing, dementia, myelofibrosis, myelodysplasia (e.g., myelodysplasic syndromes or MDS), a renal disease (e.g., end-stage renal disease or ESRD), unilateral ureteral obstruction (UUO), tooth loss and/or degeneration, endothelial proliferation syndromes, asthma and allergy, gastrointestinal disorders, anemia of the aging, aortic aneurysm, orphan indications (such as Marfan's syndrome and Camurati-Engelmann disease), obesity, diabetes, arthritis, multiple sclerosis, muscular dystrophy (e.g., Myotonic muscular dystrophy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy and Emery-Dreifuss muscular dystrophy), amyotrophic lateral sclerosis (ALS), Parkinson's disease, osteoporosis, osteoarthritis, osteopenia, metabolic syndromes, nutritional disorders, organ atrophy, chronic obstructive pulmonary disease (COPD), and anorexia.

TGFβ-related indications may also include conditions in which major histocompatibility complex (MHC) class I is deleted or deficient (e.g., downregulated). Such conditions include genetic disorders in which one or more components of the MHC-mediated signaling is impaired, as well as conditions in which MHC expression is altered by other factors, such as cancer, infections, fibrosis, and medications.

For example, MHC I downregulation in tumor is associated with tumor escape from immune surveillance. Indeed, immune escape strategies aimed to avoid T-cell recognition, including the loss of tumor MHC class I expression, are commonly found in malignant cells. Tumor immune escape has been observed to have a negative effect on the clinical outcome of cancer immunotherapy, including treatment with antibodies blocking immune checkpoint molecules (reviewed in, for example: Garrido et al. (2017)Curr Opin Immunol 39:44-51. “The urgent need to recover MHC class I in cancers for effective immunotherapy”, incorporated by reference herein). Thus, the isoform-selective, TGFβ1 inhibitors encompassed by the present disclosure may be administered either as a monotherapy or in conjunction with another therapy (such as checkpoint inhibitor, chemotherapy, radiation therapy, etc.) to unleash or boost anti-cancer immunity and/or enhance responsiveness to or effectiveness of another therapy.

Downregulation of MHC class I proteins are also associated with certain infectious diseases, including viral infections such as HIV. See for example, Cohen et al. (1999) Immunity 10(6): 661-671. “The selective downregulation of class I major histocompatibility complex proteins by HIV-1 protects HIV-infected cells from NK Cells”, incorporated herein by reference. Thus, the isoform-selective, TGFβ1 inhibitors encompassed by the present disclosure may be administered either as a monotherapy or in conjunction with another therapy (such as anti-viral therapy, protease inhibitor therapy, etc.) to unleash or boost host immunity and/or enhance responsiveness to or effectiveness of another therapy.

### Fibrotic Conditions

In response to tissue injury due physical damage/trauma, toxic substances, and/or infection, a natural reparative process begins which involves several cell types including fibroblasts, several different types of immune cells, and resident epithelial and endothelial cells. However, if left unchecked, this process can lead to excessive accumulation of extracellular matrix (ECM) and fibrosis, which in turn can lead to progressive loss of tissue function and organ failure (Caja et al., Int. J. Mol. Sci. 2018, 19, 1294).

Fibrosis can occur in several different organs, including lung, kidney, liver, heart, and skin. Independent of the organ, the fibrotic response is characterized by inflammation, altered epithelial-mesenchymal interactions, and proliferation of fibroblasts. One of the hallmarks of fibrosis is the differentiation of fibroblasts into myofibroblasts, which greatly contribute to the dysregulation of the ECM. However, myofibroblasts have also been proposed to come from other cellular sources (e.g., endothelial cells, epithelial cells, and mesenchymal stem cells (Kim, K. K. et al, Cold Spring Harb. Perspect. Biol., 2017; Okabe, H. Histol. Histophathol., 2016, 31, 141-148; and Li, C et al, Nat Commun., 2016, 7, 11455 and). Moreover, immune cells play an important role in the process by secreting cytokines and chemokines which promote differentiation of myofibroblasts, stimulate ECM deposition, and recruit additional immune cells to the damaged tissue (Caja et al., Int. J. Mol. Sci. 2018, 19, 1294).

Similar to fibrotic tissue, activation of cancer-associate fibroblasts can occur in the tumor milieu, which produces excessive amounts of ECM. The ECM provides a scaffold for the infiltration of other cells (e.g., pro-tumorigenic immune cells) and a substrate for cell migration. In other cases, excessive ECM may act as a barrier against anti-tumorigenic immune cells.

TGFβ is recognized as the central orchestrator of the fibrotic response. TGFβ can promote myofibroblast differentiation, recruit immune cells, and affect epithelial and endothelial cell differentiation. Particularly, TGFβ upregulates the production of ECM and basement membrane proteins, such as fibronectin, collagen, laminin, osteopontin, tenascin, elastin, decorin. TGFβ-induced myofibroblast differentiation can lead to additional deposition of ECM proteins, secretion of matric metallopoteinases (MMPs), and myofibroblast proliferation (Fabregat et al, FEBS J. 2016, 283, 2219-2232; Meng et al, Nat. Rev. Nephrol. 2016, 12, 325-338; and Kulkarni et al., Am. J. Respir. Cell Mol. Biol., 2016, 54, 751-760). Additionally, TGFβ mediates phenotypic changes affecting contractile proteins and collagen I in vascular smooth muscle cells (VSCM), and can activate myofibroblasts and other stromal cells to enhance the synthesis of collagen cross-linking proteins, such as lysyl oxidase (LOX) family of matrix-remodeling enzymes (Busnadiego et al., Mol. Cell. Biol. 2013, 33, 2388-2401). Moreover, TGFβ has been shown to regulate both EMT and EndMT, which contributes to the differentiation of pro-fibrotic cell types, such as myofibroblasts and CAFs. Moreover, TGFβ has been shown to induce epithelial apoptosis, which can promote lung and liver fibrosis among other tissues (Barbas-Filho et al., J. Clin. Pathol. 2001, 54, 132-138; and Wang et al., Dev. Dyn. 2017, 247, 492-508).

Whether innate or recruited, macrophages play an important role in responding to tissue damage and repair. However, upon certain signals they can become pro-fibrotic. TGFβ, among other cytokines, has also been shown to activate M2 macrophages, which are pro-inflammatory. Upon activation, these macrophages secrete their own cytokines, including TGFβ, ECM components, angiogenic factors, and chemotactic factors. M2 macrophages have been shown to be essential for TGFβ-driven lung fibrosis (Murray et al., Int. J. Biochem. Cell Biol. 2011, 43, 154-162).

Thus, according to the invention, isoform-specific, inhibitors TGFβ1 such as those described herein are used in the treatment of fibrosis (e.g., fibrotic indications, fibrotic conditions) in a subject. Suitable inhibitors to carry out the present invention include antibodies and/or compositions according to the present disclosure which may be useful for altering or ameliorating fibrosis. More specifically, such antibodies and/or compositions are selective antagonists of TGFβ1 that are capable of targeting TGFβ1 presented by various types of presenting molecules.

Antibodies targeting TGFβ decrease fibrosis in numerous preclinical models. Such antibodies and/or antibody-based compounds include LY2382770 (Eli Lilly, Indianapolis, Ind.). Also included are those described in U.S. Pat. Nos. 6,492,497, 7,151,169, 7,723,486 and U.S. Appl. Publ. No. 2011/0008364, the contents of each of which are herein incorporated by reference in their entirety. Prior art TGFβ antagonists include, for example, agents that target and block integrin-dependent activation of TGFβ.

However, evidence suggests that such prior art agents may not mediate isoform-specific inhibition and may cause unwanted effects by inadvertently blocking normal function of TGFβ2 and/or TGFβ3. Indeed, data presented herein support this notion. Normal (undiseased) lung tissues contain relatively low but measurable levels of TGFβ2 and TGFβ3, but notably less TGFβ1. In comparison, in certain disease conditions such as fibrosis, TGFβ1 becomes preferentially upregulated relative to the other isoforms. Preferably, TGFβ antagonists for use in the treatment of such conditions exert their inhibitory activities only towards the disease-induced or disease-associated isoform, while preserving the function of the other isoforms that are normally expressed to mediate tonic signaling in the tissue. Prior art inhibitors (LY2109761, a small molecule TGFβ receptor antagonist, and a monoclonal antibody that targets αVβ6 integrin) both are shown to inhibit TGFβ downstream tonic signaling in non-diseased rat BAL, raising the possibility that these inhibitors may cause unwanted side effects. Alternatively or additionally, agents that target and block integrin-dependent activation of TGFβ may be capable of blocking only a subset of integrins responsible for disease-associated TGFβ1 activation, among numerous integrin types that are expressed by various cell types and playa role in the pathogenesis. Furthermore, even where such antagonists may selectively block integrin-mediated activation of the TGFβ1 isoform, it may be ineffective in blocking TGFβ1 activation triggered by other modes, such as protease-dependent activation. By contrast, the isoform-specific, inhibitors of TGFβ1 such as those described herein are aimed to prevent the activation step of TGFβ1 regardless of the particular mode of activation, while maintaining isoform selectivity.

It is further contemplated that isoform-specific TGFβ1 inhibitors that preferentially inhibit matrix-associated over cell-associated antigen complexes (i.e., display context-bias) may offer a therapeutic advantage in certain clinical situations. For example, TGFβ1 inhibitors (which target all four antigen complexes), may increase immune activation through the targeting of cell-associated TGFβ1 (e.g., GARP-TGFβ1 which is expressed on regulatory T cells). Immune activation may be disadvantageous for certain patients, e.g., patients with autoimmune disease or who are at risk of sepsis. Accordingly, context-bias antibodies may be useful for treating diseases associate with matrix-associated TGFβ1 complexes (e.g., fibrosis), while minimizing immune activation.

It is further contemplated that isoform-specific TGFβ3 inhibitors may offer a therapeutic benefit in particular disease states. For example, certain fibrotic diseases to be treated with a TGFβ1 inhibitor may also be TGFβ3-positive (i.e., TGFβ1+/TGFβ3+ fibrotic tissue) characterized in that the disease tissue (e.g., fibrotic tissue) expresses both the isoforms. Accordingly, the invention includes the use of isoform-selective TGFβ1 inhibitor in conjunction with an isoform-selective TGFβ3 inhibitor in the treatment of such conditions. Such TGFβ3 inhibitors may be context-independent or context-bias. Fibrotic indications for which antibodies and/or compositions of the present disclosure may be used therapeutically include, but are not limited to lung indications (e.g. idiopathic pulmonary fibrosis (IPF), chronic obstructive pulmonary disorder (COPD), allergic asthma, acute lung injury, eosinophilic esophagitis, pulmonary arterial hypertension and chemical gas-injury), kidney indications (e.g., diabetic glomerulosclerosis, focal segmental glomeruloclerosis (FSGS), chronic kidney disease (CKD), fibrosis associated with kidney transplantation and chronic rejection, IgA nephropathy, and hemolytic uremic syndrome), liver fibrosis (e.g., associated with or caused by non-alcoholic steatohepatitis (NASH), chronic viral hepatitis, parasitemia, inborn errors of metabolism, toxin-mediated fibrosis, such as alcohol fibrosis, non-alcoholic steatohepatitis-hepatocellular carcinoma (NASH-HCC), primary biliary cirrhosis, and sclerosing cholangitis), cardiovascular fibrosis (e.g., cardiomyopathy, hypertrophic cardiomyopathy, atherosclerosis and restenosis) systemic sclerosis, skin fibrosis (e.g. skin fibrosis in systemic sclerosis, diffuse cutaneous systemic sclerosis, scleroderma, pathological skin scarring, keloid, post-surgical scarring, scar revision surgery, radiation-induced scarring and chronic wounds), eye-related conditions such as subretinal fibrosis, uveitis syndrome, uveitis associated with idiopathic retroperitoneal fibrosis, extraocular muscle fibrosis, eye diseases associated with the major histocompatibility complex (MHC class I) or histocompatibility antigens, subretinal fibrosis in macular degeneration (e.g., age-related macular degeneration), and cancers or secondary fibrosis (e.g. myelofibrosis, head and neck cancer, M7 acute megakaryoblastic leukemia and mucositis). Other diseases, disorders or conditions related to fibrosis (including degenerative disorders) that may be treated using compounds and/or compositions of the present disclosure, include, but are not limited to adenomyosis, endometriosis, Marfan's syndrome, stiff skin syndrome, scleroderma, rheumatoid arthritis, bone marrow fibrosis, Crohn's disease, ulcerative colitis, systemic lupus erythematosus, muscular dystrophy (such as DMD), Parkinson's disease, ALS, Dupuytren's contracture, Camurati-Engelmann disease, neural scarring, dementia, proliferative vitreoretinopathy, corneal injury, complications after glaucoma drainage surgery, and multiple sclerosis (MS). Many such fibrotic indications are also associated with inflammation of the affected tissue(s), indicating involvement of an immune component. Such inflammation may be accompanied by aberrant immune cell populations, such as increased numbers of Th17 cells, reduced numbers of Treg cells, and/or both. In each case, the affected patient may exhibit increased Th17/Treg cell ratios.

In some embodiments, fibrotic indications that may be treated with the compositions and/or methods described herein include organ fibrosis, such as fibrosis of the lung (e.g., IPF), fibrosis of the kidney (e.g., fibrosis associated with CKD), fibrosis of the liver (e.g., associated with or due to NASH), fibrosis of the heart or cardiac tissues, fibrosis of the skin (e.g., scleroderma), fibrosis of the uterus (e.g., endometrium, myometrium), fibrosis of muscle (e.g., skeletal muscle), and fibrosis of the bone marrow. In some embodiments, such therapy may reduce or delay the need for organ transplantation in patients. In some embodiments, such therapy may prolong the survival of the patients.

To treat IPF, patients who may benefit from the treatment include those with familial IPF and those with sporadic IPF. Administration of a therapeutically effective amount of an isoform-specific, inhibitor of TGFβ1 may reduce myofibroblast accumulation in the lung tissues, reduce collagen deposits, reduce IPF symptoms, improve or maintain lung function, and prolong survival. In some embodiments, the inhibitor blocks activation of ECM-associated TGFβ1 (e.g., pro/latent TGFβ1 presented by LTBP1/3) within the fibrotic environment of IPF. The inhibitor may optionally further block activation of macrophage-associated TGFβ1 (e.g., pro/latent TGFβ1 presented by LRRC33), for example, alveolar macrophages. As a result, the inhibitor may suppress fibronectin release and other fibrosis-associated factors. In some embodiments, the inhibitor blocks hepatic stellate cell activation.

It is well-established that the activation of hepatic stellate cells (HSCs) are the central drivers of fibrosis in liver injury. In this process, quiescent, vitamin-A-storing cells, transdifferentiated into proliferative, fibrogenic myofibroblasts (the principal source of extracellular matrix(ECM) protein accumulation). However, this process has been shown to be mediated by many different pathways, including autophagy, endoplasmic reticulum stress, oxidative stress, retinol and cholesterol metabolism, epigenetics, and receptor-mediated signals. Moreover, inflammatory cells including macrophages, hepatocytes, livers inusoidal endothelial cells, natural killer cells, natural killer T cells, platelets and B cells have also been shown to modulate HSC activation (Tsuchida and Friedman, Nature Reviews Gastroenterology& Hepatology volume 14, pages 397-411 (2017)). In just one particular example, Seki et al demonstrated that TLR4 (which recognizes LPS presented by bacteria) activation leads to upregulation of chemokine secretion and induces chemotaxis of Kupffer cells, and also sensitizes HSCs to TGFβ-induced signals and allows for unrestricted activation of Kupffer cells (Seki et al. Nature Medicine volume 13, pages 1324-1332 (2007)).

It is well known that inflammation plays a key role in liver fibrosis development and progression. Specifically, liver injury leads to inflammation and the recruitment of monocytes/macrophages (as well as lymphocytes, eosinophils, and plasma cells) which produce pro-fibrotic factors, including TGFβ. Moreover, the research indicates that both hepatic tissue-resident macrophages (Kupffer cells) and bone marrow-derived recruited macrophages play important roles in the progression of liver fibrosis, and that the TGFβ pathway can promote the polarization and pro-fibrotic functions of macrophages during liver fibrosis. Indeed, it has been shown that both Kupffer cells and recruited macrophages can activate HSCs and induce their transdifferentiation into myofibroblasts by paracrine mechanisms, including TGFβ. The myofibroblasts in turn produce and deposit ECM components leading to fibrosis (Fabregat and Caballero-Diaz, Front Oncol. 2018; 8: 357).

However, myofibroblasts may come from other sources as well, including portal and resident fibroblasts, bone marrow-derived fibrocytes, liver epithelial cells that undergo EMT, endothelial cells that undergo EndMT, and vascular smooth muscle cells and pericytes. Indeed, TGFβ has also been shown to regulate both EndMT and EMT resulting in increased myofibroblasts, which drive liver fibrosis. (Pardali et al., Int J Mol Sci. 2017 October; 18(10): 2157). Accordingly, targeting TGFβ has been an attractive therapeutic target for the treatment of fibrotic conditions.

TGFβ has been shown to play many roles in liver fibrosis and disease progression. For example, TGFβ has been shown to be responsible for the activation HSCs to myofibroblasts. TGFβ also has been shown to mediate epithelial-mesenchymal transition (EMT) in hepatocytes that may contribute to increase the myofibroblast population. Moreover, TGFβ has been shown to induce changes in tumor cell plasticity (Fabregat and Caballero-Diaz, Front Oncol. 2018; 8: 357).

Although TGFβ can be found on many different cellular sources in the fibrotic and/or tumor microenvironment, thus suggesting TGFb presentation by multiple different presenting molecules (e.g., LTBP1, LTBP3, GARP, and/or LRRC33), it may be beneficial in certain situations to target particular sources of TGFβ over others. For example, Henderson et al, showed that deleting αv integrin in HSCs, protected mice form CCL₄-induced liver fibrosis (Henderson et al, Nat. Med. 2013, 19, 1617-16-24). Because integrins are the main activators of LTBP-presented TGFβ, this result suggests that targeting LTBP-presented TGFβ may be sufficient to treat fibrosis in certain situations. However, because immune cells play an important role in the fibrotic response, TGFβ inhibitors that target TGFβ presented by most or all of the presenting-molecule TGFβ complexes may be beneficial.

In recent years, the treatment of liver fibrosis has become an area interest due to its increasing prevalence around the world. For example, non-alcoholic fatty liver disease (NAFLD) is associated with metabolic abnormalities such as obesity, insulin resistance, fasting hyperglycemia, dyslipidaemia, and altered adipokine profiles. NAFLD is characterized by excessive lipid accumulation in hepatocytes and is a spectrum of diseases progressing from liver steatosis (lipid/fat droplet accumulation in hepatocytes) to non-alcoholic steatohepatitis (NASH), liver fibrosis, and eventually cirrhosis in the most severe cases. NASH with fibrosis or cirrhosis increases the risk of developing hepatocellular carcinoma (HCC) (Starley B Q, et al. Hepatology 2010; 51: 1820-1832). The progression from steatosis to NASH has been proposed to be regulated by a ‘multiple-hit’ model, wherein the first hit is insulin resistance and metabolic disturbance, which leads to liver steatosis, followed by oxdative stress, proinflammatory cytokine-mediated hepatocyte injury, altered lipid partitioning and hepatoxcity mediated by free fatty acids, abnormal intrahepatic cholesterol loading, hyperinsulinaemia, hyperleptinaemia, and hypoadiponectinaemia (Tilg H, Moschen A R, Hepatology 2010; 52: 1836-1846; and Yilmaz Y., Aliment Pharmacol Ther 2012; 36: 815-823).

There are many animal models that have been develop to study liver fibrosis. For example, a high fat diet in mice has been shown to mimic both the histopathology and pathogenesis of human NAFLD. Moreover, some genetic models also display features of human metabolic syndrome and NAFLD, such as db/db and ob/ob mouse models. There are also animal models for the study of NASH, which mainly consist of various diet-induced models, including, but not limited to, methionine and choline-deficient diet (MCD), high-cholesterol diet (HCD), choline-deficient high fat diet (CDHFD), choline-deficient L-amino acid-deficient diet, choline-deficient L-amino acid-deficient diet+carbon tetrachloride, high-fat diet+streptozotocin, high fat+high cholesterol diet (HFHC), high-fructose diet (HFD), and high-fructose high fat diet (HFHF). Genetic mouse models for the study of NASH include, but are not limited to foz/foz mice, Hepatocyte-specific PTEN-deficient mice, Db/db mice+diethylnitrosamine (DEN), and db/db mice+MCD. The details of all of these models, including the pluses and minus of each, are outlined in Jennie Ka Ching Lau et al., J Pathol 2017; 241: 36-44; the contents of which are incorporated herein by reference.

Another model useful for testing the efficacy of isoform-specific TGFβ inhibitors in liver fibrosis include the carbon tetrachloride (CCL₄) model. Another model useful for testing the efficacy of isoform-specific TGFβ inhibitors in liver fibrosis include the bile duct ligation (BDL) model (see, e.g., Tag et al., J Vis Exp. 2015; (96): 52438).

The isoform-specific, TGFβ1 inhibitors such as those provided herein may be used to treat fibrotic conditions of the liver, such as fatty liver (e.g., non-alcoholic fatty liver disease (NAFLD) and non-alcoholic steatohepatitis (NASH). The fatty liver may or may not be inflamed. Inflammation of the liver due to fatty liver (i.e., steatohepatitis) may develop into scarring (fibrosis), which then often progresses to cirrhosis (scarring that distorts the structure of the liver and impairs its function). The inhibitor may therefore be used to treat such conditions. In some embodiments, the inhibitor blocks activation of ECM-associated TGFβ1 (e.g., pro/latent TGFβ1 presented by LTBP1/3) within the fibrotic environment of the liver. The inhibitor may optionally further block activation of macrophage-associated TGFβ1 (e.g., pro/latent TGFβ1 presented by LRRC33), for example, Kupffer cells (also known as stellate macrophages) as well as infiltrating monocyte-derived macrophages and MDSCs. As a result, the inhibitor may suppress fibrosis-associated factors (e.g., fibrotic markers described herein). Administration of the inhibitor in a subject with such conditions may reduce one or more symptoms, prevent or retard progression of the disease, reduce or stabilize fat accumulations in the liver, reduce disease-associated biomarkers (such as serum collagen fragments), reduce liver scarring, reduce liver stiffness, and/or otherwise produce clinically meaningful outcome in a patient population treated with the inhibitor, as compared to a control population not treated with the inhibitor. In some embodiments, an effective amount of the inhibitor may achieve both reduced liver fat and reduced fibrosis (e.g., scarring) in NASH patients. In some embodiments, an effective amount of the inhibitor may achieve improvement in fibrosis by at least one stage with no worsening steatohepatitis in NASH patients. In some embodiments, an effective amount of the inhibitor may reduce the rate of occurrence of liver failure and/or liver cancer in NASH patients.

In some embodiments, an effective amount of the inhibitor may normalize, as compared to control, the levels of multiple inflammatory or fibrotic serum biomarkers as assessed following the start of the therapy, at, for example, 12-36 weeks. In some embodiments, inflammatory or fibrotic biomarkers may be used to assess severity of NAFLD (by measure levels of hepatic steatosis), select patients for treatment, and/or monitor disease progression or treatment response. For example, blood biomarkers and panels may include, but are not limited to:
i) the Fatty liver index (BMI, waist circumference, serum triglycerides, and gamma-glutamyltransferase (GGT);
ii) the Hepatic steatosis index(serum aspartate aminotransferase (AST):alanine aminotransferase (ALT) ratio, BMI, gender, and presence of diabetes mellitus);
i) the NAFLD liver fat score (serum ALT, HDL cholesterol, triglicerides, haemoglobin A_{1c} and leukocyte count);
ii) the SteatoTest (BioPredictive) (serum levels of total bilirubin, GGT, α2-macroglobin, haptoglobin, ALT, apolipoprotein Al, total cholesterol, triglycerides, glucose (adjusted for age and gender) and BMI); and
iii) the NAFLD ridge score (serum levels of ALT, HDL cholesterol, triglycerides, haemoglobin A_{1c}, leukocyte count, and comorbiditydata (and the presence of hypertension)).

In some embodiments, imaging biomarkers can be used to assess levels of hepaticsteatosis. For example, imaging biomarkers may include but are not limited to: ultrasonography, controlled attenuation parameter (CAP), MRI-estimated proton density fat fraction (MRI-PDFF), and magnetic resonance spectroscopy (MRS).

Liver biopsies are the current standard for diagnosis NASH, however, variability among pathologists limits the effectiveness of such diagnostic method. Accordingly, use of the Fatty Liver Inhibition of Progression (FLIP) algorithm (comprising histological steatosis, activity and fibrosis scores) may be used to improve the consistency of NASH diagnosis by biopsy. Moreover, many noninvasive biomarkers may also be useful for diagnosing and monitoring disease. Accordingly, in some embodiments, inflammatory or fibrotic biomarkers may be used to assess severity of NASH, select patients for treatment, and/or monitor disease progression or treatment response. Blood biomarkers may include:
i) apoptosis markers, such as CK18 fragments, total cytokeratin and sFAS;
ii) inflammatory markers, such as CRP, TNF, IL-8, and CXCL10;
iii) lipid oxidation products, such as 11-HETE, 9-HODE, 13-HODE, 12-oxo-ODE, LA-13-HODE (oxNASHscore), and 11,12-diHETrE;
iv) lysosomal enzymes, such as cathepsin D; and
v) combination panels, such as NASHTest (BioPredictive) and NASH Diagnostics Panel (comprising, presence of diabetes mellitus, sex, BMI, and serum levels of triglyceride, CK18 fragments, and total CK18).

In some embodiments, biomarkers and related panels may be useful in diagnosis levels of fibrosis and/or cirrhosis, select patients for treatment, and/or monitor disease progression or treatment response. For example, noninvasive tests of liver fibrosis and cirrhosis include, but are not limited to: AST:ALT ratio, AST:platelet ratio index, fibrosis-4 index (age, AST, ALT, and platelet count), NAFLD fibrosis score (age, BMI, impaired fasting glucose and/or diabetes, AST ALT, platelet count, and albumin), BARD score (AST, ALT, BMI, and diabetes).

Specific fibrosis markers and panels may also be useful, and include, but are not limited to: hyaluronic acid; PIIPNP; Pro-C3; TIMP1; Laminin; enhanced liver fibrosis (ELF) panel (PIINP, hyaluronic acid, TIMP1); FibroTest (GGT, total bilirubin, α₂m, apolipoprotein Al, and haptoglobin); and FibroMeter NAFLD (body weight, prothrombin index, ALT, AST, ferritin, and fasting glucose). Imaging biomarkers for liver fibrosis may include, but are not limited to: FibroScan (TE), point shear wave elastography (pSWE) (aka acoustic radiation force impulse (ARFI)), 2D-3D SWE, magnetic resonance elastography (MRE), and multiparameteric MRI.

In some embodiments, genetic and genomic biomarkers may be useful in assessing NAFLD risk and severity, which include the assessment of various SNPs, cell-free ncRNAs, and miRNAs. A comprehensive review of known genetic and genomic biomarkers, as well as the above-discussed blood biomarkers, panels, imaging biomarkers, and tests are summarized in VWS Wong et al., Nat Rev Gastroenterol Hepatol. 2018 August; 15(8):461-478; the contents of which are incorporated herein by reference.

In some embodiments in NASH patients, the isoform-specific, TGFβ1 inhibitors may be administered in patients who receive one or more additional therapies, including, but are not limited to myostatin inhibitors, which may generally enhance metabolic regulation in patients with clinical manifestation of metabolic syndrome, including NASH and NAFLD.

In some embodiments, the additional therapy may be a TGFβ3 inhibitor. In some embodiments, the TGFβ3 inhibitor is an isoform-specific TGFβ3 inhibitor. In some embodiments, the TGFβ3 inhibitor is a context-independent or context-bias TGFβ3 inhibitor. In some embodiments, the NASH patient has TGFβ1-positive and TGFβ3-positive fibrotic tissue. In some embodiments, the NASH patient is, or has been determined to be, partially responsive to the TGFβ1 inhibitor therapy.

In some embodiments, in NASH patients, the isoform-specific, TGFβ1 inhibitors may be administered in patients who receive an Acetyl CoA Carboxylase inhibitor (ACCi) (e.g., firsocostat (aka GS-0976) or PF-05221304). Other therapeutics which may be useful in combination with the improved isoform-specific TGFβ1 inhibitors described herein, include, but are not limited to: GLP-1 receptor agonists or analgues (e.g., semaglutide), farnesoid X receptor (FXR) agonists (e.g., GS-9674; aka Cilofexor), ASK1 inhibitors (e.g., selonsertib); obeticholic acid, PPAR agonists (e.g., GFT505; aka elafibranor); nitazoxanide, ketohexokinase (KHK) inhibitors (e.g., PF-06835919); and/or Diacylglycerol O-Acyltransferase 2 (DGAT2) inhibitors (e.g., PF-06865571). In some embodiments, anyone or more of the above-mentioned therapeutics can be used in combination with an isoform specific TGFβ1 inhibitor of the present disclosure, for example, an isoform-specific TGFβ1 inhibitor in combination with a FXR agonist, an ACC inhibitor, and/or a GLP-1 analogue.

In some embodiments, treatment with the isoform specific TGFβ1 inhibitors alone or in combination with one or more additional therapeutics reduces hepatic fat as measured by MRI-PDFF. In some embodiments, the reduction of hepatic fat is at least 20%, e.g., ≥20%, ≥25%, ≥30%, ≥35%, ≥40%, ≥45%, or ≥50%. In some embodiments, treatment with the isoform specific TGFβ1 inhibitors alone or in combination with one or more additional therapeutics reduces serum ALT and/or GGT by at least 20%, e.g., ≥20%, ≥25%, ≥30%, ≥35%, ≥40%, ≥45%, or ≥50%. In some embodiments, treatment with the isoform specific TGFβ1 inhibitors alone or in combination with one or more additional therapeutics reduces bile acid synthesis.

In some embodiments, the NASH patients may have advanced liver fibrosis (stage F3/F4). In some embodiments, such patients have stage F3 advanced liver fibrosis. In some embodiments, such patients have stage F4 liver fibrosis characterized by cirrhosis. In some embodiments, the NASH patients develop or at risk of developing hepatocellular carcinoma and/or esophageal varices.

Fibrosis staging in non-alcoholic fatty liver disease according to the classification derived by the Nonalcoholic Steatohepatitis Clinical Research Network Pathology Committee is provided below:

| Fibrotic manifestation | Fibrosis Stage |
|---|---|
| Perisinusoidal or periportal fibrosis | 1 |
| Mild perisinusoidal fibrosis (zone 3) | 1A |
| Moderate perisinusoidal fibrosis (zone 3) | 1B |
| Portal/periportal fibrosis | 1C |
| Perisinusoidal and portal/periportalfibrosis | 2 |
| Bridging fibrosis | 3 |
| Cirrhosis | 4 |

To enable assessment of the various histologic features during therapy and encompass the whole spectrum of NAFLD, the NASH Clinical Research Network (CRN) Pathology Committee performed a thorough univariate and multivariate analysis on the associations between the different histologic features observed in NASH and the diagnosis of NASH according to the Pathology Committee. The result was a scoring system of both NASH activity (Grade), and collagen deposition plus architectural remodeling (Stage). The grading system, the NASH Activity Score (NAS), was the unweighted sum of three histological components: steatosis (0-3), lobular inflammation (0-3) and ballooning degeneration (0-2). It ranged from 0 to 8. NAS includes the features of active injury that are potentially reversible. Additionally, the fibrosis staging system of Brunt et al. was further developed. In the NASH CRN system, the fibrosis score for stage 1 was subdivided into delicate (1A) and dense (1B) peri-sinusoidal fibrosis, whereas stage 1C was defined as portal fibrosis without concomitant peri-sinusoidal fibrosis (reviewed by Stäl, World J Gastroenterol. 2015 Oct. 21; 21(39): 11077-11087, incorporated by reference herein).

The isoform-specific, TGFβ1 inhibitors such as those provided herein may be used to treat fibrotic conditions of the kidney, e.g., diseases characterized by extracellular matrix accumulation (IgA nephropathy, focal and segmental glomerulosclerosis, crescentic glomerulonephritis, lupus nephritis and diabetic nephropathy) in which significantly increased expression of TGFβ in glomeruli and the tubulointerstitium has been observed. While glomerular and tubulointerstitial deposition of two matrix components induced by TGFβ, fibronectin EDA+ and PAI-1, was significantly elevated in all diseases with matrix accumulation, correlation analysis has revealed a close relationship primarily with the TGFβ1 isoform. Accordingly, the isoform-specific, TGFβ1 inhibitors are useful as therapeutic for a spectrum of human glomerular disorders, in which TGFβ is associated with pathological accumulation of extracellular matrix.

In some embodiments, the fibrotic condition of the kidney is associated with chronic kidney disease (CKD). CKD is caused primarily by high blood pressure or diabetes and claims more than one million lives each year. CKD patients require lifetime medical care that ranges from strict diets and medications to dialysis and transplants. In some embodiments, the TGFβ1 inhibitor therapy described herein may reduce or delay the need for dialysis and/or transplantation. In some embodiments, such therapy may reduce the need (e.g., dosage, frequency) for other treatments. In some embodiments, the isoform-specific, TGFβ1 inhibitors may be administered in patients who receive one or more additional therapies, including, but are not limited to myostatin inhibitors, which may generally enhance metabolic regulation in patients with CKD.

Fibrotic conditions that may be treated with the TGFβ1 inhibitor of the present disclosure include conditions involving fibrosis and/or chronic inflammation. Such conditions may be neuromuscular disorders, including but are not limited to Duchenne muscular dystrophy(DMD), and other genetic disorders such as multiple sclerosis (MS) and cystic fibrosis (CF). Through the inhibition of both the ECM- and immune cell-associated TGFβ1 arms, the TGFβ1 inhibitor such as those described herein is thought to suppress fibrotic progression and restore M1/M2 macrophage polarization.

Models useful for studying CKD and kidney fibrosis include but are not limited to, NZB/W, MRL/Ipr and BXSB mouse strains, anti-GBM models, anti-Thy1 models, 5/6 nephrectomy, Radiation nephropathy, puromycin aminonucleoside nephrosis (PAN) and adriamycin nephropathy, Folic acid nephropathy, CyA nephropathy, DOCA-salt nephropathy, HIV-associated nephropathy (HIVAN) transgenic mouse model, Spontaneously hypertensive rats (SHR), Buffalo/mna rats, Munich Wistar Frömter (MWF) rat, unilateral ureteral obstruction (UUO), Col4A knock-out mice (Alport Syndrome) (see Yang et al. Drug DiscovToday Dis Models. 2010; 7(1-2): 13-19; the contents of which are incorporated herein by reference).

The organ fibrosis which may be treated with the methods provided herein includes cardiac (e.g., cardiovascular) fibrosis. In some embodiments, the cardiac fibrosis is associated with heart failure, e.g., chronic heart failure (CHF). In some embodiments, the heart failure may be associated with myocardial diseases and/or metabolic diseases. In some embodiments, the isoform-specific, TGFβ1 inhibitors may be administered in patients who receive one or more additional therapies, including, but are not limited to myostatin inhibitors in patients with cardiac dysfunction that involves heart fibrosis and metabolic disorder.

Genetic models useful for studying cardiac fibrosis include but are not limited to, cardiac myocyte-specific FAK-KO mouse, genetically modified SR-BI/apoE double KO (dKO) mice, syndecan-1 null mice, EC-SOD-overexpressing mice, PKC-δ knockout mice. Surgical mouse models useful for studying cardiac fibrosis include but are not limited to, coronary artery ligation, ischemic-reperfusion model (open and closed chest), Chronic ischemia model, ischemia-reperfusion with ischemic preconditioning model, Langendorff model, traverse aortic constriction (TAC), ascending aortic constriction, abdominal aorta constriction, pulmonary artery banding, TAC with distal left anterior coronary ligation, aortocaval fistula (ACF) model, and aortic insufficiency model (see Rai et al., Mol Cell Biochem. 2017 January; 424(1-2): 123-145; the contents of which are incorporated herein by reference).

In some embodiments, fibrotic conditions that may be treated with the compositions and/or methods described herein include desmoplasia. Desmoplasia may occur around a neoplasm, causing dense fibrosis around the tumor (e.g., desmoplastic stroma), or scar tissue within the abdomen after abdominal surgery. In some embodiments, desmoplasia is associated with malignant tumor. Due to its dense formation surrounding the malignancy, conventional anti-cancer therapeutics (e.g., chemotherapy) may not effectively penetrate to reach cancerous cells for clinical effects. Isoform-specific, inhibitors of TGFβ1 such as those described herein may be used to disrupt the desmoplasia, such that the fibrotic formation can be loosened to aid effects of anti-cancer therapy. In some embodiments, the isoform-specific, inhibitors of TGFβ1 can be used as monotherapy(more below).

In some embodiments, a patient has a fibrotic solid tumor (e.g., desmoplasia) and is or has been excluded from a surgical candidate pool, such that the fibrotic solid tumor is considered to be non-resectable or non-operative. Such patient may be a candidate for receiving a TGFβ1 inhibition therapy of the present disclosure. The TGFβ1 inhibitor of the present invention may render the tumor become resectable or operable after administration so that the patient may become a candidate for surgical resection.

To treat patients with fibrotic conditions, TGFβ1 isoform-specific, inhibitors are administered to a subject in an amount effective to treat the fibrosis. The effective amount of such an antibody is an amount effective to achieve both therapeutic efficacy and clinical safety in the subject. In some embodiments, the inhibitor is an antibody that can block activation of an LTBP-mediated TGFβ1 localized (e.g., tethered) in the ECM and GARP-mediated TGFβ1 localized in (e.g., tethered on) immune cells. In some embodiments, antibody is an antibody that can block activation of an LTBP-mediated TGFβ1 localized in the ECM and LRRC33-mediated TGFβ1 localized in (e.g., tethered on) monocytes/macrophages. In some embodiments, the LTBP is LTBP1 and/or LTBP3. In some embodiments, targeting and inhibiting TGFβ1 presented by LRRC33 on profibrotic, M2-like macrophages in the fibrotic microenvironment may be beneficial.

Assays useful in determining the efficacy of the antibodies and/or compositions of the present disclosure for the alteration of fibrosis include, but are not limited to, histological assays for counting fibroblasts and basic immunohistochemical analyses known in the art.

In some embodiments, circulating LAP fragment(s) may be used as a serum marker of fibrogenesis. See for example, U.S. Pat. No. 8,198,412, the contents of which are incorporated herein by reference.

### Disease Involving ECM Dysregulation

The extracellular matrix is a cell-secreted network that surrounds cells and is primarily composed of proteoglycans and fibrous proteins, the most abundant of which is collagen. The novel antibodies disclosed herein may be used in the treatment of diseases associated with extracellular matrix dysregulation. The diseases associated with extracellular matrix dysregulation are typically myofibroblast-driven pathologies and include cancer, fibrosis, and cardiovascular disease (reviewed, for example, in: Lampi and Reinhart-King (2018) “Targeting extracellular matrix stiffness to attenuate disease: From molecular mechanisms to clinical trials” Sci Tarnsl Med 10(422): eaao0475). Progression of fibrotic conditions involves increased levels of matrix components deposited into the ECM and/or maintenance/remodeling of the ECM. TGFβ1 at least in part contributes to this process. This is supported, for example, by the observation that increased deposition of ECM components such as collagens can alter the mechanophysical properties of the ECM (e.g., the stiffness of the matrix/substrate) and this phenomenon is associated with TGFβ1 signaling. The inhibitors of TGFβ1, such as those described herein may be used to block this process to counter disease progression involving ECM alterations, such as fibrosis, tumor growth, invasion, metastasis and desmoplasia. The LTBP-arm of such inhibitors can directly block ECM-associated pro/latent TGFβ complexes which are presented by LTBP1 and/or LTBP3, thereby preventing activation/release of the growth factor from the complex in the disease niche. In some embodiments, the isoform-specific TGFβ1 inhibitors such as those described herein may normalize ECM stiffness to treat a disease that involves integrin-dependent signaling. In some embodiments, the integrin comprises an α11 chain, P1 chain, or both.

Thus, the antibody may be administered to a subject diagnosed with a disease with extracellular matrix dysregulation in an amount effective to treat the disease. Therapeutically effective amount of the antibody may be an amount sufficient to reduce expression of one or more markers of myofibroblasts, such as α-SMA. The amount m ay be an amount sufficient to reduce the stiffness of the extracellular matrixofan affected tissue (e.g., fibrotic tissues). The amount may be an amount sufficient to reduce TGFβ1 downstream effectors, such as phosphorylation of SMAD2 and/or SMAD3.

### Disease Involving Endothelial-to-Mesenchymal Transition (EndMT)

Similarly, TGFβ is also a key regulator of the endothelial-mesenchymal transition (EndMT) observed in normal development, such as heart formation. However, the same or similar phenomenon is also seen in many diseases, such as cancer stroma. In some disease processes, endothelial markers such as CD31 become downregulated upon TGFβ1 exposure and instead the expression of mesenchymal markers such as FSP-1, α-SMA and fibronectin becomes induced. Indeed, stromal CAFs may be derived from vascular endothelial cells. Thus, isoform-specific inhibitors of TGFβ1, such as those described herein, may be used to treat a disease that is initiated or driven by EndMT.

### Disease Involving Epithelial-to-Mesenchymal Transition (EMT)

EMT (epithelial mesenchymal transition) is the process by which epithelial cells with tight junctions switch to mesenchymal properties (phenotypes) such as loose cell-cell contacts. The process is observed in a number of normal biological processes as well as pathological situations, including embryogenesis, wound healing, cancer metastasis and fibrosis (reviewed in, for example, Shiga et al. (2015) “Cancer-Associated Fibroblasts: Their Characteristics and Their Roles in Tumor Growth.” Cancers, 7: 2443-2458). Generally, it is believed that EMT signals are induced mainly by TGFβ.

Epithelial cells have also been proposed to give rise to myofibroblasts by undergoing the process of EMT in several fibrotic tissues such as kidney, lung and in the liver. EMT takes place when epithelial cells lose their cuboidal shape, lose the expression of adherence and tight junction proteins, which leads to weak cell-cell contacts and reorganization of their actin cytoskeleton; while the cells acquire the expression of mesenchymal proteins (fibronectin, vimentin, N-cadherin), they adopt a fibroblast-like architecture favouring cell migration and invasion. EMT is induced by many growth factors, among them TGFβ being a very potent inducer, which regulate the expression and activity of several transcription factors known as EMT-TFs (Snail1/Snail, Snail2/Slug, ZEB1, ZEB2, Twist1/Twist and more) that are the responsible actors to execute the change in cell differentiation that is EMT. The gene and protein markers used to identify the generation of mesenchymal cells after EMT in the context of fibrosis are FSP1 (Fibroblast-specific protein 1), α-SMA and collagen I along with vimentin and desmin, whose expression increases concomitant with a reduction in levels of expression of epithelial markers (E-cadherin and certain cytokeratins). Cells that co-express epithelial and mesenchymal markers represent an intermediate stage of EMT (reviewed by, for example: Caja et al. Int. J. Mol. Sci. 2018, 19(5), 1294).

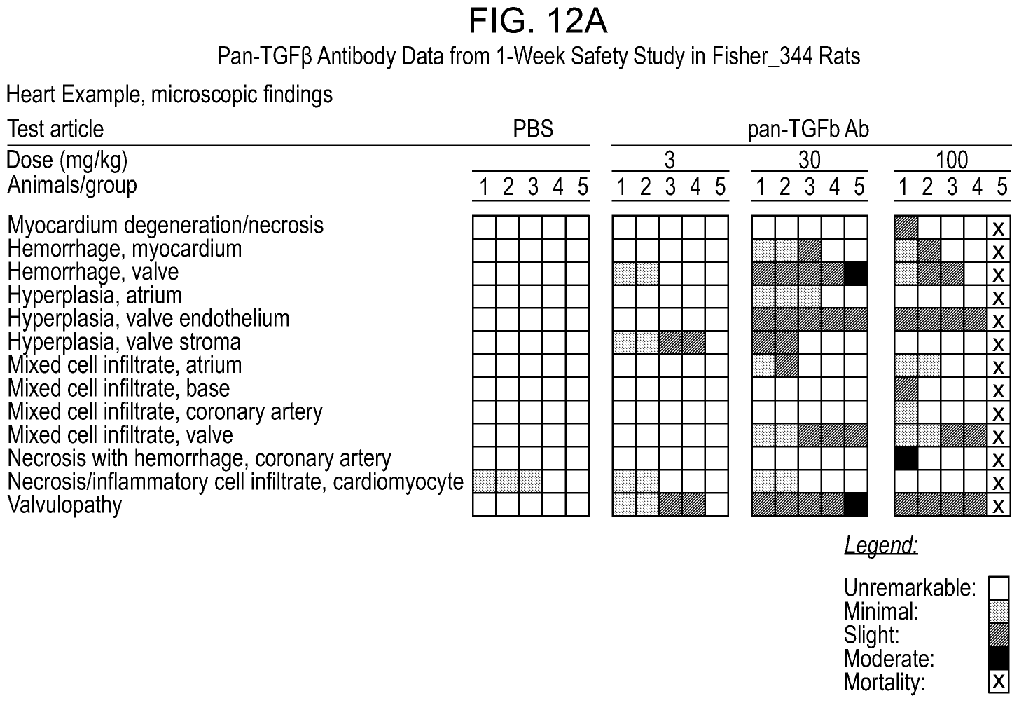

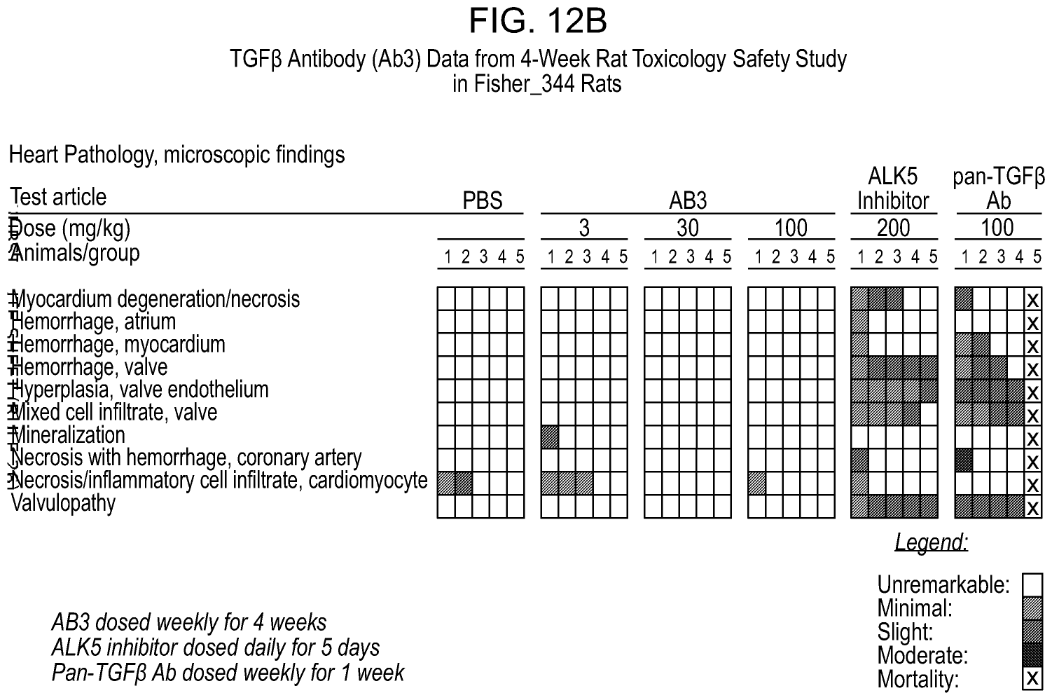

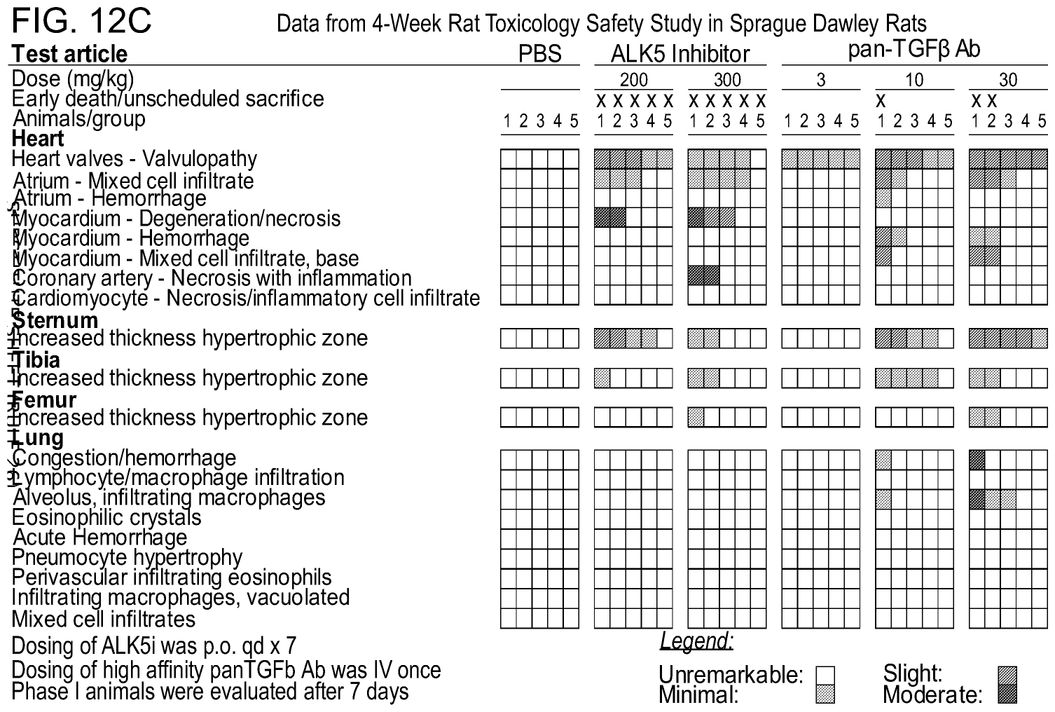

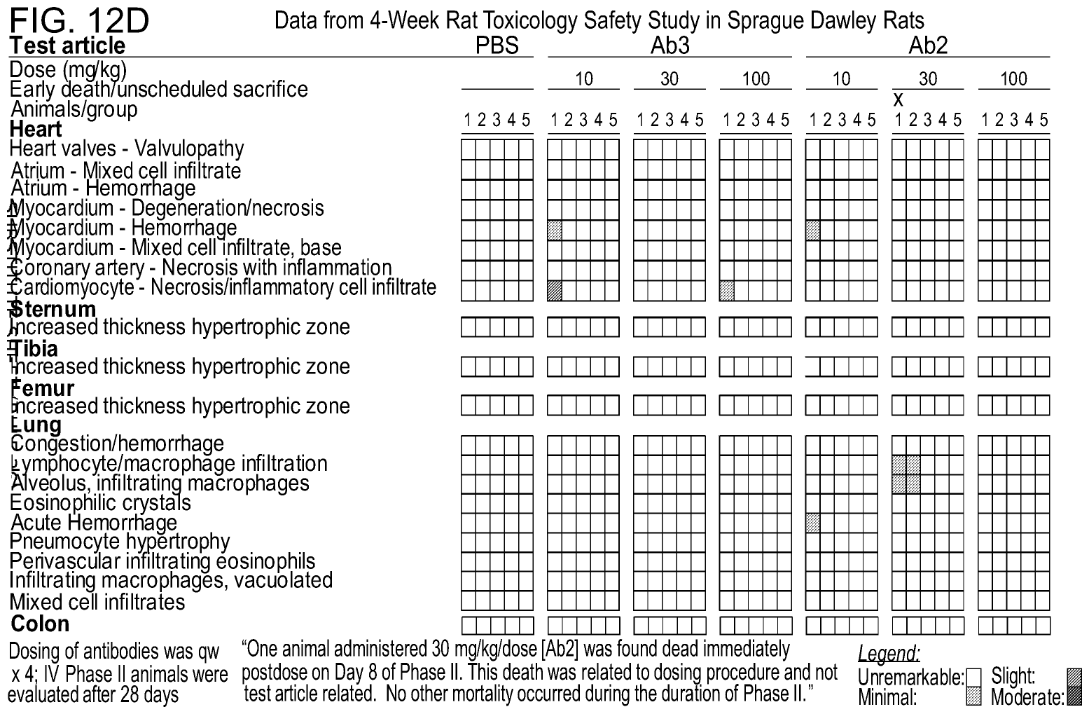

Many types of cancer, for example, appear to involve transdifferentiation of cells towards mesenchymal phenotype (such as CAFs) which correlate with poorer prognosis. Thus, isoform-specific inhibitors of TGFβ1, such as those described herein, may be used to treat a disease that is initiated or driven by EMT. Indeed, data exemplified herein (e.g., FIGS. 12 and 13) show that such inhibitors have the ability to suppress expression of CAF markers in vivo, such as α-SMA, Col1 (Type I collagen), and FN (fibronectin).

### Disease Involving Proteases

Activation of TGFβ from its latent complex may be triggered by integrin in a force-dependent manner, and/or by proteases. Evidence suggests that certain classes of proteases may be involved in the process, including but are not limited to Ser/Thr proteases such as Kallikreins, chemotrypsin, elastases, plasmin, as well as zinc metalloproteases of ADAM family such as ADAM 10 and ADAM 17, as well as MMP family, such as MMP-2, MMP-9 and MMP-13. MMP-2 degrades the most abundant component of the basement membrane, Collagen IV, raising the possibility that it may play a role in ECM-associated TGFβ1 regulation. MMP-9 has been implicated to playa central role in tumor progression, angiogenesis, stromal remodeling and metastasis. Thus, protease-dependent activation of TGFβ1 in the ECM may be important for treating cancer.

Kallikreins (KLKs) are trypsin-orchymotrypsin-like serine proteases that include plasma Kallikreins and tissue Kallikreins. The ECM plays a role in tissue homeostasis acting as a structural and signaling scaffold and barrier to suppress malignant outgrowth. KLKs may play a role in degrading ECM proteins and other components which may facilitate tumor expansion and invasion. For example, KLK1 is highly upregulated in certain breast cancers and can activate pro-MMP-2 and pro-MMP-9. KLK2 activates latent TGFβ1, rendering prostate cancer adjacent to fibroblasts permissive to cancer growth. KLK3 has been widely studied as a diagnostic marker for prostate cancer (PSA). KLK3 may directly activate TGFβ1 by processing plasminogen into plasmin, which proteolytically cleaves LAP. KLK6 may be a potential marker for Alzheimer's disease.

Known activators of TGFβ1, such as plasmin, TSP-1 and αVβ6 integrin, all interact directly with LAP. It is postulated that proteolytic cleavage of LAP may destabilize the LAP-TGFβ interaction, thereby releasing active TGFβ1. It has been suggested that the region containing 54-LSKLRL-59 is important for maintaining TGFβ1 latency. Thus, agents (e.g., antibodies) that stabilize the interaction, or block the proteolytic cleavage of LAP may prevent TGFβ activation.

Many of these proteases associated with pathological conditions (e.g., cancer) function through distinct mechanisms of action. Thus, targeted inhibition of particular proteases, or combinations of proteases, may provide therapeutic benefits for the treatment of conditions involving the protease-TGFβ axis. Accordingly, it is contemplated that inhibitors (e.g., TGFβ1 antibodies) that selectively inhibit protease-induced activation of TGFβ1 may be advantageous in the treatment of such diseases (e.g., fibrosis and cancer). Similarly, selective inhibition of TGFβ1 activation by one protease over another protease may also be preferred, depending on the condition being treated.

Plasmin is a serine protease produced as a precursor form called Plasminogen. Upon release, Plasmin enters circulation and therefore is detected in serum. Elevated levels of Plasmin appear to correlate with cancer progression, possibly through mechanisms involving disruption of the extracellular matrix (e.g., basement membrane and stromal barriers) which facilitates tumor cell motility, invasion, and metastasis. Plasmin may also affect adhesion, proliferation, apoptosis, cancer nutrition, oxygen supply, formation of blood vessels, and activation of VEGF (Didiasova et al., Int. J. Mol. Sci, 2014, 15, 21229-21252). In addition, Plasmin may promote the migration of macrophages into the tumor microenvironment (Philips et al., Cancer Res. 2011 Nov. 1; 71(21):6676-83 and Choong et al., Clin. Orthop. Relat. Res. 2003, 415S, S46-S58). Indeed, tumor-associated macrophages (TAMs) are well characterized drivers of tumorigenesis through their ability to promote tumor growth, invasion, metastasis, and angiogenesis.

Plasmin activities have been primarily tied to the disruption of the ECM. However, there is mounting evidence that Plasmin also regulate downstream MMP and TGF beta activation. Specifically, Plasmin has been suggested to cause activation of TGF beta through proteolytic cleavage of the Latency Associated Peptide (LAP), which is derived from the N-terminal region of the TGF beta gene product (Horiguchi et al., J Biochem. 2012 October; 152(4):321-9), resulting in the release of active growth factor. Since TGFβ1 may promote cancer progression, this raises the possibility that plasmin-induced activation of TGFb may at least in part mediate this process.

TGFβ1 has also been shown to regulate expression of uPA, which is a critical player in the conversion of Plasminogen into Plasmin (Santibanez, Juan F., ISRN Dermatology, 2013: 597927). uPA has independently been shown to promote cancer progression (e.g., adhesion, proliferation, and migration) by binding to its cell surface receptor (uPAR) and promoting conversion of Plasminogen into Plasmin. Moreover, studies have shown that expression of uPA and/or plasminogen activator inhibitor-1 (PAI-1) are predictors of poor prognosis in colorectal cancer (D. Q. Seetoo, et al., Journal of Surgical Oncology, vol. 82, no. 3, pp. 184-193, 2003), breast cancer (N. Harbeck et al., Clinical Breast Cancer, vol. 5, no. 5, pp. 348-352, 2004), and skin cancer (Santibanez, Juan F., ISRN Dermatology, 2013: 597927). Thus, without wishing to be bound by a particular theory, the interplay between Plasmin, TGFβ1, and uPA may create a positive feedback loop towards promoting cancer progression. Accordingly, inhibitors that selectively inhibit Plasmin-dependent TGFβ1 activation may be particularly suitable for the treatment of cancers reliant on the Plasmin/TGFβ1 signaling ads.

In one aspect of the invention, the isoform-specific inhibitors of TGFβ1 described herein include inhibitors that can inhibit protease-dependent activation of TGFβ1. In some embodiments, the inhibitors can inhibit protease-dependent TGFβ1 activation in an integrin-independent manner. In some embodiments, such inhibitors can inhibit TGFβ1 activation irrespective of the mode of activation, e.g., inhibit both integrin-dependent activation and protease-dependent activation of TGFβ1. In some embodiments, the protease is selected from the group consisting of: serine proteases, such as Kallikreins, Chemotryps in, Trypsin, Elastases, Plasmin, as well as zinc metalloproteases (MMP family) such as MMP-2, MMP-9 and MMP-13.

In some embodiments, the inhibitors can inhibit Plasmin-induced activation of TGFβ1. In some embodiments, the inhibitors can inhibit Plasmin- and integrin-induced TGFβ1 activation. In some embodiments, the inhibitors are monoclonal antibodies that specifically bind TGFβ1. In some embodiments, the antibody is a monoclonal antibody that specifically binds proTGFβ1. In some embodiments, the antibody binds latent proTGFβ1 thereby inhibiting release of mature growth factor from the latent complex. In some embodiments, the inhibitor of TGFβ1 activation suitable for use in the method of inhibiting Plasmin-dependent activation of TGFβ1 is any one of the isoform-specific inhibitors disclosed herein.

In some embodiments, the inhibitor (e.g., TGFβ1 antibody) inhibits cancer cell migration. In some embodiments, the inhibitor inhibits monocyte/macrophage migration. In some embodiments, the inhibitor inhibits accumulation of TAMs.

In another aspect, provided herein is a method for treating cancer in a subject in need thereof, the method comprising administering to the subject an effective amount of an TGFβ1 inhibitor (e.g., TGFβ1 antibody), wherein the inhibitor inhibits protease-induced activation of TGFβ1 (e.g., Plasmin), thereby treating cancer in the subject.

In another aspect, provided herein is a method of reducing tumor growth in a subject in need thereof, the method comprising administering to the subject an effective amount of an TGFβ1 inhibitor (e.g., TGFβ1 antibody), wherein the inhibitor inhibits protease-induced activation of TGFβ1 (e.g., Plasmin), thereby reducing tumor growth in the subject.

### Disease Involving Aberrant Gene Expression

It has been observed that abnormal activation of the TGFβ1 signal transduction pathway in various disease conditions is associated with altered gene expression of a number of markers. These gene expression markers (e.g., as measured by mRNA) include, but are not limited to: Serpine 1 (encoding PAI-1), MCP-1 (also known as CCL2), Col1a1, Col3a1, FN1, TGFβ1, CTGF, ACTA2 (encoding α-SMA), SNAI1 (drives EMT in fibrosis and metastasis by downregulating E-cadherin (Cdh1), MMP2 (matrix metalloprotease associated with EMT), MMP9 (matrix metalloprotease associated with EMT), TIMP1 (matrix metalloprotease associated with EMT), FOXP3 (marker of Treg induction), CDH1 (E cadherin (marker of epithelial cells) which is downregulated by TGFβ), and, CDH2 (N cadherin (marker of mesenchymal cells) which is upregulated by TGFβ). Interestingly, many of these genes are implicated to playa role in a diverse set of disease conditions, including various types of organ fibrosis, as well as in many cancers, which include myelofibrosis. Indeed, pathophysiological link between fibrotic conditions and abnormal cell proliferation, tumorigenesis and metastasis has been suggested. See for example, Cox and Erler (2014) Clinical Cancer Research 20(14): 3637-43 “Molecular pathways: connecting fibrosis and solid tumor metastasis”; Shiga et al. (2015) Cancers 7:2443-2458 “Cancer-associated fibroblasts: their characteristics and their roles in tumor growth”; Wynn and Barron (2010) Semin. Liver Dis. 30(3): 245-257 “Macrophages: master regulators of inflammation and fibrosis”, contents of which are incorporated herein by reference. Without wishing to be bound by a particular theory, the inventors of the present disclosure contemplate that the TGFβ1 signaling pathway may in fact be a key link between these broad pathologies.

The ability of chemotactic cytokines (or chemokines) to mediate leukocyte recruitment (e.g., monocytes/macrophages) to injured or disease tissues has crucial consequences in disease progression. Members of the C-C chemokine family, such as monocyte chemoattractant protein 1 (MCP-1), also known as CCL2, macrophage inflammatory protein 1-alpha (MIP-1a), also known as CCL3, and MIP-1β, also known as CCL4, have been implicated in this process.

For example, MCP-1/CCL2 is thought to playa role in both fibrosis and cancer. MCP-1/CCL2 is characterized as a profibrotic chemokine and is a monocyte chemoattractant, and evidence suggests that it may be involved in both initiation and progression of cancer. In fibrosis, MCP-1/CCL2 has been shown to play an important role in the inflammatory phase of fibrosis. For example, neutralization of MCP-1 resulted in a dramatic decrease in glomerular crescent formation and deposition of type I collagen. Similarly, passive immunotherapy with either anti-MCP-1 or anti-MIP-1 alpha antibodies is shown to significantly reduce mononuclear phagocyte accumulation in bleomycin-challenged mice, suggesting that MIP-1 alpha and MCP-1 contribute to the recruitment of leukocytes during the pulmonary inflammatory response (Smith, Biol Signals. 1996 July-August; 5(4):223-31, “Chemotactic cytokines mediate leukocyte recruitment in fibrotic lung disease”). Elevated levels of MIP-1 alpha in patients with cystic fibrosis and multiple myeloma have been reported (see, for example: Mrugacz et al., J Interferon Cytokine Res. 2007 June; 27(6):491-5), supporting the notion that MIP-1α is associated with localized or systemic inflammatory responses.

Lines of evidence point to the involvement of C-C chemokines in tumor progression. For example, tumor-derived MCP-1/CCL2 can promote “pro-cancer” phenotypes in macrophages. For example, in lung cancer, MCP-1/CCL2 has been shown to be produced by stromal cells and promote metastasis. In human pancreatic cancer, tumors secrete CCL2, and immunosuppressive CCR2-positive macrophages infiltrate these tumors. Patients with tumors that exhibit high CCL2 expression/low CD8 T-cell infiltrate have significantly decreased survival. Without wishing to be bound by particular theory, it is contemplated that monocytes that are recruited to an injured or diseased tissue environment may subsequently become polarized in response to local cues (such as in response to tumor-derived cytokines), thereby further contributing to disease progression. These M2-like macrophages are likely to contribute to immune evasion by suppressing effector cells, such as CD4+ and CD8+ T cells. In some embodiments, this process is in part mediated by LRRC33-TGFβ1 expressed by activated macrophages. In some embodiments, the process is in part mediated by GARP-TGFβ1 expressed by Tregs.

Similarly, involvement of PAI-1/Serpine1 has been implicated in a variety of cancers, angiogenesis, inflammation, neurodegenerative diseases (e.g., Alzheimer's Disease). Elevated expression of PAI-1 in tumor and/or serum is correlated with poor prognosis (e.g., shorter survival, increased metastasis) in various cancers, such as breast cancer and bladder cancer (e.g., transitional cell carcinoma) as well as myelofibrosis. In the context of fibrotic conditions, PAI-1 has been recognized as an important downstream effector of TGFβ1-induced fibrosis, and increased PAI-1 expression has been observed in various forms of tissue fibrosis, including lung fibrosis (such as Idiopathic Pulmonary Fibrosis (IPF)), kidney fibrosis, liver fibrosis and scleroderma. In some embodiments, the process is in part mediated by ECM-associated TGFβ1, e.g., via LTBP1 and/or LTBP3.

In some embodiments, in vivo effects of the TGFβ1 inhibitor therapy may be assessed by measuring changes in gene markers. Suitable markers include TGFβ (e.g., TGFβ1, TGFβ2, and TGFβ3). Suitable markers may also include one or more presenting molecules for TGFβ (e.g., TGFβ1, TGFβ2, and TGFβ3), such as LTBP1, LTBP3, GARP (or LRRC32) and LRRC33. In some embodiments, suitable markers include mesenchymal transition genes (e.g., fibronectin, vimentin, N-cadherin, AXL, ROR2, WNT5A, LOXL2, TWIST2, TAGLN, and/or FAP), immunosuppressive genes (e.g., IL10, VEGFA, VEGFC), monocyte and macrophage chemotactic genes (e.g., CCL2, CCL3, CCL4, CCL7, CCL8 and CCL13), and/or various fibrotic markers discussed herein. Preferred markers are plasma markers.

As shown in the Examples herein, isoform-specific inhibitors of TGFβ1 described herein can reduce expression levels of many of these markers in a mechanistic animal model, such as UUO, which has been shown to be TGFβ31-dependent. Therefore, such inhibitors may be used to treat a disease or disorder characterized by abnormal expression (e.g., overexpression/upregulation or under expression/downregulation) of one or more of the gene expression markers.

Thus, in some embodiments, an isoform-specific inhibitor of TGFβ1 is used in the treatment of a disease associated with overexpression of one or more of the following: PAI-1 (encoded by Serpine1), MMP2, MMP9, MCP-1 (also known as CCL2), Col1a1, Col3a1, FN1, TGFβ1, CTGF, α-SMA, ITGA11, and ACTA2, wherein the treatment comprises administration of the inhibitor to a subject suffering from the disease in an amount effective to treat the disease. In some embodiments, the inhibitor is used to treat a disease associated with overexpression of PAI-1, MCP-1/CCL2, CTGF, and/or α-SMA. In some embodiments, the disease is myelofibrosis. In some embodiments, the disease is cancer, for example, cancer comprising a solid tumor. In some embodiments, the disease is organ fibrosis, e.g., fibrosis of the liver, the kidney, the lung, the muscle, the skin and/or the cardiac or cardiovascular tissue. In some embodiments, the disease is Alport Syndrome. In some embodiments, the inhibitor reduces expression of one or more of the following: PAI-1 (encoded by Serpine1), MMP2, MMP9, MCP-1 (also known as CCL2), Col1a1, Col3a1, FN1, TGFβ1, CTGF, α-SMA, ITGA11, and ACTA2.

Another biomarker which may be used to assess the in vivo effects of the TGFβ1 inhibitor therapy is blood urea nitrogen (BUN). Urea is naturally formed in the body as a by-product of protein breakdown. The urea travels from you liver to your kidneys where it is filtered/removed from the blood. Accordingly, BUN levels may increase in situations when a patient's kidneys are not functioning properly. For example, patients having kidney fibrosis may display increased BUN. Accordingly, in some embodiments, BUN is measured to assess the in vivo effects of the isoform-specific inhibitors of TGFβ1 as described herein. In other embodiments, an isoform-specific inhibitor of TGFβ1 is used in the treatment of a disease associated with increased BUN (e.g., kidney fibrosis and/or acute or chronic kidney disease, damage, or failure). In a particular embodiment, the disease associated with increased BUN is Alport Syndrome.

Accordingly, the present disclosure includes a method of selecting a candidate patient or patient population likely to respond to a TGFβ1 inhibition therapy. Such method may comprise a step of testing a biological sample collected from the patient (or patient population), such as biopsy samples, for the expression of one or more of the markers discussed herein. Similarly, such genetic marker(s) may be used for purposes of monitoring the patient's responsiveness to a therapy. Monitoring may include testing two or more biological samples collected from the patient, for example, before and after administration of a therapy, and during the course of a therapeutic regimen over time, to evaluate changes in gene expression levels of one or more of the markers, indicative of therapeutic response or effectiveness.

In some embodiments, a method of selecting a candidate patient or patient population likely to respond to a TGFβ1 inhibition therapy may comprise a step of identifying a patient or patient population previously tested for the genetic marker(s), such as those described herein, which showed aberrant expression thereof. In some embodiments, the aberrant marker expression includes elevated levels of at least one of the following: TGFβ1, LRRC33, GARP, LTBP1, LTBP3, CCL2, CCL3, PAI-1/Serpine1, MMP2, MMP9, Col1a1, Col3a1, FN1, CTGF, α-SMA, ITGA11, and ACTA2. In some embodiments, the patient or patient population (e.g., biological samples collected therefrom) shows elevated TGFβ1 activation, phospho-Smad2/3, or combination thereof. In some embodiments, the patient or patient population shows elevated BUN.

In some embodiments, the patient or patient population (e.g., biological samples collected therefrom) shows elevated MDSCs. In some embodiments, such patient or patient population has cancer, which may comprise a solid tumor. The solid tumor may be a TGFβ1-dominant tumor, in which TGFβ1 is the predominant isoform expressed in the tumor, relative to the other isoforms. In some embodiments, such patient or patient population exhibits resistance to a cancer therapy, such as chemotherapy, radiation therapy and/or immune checkpoint therapy, e.g., anti-PD-1 (e.g., Pembrolizumab and Nivolumab), anti-PD-L1 (e.g., Atezolizumab), anti-CTLA4 (e.g., Ipilimumab), engineered immune cell therapy (e.g., CAR-T), and cancer vaccines, etc. According to the invention, the isoform-specific TGFβ1 inhibitor, such as those disclosed herein, overcomes the resistance by unblocking immunosuppression so as to allow effector cells to gain access to cancer cells thereby achieving anti-tumor effects.

### Proliferative Disorders (e.g., Myeloproliferative Disorder)

In some embodiments, the antibodies described herein, may be used to treat a proliferative disorder. In some embodiments, the proliferative disorder is a cancer or a myeloproliferative disorder. In some embodiments, the myeloproliferative disorder is myelofibrosis.

Myelofibrosis, also known as osteomyelofibrosis, is a relatively rare bone marrow proliferative disorder (cancer), which belongs to a group of diseases called myeloproliferative disorders. Myelofibrosis is classified into the Philadelphia chromosome-negative (−) branch of myeloproliferative neoplasms. Myelofibrosis is characterized by clonal myeloproliferation, aberrant cytokine production, extramedullary hematopoiesis, and bone marrow fibrosis. The proliferation of an abnormal clone of hematopoietic stem cells in the bone marrow and other sites results in fibrosis, or the replacement of the marrow with scar tissue. The term myelofibrosis, unless otherwise specified, refers to primary myelofibrosis (PMF). This may also be referred to as chronic idiopathic myelofibrosis (cIMF) (the terms idiopathic and primary mean that in these cases the disease is of unknown or spontaneous origin). This is in contrast with myelofibrosis that develops secondary to polycythemia vera or essential thrombocythaemia. Myelofibrosis is a form of myeloid metaplasia, which refers to a change in cell type in the blood-forming tissue of the bone marrow, and often the two terms are used synonymously. The terms agnogenicmyeloid metaplasia and myelofibrosis with myeloid metaplasia (MMM) are also used to refer to primary myelofibrosis. In some embodiments, the hematologic proliferative disorders which may be treated in accordance with the present invention include myeloproliferative disorders, such as myelofibrosis. So-called “classical” group of BCR-ABL (Ph) negative chronic myeloproliferative disorders includes essential thrombocythemia (ET), polycythemia vera (PV) and primary myelofibrosis (PMF).

Myelofibrosis disrupts the body's normal production of blood cells. The result is extensive scarring in the bone marrow, leading to severe anemia, weakness, fatigue and often an enlarged spleen. Production of cytokines such as fibroblast growth factor by the abnormal hematopoietic cell clone (particularly by megakaryocytes) leads to replacement of the hematopoietic tissue of the bone marrow by connective tissue via collagen fibrosis. The decrease in hematopoietic tissue impairs the patient's ability to generate new blood cells, resulting in progressive pancytopenia, a shortage of all blood cell types. However, the proliferation of fibroblasts and deposition of collagen is thought to be a secondary phenomenon, and the fibroblasts themselves may not be part of the abnormal cell clone.

Myelofibrosis may be caused by abnormal blood stem cells in the bone marrow. The abnormal stem cells produce mature and poorly differentiated cells that grow quickly and take over the bone marrow, causing both fibrosis (scar tissue formation) and chronic inflammation.

Primary myelofibrosis is associated with mutations in Janus kinase 2 (JAK2), thrombopoietin receptor (MPL) and calreticulin (CALR), which can lead to constitutive activation of the JAK-STAT pathway, progressive scarring, or fibrosis, of the bone marrow occurs. Patients may develop extramedullary hematopoiesis, i.e., blood cell formation occurring in sites other than the bone marrow, as the haemopoetic cells are forced to migrate to other areas, particularly the liver and spleen. This causes an enlargement of these organs. In the liver, the abnormal size is called hepatomegaly. Enlargement of the spleen is called splenomegaly, which also contributes to causing pancytopenia, particularly thrombocytopenia and anemia. Another complication of extramedullary hematopoiesis is poikilocytosis, or the presence of abnormally shaped red blood cells.

The principal site of extramedullary hematopoiesis in myelofibrosis is the spleen, which is usually markedly enlarged in patients suffering from myelofibrosis. As a result of massive enlargement of the spleen, multiple subcapsular infarcts often occur in the spleen, meaning that due to interrupted oxygen supply to the spleen partial or complete tissue death happens. On the cellular level, the spleen contains red blood cell precursors, granulocyte precursors and megakaryocytes, with the megakaryocytes prominent in their number and in their abnormal shapes. Megakaryocytes may be involved in causing the secondary fibrosis seen in this condition.

It has been suggested that TGFβ may be involved in the fibrotic aspect of the pathogenesis of myelofibrosis (see, for example, Agarwal et al., “Bone marrow fibrosis in primary myelofibrosis: pathogenic mechanisms and the role of TGFβ” (2016) Stem Cell Investig 3:5). Bone marrow pathology in primary myelofibrosis is characterized by fibrosis, neoangeogenesis and osteosclerosis, and the fibrosis is associated with an increase in production of collagens deposited in the ECM.

A number of biomarkers have been described, alternations of which are indicative of or correlate with the disease. In some embodiments, the biomarkers are cellular markers. Such disease-associated biomarkers are useful for the diagnosis and/or monitoring of the disease progression as well as effectiveness of therapy (e.g., patients' responsiveness to the therapy). These biomarkers include a number of fibrotic markers, as well as cellular markers. In lung cancer, for example, TGFβ1 concentrations in the bronchoalveolar lavages (BAL) fluid are reported to be significantly higher in patients with lung cancer compared with patients with benign diseases (˜2+ fold increase), which may also serve as a biomarker for diagnosing and/or monitoring the progression or treatment effects of lung cancer.

Because primary myelofibrosis is associated with abnormal megakaryocyte development, certain cellular markers of megakaryocytes as well as their progenitors of the stem cell lineage may serve as markers to diagnose and/or monitor the disease progression as well as effectiveness of therapy. In some embodiments, useful markers include, but are not limited to: cellular markers of differentiated megakaryocytes (e.g., CD41, CD42 and Tpo R), cellular markers of megakaryocyte-erythroid progenitor cells (e.g., CD34, CD38, and CD45RA-), cellular markers of common myeloid progenitor cells (e.g., IL-3α/CD127, CD34, SCF R/c-kit and Flt-3/Flk-2), and cellular markers of hematopoieticstem cells (e.g., CD34, CD38-, Flt-3/Flk-2). In some embodiments, useful biomarkers include fibrotic markers. These include, without limitation: TGFβ1, PAI-1 (also known as Serpine1), MCP-1 (also known as CC L2), Col1a1, Col3a1, FN1, CTGF, α-SMA, ACTA2, Timp1, Mmp8, and Mmp9. In some embodiments, useful biomarkers are serum markers (e.g., proteins or fragments found and detected in serum samples).

Based on the finding that TGFβ is a component of the leukemic bone marrow niche, it is contemplated that targeting the bone marrow microenvironment with TGFβ inhibitors may be a promising approach to reduce leukemic cells expressing presenting molecules that regulate local TGFβ availability in the effected tissue.

Indeed, due to the multifaceted nature of the pathology which manifests TGFβ-dependent dysregulation in both myelo-proliferative and fibrotic aspects (as the term “myelofibrosis” itself suggests), isoform-specific, inhibitors of TGFβ1, which target matrix- and cell-associated TGFβ1 complexes, such as those described herein, may provide particularly advantageous therapeutic effects for patients suffering from myelofibrosis. It is contemplated that the LTBP-arm of such inhibitor can target ECM-associated TGFβ1 complex in the bone marrow, whilst the LRRC33-arm of the inhibitor can block myeloid cell-associated TGFβ1. In addition, abnormal megakaryocyte biology associated with myelofibrosis may involve both GARP- and LTBP-mediated TGFβ1 activities. The isoform-specific inhibitor of TGFβ1 is capable of targeting such complexes thereby inhibiting release of active TGFβ1 in the niche.

Thus, such TGFβ1 inhibitors are useful for treatment of patients with polycythemia vera who have had an inadequate response to or are intolerant of other (or standard-of-care) treatments, such as hydroxyurea and JAK inhibitors. Such inhibitors are also useful for treatment of patients with intermediate or high-risk myelofibrosis (MF), including primary MF, post-polycythemia vera MF and post-essential thrombocythemia MF.

Accordingly, one aspect of the invention relates to methods for treating primary myelofibrosis. The method comprises administering to a patient suffering from primary myelofibrosis a therapeutically effective amount of a composition comprising a TGFβ inhibitor that causes reduced TGFβ availability. In some embodiments, an inhibitor of TGFβ1 activation is administered to patients with myelofibrosis. Such antibody may be administered at dosages ranging between 0.1 and 100 mg/kg, such as between 1 and 30 mg, e.g., 1 mg/kg, 3 mg/kg, 5 mg/kg, 10 mg/kg, 15 mg/kg, 20 mg/kg, etc. For example, suitable dosing regimens include between 1-20 mg/kg administered weekly. Preferred routes of administration of a pharmaceutical composition comprising the antibody is intravenous or subcutaneous administration. When the composition is administered intravenously, the patient may be given the therapeutic over a suitable duration of time, e.g., approximately 30-120 minutes (e.g., 30 min, 60 min, 75 min, 90 min, and 120 min), per treatment, and then repeated every several weeks, e.g., 3 weeks, 4 weeks, 6 weeks, etc., for a total of several cycles, e.g., 4 cycles, 6, cycles, 8 cycles, 10 cycles, 12 cycles, etc. In some embodiments, patients are treated with a composition comprising the inhibitory antibody at dose level of 1-10 mg/kg (e.g., 1, 2, 3, 4, 5, 6, 7, 8, 9, or 10 mg/kg per dosing) via intravenous administration every 28 days (4 weeks) for 6 cycles or 12 cycles. In some embodiments, such treatment is administered as a chronic (long-term) therapy (e.g., to be continued indefinitely, as long as deemed beneficial) in lieu of discontinuing following a set number of cycles of administration.

While myelofibrosis is considered a type of leukemia, itis also characterized by the manifestation of fibrosis. Because TGFβ is known to regulate aspects of ECM homeostasis, the dysregulation of which can lead to tissue fibrosis, it is desirable to inhibit TGFβ activities associated with the ECM. Accordingly, the antibodies or fragments described herein inhibit proTGFβ presented by LTBPs (such as LTBP1 and LTBP3), as well as inhibit proTGFβ presented by GARP and LRRC33.

Early in vivo data indicate that an isoform-selective inhibitor of TGFβ1, such as those described herein, can be used to treat myelofibrosis in a translatable murine model of primary myelofibrosis. Unlike the current standard of care JAK2 inhibitor, which only provides symptomatic relief but does not provide clinical or survival benefits, the isoform-selective inhibitor of TGFβ1 achieves significant anti-fibrotic effects in the bone marrow of the diseased mice and may also prolong survival, supporting the notion that the TGFβ1 inhibitor may be effective to treat myeloproliferative disorders in human patients.

Suitable patient populations of myeloproliferative neoplasms who may be treated with the compositions and methods described herein may include, but are not limited to: a) a patient population that is Philadelphia (+); b) a patient population that is Philadelphia (−); c) a patient population that is categorized “classical” (PV, ET and PMF); d) a patient population carrying the mutation JAK2V617F(+); e) a patient population carrying JAK2V617F(−); f) a patient population with JAK2 exon 12(+); g) a patient population with MPL(+); and h) a patient population with CALR(+).

In some embodiments, the patient population includes patients with intermediate-2 or high-risk myelofibrosis. In some embodiments, the patient population comprises subjects with myelofibrosis who are refractory to or not candidates for available therapy. In some embodiments, the subject has platelet counts between 100-200×10⁹/L. In some embodiments, the subject has platelet counts >200×10⁹/L prior to receiving the treatment.

In some embodiments, a subject to receive (and who may benefit from receiving) an isoform-specific, TGFβ1 inhibitor therapy is diagnosed with intermediate-1 or higher primary myelofibrosis (PMF), or post-polycythemmia vera/essential thrombocythemia myelofibrosis (post-PV/ETMF). In some embodiments, the subject has documented bone marrow fibrosis prior to the treatment. In some embodiments, the subject has MF-2 or higher as assessed by the European consensus grading score and grade 3 or higher by modified Bauermeister scale prior to the treatment. In some embodiments, the subject has the ECOG performance status of 1 prior to the treatment. In some embodiments, the subject has white blood cell count (10⁹/L) ranging between 5 and 120 prior to the treatment. In some embodiments, the subject has the JAK2V617F allele burden that ranges between 10-100%.

In some embodiments, a subject to receive (and who may benefit from receiving) an isoform-specific, TGFβ1 inhibitor therapy is transfusion-dependent (prior to the treatment) characterized in that the subject has a history of at least two units of red blood cell transfusions in the last month fora hemoglobin level of less than 8.5 g/dL that is not associated with clinically overt bleeding.

In some embodiments, a subject to receive (and who may benefit from receiving) an isoform-specific, TGFβ1 inhibitor therapy previously received a therapy to treat myelofibrosis. In some embodiments, the subject has been treated with one or more of therapies, including but are not limited to: AZD1480, panobinostat, EPO, IFNα, hydroxyurea, pegylated interferon, thalidomide, prednisone, and JAK2 inhibitor (e.g., Lestaurtinib, CEP-701).

In some embodiments, the patient has extramedullary hematopoiesis. In some embodiments, the extramedullary hematopoiesis is in the liver, lung, spleen, and/or lymph nodes. In some embodiments, the pharmaceutical composition of the present invention is administered locally to one or more of the localized sites of disease manifestation.

The isoform-specific, TGFβ1 inhibitor is administered to patients in an amount effective to treat myelofibrosis. The therapeutically effective amount is an amount sufficient to relieve one or more symptoms and/or complications of myelofibrosis in patients, including but are not limited to: excessive deposition of ECM in bone marrow stroma, neoangiogenesis, osteosclerosis, splenomegaly, hematomegaly, anemia, bleeding, bone pain and other bone-related morbidity, extramedullary hematopoiesis, thrombocytosis, leukopenia, cachexia, infections, thrombosis and death.

In some embodiments, the amount is effective to reduce TGFβ1 expression and/or secretion (such as of megakaryocytic cells) in patients. Such inhibitor may therefore reduce TGFβ1 mRNA levels in treated patients. In some embodiments, such inhibitor reduces TGFβ1 mRNA levels in bone marrow, such as in mononuclear cells. PMF patients typically show elevated plasma TGFβ1 levels of above ˜2,500 pg/mL, e.g., above 3,000, 3,500, 4,000, 4,500, 5,000, 6,000, 7,000, 8,000, 9,000, and 10,000 pg/mL (contrast to normal ranges of ˜600-2,000 pg/mL as measured by ELISA) (see, for example, Mascaremhas et al. (Leukemia & Lymphoma, 2014, 55(2): 450-452)). Zingariello (Blood, 2013, 121(17): 3345-3363) quantified bioactive and total TGFβ1 contents in the plasma of PMF patients and control individuals. According to this reference, the median bioactive TGFβ1 in PMF patients was 43 ng/mL (ranging between 4-218 ng/mL) and total TGFβ1 was 153 ng/mL (32-1000 ng/mL), while in control counterparts, the values were 18 (0.05-144) and 52 (8-860), respectively. Thus, based on these reports, plasma TGFβ1 contents in PMF patients are elevated by several fold, e.g., 2-fold, 3-fold, 4-fold, 5-fold, etc., as compared to control or healthy plasma samples. Treatment with the inhibitor, e.g., following 4-12 cycles of administration (e.g., 2, 4, 6, 8, 10, 12 cycles) or chronic or long-term treatment, for example every 4 weeks, at dosage of 0.1-100 mg/kg, for example, 1-30 mg/kg monoclonal antibody) described herein may reduce the plasma TGFβ1 levels by at least 10% relative to the corresponding baseline (pre-treatment), e.g., at least 15%, 20%, 25%, 30%, 35%, 40%, 45%, and 50%.

Some of the therapeutic effects may be observed relatively rapidly following the commencement of the treatment, for example, after 1 week, 2 weeks, 3 weeks, 4 weeks, 5 weeks or 6 weeks. For example, the inhibitor may effectively increase the number of stem cells and/or precursor cells within the bone marrow of patients treated with the inhibitor within 1-8 weeks. These include hematopoietic stem cells and blood precursor cells. A bone marrow biopsy may be performed to assess changes in the frequencies/number of marrow cells. Correspondingly, the patient may show improved symptoms such as bone pain and fatigue.

One of the morphological hallmarks of myelofibrosis is fibrosis in the bone marrow (e.g., marrow stroma), characterized in part by aberrant ECM. In some embodiments, the amount is effective to reduce excessive collagen deposition, e.g., by mesenchymal stromal cells. In some embodiments, the inhibitor is effective to reduce the number of CD41-positive cells, e.g., megakaryocytes, in treated subjects, as compared to control subjects that do not receive the treatment. In some embodiments, baseline frequencies of megakaryocytes in PMF bone marrow may range between 200-700 cells per square millimeters (mm), and between 40-300 megakaryocites per square-millimeters (mm²) in PMF spleen, as determined with randomly chosen sections. In contrast, megakaryocyte frequencies in bone marrow and spleen of normal donors are fewer than 140 and fewer than 10, respectively. Treatment with the inhibitor may reduce the number (e.g., frequencies) of megakaryocytes in bone marrow and/or spleen. In some embodiments, treatments with the inhibitor can cause reduced levels of downstream effector signaling, such as phosphorylation of SMAD2/3.

Patients with myelofibrosis may suffer from enlarged spleen. Thus, clinical effects of a therapeutic may be evaluated by monitoring changes in spleen size. Spleen size may be examined by known techniques, such as assessment of the spleen length by palpation and/or assessment of the spleen volume by ultrasound. In some embodiments, the subject to be treated with an isoform-specific, inhibitor of TGFβ1 has a baseline spleen length (prior to the treatment) of 5 cm or greater, e.g., ranging between 5 and 30 cm as assessed by palpation. In some embodiments, the subject to be treated with an isoform-specific, inhibitor of TGFβ1 has a baseline spleen volume (prior to the treatment) of 300 mL or greater, e.g., ranging between 300-1500 mL, as assessed by ultrasound. Treatment with the inhibitor, e.g., following 4-12 cycles of administration (e.g., 2, 4, 6, 8, 10, 12 cycles), for example every 4 weeks, at dosage of 0.1-30 mg/kg monoclonal antibody) described herein may reduce spleen size in the subject. In some embodiments, the effective amount of the inhibitor is sufficient to reduce spleen size in a patient population that receives the inhibitor treatment by at least 10%, 20%, 30%, 35%, 40%, 50%, and 60%, relative to corresponding baseline values. For example, the treatment is effective to achieve a ≥35% reduction in spleen volume from baseline in 12-24 weeks as measured by MRI or CT scan, as compared to placebo control. In some embodiments, the treatment is effective to achieve a ≥35% reduction in spleen volume from baseline in 24-48 weeks as measured by MRI or CT scan, as compare to best available therapy control. Best available therapy may include hydroxyurea, glucocorticoids, as well as no medication, anagrelide, epoetin alfa, thalidomide, lenalidomide, mercaptopurine, thioguanine, danazol, peginterferon alfa-2a, interferon-α, melphalan, acetylsalicylic acid, cytarabine, and colchicine.

In some embodiments, a patient population treated with an isoform-specific, TGFβ1 inhibitor such as those described herein, shows a statistically improved treatment response as assessed by, for example, International Working Group for Myelofibrosis Research and Treatment (IWG-MRT) criteria, degree of change in bone marrow fibrosis grade measured by the modified Bauermeister scale and European consensus grading system after treatment (e.g., 4, 6, 8, or 12 cycles), symptom response using the Myeloproliferative Neoplasm Symptom Assessment Form (MPN-SAF).

In some embodiments, the treatment with an isoform-specific, TGFβ1 inhibitor such as those described herein, achieves a statistically improved treatment response as assessed by, for example, modified Myelofibros is Symptom Assessment Form (MFSAF), in which symptoms are measured by the MFSAF tool (such as v2.0), a daukt diary capturing the debilitating symptoms of myelofibrosis (abdominal discomfort, early satiety, pain under left ribs, pruritus, night sweats, and bone/muscle pain) using a scale of 0 to 10, where 0 is absent and 10 is the worst imaginable. In some embodiments, the treatment is effective to achieve a 50%2 reduction in total MFSAF score from the baseline in, for example, 12-24 weeks. In some embodiments, a significant fraction of patients who receive the therapy achieves a ≥50% improvement in Total Symptom Score, as compared to patients taking placebo. For example, the fraction of the patient pool to achieve ≥50% improvement may be over 40%, 50%, 55%, 60%, 65%, 70%, 75% or 80%.

In some embodiments, the therapeutically effective amount of the inhibitor is an amount sufficient to attain clinical improvement as assessed by an anemia response. For example, an improved anemia response may include longer durations of transfusion-independence, e.g., 8 weeks or longer, following the treatment of 4-12 cycles, e.g., 6 cycles.

In some embodiments, the therapeutically effective amount of the inhibitor is an amount sufficient to maintain stable disease for a duration of time, e.g., 6 weeks, 8 weeks, 12 weeks, six months, etc. In some embodiments, progression of the disease may be evaluated by changes in overall bone marrow cellularity, the degree of reticulin or collagen fibrosis, and/or a change in JAK2V617F allele burden.

In some embodiments, a patient population treated with an isoform-specific, TGFβ1 inhibitor such as those described herein, shows statistically improved survival, as compared to a control population that does not receive the treatment. For example, in control groups, median survival of PMF patients is approximately six years (approximately 16 months in high-risk patients), and fewer than 20% of the patients are expected to survive 10 years or longer post-diagnosis. Treatment with the isoform-specific TGFβ1 inhibitor such as those described herein, may prolong the survival time by, at least 6 months, 12 months, 18 months, 24 months, 30 months, 36 months, or 48 months. In some embodiments, the treatment is effective to achieve improved overall survival at 26 weeks, 52 weeks, 78 weeks, 104 weeks, 130 weeks, 144 weeks, or 156 weeks, as compared to patients who receive placebo.

Clinical benefits of the therapy, such as those exemplified above, may be seen in patients with or without new onset anemia.

One of the advantageous features of the isoform-specific, TGFβ1 inhibitors is that they maintain improved safety profiles enabled by isoform selectivity, as compared to conventional TGFβ antagonists that lack the selectivity. Therefore, it is anticipated that treatment with an isoform-specific, inhibitor of TGFβ1, such as those described herein, may reduce adverse events in a patient population, in comparison to equivalent patient populations treated with conventional TGFβ antagonists, with respect to the frequency and/or severity of such events. Thus, the isoform-specific, TGFβ1 inhibitors may provide a greater therapeutic window as to dosage and/or duration of treatment.

Adverse events may be graded by art-recognized suitable methods, such as Common Terminology Criteria for Adverse Events (CTCAE) version 4. Previously reported adverse events in human patients who received TGFβ antagonists, such as GC1008, include: leukocytosis (grade 3), fatigue (grade 3), hypoxia (grade 3), asystole (grade 5), leukopenia (grade 1), recurrent, transient, tender erythematous, nodular skin lesions, suppurative dermatitis, and herpes zoster.

The isoform-specific, TGFβ1 inhibitor therapy may cause less frequent and/or less severe adverse events (side effects) as compared to JAK inhibitor therapy in myelofibrosis patients, with respect to, for example, anemia, thrombocytopenia, neutropenia, hypercholesterolemia, elevated alanine transaminase (ALT), elevated aspartate transaminase (AST), bruising, dizziness, and headache, thus offering a safer treatment option.

It is contemplated that inhibitors of TGFβ1 signaling may be used in conjunction with one or more therapeutics for the treatment of myelofibrosis as a combination therapy. In some embodiments, an inhibitor of TGFβ1 activation described herein is administered to patients suffering from myelofibrosis, who have received a JAK1 inhibitor, JAK2 inhibitor or JAK1/JAK2 inhibitor. In some embodiments, such patients are responsive to the JAK1 inhibitor, JAK2 inhibitor or JAK1/JAK2 inhibitor therapy, while in other embodiments such patients are poorly responsive or not responsive to the JAK1 inhibitor, JAK2 inhibitor or JAK1/JAK2 inhibitor therapy. In some embodiments, use of an isoform-specific inhibitor of TGFβ1 described herein may render those who are poorly responsive or not responsive to the JAK1 inhibitor, JAK2 inhibitor or JAK1/JAK2 inhibitor therapy more responsive. In some embodiments, use of an isoform-specific inhibitor of TGFβ1 described herein may allow reduced dosage of the JAK1 inhibitor, JAK2 inhibitor or JAK1/JAK2 inhibitor which still produces equivalent clinical efficacy in patients but fewer or lesser degrees of drug-related toxicities or adverse events (such as those listed above). In some embodiments, treatment with the inhibitor of TGFβ1 activation described herein used in conjunction with JAK1 inhibitor, JAK2 inhibitor or JAK1/JAK2 inhibitor therapy may produce synergistic or additive therapeutic effects in patients. In some embodiments, treatment with the inhibitor of TGFβ1 activation described herein may boost the benefits of JAK1 inhibitor, JAK2 inhibitor or JAK1/JAK2 inhibitor or other therapy given to treat myelofibrosis. In some embodiments, patients may additionally receive a therapeutic to address anemia associated with myelofibrosis.

### TGFβ1-Positive Cancer:

Various cancers involve TGFβ1 activities and may be treated with antibodies and/or compositions of the present disclosure. As used herein, the term “cancer” refers to any of various TGFβ1-positive malignant neoplasms characterized by the proliferation of anaplastic cells that tend to invade surrounding tissue and metastasize to new body sites and also refers to the pathological condition characterized by such malignant neoplastic growths. Cancers may be localized (e.g., solid tumors) or systemic. In the context of the present disclosure, the term “localized” (as in “localized tumor”) refers to anatomically isolated or isolatable abnormalities/lesions, such as solid malignancies, as opposed to systemic disease (e.g., so-called liquid tumors or blood cancers). Certain cancers, such as certain leukemia (e.g., myelofibrosis) and multiple myeloma, for example, may have both a localized component(for instance the bone marrow) and a systemic component (for instance circulating blood cells) to the disease. In some embodiments, cancers may be systemic, such as hematological malignancies. Cancers that may be treated according to the present disclosure are TGFβ1-positive and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. In cancer, TGFβ (e.g., TGFβ1) may be either growth promoting or growth inhibitory. As an example, in pancreatic cancers, SMAD4 wild type tumors may experience inhibited growth in response to TGFβ, but as the disease progresses, constitutively activated type II receptor is typically present. Additionally, there are SMAD4-null pancreatic cancers. In some embodiments, antibodies, antigen-binding portions thereof, and/or compositions of the present disclosure are designed to selectively target components of TGFβ signaling pathways that function uniquely in one or more forms of cancer. Leukemias, or cancers of the blood or bone marrow that are characterized by an abnormal proliferation of white blood cells, i.e., leukocytes, can be divided into four major classifications including acute lymphoblastic leukemia (ALL), chronic lymphocytic leukemia (CLL), acute myelogenous leukemia or acute myeloid leukemia (AML) (AML with translocations between chromosome 10 and 11 [t(10, 11)], chromosome 8 and 21 [t(8;21)], chromosome 15 and 17 [t(15;17)], and inversions in chromosome 16 [inv(16)]; AML with multilineage dysplasia, which includes patients who have had a prior myelodysplastic syndrome (MDS) or myeloproliferative disease that transforms into AML; AML and myelodysplastic syndrome (MDS), therapy-related, which category includes patients who have had prior chemotherapy and/or radiation and subsequently develop AML or MDS; d) AML not otherwise categorized, which includes subtypes of AML that do not fall into the above categories; and e) acute leukemias of ambiguous lineage, which occur when the leukemic cells cannot be classified as either myeloid or lymphoid cells, or where both types of cells are present); and chronic myelogenous leukemia (CML).

The isoform-selective TGFβ1 inhibitors of the invention may be used to treat patients suffering from chronic myeloid leukemia, which is a stem cell disease, in which the BCR/ABL oncoprotein is considered essential for abnormal growth and accumulation of neoplastic cells. Imatinib is an approved therapy to treat this condition; however, a significant fraction of myeloid leukemia patients show Imatinib-resistance. TGFβ1 inhibition achieved by the inhibitor such as those described herein may potentiate repopulation/expansion to counter BCR/ABL-driven abnormal growth and accumulation of neoplastic cells, thereby providing clinical benefit.

Isoform-specific, inhibitors of TGFβ1, such as those described herein, may be used to treat multiple myeloma. Multiple myeloma is a cancer of B lymphocytes (e.g., plasma cells, plasmablasts, memory B cells) that develops and expands in the bone marrow, causing destructive bone lesions (i.e., osteolytic lesion). Typically, the disease manifests enhanced osteoclastic bone resorption, suppressed osteoblast differentiation (e.g., differentiation arrest) and impaired bone formation, characterized in part, by osteolytic lesions, osteopenia, osteoporosis, hypercalcemia, as well as plasmacytoma, thrombocytopenia, neutropenia and neuropathy. The TGFβ1-selective, inhibitor therapy described herein may be effective to ameliorate one or more such clinical manifestations or symptoms in patients. The TGFβ1 inhibitor may be administered to patients who receive additional therapy or therapies to treat multiple myeloma, including those listed elsewhere herein. In some embodiments, multiple myeloma may be treated with a TGFβ1 inhibitor in combination with a myostatin inhibitor or an IL-6 inhibitor. In some embodiments, the TGFβ1 inhibitor may be used in conjunction with traditional multiple myeloma therapies, such as bortezomib, lenalidomide, carfilzomib, pomalidomide, thalidomide, doxorubicin, corticosteroids (e.g., dexamethasone and prednisone), chemotherapy (e.g., melphalan), radiation therapy, stem cell transplantation, plitidepsin, Elotuzumab, Ixazomib, Masitinib, and/or Panobinostat.

The types of carcinomas which may be treated by the methods of the present invention include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

Isoform-selective, inhibitors of TGFβ1 activation, such as those described herein, may be suited for treating malignancies involving cells of neural crest origin. Cancers of the neural crest lineage (i.e., neural crest-derived tumors) include, but are not limited to: melanoma (cancer of melanocytes), neuroblastoma (cancer of sympathoadrenal precursors), ganglioneuroma (cancer of peripheral nervous system ganglia), medullary thyroid carcinoma (cancer of thyroid C cells), pheochromocytoma (cancer of chromaffin cells of the adrenal medulla), and MPNST (cancer of Schwann cells). In some embodiments, antibodies and methods of the disclosure may be used to treat one or more types of cancer or cancer-related conditions that may include, but are not limited to colon cancer, renal cancer, breast cancer, malignant melanoma and glioblastomas (Schlingensiepen et al., 2008; Ouhtit et al., 2013).

While Tregs play an important role in dampening immune responses in healthy individuals, an elevated number of Tregs in cancer has been associated with poor prognosis. For example, human ovarian cancer ascites are infiltrated with Foxp3+ GARP+ Tregs (Downs-Canner et al., Nat Commun. 2017, 8: 14649). Similarly, Tregs positively correlated with a more immunosuppressive and more aggressive phenotype in advanced hepatocellular carcinoma (Kalathil et al., Cancer Res. 2013, 73(8): 2435-44). Tregs can suppress the proliferation of effector T cells. In addition, Tregs exert contact-dependent inhibition of immune cells (e.g., naïve CD4+ T cells) through the production of TGFβ1. To combat a tumor, therefore, it is advantageous to inhibit Tregs so sufficient effector T cells can be available to exert anti-tumor activities.

Increasing lines of evidence suggest the role of macrophages in tumor/cancer progression. The present invention encompasses the notion that this is in part mediated by TGFβ1 activation in the disease environment, such as TME. Bone marrow-derived monocytes (e.g., CD11b+) are recruited to tumor sites in response to tumor-derived cytokines/chemokines (such as CCL2, CCL3, and CCL4), where monocytes undergo differentiation and polarization to acquire pro-cancer phenotype (e.g., M2-biased, TAMs or TAM-like cells). A majority of TAMs in many tumors are M2-biased. Among the M2-like macrophages, M2c and M2d subtypes, but not M1, are found to express elevated LRRC33 on the cell surface. Moreover, macrophages can be further skewed or activated by an M-CSF exposure, resulting in a marked increase in LRRC33 expression, which coincides with TGFβ1 expression. Increased circulating M-CSF (i.e., serum M-CSF concentrations) in patients with myeloproliferative disease (e.g., myelofibrosis) has also been observed. Generally, tumors with high macrophage (TAM) and/or MDSC infiltrate are associated with poor prognosis. Similarly, elevated levels of M-CSF are also indicative of poor prognosis.

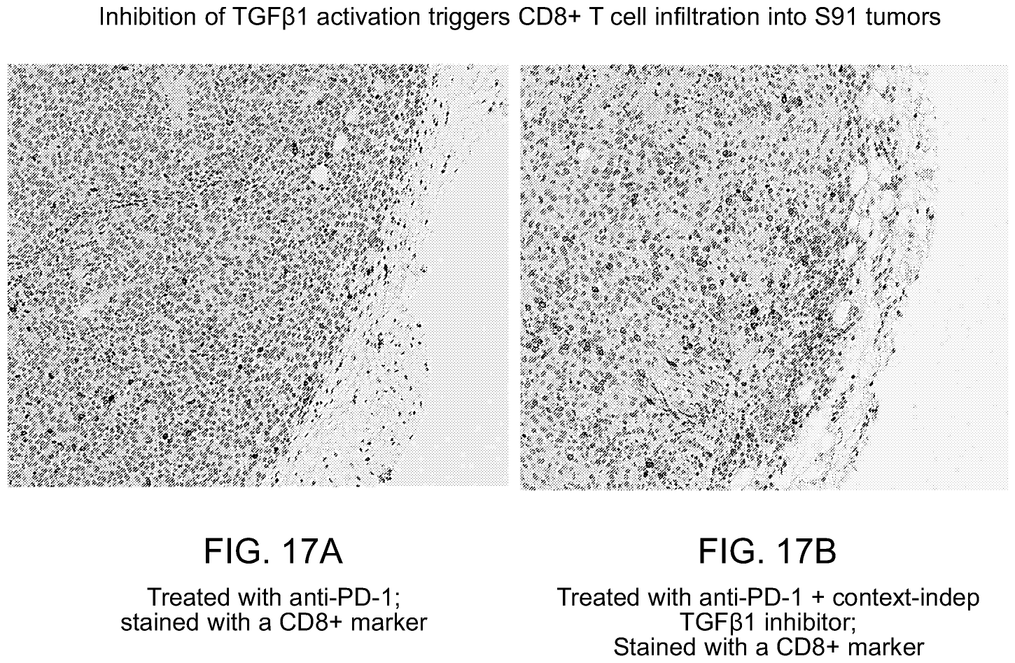

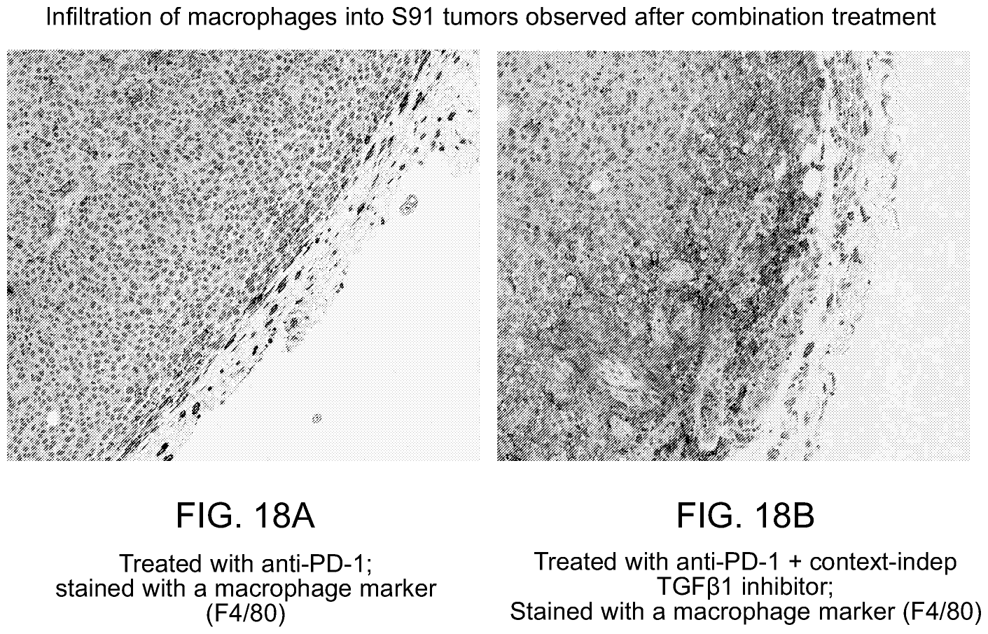

On the other hand, macrophage infiltration into a tumor may also signify effectiveness of a therapy. As exemplified in FIGS. 17A-17B and 18A-18B, tumors that are effectively penetrated by effector T cells (e.g., CD8+ T cells) following the treatment with a combination of a checkpoint inhibitor and a context-independent (or context biased)TGFβ1 inhibitor may lead to recruitment of phagocytic monocytes/macrophages that clean up cell debris. As clear from FIG. 17B, the combination of anti-PD-1 and a TGFβ1 inhibitor resulted in robust CD8 T cell influx throughout the tumor, as compared to anti-PD-1 treatment alone. Concurrently, extensive infiltration of the tumor by F4/80-positive macrophages is observed (see FIG. 18B). This may be indicative of macrophages clearing cancer cell debris generated by cytotoxic cells and is presumably a direct consequence of TGFβ1 inhibition.

As mentioned above, inhibitors of TGFβ1 activation may be used in the treatment of Melanoma. The types of melanoma that may be treated with such inhibitors include, but are not limited to: Lentigo maligna; Lentigo maligna melanoma; Superficial spreading melanoma; Acral lentiginous melanoma; Mucosal melanoma; Nodular melanoma; Polypoid melanoma and Desmoplastic melanoma. In some embodiments, the melanoma is a metastatic melanoma.

More recently, immune checkpoint inhibitors have been used to effectively treat advanced melanoma patients. In particular, anti-programmed death (PD)-1 antibodies (e.g., nivolumab and pembrolizumab) have now become the standard of care for certain types of cancer such as advanced melanoma, which have demonstrated significant activity and durable response with a manageable toxcity profile. However, effective clinical application of PD-1 antagonists is encumbered by a high rate of innate resistance (˜60-70%) (see Hugo et al. (2016) Cell 165: 35-44), illustrating that ongoing challenges continue to include the questions of patient selection and predictors of response and resistance as well as optimizing combination strategies (Perrot et al. (2013) Ann Dermatol 25(2): 135-144). Moreover, studies have suggested that approximately 25% of melanoma patients who initially responded to an anti-PD-1 therapy eventually developed acquired resistance (Ribas et al. (2016) JAMA 315: 1600-9).

The number of tumor-infiltrating CD8+ T cells expressing PD-1 and/or CTLA-4 appears to be a key indicator of success with checkpoint inhibition, and both PD-1 and CTLA-4 blockade may increase the infiltrating T cells. In patients with higherpresence of tumor-associated macrophages, however, anti-cancer effects of the CD8 cells may be suppressed.

It is contemplated that LRRC33-expressing cells, such as myeloid cells, including myeloid precursors, MDSCs and TAMs, may create or support an immunosuppressive environment (such as TME and myelofibrotic bone marrow) by inhibiting T cells (e.g., T cell depletion), such as CD4 and/or CD8 T cells, which may at least in part underline the observed anti-PD-1 resistance in certain patient populations. Indeed, evidence suggests that resistance to anti-PD-1 monotherapy was marked by failure to accumulate CD8+ cytotoxic T cells and reduced Teff/Treg ratio. Notably, the present inventors have recognized that there is a bifurcation among certain cancer patients, such as a melanoma patient population, with respect to LRRC33 expression levels: one group exhibits high LRRC33 expression (LRRC33^{high}), while the other group exhibits relatively low LRRC33 expression (LRRC33^{low}). Thus, the invention includes the notion that the LRRC33^{high} patient population may represent those who are poorly responsive to or resistant to immuno checkpoint inhibitor therapy. Accordingly, agents that inhibit LRRC33, such as those described herein, may be particularly beneficial for the treatment of cancer, such as melanoma, lymphoma, and myeloproliferative disorders, that is resistant to checkpoint inhibitor therapy (e.g., anti-PD-1).

In some embodiments, cancer/tumor is intrinsically resistant to or unresponsive to an immune checkpoint inhibitor. To give but one example, certain lymphomas appear poorly responsive to immune checkpoint inhibition such as anti-PD-1 therapy. Similarly, a subset of melanoma patient population is known to show resistance to immune checkpoint inhibitors. Without intending to be bound by particular theory, the inventors of the present disclosure contemplate that this may be at least partly due to upregulation of TGFβ1 signaling pathways, which may create an immunosuppressive microenvironment where checkpoint inhibitors fail to exert their effects. TGFβ1 inhibition may render such cancer more responsive to checkpoint inhibitor therapy. Non-limiting examples of cancer types which may benefit from a combination of an immune checkpoint inhibitor and a TGFβ1 inhibitor include: myelofibrosis, melanoma, renal cell carcinoma, bladder cancer, colon cancer, hematologic malignancies, non-s mall cell carcinoma, non-small cell lung cancer (NSCLC), lymphoma (classical Hodgkin's and non-Hodgkin's), head and neck cancer, urothelial cancer, cancer with high microsatellite instability, cancer with mismatch repair deficiency, gastric cancer, renal cancer, and hepatocellular cancer. However, any cancer (e.g., patients with such cancer) in which TGFβ1 is overexpressed or is the dominant isoform over TGFβ2/3, as determined by, for example biopsy, may be treated with an isoform-selective inhibitor of TGFβ1 in accordance with the present disclosure.

In some embodiments, a cancer/tumor becomes resistant over time. This phenomenon is referred to as acquired resistance or adaptive resistance. Like intrinsic resistance, in some embodiments, acquired resistance is at least in part mediated by TGFβ1-dependent pathways, Isoform-specific TGFβ1 inhibitors described herein may be effective in restoring anti-cancer immunity in these cases.

In some embodiments, combination therapy comprising an immune checkpoint inhibitor and an isoform-specific inhibitor of TGFβ1 which targets an LRRC33-proTGFβ1 complex (such as those described herein) may be effective to treat such cancer. In addition, high LRRC33-positive cell infiltrate in tumors, or otherwise sites/tissues with abnormal cell proliferation, may serve as a biomarker for host immunosuppression and immune checkpoint resistance. Similarly, effector T cells may be precluded from the immunosuppressive niche which limits the body's ability to combat cancer. Moreover, as demonstrated in the Example section below, Tregs that express GARP-presented TGFβ1 suppress effector T cell proliferation. Together, TGFβ1 is likely a key driver in the generation and maintenance of an immune inhibitory disease microenvironment (such as TME), and multiple TGFβ1 presentation contexts are relevant for tumors. In some embodiments, the combination therapy may achieve more favorable Teff/Treg ratios.

In some embodiments, the antibodies, or antigen-binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating cancer in a subject in need thereof, said method comprising administering the antibody, or antigen-binding portion thereof, to the subject such that the cancer is treated. In certain embodiments, the cancer is colon cancer.

In some embodiments, the antibodies, or antigen-binding portions thereof, as described herein, may be used in methods for treating solid tumors. In some embodiments, solid tumors may be desmoplastic tumors, which are typically dense and hard for therapeutic molecules to penetrate. By targeting the ECM component of such tumors, such antibodies may “loosen” the dense tumor tissue to disintegrate, facilitating therapeutic access to exert its anti-cancer effects. Thus, additional therapeutics, such as any known anti-tumor drugs, may be used in combination.

Additionally or alternatively, isoform-specific, antibodies for fragments thereof that are capable of inhibiting TGFβ1 activation, such as those disclosed herein, may be used in conjunction with the chimeric antigen receptor T-cell (“CAR-T”) technology as cell-based immunotherapy, such as cancer immunotherapy for combatting cancer.

In some embodiments, the antibodies, or antigen-binding portions thereof, as described herein, may be used in methods for inhibiting or decreasing solid tumor growth in a subject having a solid tumor, said method comprising administering the antibody, or antigen-binding portion thereof, to the subject such that the solid tumor growth is inhibited or decreased. In certain embodiments, the solid tumor is a colon carcinoma tumor. In some embodiments, the antibodies, or antigen-binding portions thereof useful for treating a cancer is an isoform-specific, inhibitor of TGFβ1 activation.

The invention includes the use of isoform-specific inhibitors of TGFβ1, as described herein, in the treatment of cancer comprising a solid tumor in a subject. In some embodiments, such isoform-specific inhibitor may inhibit the activation of TGFβ1. In some embodiments, the solid tumor is characterized by having stroma enriched with CD8+ T cells making direct contact with CAFs and collagen fibers. Such a tumor may create an immuno-suppressive environment that prevents anti-tumor immune cells (e.g., effector T cells) from effectively infiltrating the tumor, limiting the body's ability to fight cancer. Instead, such cells may accumulate within or near the tumor stroma. These features may render such tumors poorly responsive to an immune checkpoint inhibitor therapy. As discussed in more detail below, TGFβ1 inhibitors disclosed herein may unblock the suppression so as to allow effector cells to reach and kill cancer cells, for example, used in conjunction with an immune checkpoint inhibitor.

TGFβ1 is contemplated to play multifaceted roles in a tumor microenvironment, including tumor growth, host immune suppression, malignant cell proliferation, vascularity, angiogenesis, migration, invasion, metastasis, and chemo-resistance. Each “context” of TGFβ1 presentation in the environment may therefore participate in the regulation (or dysregulation) of disease progression. For example, the GARP axis is particularly important in Treg response that regulates effector T cell response for mediating host immune response to combat cancer cells. The LTBP1/3 axis may regulate the ECM, including the stroma, where cancer-associated fibroblasts (CAFs) playa role in the pathogenesis and progression of cancer. The LRRC33 axis may playa crucial role in recruitment of circulating monocytes to the tumor microenvironment, subsequent differentiation into tumor-associated macrophages (TAMs), infiltration into the tumor tissue and exacerbation of the disease.

The tumor microenvironment (TME) contains multiple cell types expressing TGFβ1, such as activated myofibroblast-like fibroblasts, stromal cells, infiltrating macrophages, MDSCs and other immune cells, in addition to cancer (i.e., malignant) cells. Thus, the TME represents a heterogeneous population of cells expressing and/or responsive to TGFβ1 but in association with more than one types of presenting molecules, e.g., LTBP1, LTBP3, LRRC33 and GARP, within the niche.

More recently, a phenomenon referred to as “immune exclusion” was coined to describe a tumor environment from which anti-tumor effector T cells (e.g., CD8+ T cells) are kept away (hence “excluded”) by immunosuppressive local cues, and TGFβ is thought to at least in part mediate this process. In an immune-excluded tumor, effector cells, which would otherwise be capable of attacking cancer cells by recognizing cell-surface tumor antigens, are prevented from gaining access to the site of cancer cells. In this way, cancer cells evade host immunity and immuno-oncologic therapeutics, such as checkpoint inhibitors, that exploit and rely on such immunity. Indeed, such tumors show resistance to checkpoint inhibition, such as anti-PD-1 and anti-PD-L1 antibodies, presumably because target T cells are blocked from entering the tumor hence failing to exert anti-cancer effects.

Increasing evidence suggests that TGFβ may be a primary player in creating and/or maintaining immunosuppression in disease tissues, including the immune-excluded tumor environment. Therefore, TGFβ inhibition may unblock the immunosuppression and enable effector T cells (particularly cytotoxic CD8+ T cells) to access and kill target cancer cells. In addition to tumor infiltration, TGFβ inhibition may also promote CD8+ T cell expansion. While the exact mechanism underlining this process has yet to be elucidated, it is contemplated that immunosuppression is at least in part mediated by immune cell-associated TGFβ1 activation involving regulatory T cells and activated macrophages. It has been reported that TGFβ directly promotes Foxp3 expression in CD4+ T cells, thereby converting them into a regulatory phenotype (i.e., Treg). Moreover, Tregs suppress effector T cell proliferation (see, for example, FIG. 26), thereby reducing immune responses. This process is shown to be TGFβ1-dependent and likely involves GARP-associated TGFβ1 signaling. Observations in both humans and animal models have indicated that an increase in Tregs in TME is associated with poor prognosis in multiple types of cancer. In addition, Applicant has previously shown that M2-polarized macrophages exposed to tumor-derived factors such as M-CSF dramatically upregulate cell-surface expression of LRRC33, which is a presenting molecule for TGFβ 1 (see, for example: PCT/US2018/031759). These so-called tumor-associated macrophages (or TAMs) are thought to contribute to the observed TGFβ1-dependent immunosuppression in TMEs and promote tumor growth.

A number of solid tumors are characterized by having tumor stroma enriched with myofibroblasts or myofibroblast-like cells. These cells produce collagenous matrix that surrounds or encases the tumor (such as desmoplasia), which at least in part may be caused by overactive TGFβ1 signaling. It is contemplated that the TGFβ1 activation is mediated via ECM-associated presenting molecules, e.g., LTBP1 and LTBP3 in the tumor stroma.

In some embodiments, TGFβ1-expressing cells infiltrate the tumor, creating an immunosuppressive local environment. The degree by which such infiltration is observed may correlate with worse prognosis. In some embodiments, higher infiltration is indicative of poorer treatment response to another cancer therapy, such as immune checkpoint inhibitors. In some embodiments, TGFβ1-expressing cells in the tumor microenvironment comprise Tregs and/or myeloid cells. In some embodiments, the myeloid cells include, but are not limited to: macrophages, monocytes (tissue resident or bone marrow-derived), and MDSCs.

In some embodiments, LRRC33-expressing cells in the TME are myeloid-derived suppressor cells (MDSCs). MDSC infiltration (e.g., solid tumor infiltrate) may underline at least one mechanism of immune escape, by creating an immunosuppressive niche from which host's anti-tumor immune cells become excluded. Evidence suggest that MDSCs are mobilized by inflammation-associated signals, such as tumor-associated inflammatory factors, Opon mobilization, MDSCs can influence immunosuppressive effects by impairing disease-combating cells, such as CD8+ cells and NK cells. In addition, MDSCs may induce differentiation of Tregs by secreting TGFβ and IL-10. Thus, an isoform-specific TGFβ1 inhibitor, such as those described herein, may be administered to patients with immune evasion (e.g., compromised immune surveillance) to restore or boost the body's ability to fight the disease (such as tumor). As described in more detail herein, this may further enhance (e.g., restore or potentiate) the body's responsiveness or sensitivity to another therapy, such as cancer therapy.

In some embodiments, elevated frequencies (e.g., number) of circulating MDSCs in patients are predictive of poor responsiveness to checkpoint blockade therapies, such as PD-1 antagonists and PD-L1 antagonists. For example, biomarker studies showed that circulating pre-treatment HLA-DR lo/CD14+/CD11b+ myeloid-derived suppressor cells (MDSC) were associated with progression and worse OS (p=0.0001 and 0.0009). In addition, resistance to PD-1 checkpoint blockade in inflamed head and neck carcinoma (HNC) associates with express ion of GM-CSF and Myeloid Derived Suppressor Cell (MDSC) markers. This observation suggested that strategies to deplete MDSCs, such as chemotherapy, should be considered in combination or sequentially with anti-PD-1. LRRC33 or LRRC33-TGFβ complexes represent a novel target for cancer immunotherapy due to selective expression on immunosuppressive myeloid cells. Therefore, without intending to be bound by particular theory, targeting this complex may enhance the effectiveness of standard-of-care checkpoint inhibitor therapies in the patient population.

The invention therefore provides the use of an isoform-specific, TGFβ1 inhibitor described herein for the treatment of cancer that comprises a solid tumor. Such treatment comprises administration of the isoform-specific, TGFβ1 inhibitor to a subject diagnosed with cancer that includes at least one localized tumor (solid tumor) in an amount effective to treat the cancer.

Evidence suggests that cancer progression (e.g., tumor proliferation/growth, invasion, angiogenesis and metastasis) may be at least in part driven by tumor-stroma interaction. In particular, CAFs may contribute to this process by secretion of various cytokines and growth factors and ECM remodeling. Factors involved in the process include but are not limited to stromal-cell-derived factor 1 (SCD-1), MMP2, MMP9, MMP3, MMP-13, TNF-α, TGFβ1, VEGF, IL-6, M-CSF. In addition, CAFs may recruit TAMs by secreting factors such as CCL2/MCP-1 and SDF-1/CXCL12 to a tumor site; subsequently, a pro-TAM niche (e.g., hyaluronan-enriched stromal areas) is created where TAMs preferentially attach. Since TGFβ1 has been suggested to promote activation of normal fibroblasts into myofibroblast-like CAFs, administration of an isoform-specific, TGFβ1 inhibitor such as those described herein may be effective to counter cancer-promoting activities of CAFs. Indeed, data presented herein suggest that an isoform-specific antibody that blocks activation of TGFβ1 can inhibit UUO-induced upregulation of maker genes such as CCL2/MCP-1, α-SMA. FN1 and Col1, which are also implicated in many cancers.

In certain embodiments, the antibodies, or antigen-binding portions thereof, as described herein, are administered to a subject having cancer or a tumor, either alone or in combination with an additional agent, e.g., an anti-PD-1 antibody (e.g., an anti-PD-1 antagonist). Other combination therapies which are included in the invention are the administration of an antibody, or antigen-binding portion thereof, described herein, with radiation, or a chemotherapeutic agent. Exemplary additional agents include, but are not limited to, a PD-1 antagonist, a PDL1 antagonist, a PD-L1 or PDL2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody, an anti-CD27 antibody, an anti-CD70 antibody, an anti-CD47 antibody, an anti-41 BB antibody, an anti-PD-1 antibody, an anti-CD20 antibody, an oncolytic virus, and a PARP inhibitor.

In some embodiments, determination or selection of therapeutic approach for combination therapy that suits particular cancer types or patient population may involve the following: a) considerations regarding cancer types for which a standard-of-care therapy is available (e.g., immunotherapy-approved indications); b) considerations regarding treatment-resistant subpopulations; and c) considerations regarding cancers/tumors that are “TGFβ1 pathway-active” or otherwise at least in part TGFβ1-dependent(e.g., TGFβ1 inhibition-sensitive). For example, many cancer samples show that TGFβ1 is the predominant isoform by, for instance, TCGA RNAseq analysis. In some embodiments, over 50% (e.g., over 50%, 60%, 70%, 80% and 90%) of samples from each tumor type are positive for TGFβ1 isoform expression. In some embodiments, the cancers/tumors that are “TGFβ1 pathway-active” or otherwise at least in part TGFβ1-dependent (e.g., TGFβ1 inhibition-sensitive) contain at least one Ras mutation, such as mutations in K-ras, N-ras and/or H-ras. In some embodiments, the cancer/tumor comprises at least one K-ras mutation.

In some embodiments, the isoform-specific, TGFβ1 inhibitor is administered in conjunction with checkpoint inhibitory therapy to patients diagnosed with cancer for which one or more checkpoint inhibitor therapies are approved or shown effect. These include, but are not limited to: bladder urothelial carcinoma, squamous cell carcinoma (such as head & neck), kidney clear cell carcinoma, kidney papillary cell carcinoma, liver hepatocellular carcinoma, lung adenocarcinoma, skin cutaneous melanoma, and stomach adenocarcinoma. In preferred embodiments, such patients are poorly responsive or non-responsive to the checkpoint inhibitor therapy. In some embodiments, the poor responsiveness is due to primary resistance. In some embodiments, the cancer that is resistant to checkpoint blockade shows downregulation of TCF7 expression. In some embodiments, TCF7 downregulation in checkpoint inhibition-resistant tumor may be correlated with a low number of intratumoral CD8+ T cells.

The isoform-specific, TGFβ1 inhibitor may be used in the treatment of chemotherapy- or radiotherapy-resistant cancers. Thus, in some embodiments, the isoform-specific, TGFβ1 inhibitor is administered to patients diagnosed with cancer for which they receive or have received chemotherapy and/or radiation therapy. In particular, the use of the TGFβ1 inhibitor is advantageous where the cancer (patient) is resistant to such therapy. In some embodiments, such cancer comprises quiescent tumor propagating cancer cells (TPCs), in which TGFβ signaling controls their reversible entry into a growth arrested state, which protects TPCs from chemotherapy or radiation therapy. It is contemplated that upon pharmacological inhibition of TGFβ1, TPCs with compromised fail to enter quiescence and thus rendered susceptible to chemotherapy and/or radiation therapy. Such cancer includes various carcinomas, e.g., squamous cell carcinomas. See, for example, Brown et al. (2017) “TGF-β-Induced Quiescence Mediates Chemoresistance of Tumor-Propagating Cells in Squamous Cell Carcinoma.” Cell Stem Cell. 21(5):650-664.

In some embodiments, TGFβ1-positive cancer to be treated with the TGFβ1 inhibitor is also TGFβ3-positive (i.e., TGFβ1+/TGFβ3+ cancer) characterized in that the disease tissue (e.g., tumor) expresses both the isoforms. In some embodiments, such tumors are co-dominant with both the TGFβ1 and TGFβ3 isoforms. Accordingly, the invention includes the use of isoform-selective TGFβ1 inhibitor in conjunction with an isoform-selective TGFβ3 inhibitor in the treatment of such conditions. Non-limiting examples of TGFβ1+/TGFβ3+ cancers include but are not limited to: breast carcinoma (e.g., breast invasive carcinoma), cholangiocarcinoma, glioblastoma multiforme, head & neck squamous cell carcinoma, kidney clear cell carcinoma, lung squamous cell carcinoma, mesothelioma, pancreatic adenocarcinoma, prostate adenocarcinoma, sarcoma, thymoma and uterine carconosarcoma.

Conditions that Benefit from Tissue Regeneration and Stem Cell Repopulation

Evidence suggests that TGFβ1 plays a role in regulating the homeostasis of various stem cell populations and their differentiation/repopulation within a tissue. During homeostasis, tissue-specific stem cells are held predominantly quiescent but are triggered to enter cell cycle upon certain stress. TGFβ1 is thought to function as a “break” during the process that tightly regulates stem cell differentiation and reconstitution, and the stress that triggers cell cycle entry coincides with TGFβ1 inhibition that removes the “break.” Thus, it is contemplated that isoform-selective inhibitors of TGFβ1, such as those described herein, may be used to skew or correct cell cycle and GO entry decision of stem cells/progenitor cells within a particular tissue.

Accordingly, the inventors of the present disclosure contemplate the use of isoform-selective TGFβ1 inhibitors in conditions in which: i) stem cell/progenitor cell differentiation/reconstitution is halted or perturbed due to a disease or induced as a side effect of a therapy/mediation; ii) patients are on a therapy or mediation that causes healthy cells to be killed or depleted; iii) patients may benefit from increased stem cell/progenitor cell differentiation/reconstitution; iv) disease is associated with abnormal stem cell differentiation or reconstitution.

For example, mesenchymal stromal/stem cells (MSCs) are a small population of stromal cells present in most adult connective tissues, such as bone marrow, fat tissue, and umbilical cord blood. MSCs are maintained in a relative state of quiescence in vivo but, in response to a variety of physiological and pathological stimuli, are capable of proliferating then differentiating into osteoblasts, chondrocytes, adipocytes, orothermesoderm-type lineages like smooth muscle cells (SMCs) and cardiomyocytes. Multiple signaling networks orchestrate MSCs differentiating into functional mesenchymal lineages, among which TGF-β1 has emerged as a key player (reviewed for example by Zhao & Hantash (2011. Vitam Horm 87:127-41).

Similarly, hematopoietic stem cells are required for lifelong blood cell production; to prevent exhaustion, the majority of hematopoieticstem cells remain quiescent during steady-state hematopoiesis. During hematologic stress, however, these cells are rapidly recruited into cell cycle and undergo extensive self-renewal and differentiation to meet increased hematopoietic demands. TGFβ1 may work as the “switch” to control the quiescence-repopulation transition/balance.

Thus, the isoform-selective inhibitors of TGFβ1 can be used in the treatment of conditions involving hematopoietic stem cell defects and bone marrow failure. In some embodiments, depletion or impairment of the hematopoietic stem cell reservoirleads to hematopoieticfailure or hematologicmalignancies. In some embodiments, such conditions are DNA repair disorder characterized by progressive bone marrow failure. In some embodiments, such condition is caused by stem and progenitor cell attrition. In some embodiments, such conditions are associated with anemia. In some embodiments, such condition is Fanconi Anemia (FA). In some embodiments, such conditions are characterized by hyperactive TGFβ pathway that suppresses the survival of certain cell types upon DNA damage. Thus, it is contemplated that the isoform-selective inhibitors of TGFβ1 can be used for rescuing proliferation defects of FA hematopoietic stem cells and/or bone marrow failure in subjects with FA. See, for example, Zhang et al. (2016), Cell Stem Cell, 18: 668-681, “TGF-β inhibition rescues hematopoietic stem cell defects and bone marrow failure in Fanconi Anemia.”

### Conditions Involving Treatment-Induced Hematopoietic Dysregulation

It is recognized that certain drugs which are designed to treat various disease conditions, often induce or exacerbate anemia in the patient being treated (e.g., treatment- or drug-induced anemia, such as chemotherapy-induced anemia and radiation therapy-induced anemia). In some embodiments, the patient is treated with a myelosuppressive drug that may cause side effects that include anemia. Such patient may benefit from pharmacological TGFβ1 inhibition in order to boost hematopoiesis. In some embodiments, the TGFβ1 inhibitor may promote hematopoiesis in patients by preventing entry into a quiescent state. In some embodiments, the patient may receive a G-CSF therapy (e.g., Filgrastim).

Accordingly, the invention includes the use of an isoform-selective inhibitor of TGFβ1, such as those disclosed herein, to be administered to patients who receive myelosuppressive therapy(e.g., therapy with side effects including myelosuppressive effects). Examples of myelosuppressive therapies include but are not limited to: peginterferon alfa-2a, interferon alfa-n3, peg interferon alfa-2b, aldesleukin, gemtuzumab ozogamicin, interferon alfacon-1, rituxmab, ibritumomabtiuxetan, tositumomab, alemtuzumab, bevacizumab, L-Phenylalanine, bortezomib, cladribine, carmustine, amsacrine, chlorambucil, raltitrexed, mitomycin, bexarotene, vindesine, floxuridine, tioguanine, vinorelbine, dexrazoxane, sorafenib, streptozocin, gemcitabine, teniposide, epirubicin, chloramphenicol, lenalidomide, altretamine, zidovudine, cis platin, oxaliplatin, cyclophosphamide, fluorouracil, propylthiouracil, pentostatin, methotrexate, carbamazepine, vinblastine, linezolid, imatinib, clofarabine, pemetrexed, daunorubicin, irinotecan, methimazole, etoposide, dacarbazine, temozolomide, tacrolimus, sirolimus, mechlorethamine, azacitidine, carboplatin, dactinomycin, cytarabine, doxorubicin, hydroxyurea, busulfan, topotecan, mercaptopurine, thalidomide, melphalan, fludarabine, flucytosine, capecitabine, procarbazine, arsenic trioxide, idarubicin, ifosfamide, mitoxantrone, lomustine, paclitaxel, docetaxel, dasatinib, decitabine, nelarabine, everolimus, vorinostat, thiotepa, ixabepilone, nilotinib, belinostat, trabectedin, trastuzumab emtansine, temsirolimus, bosutinib, bendamustine, cabazitaxel, eribulin, ruxolitinib, carfilzomib, tofacitinib, ponatinib, pomalidomide, obinutuzumab, tedizolid phosphate, blinatumomab, ibrutinib, palbociclib, olaparib, dinutuxmab, and colchicine.

Additional indications may include any of those disclosed in US Pub. No. 2013/0122007, U.S. Pat. No. 8,415,459 or International Pub. No. WO 2011/151432, the contents of each of which are herein incorporated by reference in their entirety.

In preferred embodiments, antibodies, antigen-binding portions thereof, and compositions of the d is clos u re may be used to treat a wide variety of diseases, disorders and/or conditions associated with TGFβ1 signaling. In some embodiment, target tissues/cells preferentially express the TGFβ1 isoform over the other isoforms. Thus, the invention includes methods for treating such a condition associated with TGFβ1 expression (e.g., dysregulation of TGFβ1 signaling and/or upregulation of TGFβ1 expression) using a pharmaceutical composition that comprises an antibody or antigen-binding portion thereof described herein.

In some embodiments, the disease involves TGFβ1 associated with (e.g., presented on or deposited from) multiple cellular sources. In some embodiments, such disease involves both an immune component and an ECM component of TGFβ1 function. In some embodiments, such disease involves: i) dysregulation of the ECM (e.g., overproduction/deposition of ECM components such as collagens and proteases; altered stiffness of the ECM substrate; abnormal or pathological activation or differentiation of fibroblasts, such as myofibroblasts and CAFs); ii) immune suppression due to increased Tregs and/or suppressed effector T cells (Teff), e.g., elevated ratios of Treg/Teff; increased leukocyte infiltrate (e.g., macrophage and MDSCs) that causes suppression of CD4 and/or CD8 T cells; and/or iii) abnormal or pathological activation, differentiation, and/or recruitment of myeloid cells, such as macrophages (e.g., bone marrow-derived monocytic/macrophages and tissue resident macrophages), monocytes, myeloid-derived suppresser cells (MDSCs), neutrophils, dendritic cells, and NK cells.

In some embodiments, the condition involves TGFβ1 presented by more than one types of presenting molecules (e.g., two or more of: GARP, LRRC33, LTBP1 and/or LTBP3). In some embodiments, an affected tissues/organs/cells that include TGFβ1 from multiple cellular sources. To give but one example, a solid tumor (which may also include a proliferative disease involving the bone marrow, e.g., myelofibrosis and multiple myeloma) may include TGFβ1 from multiple sources, such as the cancer cells, stromal cells, surrounding healthy cells, and/or infiltrating immune cells (e.g., CD45+ leukocytes), involving different types of presenting molecules. Relevant immune cells include but are not limited to myeloid cells and lymphoid cells, for example, neutrophils, eosinophils, basophils, lymphocytes (e.g., B cells, T cells, and NK cells), and monocytes.

### Treatment Regimen and Administration

To practice the methods disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, antibodies, or antigen-binding portions thereof, that specifically bind a GARP-TGFβ1 complex, a LTBP1-TGFβ1 complex, a LTBP3-TGFβ1 complex, and/or a LRRC33-TGFβ1 complex can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a TGFβ-related indication, such as those noted above. A subject having a TGFβ-related indication can be identified by routine medical examination, e.g., laboratory tests, organ functional tests, CT scans, MRI, or ultrasounds. A subject suspected of having any of such indication might show one or more symptoms of the indication. A subject at risk for the indication can be a subject having one or more of the risk factors for that indication.

As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present invention, the intended purpose may be to inhibit TGFβ-1 activation in vivo, to achieve clinically meaningful outcome associated with the TGFβ-1 inhibition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a TGFβ-related indication. Alternatively, sustained continuous release formulations of an antibody that specifically binds a GARP-TGFβ1 complex, a LTBP1-TGFβ1 complex, a LTBP3-TGFβ1 complex, and/or a LRRC33-TGFβ1 complex may be appropriate. Various formulations and devices for achieving sustained release would be apparent to the skilled artisan and are within the scope of this disclosure.

In one example, dosages for an antibody that specifically binds a GARP-TGFβ1 complex, a LTBP1-TGFβ1 complex, a LTBP3-TGFβ1 complex, and/or a LRRC33-TGFβ1 complex as described herein may be determined empirically in individuals who have been given one or more administration(s) of the antibody. Individuals are given incremental dosages of the antagonist. To assess efficacy, an indicator of the TGFβ-related indication can be followed. For example, methods for measuring for myofiber damage, myofiber repair, inflammation levels in muscle, and/or fibrosis levels in muscle are well known to one of ordinary skill in the art.

The present invention encompasses the recognition that agents capable of modulating the activation step of TGFβs in an isoform-specific manner may provide improved safety profiles when used as a medicament. Accordingly, the invention includes antibodies and antigen-binding fragments thereof that specifically bind and inhibit activation of TGFβ1, but not TGFβ2 or TGFβ3, thereby conferring specific inhibition of the TGFβ1 signaling in vivo while minimizing unwanted side effects from affecting TGFβ2 and/or TGFβ3 signaling.

In some embodiments, the antibodies, or antigen-binding portions thereof, as described herein, are not toxic when administered to a subject. In some embodiments, the antibodies, or antigen-binding portions thereof, as described herein, exhibit reduced toxcity when administered to a subject as compared to an antibody that specifically binds to both TGFβ1 and TGFβ2. In some embodiments, the antibodies, or antigen-binding portions thereof, as described herein, exhibit reduced toxcity when administered to a subject as compared to an antibody that specifically binds to both TGFβ1 and TGFβ3. In some embodiments, the antibodies, or antigen-binding portions thereof, as described herein, exhibit reduced toxcity when administered to a subject as compared to an antibody that specifically binds to TGFβ1, TGFβ2 and TGFβ3.

Generally, for administration of any of the antibodies described herein, an initial candidate dosage can be about 1-20 mg/kg per administration, e.g., weekly, every 2 weeks, every 3 weeks, monthly, etc. For example, patients may receive an injection of about 1-10 mg/kg per 1 week, per 2 weeks, per 3 weeks, or per 4 weeks, etc., in an amount effective to treat a disease (e.g., fibrosis or cancer) wherein the amount is well-tolerated (within acceptable toxicities or adverse events).

For the purpose of the present disclosure, a typical dosage (per administration, such as an injection and infusion) might range from about any of 0.1 mg/kg to 1 mg/kg to 2 mg/kg to 3 mg/kg, to 5 mg/kg to 10 mg/kg to 20 mg/kg to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a TGFβ-related indication, or a symptom thereof. An exemplary dosing regimen comprises administering an initial, followed by one or more maintenance doses. However, other dosage regimens may be useful, depending on the pattern of pharmacokinetic decay that the practitioner wishes to achieve. Pharmacokinetics experiments have shown that the serum concentration of an antibody disclosed herein (e.g., Ab3) remains stable for at least 7 days after administration to a preclinical animal model (e.g., a mouse model). Without wishing to be bound by any particular theory, this stability post-administration may be advantageous since the antibody may be administered less frequently while maintaining a clinically effective serum concentration in the subject to whom the antibody is administered (e.g., a human subject). In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. The particular dosage regimen, e.g., dose, timing and repetition, will depend on the particular individual and that individual's medical history, as well as the properties of the individual agents (such as the half-life of the agent, and other relevant considerations).

For the purpose of the present disclosure, the appropriate dosage of an antibody disclosed herein will depend on the specific antibody (or compositions thereof) employed, the type and severity of the indication, whether the antibody is administered for preventive or therapeutic purposes, previous therapy, the patient's clinical history and response to the antagonist, and the discretion of the attending physician. In some embodiments, a clinician will administer an antibody until a dosage is reached that achieves the desired result. Administration of an antibody can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a TGFβ-related indication.

Serum concentrations of the TGFβ1 antibody that are therapeutically effective to treat a TGFβ1-related indication in accordance with the present disclosure may be at least about 10 μg/mL, e.g., between about 10 μg/mL and 1.0 mg/mL. In some embodiments, effective amounts of the antibody as measured by serum concentrations are about 20-400 μg/mL. In some embodiments, effective amounts of the antibody as measured by serum concentrations are about 100-800 μg/mL. In some embodiments, effective amounts of the antibody as measured by serum concentrations are at least about 20 μg/mL, e.g., at least about 50 μg/mL, 100 μg/mL, 150 μg/mL or 200 μg/mL.

As used herein, the term “treating” refers to the application or administration of a composition including one or more active agents to a subject, who has a TGFβ-related indication, a symptom of the indication, or a predisposition toward the indication, with the purpose to cure, heal, alleviate, relieve, alter, remedy, ameliorate, improve, or affect the indication, the symptom of the indication, or the predisposition toward the indication.

Alleviating a TGFβ-related indication with an antibody disclosed herein includes delaying the development or progression of the indication, or reducing indication's severity. Alleviating the indication does not necessarily require curative results. As used therein, “delaying” the development of an indication associated with a TGFβ-related indication means to defer, hinder, slow, retard, stabilize, and/or postpone progression of the indication. This delay can be of varying lengths of time, depending on the history of the indication and/or individuals being treated. A method that “delays” or alleviates the development of an indication, or delays the onset of the indication, is a method that reduces probability of developing one or more symptoms of the indication in a given time frame and/or reduces extent of the symptoms in a given time frame, when compared to not using the method. Such comparisons are typically based on clinical studies, using a number of subjects sufficient to give a statistically significant result.

### Combination Therapy

The disclosure further encompasses pharmaceutical compositions and related methods used as combination therapies for treating subjects who may benefit from TGFβ inhibition in vivo. In any of these embodiments, such subjects may receive combination therapies that include a first composition comprising at least one TGFβ inhibitor, e.g., antibody or antigen-binding portion thereof, described herein, in conjunction with a second composition comprising at least one additional therapeutic intended to treat the same or overlapping disease or clinical condition. The first and second compositions may both act on the same cellular target, or discrete cellular targets. In some embodiments, the first and second compositions may treat or alleviate the same or overlapping set of symptoms or aspects of a disease or clinical condition. In some embodiments, the first and second compositions may treat or alleviate a separate set of symptoms or aspects of a disease or clinical condition. To give but one example, the first composition may treat a disease or condition associated with TGFβ signaling, while the second composition may treat inflammation or fibrosis associated with the same disease, etc. Such combination therapies may be administered in conjunction with each other. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

In preferred embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

Accordingly, the invention provides pharmaceutical compositions and methods for use in combination therapies for the reduction of TGFβ1 protein activation and the treatment or prevention of diseases or conditions associated with TGFβ1 signaling, as described herein. Accordingly, the methods or the pharmaceutical compositions further comprise a second therapy. In some embodiments, the second therapy may be useful in treating or preventing diseases or conditions associated with TGFβ1 signaling. The second therapy may diminish or treat at least one symptom (s) associated with the targeted disease. The first and second therapies may exert their biological effects by similar or unrelated mechanisms of action; or either one or both of the first and second therapies may exert their biological effects by a multiplicity of mechanisms of action.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered simultaneously or sequentially within described embodiments.

The one or more anti-TGFβ antibodies, or antigen-binding portions thereof, of the invention may be used in combination with one or more of additional therapeutic agents. Examples of the additional therapeutic agents which can be used with an anti-TGFβ antibody of the invention include, but are not limited to: cancer vaccines, engineered immune cell therapies, chemotherapies, radiation therapies, a modulator of a member of the TGFβ superfamily, such as a myostatin inhibitor and a GDF11 inhibitor; a VEGF agonist; an IGF1 agonist; an FXR agonist; a CCR2 inhibitor; a CCR5 inhibitor; a dual CCR2/CCR5 inhibitor; a lysyl oxidase-like-2 inhibitor; an ASK1 inhibitor; an Acetyl-CoA Carboxylase (ACC) inhibitor; a p38 kinase inhibitor; Pirfenidone; Nintedanib; an M-CSF inhibitor (e.g., M-CSF receptor antagonist and M-CSF neutralizing agents); a MAPK inhibitor (e.g., Erk inhibitor), an immune checkpoint agonist or antagonist; an IL-11 antagonist; and IL-6 antagonist, and the like. Other examples of the additional therapeutic agents which can be used with the TGFβ inhibitors include, but are not limited to, an indoleamine 2,3-dioxygenase (IDO) inhibitor, a tyrosine kinase inhibitor, Ser/Thr kinase inhibitor, a dual-specific kinase inhibitor. In some embodiments, such an agent may be a PI3K inhibitor, a PKC inhibitor, or a JAK inhibitor. In some embodiments, such an agent may be a TGFβ3-selective inhibitor.

In some embodiments, the additional agent is a checkpoint inhibitor. In some embodiments, the additional agent is selected from the group consisting of a PD-1 antagonist, a PDL1 antagonist, a PD-L1 or PDL2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody, an anti-CD27 antibody, an anti-CD70 antibody, an anti-CD47 antibody, an anti-41 BB antibody, an anti-PD-1 antibody, an oncolytic virus, and a PARP inhibitor. In some embodiments, the isoform-specific inhibitor of TGFβ1 activation disclosed herein is used in A treatment of cancer in a subject who is a poor responder or non-responder of a checkpoint inhibition therapy, such as those listed herein.

In some embodiments, the additional agent binds a T-cell costimulation molecule, such as inhibitory costimulation molecules and activating costimulation molecules. In some embodiments, the additional agent is selected from the group consisting of an anti-CD40 antibody, an anti-CD38 antibody, an anti-KIR antibody, an anti-CD33 antibody, an anti-CD137 antibody, and an anti-CD74 antibody.

In some embodiments, the additional therapy is radiation. In some embodiments, the additional agent is a chemotherapeutic agent. In some embodiments, the chemotherapeutic agent is Taxol. In some embodiments, the additional agent is an anti-inflammatory agent. In some embodiments, the additional agent inhibits the process of monocyte/macrophage recruitment and/or tissue infiltration. In some embodiments, the additional agent is an inhibitor of hepatic stellate cell activation. In some embodiments, the additional agent is a chemokine receptor antagonist, e.g., CCR2 antagonists and CCR5 antagonists. In some embodiments, such chemokine receptor antagonist is a dual specific antagonist, such as a CCR2/CCR5 antagonist. In some embodiments, the additional agent to be administered as combination therapy is or comprises a member of the TGFβ superfamily of growth factors or regulators thereof. In some embodiments, such agent is selected from modulators (e.g., inhibitors and activators) of GDF8/myostatin and GDF11. In some embodiments, such agent is an inhibitor of GDF8/myostatin signaling. In some embodiments, such agent is a monoclonal antibody that specifically binds a pro/latent myostatin complex and blocks activation of myostatin. In some embodiments, the monoclonal antibody that specifically binds a pro/latent myostatin complex and blocks activation of myostatin does not bind free, mature myostatin.

In some embodiments, an additional therapy comprises cell therapy, such as CAR-T therapy.

In some embodiments, an additional therapy is a cancer vaccine. Numerous clinical trials that tested peptide-based cancer vaccines have targeted hematological malignancies (cancers of the blood), melanoma (skin cancer), breast cancer, head and neck cancer, gastroesophageal cancer, lung cancer, pancreatic cancer, prostate cancer, ovarian cancer, and colorectal cancers. The antigens included peptides from HER2, telomerase (TERT), survivin (BIRC5), and Wilms' tumor 1 (WT1). Several trials also used “personalized” mixtures of 12-15 distinct peptides. That is, they contain a mixture of peptides from the patient's tumor that the patient exhibits an immune response against. Some trials are targeting solid tumors, glioma, glioblastoma, melanoma, and breast, cervical, ovarian, colorectal, and non-small lung cell cancers and include antigens from MUC1, IDO1 (Indoleamine 2,3-dioxygenase), CTAG1B, and two VEGF receptors, FLT1 and KDR. Notably, the IDO1 vaccine is tested in patients with melanoma in combination with the immune checkpoint inhibitor ipilimumab and the BRAF (gene) inhibitor vemurafenib.

Non-limiting examples of tumor antigens useful as cancer vaccines include: NY-ESO-1, HER2, HPV16 E7 (Papillomaviridae #E7), CEA (Carcinoembryonic antigen), WT1, MART-1, gp100, tyrosinase, URLC10, VEGFR1, VEGFR2, surviving, MUC1 and MUC2.

Activated immune cells primed by such cancer vaccine may, however, be excluded from the TME in part through TGFβ1-dependent mechanisms. To overcome the immunosuppression, use of isoform-specific TGFβ1 inhibitors, as described herein, may be considered so as to unleash the potential of the vaccine.

Combination therapies contemplated herein may advantageously utilize lower dosages of the administered therapeutic agents, thus avoiding possible toxicities or complications associated with the various monotherapies. In some embodiments, use of an isoform-specific inhibitor of TGFβ1 described herein may render those who are poorly responsive or not responsive to a therapy (e.g., standard of care) more responsive. In some embodiments, use of an isoform-specific inhibitor of TGFβ1 described herein may allow reduced dosage of the therapy (e.g., standard of care) which still produces equivalent clinical efficacy in patients but fewer or lesser degrees of drug-related toxicities or adverse events.

In some embodiments, the isoform-selective inhibitors of TGFβ1 contemplated herein may be used in conjunction with (e.g., combination therapy, add-on therapy, etc.) an isoform-selective inhibitor of TGFβ3. Such use may further comprise additional therapy, such as cancer therapy, e.g., immune checkpoint inhibitor, cancer vaccine, radiation therapy, and/or chemotherapy.

In some embodiments, the isoform-selective inhibitors of TGFβ1 contemplated herein may be used in conjunction with (e.g., combination therapy, add-on therapy, etc.) a selective inhibitor of myostatin (GDF8).

### Diagnostics, Patient Selection, Monitoring

Therapeutic methods that include TGFβ1 inhibition therapy may comprise diagnosis of a TGFβ1 indication and/or selection of patients likely to respond to such therapy. Additionally, patients who receive the TGFβ1 inhibitor may be monitored for therapeutic effects of the treatment, which typically involves measuring one or more suitable parameters which are indicative of the condition and which can be measured (e.g., assayed) before and after the treatment and evaluating treatment-related changes in the parameters. For example, such parameters may include levels of biomarkers present in biological samples collected from the patients. Biomarkers may be RNA-based, protein-based, cell-based and/or tissue-based. For example, genes that are overexpressed in certain disease conditions may serve as the biomarkers to diagnose and/or monitor the disease or response to the therapy. Cell-surface proteins of disease-associated cell populations may serve as biomarkers. Such methods may include the direct measurements of disease parameters indicative of the extent of the particular disease, such as tumor size/volume. Any suitable sampling methods may be employed, such as serum/blood samples, biopsies, and imaging. The biopsy may include tissue biopsies (such as tumor) and liquid biopsies.

While biopsies have traditionally been the standard for diagnosing and monitoring various diseases, such as fibrosis (e.g., organ fibrosis) and proliferative disorders (e.g., cancer), less invasive alternatives may be preferred. For example, many non-invasive in vivo imaging techniques may be used to diagnose, monitor, and select patients for treatment. Thus, the invention includes the use of in vivo imaging techniques to diagnose and/or monitor disease in a patient or subject. In some embodiments, the patient or subject is receiving an isoform-specific TGFβ1 inhibitor as described herein. In other embodiments, an in vivo imaging technique may be used to select patients for treatment with an isoform-specific TGFβ1 inhibitor. In some embodiments, such techniques may be used to determine if or how patients respond to a therapy, e.g., TGFβ1 inhibition therapy.

Exemplary in vivo imaging techniques used for the methods include, but are not limited to X-ray radiography, magnetic resonance imaging (MRI), medical ultrasonography or ultrasound, endoscopy, elastography, tactile imaging, thermography, medical photography. Other imaging techniques include nuclear medicine functional imaging, e.g., positron emission tomography (PET) and Single-photon emission computed tomography(SPECT). Methods for conducting these techniques and analyzing the results are known in the art.

Non-invasive imaging techniques commonly used to diagnose and monitor cancer include, but are not limited to: magnetic resonance imaging (MRI), computed tomography (CT), ultrasound, positron emission tomography (PET), single-photon emission computed tomography (SPECT), fluorescence reflectance imaging (FRI), and fluorescence mediated tomography (FM). Hybrid imaging platforms may also be used to diagnose and monitor cancer. For example, hybrid techniques include, but are not limited to: PET-CT, FMT-CT, FMT-MRI, and PET-MRI. Dynamic contrast enhanced MRI (DCE-MRI) is another imaging technique commonly used to detect breast cancers. Methods for conducting these techniques and analyzing the results are known in the art.

Non-invasive imaging techniques commonly used to diagnosis and monitor fibrosis include, but are not limited to: ultrasound (e.g., conventional or contrast-enhanced ultrasound), ultrasound elastography (e.g., transient elastography, point shear wave elastography and 2D-shear wave elastography), CT scan (e.g., conventional CT or CT perfusion imaging), magnetic resonance imaging (MRI) (e.g., conventional MRI, Magnetic resonance elastography, diffusion weighted magnetic resonance imaging, gadoxetic acid disodium, and magnetic resonance perfusion imaging).

In some embodiments, non-invasive imaging techniques are used to assess levels of liver fibrosis or hepatic steatosis. For example, imaging techniques particularly useful to assess liver fibrosis may include but are not limited to: FibroScan (transient elastography; TE), point shear wave elastography (pSWE; a.k.a. acoustic radiation force impulse (ARFI)), 2D-3D SWE, magnetic resonance elastography (MRE), and multiparameteric MRI. Imaging techniques particularly useful to assess hepatic steatosis may include but are not limited to: ultrasonography, controlled attenuation parameter (CAP) elastography, MRI-estimated proton density fat fraction (MRI-PDFF), and magnetic resonance spectroscopy(MRS). In some embodiments, the in vivo imaging technique is used to assess liver stiffness. In some embodiments, the in vivo imaging technique is used to detect and assess intrahepatic triglyceride levels. In some embodiments, in vivo imaging technique is used to assess liver surface nodularity (LSN; a.k.a. “liver score”), liver stiffness, and/or liver segmental volume ratio (LSVR), which are all beneficial in the staging of hepatic fibrosis and sub-staging cirrhosis. Methods for conducting these techniques and analyzing the results are known in the art.

More recently, non-invasive imaging methods are being developed which will allow the detection of cells of interest (e.g., cytotoxic T cells, macrophages, and cancer cells) in vivo. See for example, www.imaginab.com/technology/; Tavare et al. (2014) PNAS, 111(3): 1108-1113; Tavare et al. (2015) J Nucl Med 56(8): 1258-1264; Rashidian et al. (2017) J Exp Med 214(8): 2243-2255; Beckford Vera et al. (2018) PLoS ONE 13(3): e0193832; and Tavare et al. (2015) Cancer Res 76(1): 73-82, each of which is incorporated herein by reference. So-called “T-cell tracking” is aimed to detect and localize anti-tumor effector T-cells in vivo. This may provide useful insights into understanding the immunosuppressive phenotype of solid tumors. Tumors that are well-infiltrated with cytotoxic T cells (“inflamed” or “hot” tumors) are likely to respond to cancer therapies such as checkpoint blockade therapy (CBT). On the other hand, tumors with immunosuppressive phenotypes tend to have poor T-cell infiltration even when there is an anti-tumor immune response. These so-called “immune excluded” tumors likely fail to respond to cancer therapies such as CBT. T-cell tracking techniques may reveal these different phenotypes and provide information to guide in therapeutic approach that would likely benefit the patients. For example, patients with an “immune excluded” tumor are likely benefit from a TGFβ1 inhibitor therapy to help revers e the immunosuppressive phenotype. It is contemplate that similar techniques may be used to diagnose and monitor other diseases, for example, fibrosis. Typically, antibodies or antibody-like molecules engineered with a detection moiety (e.g., radiolabel, fluorescence, etc.) can be infused into a patient, which then will distribute and localize to sites of the particular marker (for instance CD8+ and M2 macrophages).

Non-invasive in vivo imaging techniques may be applied in a variety of suitable methods for purposes of diagnosing patients; selecting or identifying patients who are likely to benefit from TGFβ1 inhibitor therapy; and/or, monitoring patients for therapeutic response upon treatment. Any cells with a known cell-surface marker may be detected/localized by virtue of employing an antibody or similar molecules that specifically bind to the cell marker. Typically, cells to be detected by the use of such techniques are immune cells, such as cytotoxic T lymphocytes, regulatory T cells, MDSCs, disease-associated macrophages, (M2 macrophages such as TAMs and FAMs), NK cells, dendritic cells, and neutrophils.

Non-limiting examples of suitable immune cell markers include monocyte markers, macrophage markers (e.g., M1 and/or M2 macrophage markers), CTL markers, suppressive immune cell markers, MDSC markers (e.g., markers for G- and/or M-MDSCs), including but are not limited to: CD8, CD3, CD4, CD11b, CD163, CD206, CD68, CD14, CD15, CD66, CD34, CD25, and CD47.

In vivo imaging techniques described above may be employed to detect, localize and/or track certain MDSCs in a patient diagnosed with a TGFβ1-associated disease, such as cancer and fibrosis. Healthy individuals have no or low frequency of MDSCs in circulation. With the onset of or progression of such a disease, elevated levels of circulating and/or disease-associated MDSCs may be detected. For example, CCR2-positive M-MDSCs have been reported to accumulate to tissues with inflammation and may cause progression of fibrosis in the tissue (such as pulmonary fibrosis), and this is shown to correlate with TGFβ1 expression. Similarly, MDSCs are enriched in a number of solid tumors (including triple-negative breast cancer) and in part contribute to the immunosuppressive phenotype of the TME. Therefore, treatment response to TGFβ1 inhibition therapy according to the present disclosure may be monitored by localizing or tracking MDSCs. Reduction of or low frequency of detectable MDSCs is typically indicative of therapeutic benefits or better prognosis.

Many human cancers are known to cause elevated levels of MDSCs in patients, as compared to healthy control (reviewed, for example, in Elliott et al. (2017) “Human tumor-infiltrating myeloid cells: phenotypic and functional diversity” Frontiers in Immunology, Vol. 8, Article 86). These human cancers include but are not limited to: bladder cancer, colorectal cancer, prostate cancer, breast cancer, glioblastoma, hepatocellular carcinoma, head and neck squamous cell carcinoma, lung cancer, melanoma, NSCL, ovarian cancer, pancreatic cancer, and renal cell carcinoma. Elevated levels of MDSCs may be detected in biological samples such as peripheral blood mononuclear cell (PBMC) and tissue samples (e.g., tumor biopsy). For example, frequency of or changes in the number of MDSCs may be measured as: percent (%) of total PBMCs, percent (%) of CD14+ cells, percent (%) of CD45+ cells; percent (%) of mononuclear cells, percent (%) of total cells, percent (%) of CD11b+ cells, percent (%) of monocytes, percent (%) of non-lymphocytic MNCs, percent (%) of KLA-DR cells, using suitable cell surface markers (phenotype).

Additionally, using immune cell markers, in the case of cancer, it is possible to determine whether the tumor has an immune-excluded phenotype. If the tumor is determined to have an immune-excluded phenotype, cancer therapy (such as CBT) alone may not be efficacious because the tumor lacks sufficient cytotoxic cells within the tumor environment. Thus, an add-on therapy with a TGFβ1 inhibitor such as those described herein may reduce immuno-suppression thereby rendering the cancer therapy-resistant tumor more responsive to a cancer therapy. It is contemplated, that immune markers could also be used tot rack immune cells in the fibrotic context, and/or determine the immune cell composition of fibrotic tissue (e.g., to track the presence of macrophages and/or myofibroblasts).

Accordingly, the invention also includes a method for treating a TGFβ1-related disease or condition which may comprise the following steps: i) selecting a patient diagnosed with a TGFβ1-related disease or condition; and, ii) administering to the patient an antibody or the fragment encompassed herein in an amount effective to treat the disease or condition. In some embodiments, the selection step (i) comprises detection of disease markers (e.g., fibrosis or cancer markers as described herein), wherein optionally the detection comprises a biopsy analysis, serum marker analysis, and/or in vivo imaging. In some embodiments, the selection step (i) comprises an in vivo imaging technique as described herein.

In some embodiments, the TGFβ1-related disease or condition is a fibrotic condition. In some embodiments, the selection step (i) comprises detection of myofibroblasts cells, or one or more markers thereof. In some embodiments, the selection step (i) comprises detection of hepatic steatosis, hepatic triglycerides, immune cells, and/or myofibroblasts. In some embodiments, the detection comprises a biopsy analysis, serum marker analysis, and/or in vivo imaging. In some embodiments, the in vivo imaging comprises ultrasound, ultrasound elastography, CT scan, MRI, PET-SPECT, optical fluorescence/bioluminescence FibroScan (TE), pSWE, 2D-3D SWE, MRE, ultrasonography, CAP, MRI-PDFF, and/or MRS. In some embodiments, in vivo imaging comprises director indirect labeling of immune cells or antibody that binds a cell-surface marker of immune cells. In some embodiments, the in vivo imaging comprises the use of a tracer.

In some embodiments, the in vivo imaging technique measures hepatic steatosis, hepatic triglycerides, immune cells (e.g., as described below), and/or myofibroblasts. In some embodiments, the treatment reduces triglycerides, steatosis, liver surface nodules, inflammation, and/or macrophages, in the diseased tissue. In some embodiments, the selected patient has an intrahepatic triglyceride content of >5.5% of liver volume, optionally wherein the intrahepatic triglyceride content is >10% of liver volume. In some embodiments, the treatment reduces intrahepatic triglyceride content to <5.5% of liver volume. In some embodiments, the treatment reduces MDSCs in the diseased tissue. In some embodiments, the treatment reduces macrophages in the diseased tissue. In some embodiments, the effective amount is from 0.1 mg/kg to 30 mg/kg, optionally 3 mg/kg to 30 mg/kg. In some embodiments, the method further comprises monitoring the subject for a therapeutic response as described herein (e.g., reduced triglycerides, reduced steatosis, reduced liver surface nodules, reduced inflammation, reduced macrophages, and/or reduced liver score).

The invention also includes a method for treating cancer which may comprise the following steps: i)selecting a patient diagnosed with cancer comprising a solid tumor, wherein the solid tumor is or is suspected to be an immune excluded tumor; and, ii) administering to the patient an antibody or the fragment encompassed herein in an amount effective to treat the cancer. Preferably, the patient has received, or is a candidate for receiving a cancer therapy such as immune checkpoint inhibition therapies (e.g., PD-(L)1 antibodies), chemotherapies, radiation therapies, engineered immune cell therapies, and cancer vaccine therapies. In some embodiments, the selection step (i) comprises detection of immune cells or one or more markers thereof, wherein optionally the detection comprises a tumor biopsy analysis, serum marker analysis, and/or in vivo imaging. In some embodiments, the selection step (i) comprises an in vivo imaging technique as described here. In some embodiments, the method further comprises monitoring fora therapeutic response as described herein.

In some embodiments, in vivo imaging is performed for monitoring a therapeutic response to the TGFβ1 inhibition therapy in the subject. The in vivo imaging can comprises anyone of the imaging techniques described herein and measure anyone of the markers and/or parameters described herein. For example, in the case of liver fibrosis, the therapeutic response may comprise reduced liver steatosis, reduced triglyceride content, reduced ECM deposition/fibrosis, reduced cirrhosis, and/or reduced disease progression. In some embodiments, treatment with an isoform-specific TGFB1 inhibitor as described herein reduces intrahepatic triglyceride content to levels of <5.5% as measured by MRI. In the case of cancer, the therapeutic response may comprise conversion of an immune excluded tumor into an inflamed tumor (which correlates with increased immune cell infiltration into a tumor), reduced tumor size, and/or reduced disease progression. Increased immune cell infiltration may be visualized by increased intratumoral immune cell frequency or degree of detection signals, such as radiolabeling and fluorescence.

In some embodiments, the in vivo imaging used for diagnosing, selecting, treating, or monitoring patients, comprises MDSC tracking, such as G-MDSCs (also known as PMN-MDSCs) and M-MDSCs. For example, MDSCs may be enriched at a disease site (such as fibrotic tissues and solid tumors) at the baseline. Upon therapy (e.g., TGFβ1 inhibitor therapy), fewer MDSCs may be observed, as measured by reduced intensity of the label (such as radioisotope and fluorescence), indicative of therapeutic effects.

In some embodiments, the in vivo imaging comprises tracking or localization of LRRC33-positive cells. LRRC33-positive cells include, for example, MDSCs and activated M2-like macrophages (e.g., TAMs and activated macrophages associated with fibrotic tissues). For example, LRRC33-positive cells may be enriched at a disease site (such as fibrotic tissues and solid tumors) at the baseline. Upon therapy(e.g., TGFβ1 inhibitor therapy), fewer cells expressing cell surface LRRC33 may be observed, as measured by reduced intensity of the label (such as radioisotope and fluorescence), indicative of therapeutic effects.

In some embodiments, the in vivo imaging techniques described herein may comprise the use of PET-SPECT, MRI and/or optical fluorescence/bioluminescence in order to detect cells of interest.

In some embodiments, labeling of antibodies or antibody-like molecules with a detection moiety may comprise direct labeling or indirect labeling.

In some embodiments, the detection moiety may be a tracer. In some embodiments, the tracer may be a radioisotope, wherein optionally the radioisotope may be a positron-emitting isotope. In some embodiments, the radioisotope is selected from the group consisting of: ¹⁸F, ¹¹C, ¹³N, ¹⁵O, ⁶⁸Ga, ¹⁷Lu, and ⁸⁹Zr.

Thus, such methods may be employed to carry out in vivo imaging with the use of labeled antibodies in immune-PET.

Accordingly, the invention also includes a method for treating a TGFβ1 indication in a subject, which incorporates a step of diagnosis, patient selection, and/or monitoring therapeutic effects, which employs an imaging technique. In some embodiments, a high-affinity, isoform-selective TGFβ1 inhibitor according to the present disclosure is used in the treatment of a TGFβ1 indication, wherein the treatment comprises administration of an effective amount of the TGFβ1 inhibitor to treat the indication, and further comprising a step of monitoring therapeutic effects in the subject by in vivo imaging. Optionally, the subject may be selected as a candidate for receiving the TGFβ1 inhibitor therapy, using a diagnostic or selection step that comprises in vivo imaging. The TGFβ1 indication may be a proliferative disorder (such as cancer with a solid tumor and myelofibrosis) or a fibrotic dis order (such as organ fibrosis).

### Various Modifications and Variants

Non-limiting variations, modifications, and features of any of the antibodies or antigen-binding fragments thereof encompassed by the present disclosure are briefly discussed below. Embodiments of related analytical methods are also provided.

Naturally-occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human antibody light chains are typically classified as kappa and lambda light chains. Heavy chains are typically classified as mu, delta, gamma, alpha, or epsilon, and define the isotype of the antibody. An antibody can be of any type (e.g., IgM, IgD, IgG, IgA, IgY, and IgE) and class (e.g., IgG₁, IgG₂, IgG₃, IgG₄, IgM₁, IgM₂, IgA₁, and IgA₂). Within full-length light and heavy chains, typically, the variable and constant regions are joined by a “J” region of about 12 or more amino acids, with the heavy chain also including a “D” region of about 10 more amino acids (see, e.g., Fundamental Immunology, Ch. 7 (Paul, W., ed., 2nd ed. Raven Press, N.Y. (1989)) (incorporated by reference in its entirety)). The variable regions of each light/heavy chain pair typically form the antigen-binding site.

In some embodiments, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In any of the antibodies or antigen-binding fragments described herein, one or more conservative mutations can be introduced into the CDRs or framework sequences at positions where the residues are not likely to be involved in an antibody-antigen interaction. In some embodiments, such conservative mutation(s) can be introduced into the CDRs or framework sequences at position(s) where the residues are not likely to be involved in interacting with a GARP-TGFβ1 complex, a LTBP1-TGFβ1 complex, a LTBP3-TGFβ1 complex, and a LRRC33-TGFβ1 complex as determined based on the crystal structure. In some embodiments, likely interface (e.g., residues involved in an antigen-antibodyinteraction) may be deduced from known structural information on anotherantigen sharing structural similarities.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The variable regions typically exhibit the same general structure of relatively conserved framework regions (FR) joined by three hyper variable regions, also called complementarity determining regions orcs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk (1987) J. Mol. Biol. 196: 901-917; Chothia et al. (1989) Nature 342: 878-883. The CDRs of a light chain can also be referred to as L-CDR1, L-CDR2, and L-CDR3, and the CDRs of a heavy chain can also be referred to as H-CDR1, H-CDR2, and H-CDR3. In some embodiments, an antibody can comprise a small number of amino acid deletions from the carboxy end of the heavy chain(s). In some embodiments, an antibody comprises a heavy chain having 1-5 amino acid deletions in the carboxy end of the heavy chain. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In some embodiments, various methods of analysis can be employed to identify or approximate the CDR regions. Examples of such methods include, but are not limited to, the Kabat definition, the Chothia definition, the AbM definition, and the contact definition.

An “affinity matured” antibody is an antibody with one or more alterations in one or more CDRs thereof, which result an improvement in the affinity of the antibody for antigen compared to a parent antibody, which does not possess those alteration(s). Exemplary affinity matured antibodies will have nanomolar or even picomolar affinities for the target antigen. Affinity matured antibodies are produced by procedures known in the art. Marks et al. (1992) Bio/Technology 10: 779-783 describes affinity maturation by VH and VL domain shuffling. Random mutagenesis of CDR and/or framework residues is described by Barbas, et al. (1994) Proc Nat. Acad. Sci. USA 91: 3809-3813; Schier et al. (1995) Gene 169: 147-155; Yelton et al., (1995) J. Immunol. 155:1994-2004; Jackson et al. (1995) J. Immunol. 154(7): 3310-9; and Hawkins et al. (1992) J. Mol. Biol. 226: 889-896; and selective mutation at selective mutagenesis positions, contactor hypermutation positions with an activity enhancing amino acid residue is described in U.S. Pat. No. 6,914,128.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “chimeric antibody” refers to antibodies, which comprise heavy and light chain variable region sequences from one species and constant region sequences from another species, such as antibodies having murine heavy and light chain variable regions linked to human constant regions.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (L-CDR1, L-CDR2, and L-CDR3 of light chain and H-CDR1, H-CDR2, and H-CDR3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

In some embodiments, the antibody, or antigen-binding portion thereof, comprises a heavy chain immunoglobulin constant domain of a human IgM constant domain, a human IgG constant domain, a human IgG1 constant domain, a human IgG2 constant domain, a human IgG2A constant domain, a human IgG2B constant domain, a human IgG2 constant domain, a human IgG3 constant domain, a human IgG3 constant domain, a human IgG4 constant domain, a human IgA constant domain, a human IgA1 constant domain, a human IgA2 constant domain, a human IgD constant domain, or a human IgE constant domain. In some embodiments, the antibody, or antigen-binding portion thereof, comprises a heavy chain immunoglobulin constant domain of a human IgG1 constant domain or a human IgG4 constant domain. In some embodiments, the antibody, or antigen-binding portion thereof, comprises a heavy chain immunoglobulin constant domain of a human IgG4 constant domain. In some embodiments, the antibody, or antigen-binding portion thereof, comprises a heavy chain immunoglobulin constant domain of a human IgG4 constant domain having a backbone substitution of Ser to Pro that produces an IgG1-like hinge and permits formation of inter-chain disulfide bonds.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like (CPPCP (SEQ ID NO: 54)) hinge sequence. Accordingly, any of the antibodies may include a stabilizing ‘Adair’ mutation or the amino acid sequence CPPCP (SEQ ID NO: 54).

In some embodiments, the antibody or antigen-binding portion thereof, further comprises a light chain immunoglobulin constant domain comprising a human Ig lambda constant domain or a human Ig kappa constant domain.

In some embodiments, the antibody is an IgG having four polypeptide chains which are two heavy chains and two light chains.

In some embodiments, wherein the antibody is a humanized antibody, a diabody, or a chimeric antibody. In some embodiments, the antibody is a humanized antibody. In some embodiments, the antibody is a human antibody. In some embodiments, the antibody comprises a framework having a human germline amino acid sequence.

In some embodiments, the antigen-binding portion is a Fab fragment, a F(ab′)2 fragment, a scFab fragment, or an scFv fragment.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al. (2002) Crit. Rev. Immunol. 22(3): 183-200; Marchalonis et al. (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen when a binding protein specifically binds to the antigen. In an embodiment, the neutralizing binding protein binds to the antigen/target, e.g., cytokine, kinase, growth factor, cell surface protein, soluble protein, phosphatase, or receptor ligand, and reduces its biologicallyactivity by at least about 20%, 40%, 60%, 80%, 85%, 90%, 95%, 96%, 97%, 98%, 99% or more.

The term “binding protein” as used herein includes any polypeptide that specifically binds to an antigen (e.g., TGFβ1), including, but not limited to, an antibody, or antigen-binding portions thereof, a DVD-Ig™, a TVD-Ig, a RAb-Ig, a bispecific antibody and a dual specific antibody.

The term “monoclonal antibody” or “mAb” when used in a context of a composition comprising the same may refer to an antibody preparation obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is motto be construed as requiring production of the antibody by any particular method.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35:425-445; Gavilondo, J. V. and Larrick, J. W. (2002) BioTechniques 29:128-145; Hoogenboom, H. and Chames, P. (2000) Immunol. Today 21: 371-378, incorporated herein by reference), antibodies isolated from an animal (e.g., a mouse) that is transgenic for human immunoglobulin genes (s ee, Taylor, L. D. et al. (1992) Nucl. Acids Res. 20: 6287-6295; Kellermann, S-A. and Green, L. L. (2002) Cur. Opin. in Biotechnol. 13: 593-597; Little, M. et al. (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In some embodiments, the antibody or antigen-binding portion, is an antibody fragment, e.g., (i) Fab fragments, a monovalent fragment consisting of the VL, VH, CL and CH1 domains; (ii) F(ab′)2 fragment, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv(scFv) molecules; (vi) dAb fragments; or(vii) minimal recognition units consisting of the amino acid residues that mimic the hypervariable region of an antibody (e.g., an isolated complementarity determining region (CDR)). In some embodiments, the antibody or antigen-binding portion, is (i) a “Dual Variable Domain Immunoglobulin” or “DVD-Ig™,” (ii) a “Triple Variable Domain Immunoglobulin” or “TVD-Ig”, (iii) a “Receptor-Antibody Immunoglobulin” or “RAb-Ig,” (iv) a “bispecific antibody,” or (v) a “dual-specific antibody,”

As used herein, “Dual Variable Domain Immunoglobulin” or “DVD-Ig™” and the like include binding proteins comprising a paired heavy chain DVD polypeptide and a light chain DVD polypeptide with each paired heavy and light chain providing two antigen-binding sites. Each binding site includes a total of 6 CDRs involved in antigen-binding per antigen-binding site. A DVD-Ig™ is typically has two arms bound to each other at least in part by dimerization of the CH3 domains, with each arm of the DVD being bispecific, providing an immunoglobulin with four binding sites. DVD-Ig™ are provided in US Patent Publication Nos. 2010/0260668 and 2009/0304693, each of which are incorporated herein by reference including sequence listings.

As used herein, “Triple Variable Domain Immunoglobulin” or “TVD-Ig” and the like are binding proteins comprising a paired heavy chain TVD binding protein polypeptide and a light chain TVD binding protein polypeptide with each paired heavy and light chain providing three antigen-binding sites. Each binding site includes a total of 6 CDRs involved in antigen-binding per antigen-binding site. A TVD binding protein may have two arms bound to each other at least in part by dimerization of the CH3 domains, with each arm of the TVD binding protein being trispecific, providing a binding protein with six binding sites.

As used herein, “Receptor-Antibody Immunoglobulin” or “RAb-Ig” and the like are binding proteins comprising a heavy chain RAb polypeptide, and a light chain RAb polypeptide, which together form three antigen-binding sites in total. One antigen-binding site is formed by the pairing of the heavy and light antibody variable domains present in each of the heavy chain RAb polypeptide and the light chain RAb polypeptide to form a single binding site with a total of 6 CDRs providing a first antigen-binding site. Each the heavy chain RAb polypeptide and the light chain RAb polypeptide include a receptor sequence that independently binds a ligand providing the second and third “antigen” binding sites. A RAb-Ig is typically has two arms bound to each other at least in part by dimerization of the CH3 domains, with each arm of the RAb-Ig being trispecific, providing an immunoglobulin with six binding sites. RAb-Igs are described in US Patent Application Publication No. 2002/0127231, the entire contents of which including sequence listings are incorporated herein by reference).

The term “bispecific antibody,” as used herein, and as differentiated from a “bispecific half-Ig binding protein” or “bispecific (half-Ig) binding protein”, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al. (1985) Nature 314(6012): 628-631), or by knob-into-hole or similar approaches, which introduce mutations in the Fc region that do not inhibit CH3-CH3 dimerization (see Holliger, P. et al. (1993) Proc. Natl. Acad. Sci USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen it binds to.

The term “dual-specific antibody,” as used herein, and as differentiated from a bispecific half-Ig binding protein or bispecific binding protein, refers to full-length antibodies that can bind two different antigens (or epitopes) in each of its two binding arms (a pair of HC/LC) (see PCT Publication No. WO 02/02773). Accordingly, a dual-specific binding protein has two identical antigen-binding arms, with identical specificity and identical CDR sequences, and is bivalent for each antigen to which it binds.

The term “Kon,” as used herein, is intended to refer to the on rate constant for association of a binding protein (e.g., an antibody) to the antigen to form the, e.g., antibody/antigen complex as is known in the art. The “Kon” also is known by the terms “association rate constant,” or “ka,” as used interchangeably herein. This value indicating the binding rate of an antibody to its target antigen or the rate of complex formation between an antibody and antigen also is shown by the equation: Antibody (“Ab”)+Antigen (“Ag”)→Ab−Ag.

The term “Koff” as used herein, is intended to refer to the off rate constant for dissociation of a binding protein (e.g., an antibody) from the, e.g., antibody/antigen complex as is known in the art. The “Koff” also is known by the terms “dissociation rate constant” or“kdis” as used interchangeably herein. This value indicates the dissociation rate of an antibody from its target antigen or separation of Ab−Ag complex overtime into free antibody and antigen as shown by the equation: Ab+Ag←Ab−Ag.

The terms “equilibrium dissociation constant” or “KD,” as used interchangeably herein, refer to the value obtained in a titration measurement at equilibrium, or by dividing the dissociation rate constant (koff) by the association rate constant (kon). The association rate constant, the dissociation rate constant, and the equilibrium dissociation constant are used to represent the binding affinity of a binding protein, e.g., antibody, to an antigen. Methods for determining association and dissociation rate constants are well known in the art. Using fluorescence-based techniques offers high sensitivity and the ability to examine samples in physiological buffers at equilibrium. Other experimental approaches and instruments, such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The terms “crystal” and “crystallized” as used herein, refer to a binding protein (e.g., an antibody), or antigen-binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 201-16, Oxford University Press, New York, N.Y., (1999).

The term “linker” is used to denote polypeptides comprising two or more amino acid residuesjoined bypeptide bonds and are used to link one or more antigen-binding portions. Such linker polypeptides are well known in the art (see, e.g., Holliger, P. et al. (1993) Proc. Natl. Acad. Sci. USA 90: 6444-6448; Poljak, R. J. et al. (1994) Structure 2:1121-1123). Exemplary linkers include, but are not limited to, ASTKGPSVFPLAP (SEQ ID NO: 55), ASTKGP (SEQ ID NO: 56); TVAAPSVFIFPP (SEQ ID NO: 57); TVAAP (SEQ ID NO: 58); AKTTPKLEEGEFSEAR (SEQ ID NO: 59); AKTTPKLEEGEFSEARV (SEQ ID NO: 60); AKTTPKLGG (SEQ ID NO: 61); SAKTTPKLGG (SEQ ID NO: 62); SAKTTP (SEQ ID NO: 63); RADAAP (SEQ ID NO: 64); RADAAPTVS (SEQ ID NO: 65); RADAAAAGGPGS (SEQ ID NO: 66); RADAAAA(G4S)4 (SEQ ID NO: 67); SAKTTPKLEEGEFSEARV (SEQ ID NO: 68); ADAAP (SEQ ID NO: 69); ADAAPTVSIFPP (SEQ ID NO: 70); QPKAAP (SEQ ID NO: 71); QPKAAPSVTLFPP (SEQ ID NO: 72); AKTTPP (SEQ ID NO: 73); AKTTPPSVTPLAP (SEQ ID NO: 74); AKTTAP (SEQ ID NO: 75); AKTTAPSVYPLAP (SEQ ID NO: 76); GGGGSGGGGSGGGGS (SEQ ID NO: 77); GENKVEYAPALMALS (SEQ ID NO: 78); GPAKELTPLKEAKVS (SEQ ID NO: 79); GHEAAAVMQVQYPAS (SEQ ID NO: 80); TVAAPSVFIFPPTVAAPSVFIFPP (SEQ ID NO: 81); and ASTKGPSVFPLAPASTKGPSVFPLAP (SEQ ID NO: 82).

“Label” and “detectable label” or “detectable moiety” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Thus, the term “labeled binding protein” as used herein, refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³S, ⁹⁰Y, ⁹⁹Tc, ¹¹¹In, ¹²⁵I, ¹³¹I, ¹⁷⁷Lu, ¹⁶⁶Ho, and ¹⁵³Sm); chromogens; fluorescent labels (e.g., FITC, rhodamine, and lanthanide phosphors); enzymatic labels (e.g., horseradish peroxidase, luciferase, and alkaline phosphatase); chemiluminescent markers; biotinyl groups; predetermined polypeptide epitopes recognized by a secondary reporter (e.g., leucine zipper pair sequences, binding sites for secondary antibodies, metal binding domains, and epitope tags); and magnetic agents, such as gadolinium chelates. Representative examples of labels commonly employed for immunoassays include moieties that produce light, e.g., acridinium compounds, and moieties that produce fluorescence, e.g., fluorescein. Other labels are described herein. In this regard, the moiety its elf may not be detectably labeled but may become detectable upon reaction with yet another moiety. Use of “detectably labeled” is intended to encompass the latter type of detectable labeling.

In some embodiments, the binding affinity of an antibody, or antigen-binding portion thereof, to an antigen (e.g., protein complex), such as presenting molecule-proTGFβ1 complexes, is determined using an Octet ass ay. In some embodiments, an Octet assay is an assay that determines one or more a kinetic parameters indicative of binding between an antibody and antigen. In some embodiments, an Octet® system (ForteBio, Menlo Park, Calif.) is used to determine the binding affinity of an antibody, or antigen-binding portion thereof, to presenting molecule-proTGFβ1 complexes. For example, binding affinities of antibodies may be determined using the fortéBio Octet QKe dip and read label free assay system utilizing bio-layer interferometry. In some embodiments, antigens are immobilized to biosensors (e.g., streptavidin-coated biosensors) and the antibodies and complexes (e.g., biotinylated presenting molecule-proTGFβ1 complexes) are presented in solution at high concentration (50 μg/mL) to measure binding interactions. In some embodiments, the binding affinity of an antibody, or antigen-binding portion thereof, to a presenting molecule-proTGFβ1 complex is determined using the protocol outlined in Table 14.

The antibodies according to the present disclosure include pH-sensitive antibodies. In some embodiments, such antibodies or fragments thereof bind the target complex in a pH-dependent manner such that relatively high affinity binding occurs at a neutral or physiological pH, but the antibody dissociates from its antigen more rapidly at an acidic pH; or, dissociation rates are higher at acidic pH than at neutral pH. Such antibodies or fragments thereof may function as recycling antibodies. Such antibodies may also be referred to as “pH-sensitive” antibodies.

Thus, the invention encompasses pH-sensitive antibodies that selectively bind a proTGFβ1 complex characterized in that the antibodies have lower dissociation rates at a neutral pH (e.g., around pH 7) as compared to at an acidic pH (e.g., around pH 5). PH-dependent binding profiles of one such antibody are provided in Example 14 herein.

In some embodiments, the antibodies according to the present disclosure may induce internalization of the complex comprising proTGFβ1 bound to LRRC33 or GARP on cell surface. In some embodiments, the antibodies are inhibitors of cell-associated TGFβ1 (e.g., GARP-presented proTGFβ1 and LRRC33-presented proTGFβ1) according to the invention include antibodies or fragments thereof that specifically bind such complex (e.g., GARP-pro/latent TGFβ1 and LRRC33-pro/latent TGFβ1), thereby triggering internalization of the complex (e.g., endocytosis). This mode of action causes removal or depletion of the inactive TGFβ1 complexes from the cell surface (e.g., Treg, macrophages, MDSCs, etc.), hence reducing latent TGFβ1 available for activation. Such antibodies or fragments thereof may function as recycling antibodies. Such antibodies may also be referred to as “pH-sensitive” antibodies.

In some embodiments, such “pH sensitive” antibodies have a K_{dis} (a.k.a. K_{off}) of ≤5×10⁻³ s⁻¹ (e.g., ≥5.1×10⁻³, ≥5.2×10⁻³, ≥5.3×10⁻³, ≥5.4×10⁻³, ≥5.5×10⁻³, ≥5.6×10⁻³, ≥5.7×10⁻³, ≥5.8×10⁻³, ≥5.9×10⁻³, or ≥6.0×10⁻³) at pH 5, as measured by a suitable affinity assay (e.g., biolayer interferometry, surface plasmon resonance, and/or solution equilibrium titration). In a particular embodiment, such “pH-sensitive” antibodies have a K_{dis}5.6×10⁻³ at pH 5.

In some embodiments, such “pH-sensitive” antibodies have a pH 5 K_{dis} to pH 7 K_{dis} ratio (i.e., K_{dis} at pH 5:K_{dis} at pH7) of ≥1.5 (e.g., ≥1.6, ≥1.7, ≥1.8, ≥1.9, or ≥2.0), as measured by a suitable affinity assay (e.g., biolayer interferometry, surface plasmon resonance, and/or solution equilibrium titration). In a particular embodiment, such “pH-sensitive” antibodies have a K_{dis} ratio of ≥2.0, as measured by biolayer interferometry.

### Process of Screening; Manufacture

The invention encompasses screening methods, production methods and manufacture processes of antibodies or fragments thereof which bind each of: a GARP-proTGFβ1 complex, a LTBP1-proTGFβ1 complex, a LTBP3-proTGFβ1 complex, and a LRRC33-proTGFβ1 complex, and pharmaceutical compositions and related kits comprising the same.

Numerous methods may be used for obtaining antibodies, or antigen-binding fragments thereof, of the disclosure. For example, antibodies can be produced using recombinant DNA methods. Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET® or BIACORE) analysis, to identify one or more hybridomas that produce an antibody that specifically binds to a specified antigen. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597; WO 92/18619; WO 91/17271; WO 92/20791; WO 92/15679; WO 93/01288; WO 92/01047; WO 92/09690; and WO 90/02809.

In addition to the use of display libraries, the specified antigen (e.g., presenting molecule-TGFβ1 complexes) can be used to immunize a non-human host, e.g., rabbit, guinea pig, rat, mouse, hamster, sheep, goat, chicken, camelid, as well as non-mammalian hosts such as shark. In one embodiment, the non-human animal is a mouse.

In another embodiment, a monoclonal antibody is obtained from the non-human animal, and then modified, e.g., chimeric, using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

For additional antibody production techniques, see Antibodies: A Laboratory Manual, eds. Harlow et al., Cold Spring Harbor Laboratory, 1988. The present disclosure is not necessarily limited to any particular source, method of production, or other special characteristics of an antibody.

Some aspects of the present disclosure relate to host cells transformed with a polynucleotide or vector. Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen-binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen-binding fragments. Furthermore, transgenic animals, preferably mammals, comprising the aforementioned host cells may be used for the large scale production of the antibody or antibody fragments.

The transformed host cells can be grown in fermenters and cultured using any suitable techniques to achieve optimal cell growth. Once expressed, the whole antibodies, their dimers, individual light and heavy chains, other immunoglobulin forms, or antigen-binding fragments, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen-binding fragments can then be isolated from the growth medium, cellular lysates, or cellular membrane fractions. The isolation and purification of the, e.g., microbially expressed antibodies or antigen-binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen-binding fragment may be undertaken by culturing a modified recombinant host under culture conditions appropriate for the growth of the host cells and the expression of the coding sequences. The antibodies or antigen-binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen-binding fragments may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter(Rous sarcoma virus), CMV-enhancer, SV40-enhancer or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, polynucleotides may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen-binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen-binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

The screening methods may include a step of evaluating or confirming desired activities of the antibody or fragment thereof. In some embodiments, the step comprises selecting for the ability to inhibit target function, e.g., inhibition of release of mature/soluble growth factor(e.g., TGFβ1) from a latent complex. In preferred embodiments, such step comprises a cell-based potency assay, in which inhibitory activities of test antibody or antibodies are assayed by measuring the level of growth factor released in the medium (e.g., assay solution) upon activation, when proTGFβ complex is expressed on cell surface or present in the ECM. The level of growth factor released into the medium/solution can be assayed by, for example, measuring TGFβ activities. Non-limiting examples of useful cell-based potency assays are described in below (see, e.g., “Cell-Based Assays for Measuring TGFβ Activation and/or Inhibitory Potency”) and Example 3 herein.

In some embodiments, the screening method comprises the step foremoving antibodies that have an IC50 of greater than 5 nM (e.g., greater than 10 nM) as measured by a suitable cell-based potency assay. In some embodiments, the screening method comprises the step foremoving antibodies that have an IC50 of greater than 5 nM (e.g., greater than 10 nM) as measured by a suitable cell-based potency assay against a LTBP1-TGFβ1, LTBP3-TGFβ1, GARP-TGFβ1, and/or LRRC33-TGFβ1.

In some embodiments, the screening method comprises the step of selecting antibodies based on their bias (or non-bias) for one or more presenting molecule-TGFb1 affinities. Accordingly, in some embodiments, the screening method comprises selecting antibodies having a bias for matrix-associated TGFβ1 complexes. In some embodiments, the screening method comprises selecting antibodies having relatively equivalent affinities for a GARP-TGFβ1 complex, a LTBP1-TGFβ1 complex, a LTBP3-TGFβ1 complex, and a LRRC33-TGFβ1 complex.

In some embodiments, the screening method comprises the step of selecting for antibodies or fragments thereof that induce ADCC. In some embodiments, the step comprises selecting for antibodies or fragments thereof that accumulate to a desired site(s) in vivo (e.g., cell type, tissue or organ). In some embodiments, the step comprises selecting for antibodies or fragments thereof with the ability to cross the blood brain barrier. The methods may optionally include a step of optimizing one or more antibodies or fragments thereof to provide variant counterparts that possess desirable profiles, as determined by criteria such as stability, binding affinity, functionality (e.g., inhibitory activities, Fc function, etc.), immunogenicity, pH sensitivity and developability(e.g., high solubility, low self-association, etc.).

In some embodiments, the screening method comprises the step of selecting antibodies that are pH-sensitive. In some embodiments, the screening method comprises the step of selecting antibodies that have a K_{dis} of ≥5×10⁻³ s⁻¹ (e.g., ≥5.1×10⁻³, ≥5.2×10⁻³, ≥5.3×10⁻³, ≥5.4×10⁻³, ≥5.5×10⁻³, ≥5.6×10⁻³, ≥5.7×10⁻³, ≥5.8×10⁻³, ≥5.9×10⁻³, or ≥6.0×10⁻³) at pH 5, as measured by a suitable affinity assay (e.g., biolayer interferometry, surface plasmon resonance, and/or solution equilibrium titration). In some embodiments, the screening method comprises the step of selecting antibodies that have a pH 5 K_{dis} to pH 7 K_{dis} ratio (i.e., K_{dis} at pH 5:K_{dis} at pH7) of ≥1.5 (e.g., ≥1.6, ≥1.7, ≥1.8, ≥1.9, or ≥2.0), as measured by a suitable affinity assay (e.g., biolayer interferometry, surface plasmon resonance, and/or solution equilibrium titration).

In some embodiments, the screening method comprises the step of selecting antibodies that are cross-reactive with proTGFβ1 from other species (e.g., mouse, rat, and/or cynomolgus).

### Modifications

Antibodies, or antigen-binding portions thereof, of the disclosure may be modified with a detectable label or detectable moiety, including, but not limited to, an enzyme, prosthetic group, fluorescent material, luminescent material, bioluminescent material, radioactive material, positron emitting metal, nonradioactive paramagnetic metal ion, and affinity label for detection and isolation of a GARP-proTGFβ1 complex, a LTBP1-proTGFβ1 complex, a LTBP3-proTGFβ1 complex, and/or a LRRC33-proTGFβ1 complex. The detectable substance or moiety may be coupled or conjugated either directly to the polypeptides of the disclosure or indirectly, through an intermediate (such as, for example, a linker (e.g., a cleavable linker)) using suitable techniques. Non-limiting examples of suitable enzymes include horseradish peroxidase, alkaline phosphatase, ρ-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (¹³¹I, ¹²⁵I, ¹²³I, ¹²¹I), carbon (¹⁴C), sulfur(³⁵S), tritium (³H), indium (¹¹⁵mIn, ¹¹³mIn, ¹¹²In ¹¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, Lu, ¹⁵⁹Gd, ¹⁴⁹Pm, ¹⁴⁰La, ¹⁷⁵Yb, ¹⁶⁶Ho, ⁹⁰Y, ⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵⁹Gd, ¹⁶⁹Yb, ⁵¹Cr, ⁵⁴Mn, ⁷⁵Se, zirconium (⁸⁹Zr) and tin (¹¹³Sn, ¹¹⁷Sn). The detectable substance may be coupled or conjugated either directly to the antibodies of the disclosure that bind specifically to a GARP-proTGFβ1 complex, a LTBP1-proTGFβ1 complex, a LTBP3-proTGFβ1 complex, and/or a LRRC33-proTGFβ1 complex, or any components thereof, or indirectly, through an intermediate (such as, for example, a linker) using suitable techniques. Any of the antibodies provided herein that are conjugated to a detectable substance may be used foray suitable diagnostic assays, such as those described herein. Such assays include in vivo imaging, which may be used for monitoring disease progression and/or response to a therapy, such as TGFβ1 inhibition therapy described herein.

In addition, antibodies, or antigen-binding portions thereof, of the disclosure may also be modified with a drug. The drug may be coupled or conjugated either directly to the polypeptides of the disclosure, or indirectly, through an intermediate (such as, for example, a linker(e.g., a cleavable linker)) using suitable techniques.

### Targeting Agents

In some embodiments methods of the present disclosure comprise the use of one or more targeting agents to target an antibody, or antigen-binding portion thereof, as disclosed herein, to a particular site in a subject for purposes of modulating mature TGFβ release from a GARP-proTGFβ1 complex, a LTBP1-proTGFβ1 complex, a LTBP3-proTGFβ1 complex, and/or a LRRC33-proTGFβ1 complex. For example, LTBP1-proTGFβ1 and LTBP3-proTGFβ1 complexes are typically localized to extracellular matrix. Thus, in some embodiments, antibodies disclosed herein can be conjugated to extracellular matrix targeting agents for purposes of localizing the antibodies to sites where LTBP-associated TGFβ1 complexes reside. In such embodiments, selective targeting of antibodies leads to selective modulation of LTBP1-proTGFβ1 and LTBP3-proTGFβ1 complexes. In some embodiments, extracellular matrix targeting agents include heparin binding agents, matrix metalloproteinase binding agents, lysyl oxidase binding domains, fibrillin-binding agents, hyaluronic acid binding agents, and others.

Similarly, GARP-proTGFβ1 and LRRC33-proTGFβ1 complexes are typically localized and anchored to the surface of cells. The former is expressed on activated FOXP3+ regulatory T cells (Tregs), while the latter is expressed on myeloid cells and some cancer cells such as AML. Thus, in some embodiments, antibodies disclosed herein can be conjugated to immune cell (e.g., Treg cell, activated macrophages, etc.) binding agents for purposes of localizing antibodies to sites where these cell-associated proTGFβ1 complexes reside. In such embodiments, selective targeting of antibodies leads to selective inhibition of cell associated-proTGFβ1 complexes (e.g., selective inhibition of the release of mature TGFβ1 for purposes of immune modulation, e.g., in the treatment of cancer). In such embodiments, immune cell targeting agents may include, for example, CCL22 and CXCL12 proteins or fragments thereof.

In some embodiments, bispecific antibodies may be used having a first portion that selectively binds a proTGFβ1 complex and a second portion that selectively binds a component of a target site, e.g., a component of the ECM (e.g., fibrillin) or a component of a Treg cell (e.g., CTLA-4).

As further detailed herein, the present invention contemplates that isoform-selective TGFβ1 inhibitors, such as those described herein, may be used for promoting or restoring hematopoiesis in the bone marrow. Accordingly, in some embodiments, a composition comprising such an inhibitor (e.g., high-affinity, isoform-selective inhibitor of TGFβ1) may be targeted to the bone marrow. One mode of achieving bone marrow targeting is the use of certain carriers that preferentially target the bone marrow localization or accumulation. For example, certain nanoparticle-based carriers with bone marrow-targeting properties may be employed, e.g., lipid-based nanoparticles or liposomes. See, for example, Sou (2012) “Advanced drug carriers targeting bone marrow”, Research Gate publication 232725109.

### Pharmaceutical Compositions and Formulations

The invention further provides pharmaceutical compositions used as a medicament suitable for administration in human and non-human subjects. One or more isoform-specific antibodies encompassed by the invention can be formulated or admixed with a pharmaceutically acceptable carrier(excipient), including, for example, a buffer, to form a pharmaceutical composition. Such formulations may be used for the treatment of a disease or disorder that involves TGFβ signaling. In particularly preferred embodiments, such formulations may be used for immuno-oncology applications.

The pharmaceutical compositions of the invention may be administered to patients for alleviating a TGFβ-related indication (e.g., fibrosis, immune disorders, and/or cancer). “Acceptable” means that the carrier is compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. In one example, a pharmaceutical composition described herein contains more than one antibody that specifically binds a GARP-proTGFβ1 complex a LTBP1-proTGFβ1 complex a LTBP3-proTGFβ1 complex and a LRRC33-proTGFβ1 complex where the antibodies recognize different epitopes/residues of the complex.

The pharmaceutical compositions to be used in the present methods can comprise pharmaceutically acceptable carriers, excipients, or stabilizers in the form of lyophilized formulations or aqueous solutions (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The invention also includes pharmaceutical compositions that comprise an antibody or fragment thereof according to the present invention, and a pharmaceutically acceptable excipient.

Thus, the antibody or a molecule comprising an antigen-binding fragment of such antibody can be formulated into a pharmaceutical composition suitable for human administration.

The pharmaceutical formulation may include one or more excipients. In some embodiments, excipient(s) may be selected from the list provided in the following: https://www.accessdata.fda.gov/scripts/cder/iig/index.Cfm?event=browseByLetter.page&Letter=A

The pharmaceutical composition is typically formulated to a final concentration of the active biologic (e.g., monoclonal antibody, engineered binding molecule comprising an antigen-binding fragment, etc.) to be between about 2 mg/mL and about 200 mg/mL. For example, the final concentration (wt/vol) of the formulations may range between about 2-200, 2-180, 2-160, 2-150, 2-120, 2-100, 2-80, 2-70, 2-60, 2-50, 2-40, 5-200, 5-180, 5-160, 5-150, 5-120, 5-100, 5-80, 5-70, 5-60, 5-50, 5-40, 10-200, 10-180, 10-160, 10-150, 10-120, 10-100, 10-80, 10-70, 10-60, 10-50, 10-40, 20-200, 20-180, 20-160, 20-150, 20-120, 20-100, 20-80, 20-70, 20-60, 20-50, 20-40, 30-200, 30-180, 30-160, 30-150, 30-120, 30-100, 30-80, 30-70, 30-60, 30-50, 30-40, 40-200, 40-180, 40-160, 40-150, 40-120, 40-100, 40-80, 40-70, 40-60, 40-50, 50-200, 50-180, 50-160, 50-150, 50-120, 50-100, 50-80, 50-70, 50-60, 60-200, 60-180, 60-160, 60-150, 60-120, 60-100, 60-80, 60-70, 70-200, 70-180, 70-160, 70-150, 70-120, 70-100, 70-80 mg/mL. In some embodiments, the final concentration of the biologic in the formulation is about 10, 15, 20, 25, 30, 35, 40, 45, 50, 55, 60, 65, 70, 75, 80, 85, 90, 95, 100, 110, 120, 130, 140, 150, 160, 170, 180, 190, or 200 mg/mL.

The pharmaceutical compositions of the present invention are preferably formulated with suitable buffers. Suitable buffers include but are not limited to: phosphate buffer, citric buffer, and histidine buffer.

The final pH of the formulation is typically between pH 5.0 and 8.0. For example, the pH of the pharmaceutical composition may be about 5.0, 5.2, 5.5, 6.0, 6.2, 6.5, 6.8, 7.0, 7.2, 7.4, 7.5, 7.6, or 7.8.

The pharmaceutical composition of the present disclosure may comprise a surfactant, such as nonionic detergent, approved for the use in pharmaceutical formulations. Such surfactants include, for example, polysorbates, such as Polysorbate 20 (Tween-20), Polysorbate 80 (Tween-80) and NP-40.

The pharmaceutical composition of the present disclosure may comprise a stabilizer. For liquid-protein preparations, stability can be enhanced by election of pH-buffering salts, and often amino acids can also be used. It is often interactions at the liquid/air interface or liquid/solid interface (with the packaging) that lead to aggregation following adsorption and unfolding of the protein. Suitable stabilizers include but are not limited to: sucrose, maltose, sorbitol, as well as certain amino acids such as histidine, glycine, methionine and arginine.

The pharmaceutical composition of the present disclosure may contain one or any combinations of the following excipients: Sodium Phosphate, Arginine, Sucrose, Sodium Chloride, Tromethamine, Mannitol, Benzyl Alcohol, Histidine, Sucrose, Polysorbate 80, Sodium Citrate, Glycine, Polysorbate 20, Trehalose, Poloxamer 188, Methionine, Trehalose, rhHyaluronidase, Sodium Succinate, Potassium Phosphate, Disodium Edetate, Sodium Chloride, Potassium Chloride, Maltose, Histidine Acetate, Sorbitol, Pentetic Acid, Human Serum Albumin, Pentetic Acid.

In some embodiments, the pharmaceutical composition of the present disclosure may contain a preservative.

The pharmaceutical composition of the present disclosure is typically presented as a liquid or a lyophilized form. Typically, the products can be presented in Vial (e.g., glass Vial). Products available in syringes, pens, or autoinjectors may be presented as pre-filled liquids in these container/closure systems.

In some examples, the pharmaceutical composition described herein comprises liposomes containing an antibody that specifically binds a GARP-proTGFβ1 complex, a LTBP1-proTGFβ1 complex, a LTBP3-proTGFβ1 complex, and a LRRC33-proTGFβ1 complex, which can be prepared by any suitable method, such as described in Epstein et al., Proc. Natl. Acad. Sci. USA 82:3688 (1985); Hwang et al. Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, liposomes with targeting properties are selected to preferentially deliver or localize the pharmaceutical composition to certain tissues or cell types. For example, certain nanoparticle-based carriers with bone marrow-targeting properties may be employed, e.g., lipid-based nanoparticles or liposomes. See, for example, Sou (2012) “Advanced drug carriers targeting bone marrow”, Research Gate publication 232725109.

In some embodiments, pharmaceutical compositions of the invention may comprise or may be used in conjunction with an adjuvant. It is contemplated that certain adjuvant can boost the subject's immune responses to, for example, tumor antigens, and facilitate T effector function, DC differentiation from monocytes, enhanced antigen uptake and presentation by APCs, etc. Suitable adjuvants include but are not limited to retinoic acid-based adjuvants and derivatives thereof, oil-in-water emulsion-based adjuvants, such as MF59 and other squalene-containing adjuvants, Toll-like receptor(TRL) ligands, α-tocopherol (vitamin E) and derivatives thereof.

The antibodies described herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

Suitable surface-active agents include, in particular, non-ionic agents, such as polyoxyethylene sorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared BMXing an antibody of the invention with Intralipid™ or the components thereof (soybean oil, egg phospholipids, glycerol and water).

### Inhibition of TGFβ1 Activity

Methods of the present disclosure include methods of inhibiting TGFβ1 growth factor activity in one or more biological system. Such methods may include contacting one or more biological system with an antibody and/or composition of the disclosure. In some cases, these methods include modifying the level of free growth factor in a biological system (e.g. in a cell niche or subject). Antibodies and/or compositions according to such methods may include, but are not limited to biomolecules, including, but not limited to recombinant proteins, protein complexes and/or antibodies, or antigen portions thereof, described herein.

In some embodiments, methods of the present disclosure may be used to reduce or eliminate growth factor activity, termed “inhibiting methods” herein. Some such methods may comprise mature growth factor retention in a TGFβ complex (e.g., a TGFβ1 complexed with GARP, LTBP1, LTBP3 and/or LRRC33) and/or promotion of reassociation of growth factor into a TGFβ complex. In some cases, inhibiting methods may comprise the use of an antibody disclosed herein. According to some inhibiting methods, one or more inhibiting antibody is provided.

In some embodiments, antibodies, antigen-binding portions thereof, and compositions of the disclosure may be used for inhibiting TGFβ1 activation. In some embodiments, provided herein is a method for inhibiting TGFβ1 activation comprising exposing a GARP-TGFβ1 complex, a LTBP1-TGFβ1 complex, a LTBP3-TGFβ1 complex, and/or a LRRC33-TGFβ1 complex to an antibody, an antigen-binding portion thereof, or a pharmaceutical composition described herein. In some embodiments, the antibody, antigen-binding portion thereof, or pharmaceutical composition, inhibits the release of mature TGFβ1 from the GARP-TGFβ1 complex, the LTBP1-TGFβ1 complex, a LTBP3-TGFβ1 complex, and/or the LRRC33-TGFβ1 complex. In some embodiments, the method is performed in vitro. In some embodiments, the method is performed in vivo. In some embodiments, the method is performed ex vivo.

In some embodiments, the GARP-TGFβ1 complex or the LRRC33-TGFβ1 complex is present at the outer surface of a cell.

In some embodiments, the cell expressing the GARP-TGFβ1 complex or the LRRC33-TGFβ1 complex is a fibroblast, a myofibroblast, a macrophage, a T-cell, a monocyte, a dendritic cell, an antigen presenting cell, a neutrophil, a myeloid-derived suppressor cell (MDSC), a lymphocyte, a mast cell, or a microglia. The myofibroblast may be a fibrosis-associated fibroblast (FAF) of a cancer-associated fibroblasts (CAF). The T-cell may be a regulatory T cell (e.g., immunosuppressive T cell). The neutrophil may be an activated neutrophil. The macrophage may be a resident macrophage (e.g, a liver kupffer cell) or an infiltrating macrophage. The macrophage may be an activated (e.g., polarized) macrophage, including profibrotic and/or tumor-associated macrophages (TAM), e.g., M2c subtype and M2d subtype macrophages. In some embodiments, macrophages are exposed to tumor-derived factors (e.g., cytokines, growth factors, etc.) which may further induce pro-cancer phenotypes in macrophages. In some embodiments, such tumor-derived factor is CSF-1/M-CSF.

In some embodiments, the cell expressing the GARP-TGFβ1 complex or the LRRC33-TGFβ1 complex is a cancer cell, e.g., circulating cancer cells and tumor cells.

In some embodiments, the LTBP1-TGFβ1 complex or the LTBP3-TGFβ1 complex is bound to an extracellular matrix (i.e., components of the ECM). In some embodiments, the extracellular matrix comprises fibrillin and/or fibronectin. In some embodiments, the extracellular matrix comprises a protein comprising an RGD motif.

LRRC33 is expressed in selective cell types, in particular those of myeloid lineage, including monocytes and macrophages. Monocytes originated from progenitors in the bone marrow and circulate in the bloodstream and reach peripheral tissues. Circulating monocytes can then migrate into tissues where they become exposed to the local environment (e.g., tissue-specific, disease-associated, etc.) that includes a panel of various factors, such as cytokines and chemokines, triggering differentiation of monocytes into macrophages, dendritic cells, etc. These include, for example, alveolar macrophages in the lung, osteoclasts in bone marrow, microglia in the CNS, histiocytes in connective tissues, Kupffer cells in the liver, and brown adipose tissue macrophages in brown adipose tissues. In fibrotic tissues, infiltrated myeloid cells may include fibrosis-associated macrophages (FAM) which are typically M2-like macrophages, as well as MDSCs. In a solid tumor, infiltrated macrophages may be tumor-associated macrophages (TAMs), tumor-associated neutrophils (TANs), and myeloid-derived suppressor cells (MDSCs), etc. Such macrophages may activate and/or be associated with activated fibroblasts, such as carcinoma-associated (or cancer-associated) fibroblasts (CAFs) and/or the stroma. Thus, inhibitors of TGFβ1 activation described herein which inhibits release of mature TGFβ1 from LRRC33-containing complexes can target any of these cells expressing LRRC33-proTGFβ1 on cell surface.

In some embodiments, the LRRC33-TGFβ1 complex is present at the outer surface of profibrotic (M2-like) macrophages. In some embodiments, the profibrotic (M2-like) macrophages are present in the fibrotic microenvironment. In some embodiments, targeting of the LRRC33-TGFβ1 complex at the outer surface of profibrotic (M2-like) macrophages provides a superior effect as compared to solely targeting LTBP1-TGFβ1 and/or LTBP1-TGFβ1 complexes. In some embodiments, M2-like macrophages, are further polarized into multiple subtypes with differential phenotyles, such as M2c and M2d TAM-like macrophages. In some embodiments, macrophages may become activated by various factors (e.g., growth factors, chemokines, cytokines and ECM-remodeling molecules) present in the tumor microenvironment, including but are not limited to TGFβ1, CCL2 (MCP-1), CCL22, SDF-1/CXCL12, M-CSF (CSF-1), IL-6, IL-8, IL-10, IL-11, CXCR4, VEGF, PDGF, prostaglandin-regulating agents such as arachidonic acid and cyclooxygenase-2 (COX-2), parathyroid hormone-related protein (PTHrP), RUNX2, HIF1α, and metalloproteinases. Exposures to one or more of such factors may further drive monocytes/macrophages into pro-tumor phenotypes. To give but one example, CCL2 and VEGF co-expression in tumors has been shown to be correlated with increased TAM and poor diagnosis. In turn, these activated tumor-associated cells may also facilitate recruitment and/or differentiation of other cells into pro-tumor cells, e.g., CAFs, TANs, MDSCs, and the like. Stromal cells may also respond to macrophage activation and affect ECM remodeling, and ultimately vascularization, invasion, and metastasis. For example, CCL2 not only functions as a monocyte attractant but also promotes cell adhesion by upregulating MAC-1, which is a receptor for ICAM-1, expressed in activated endothelium. This may lead to CCL2-dependent arteriogenesis and cancer progression. Thus, TGFβ1 inhibitors described herein may be used in a method for inhibiting arteriogenesis by interfering with the CCL2 signaling ads.

In some embodiments, the GARP-TGFβ1 complex, the LTBP1-TGFβ1 complex, the LTBP3-TGFβ1 complex, and/or the LRRC33-TGFβ1 complex is bound to an extracellular matrix. In some embodiments, the extracellular matrix comprises fibrillin. In some embodiments, the extracellular matrix comprises a protein comprising an RGD motif.

In some embodiments, provided herein is a method for reducing TGFβ1 protein activation in a subject comprising administering an antibody, an antigen-binding portion thereof, or a pharmaceutical composition described herein to the subject, thereby reducing TGFβ1 protein activation in the subject. In some embodiments, the subject has or is at risk of having fibrosis. In some embodiments, the subject has or is at risk of having cancer. In some embodiments, the subject has or is at risk of having dementia.

In some embodiments, the antibodies, or the antigen-binding portions thereof, as described herein, reduce the suppressive activity of regulatory T cells (Tregs).

Cell-Based Assays for Measuring TGFβ Activation and/or Inhibitory Potency

Activation of TGFβ (and inhibition thereof by a TGFβ test inhibitor, such as an antibody) may be measured by any suitable method known in the art. For example, integrin-mediated activation of TGFβ can be utilized in a cell-based assay, such as the “CAGA12” luciferase assay, described in more detail herein.

The development of novel context-dependent cell-based assays of TGFβ1 activation is described in U.S. Provisional Patent Application No. 62/538,476 and the corresponding International Application WO 2019/023661, each of which are incorporated by reference in their entirety herein. Previous assay formats could not differentiate between the activation of proTGFβ1 presented by endogenous presenting molecules and the activation of proTGFβ1 presented by exogenous LTBPs. By directly transfecting integrin-expressing cells, the novel assays disclosed in International Application WO 2019/023661, and used herein, establish a window between endogenous presenter-proTGFβ1 activity and exogenous LTBP-proTGFβ1 activity.

As opposed to GARP- or LRRC33-proTGFβ1 complexes, which are presented on the surface of cells, LTBP-proTGFβ1 complexes are embedded in the extracellular matrix. Thus, the assay plate coating is an important component of the assay when assessing activation of proTGFβ1 in complex with LTBP (e.g., LTBP1/3). In this regard, it has been shown, fibronectin and fibrillin are two ECM components that appear to be critical for LTBP association with the matrix and activation of latent TGFβ (Robertson et al., Matrix Biol. 2015 September; 47:44-53). For example, LTBP3 ECM-incorporation appears to be dependent on fibrillin expression in both in vitro and in vivo models (Zilberberg et al., J Cell Physiol. 2012; 227(12):3828-3836).

On the other hand, LTBP1 has been shown to interact with fibrillin microfibrils and fibronectin via its C- and N-termini, respectively (Dallas et al., J Biol Chem. 2005; 280(19):18871-18880; Fontana et al., FASEB J. 2005; 19(13):1798-1808; and Kantola et al., Exp Cell Res. 2008; 314(13):2488-2500). Moreover, in the absence of fibrillin, LTBP1 still co-localizes with fibronectin fibers (Robertson et al., Matrix Biol. 2015 September; 47:44-53). LTBP1 has also been shown to interact with ADAMTSL2 and 3 (Sengle et al., PLoS Genet. 2012; 8(1):e1002425), IGFBP3 (Gui and Murphy, Mol Cell Biochem. 2003; 250(1-2):189-195), fibulin-4 (Massam-Wu et al., J Cell Sci. 2010 Sep. 1; 123(Pt 17):3006-18), and heparin (Chen et al. J Biol Chem. 2007; 282(36):26418-26430).

Beyond traditional ECM components/proteins, tissue transglutaminase (TG2) may also playa critical role in TGFβ localization in the ECM. TG2 is known to catalyze inter- and intramolecular isopeptide bonds which cross-link ECM fibrils, effectively stiffening the ECM and protecting the ECM from proteolytic degradation (Benn et al., Current Opinion in Biomedical Engineering., 2019, https://doi.org/10.1016/j.cobme.2019.06.003). Moreover, TG2 can cross-link LTBP1 to the ECM, thus promoting a matrix reservoir of TGFβ (Nunes et al, J Cell Biol. 1997; 136(5):1151-1163).

Accordingly, the N-terminus of LTBPs may be covalently bound to the ECM via an isopeptide bond, the formation of which may be catalyzed by transglutaminases. The structural integrity of the ECM is believed to be important in mediating LTBP-associated TGFβ1 activity. For example, stiffness of the matrix can significantly affect TGFβ1 activation. In addition, incorporating fibronectin and/or fibrillin in the scaffold may significantly increase the LTBP-mediated TGFβ1 activation. Similarly, presence of fibronectin and/or fibrillin in LTBP assays (e.g., cell-based potency assays) may increase an assay window.

Accordingly, a cell-based assay for measuring TGFβ1 activation may comprise the following components: i) a source of TGFβ (recombinant, endogenous or transfected); ii) a source of activator such as integrin (recombinant, endogenous, or transfected); and iii) a reporter system that responds to TGFβ activation, such as cells expressing TGFβ receptors capable of responding to TGFβ and translating the signal into a readable output (e.g., luciferase activity in CAGA12 cells or other reporter cell lines). In some embodiments, the reporter cell line comprises a reporter gene (e.g., a luciferase gene) under the control of a TGFβ-responsive promoter (e.g., a PAI-1 promoter). In some embodiments, certain promoter elements that confer sensitivity may be incorporated into the reporter system. In some embodiments, such promoter element is the CAGA12 element. Reporter cell lines that may be used in the assay have been described, for example, in Abe et al. (1994) Anal Biochem. 216(2): 276-84, incorporated herein by reference. In some embodiments, each of the aforementioned assay components are provided from the same source (e.g., the same cell). In some embodiments, two of the aforementioned assay components are provided from the same source, and a third assay component is provided from a different source. In some embodiments, all three assay components are provided from different sources. For example, in some embodiments, the integrin and the latent TGFβ complex (proTGFβ and a presenting molecule) are provided for the assay from the same source (e.g., the same transfected cell line). In some embodiments, the integrin and the TGF are provided for the assay from separate sources (e.g., two different cell lines, a combination of purified integrin and transfected cell). When cells are used as the source of one or more of the assay components, such components of the assay may be endogenous to the cell, stably expressed in the cell, transiently transfected, or any combination thereof. In some embodiments, the assay is performed in a tissue culture plate or dish. In some embodiments, the tissue culture plate or dish is coated with a component of the extracellular matrix (ECM). In some embodiments, the tissue culture plate or dish is coated with fibronectin and/or fibrillin. In some embodiments, the cell-based assay further comprises a fourth component comprising a source of TG2. In some embodiments, the TG2 component is provided from the same, or different, source as any one of the above-mentioned components. The results from a non-limiting exemplary embodiment of a cell-based assay for measuring TGFβ activation demonstrating the inhibition of LTBP1-proTGFβ1 complex, LTBP3-proTGFβ1 complex, GARP-proTGFβ1 complex, or LRRC33-proTGFβ1 complex are disclosed herein (see, e.g., Example 3).

A skilled artisan could readily adapt such assays to various suitable configurations. For instance, a variety of sources of TGFβ may be considered. In some embodiments, the source of TGFβ is a cell that expresses and deposits TGFβ (e.g., a primary cell, a propagated cell, an immortalized cell or cell line, etc.). In some embodiments, the source of TGFβ is purified and/or recombinant TGFβ immobilized in the assay system using suitable means. In some embodiments, TGFβ immobilized in the assay system is presented within an extracellular matrix (ECM) composition on the assay plate, with or without de-cellularization, which mimics fibroblast-originated TGFβ. In some embodiments, TGFβ is presented on the cell surface of a cell used in the assay. Additionally, a presenting molecule of choice may be included in the assay system to provide suitable latent-TGFβ complex. One of ordinary skill in the art can readily determine which presenting molecule(s) may be present or expressed in certain cells or cell types. Using such assay systems, relative changes in TGFβ activation in the presence or absence of a test agent (such as an antibody) may be readily measured to evaluate the effects of the test agent on TGFβ activation in vitro. Data fro m exemplary cell-based assays are provided in the Example section below.

Such cell-based assays may be modified or tailored in a number of ways depending on the TGFβ isoform being studied, the type of latent complex (e.g., presenting molecule), and the like. In some embodiments, a cell known to express integrin capable of activating TGFβ may be used as the source of integrin in the assay. Such cells typically include LN229 cells. Other suitable cells include SW480/P6 cells (e.g., clone 1E7). In some embodiments, the cell-line(s) may be modified to reduce or eliminate expression of one or more presenting molecules (e.g., through CRISPR-mediated gene ablation). For example, the cell-line may be a LTBP1 knock-out cell-line (e.g., CRISPR-mediated gene ablation by targeting exon 7). In other embodiments, the cell-line may be a LTBP3 knock-out cell-line. In other embodiments, the cell-line may be a GARP knock-out cell-line. In other embodiments, the cell-line may be a LRRC33 knock-out cell-line.

In some embodiments, integrin-expressing cells may be co-transfected with a plasmid encoding a presenting molecule of interest (such as GARP, LRRC33, LTBP (e.g., LTBP1 or LTBP3), etc.) and a plasmid encoding a pro-form of the TGFβ isoform of interest(such as proTGFβ1). After transfection, the cells are incubated for sufficient time to allow for the expression of the transfected genes (e.g., about 24 hours), cells are washed, and incubated with serial dilutions of a test agent (e.g., an antibody). Then, a reporter cell line (e.g., CAGA12 cells) is added to the assay system, followed by appropriate incubation time to allow TGFβ signaling. After an incubation period (e.g., about 18-20 hours) following the addition of the test agent, signal/read-out(e.g., luciferase activity) is detected using suitable means (e.g., for luciferase-expressing reporter cell lines, the Bright-Glo reagent(Promega) can be used). In some embodiments, Luciferase fluorescence may be detected using a BioTek (Synergy H1) plate reader, with autogain settings.

Data demonstrate that exemplary antibodies of the invention which are capable of selectively inhibiting the activation of TGFβ1 (see, e.g., Example 3 and Table 15).

### Nucleic Acids

In some embodiments, antibodies, antigen-binding portions thereof, and/or compositions of the present disclosure may be encoded by nucleic acid molecules. Such nucleic acid molecules include, without limitation, DNA molecules, RNA molecules, polynucleotides, oligonucleotides, mRNA molecules, vectors, plasmids and the like. In some embodiments, the present disclosure may comprise cells programmed or generated to express nucleic acid molecules encoding compounds and/or compositions of the present disclosure. In some cases, nucleic acids of the disclosure include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety.

The present invention is further illustrated by the following examples, which are not intended to be limiting in any way. The entire contents of all references, patents and published patent applications cited throughout this application, as well as the Figures, are hereby incorporated herein by reference.

Kits for Use in Alleviating Diseases/Disorders Associated with a TGFβ-Related Indication

The present disclosure also provides kits for use in alleviating diseases/disorders associated with a TGFβ-related indication. Such kits can include one or more containers comprising an antibody, or antigen-binding portion thereof, as described herein.

In some embodiments, the kit can comprise instructions for use in accordance with any of the methods described herein. The included instructions can comprise a description of administration of the antibody, or antigen-binding portion thereof, to treat, delay the onset, or alleviate a target disease as those described herein. The kit may further comprise a description of selecting an individual suitable for treatment based on identifying whether that individual has the target disease. In still other embodiments, the instructions comprise a description of administering an antibody, or antigen-binding portion thereof, to an individual at risk of the target disease.

The instructions relating to the use of antibodies, or antigen-binding portions thereof, generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder associated with a TGFβ-related indication. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a Vial having a stopper pierceable by a hypodermic injection needle). The container may also have a sterile access port (for example the container may be an intravenous solution bag or a Vial having a stopper pierceable by a hypodermic injection needle). At least one active agent in the composition is an antibody, or antigen-binding portion thereof, as described herein.

Kits may optionally provide additional components such as buffers and interpretive information. Normally, the kit comprises a container and a label or package insert(s) on or associated with the container. In some embodiments, the disclosure provides articles of manufacture comprising contents of the kits described above.

This invention is further illustrated by the following examples which should note construed as limiting. The examples refer to the following figures:

### EXAMPLES

### Example 1: Antibody Screening and Selection Methodology

Ab3 was affinity optimized using standard affinity optimization protocols. Briefly, Ab3 was subjected to multiple sequential cycles of library based antibody engineering approaches to achieve affinity improvements. In the first cycle the antibody was put through Light Chain Shuffle (LCS) and H-CDR1 and H-CDR2 sequence diversification. The best clones from this cycle were moved into the next cycle of affinity optimization where antibodies with new light chains with or without sequence diversification in H-CDR1 & 2 was subjected to H-CDR3 sequence diversification. In all cycles of affinity optimization the libraries were subjected to multiple sequential rounds of selections on all four proTGFb1 large latent complexes with affinity pressure applied using antigen titration and cold antigen competition strategies.

### Example 2. In Vitro Binding Profiles

The affinities of Ab1, Ab2 and Ab3 were measured by Octet® assay on human proTGFβ1 cells, while activity was measured by CAGA12 reporter cells testing human proTGFβ1 inhibition. The protocol used to measure the affinity of the antibodies to the complexes provided herein is summarized in Table 14 below, and a summary list of affinity profiles of exemplary antibodies of the present disclosure is provided in Table 6 herein.

TABLE 14
Protocol for performing Octet binding assay

Materials:
96 well black polypropylene plates
Streptavidin-coated tips for Octet
10x kinetics buffer (diluted 1:10 in PBS)

| | |
|---|---|
| 1. | Soak required amount of streptavidin tips in 1X kinetics buffer; place in machine to equilibrate |
| 2. | Load sample plate: |
| | 200 μl of buffer or antibody dilution to each well |

| | |
|---|---|
| a. | Column 1 - baseline (buffer) |
| b. | Column 2 - biotinylated protein (e.g., sGARP-proTGFβ1 |
| | μor LTBP1-proTGFβ1); diluted to 5 g/mL |
| c. | Column 3 - baseline 2 (buffer) |
| d. | Column 4 - antibody association for Ab |
| e. | Column 5 - antibody association for Ab |
| f. | Column 6 - dissociation Ab (buffer) |
| g. | Column 7 - dissociation Ab (buffer) |

| | |
|---|---|
| 3. | Make dilutions in the 96 well plate: |

| | |
|---|---|
| a. | Dilute both antibodies to 50 μg/mL in |
| | 300 μl of 1x buffer in row A. |
| b. | Add 200 μl of buffer to the rest of each |
| | column |
| c. | Transfer 100 μl down the column to |
| | make 3-fold dilutions |

| | |
|---|---|
| 4. | Place the sample plate in the machine next to the tips plate |
| 5. | Set up the software |

| | |
|---|---|
| a. | Indicate buffer, load, sample (one assay per antibody tested) |
| b. | Indicate steps of the protocol (baseline, load, association, |
| | dissociation) for set amounts of time: |

Baseline: 60 seconds
Loading: 300 seconds
Baseline 2: 60 seconds
Association: 300 seconds
Dissociation: 600 seconds

| | |
|---|---|
| 6. | Analyze data |

| | |
|---|---|
| a. | Subtract baseline from reference well |
| b. | Set normalization to last five seconds of baseline |
| c. | Align to dissociation |
| d. | Analyze to association and dissociation |
| | (1:1 binding model, fit curves) |
| e. | Determine the best R² values; include |
| | concentrations with best R² values |
| f. | Select global fit |
| g. | Set colors of samples by sensor type |
| h. | Analyze |
| i. | Save table and export |

The affinities of Ab2, Ab3, Ab11, Ab12, Ab19, Ab20, and Ab35, C1, and C2 were also measured by Meso-Scale Discovery (MSD) Solution Equilibrium Titration (SET). MSD-SET is a well-characterized technique which can be used for the determination of solution-phase equilibrium K_{D}. Solution-based equilibrium assays such as MSD-SET are based on the principle of kinetic exclusion, in which free ligand binding at equilibrium rather than real-time association and dissociation rates is measured to determine affinity.

In brief, standard MSD plates were coated with 20 nM solution of monoclonal antibody of interest (capture antibody) for 30 minutes at room temperature or overnight at 4° C. On a separate non-binding 96 well plate, the same monoclonal antibody used as the capture antibody is then titrated from μM to fM concentrations and incubated with one set concentration of biotinylated antigen overnight at room temperature without shaking. After 20-24 hours of equilibration, the capture antibody plate is blocked and washed before adding the equilibrated antibody-antigen sample solutions to the plate for exactly 150 seconds. The plate is then washed prior to addition of streptavidin-sulfotag secondary reagent for 3 minutes. Plates are washed prior to reading in MSD read buffer using the MESO® QuickPlex SQ 120. The affinity profiles by MSD-SET as described above are provided in Table 7 herein.

### Example 3: Functional Assays to Detect Inhibition of Activated Recombinant Latent TGFβ1

The development of novel context-dependent cell-based assays of TGFβ1 activation is described in U.S. Provisional Patent Application No. 62/538,476, incorporated by reference in its entirety herein. Previous assay formats could not differentiate between the activation of proTGFβ1 presented by endogenous presenting molecules and the activation of proTGFβ1 presented by exogenous LTBPs. By directly transfecting integrin-expressing cells, the novel assays disclosed in U.S. Provisional Patent Application No. 62/538,476, and used herein, establish a window between endogenous presenter-proTGFβ1 activity and exogenous LTBP-proTGFβ1 activity. As LTBP-proTGFβ1 complexes are embedded in the extracellular matrix, the assay plate coating is also an important component of the assay. The use of tissue culture plates, coated with the ECM protein Fibronectin, made the LTBP assays more robust.

To determine if any of the isoform-specific antibodies described herein were functional, cell-based assays of αVβ integrin activation of TGFβ1 large latent complex (LLC) were developed, which are specific for each known presenting molecule: LTBP1, LTBP3, GARP and LRRC33. Through the process of assay development and optimization, it was determined that fibronectin is a critical ECM protein for the integrin-dependent in vitro activation of LTBP presented TGFβ1 LLCs.

Assay I. Activation of Latent TGFβ1 Deposited in the ECM

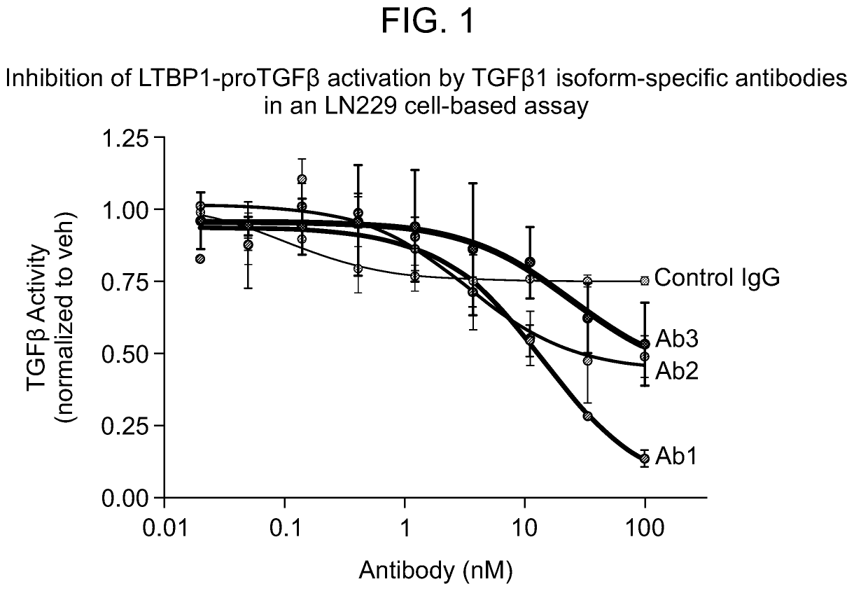

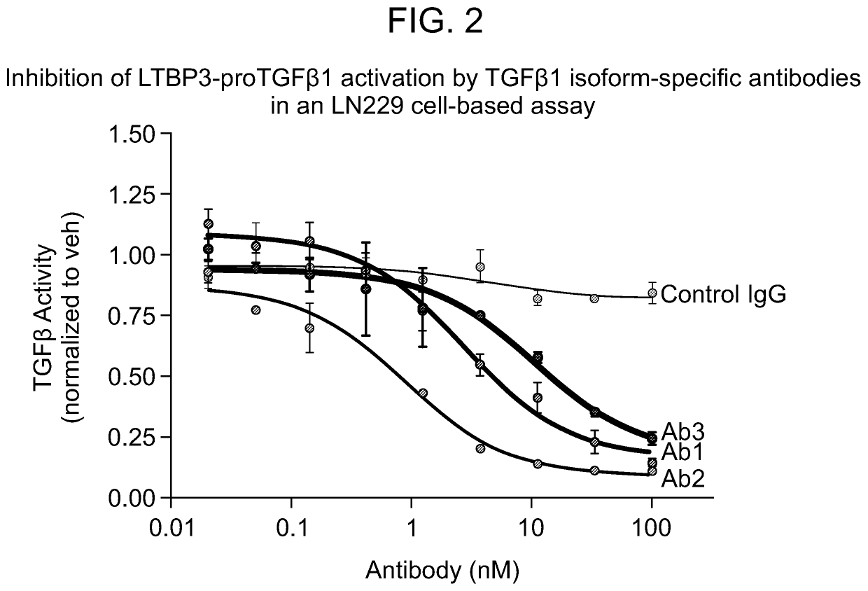

For the assays depicted in FIG. 1 and FIG. 2, the following protocol was developed. This assay is optimal for extracellular matrix (LTBP presented) activation by integrin cells.

Materials:
MvLu1-CAGA12 cells (Clone 4A4)
SW480/β6 cells (Clone 1E7) (αV subunit is endogenously expressed at high levels; P6 subunit is stably overexpressed)
LN229 cell line (high levels of endogenous αVβ8 integrin)
Costar white walled TC treated 96 well assay plate #3903
Greiner Bio-One High Binding white ρclear 96 well assay plate #655094
Human Fibronectin (Corning #354008)
P200 multichannel pipet
P20, P200, and P1000 pipets with sterile filter tips for each
Sterile microfuge tubes and rack
Sterile reagent reservoirs
0.4% trypan blue
2 mL, 5 mL, 10 mL, and 25 mL sterile pipets
Tissue culture treated 100 mm or 150 mm plates
70% Ethanol
Opti-MEM reduced serum media (Life Tech #31985-070)
Lipofectamine 3000 (Life Tech #L3000015)
Bright-Glo luciferase assay reagent (Promega #E2620)
0.25% Tryspin+0.53 mM EDTA
proTGFβ1 expression plasmid, human
LTBP1 S expression plasmid, human
LTBP3 expression plasmid, human
LRRC32 (GARP) expression plasmid, human
LRRC33 expression plasmid, human

Equipment:
BioTek Synergy H1 plate reader
TC hood
Bench top centrifuge
CO2 incubator 37C 5% CO2
37 C water/bead bath
Platform shaker
Microscope
Hemocytometer/countess

Definitions:
CAGA12 4A4 cells: Derivative of MvLu1 cells (Mink Lung Epithelial Cells), stably transfected with CAGA12 synthetic promoter, driving luciferase gene expression
DMEM-0.1% BSA: Assay media; base media is DMEM (Gibco Cat #11995-065), media also contains BSA diluted to 0.1% w/v, penicillin/streptinomycin, and 4 mM glutamine
D10:DMEM 10% FBS, P/S, 4 mM glutamine, 1% NEAA, 1× GlutaMAX (Gibco Cat #35050061)
SW480/β6 Media: D10+1000 ug/mL G-418
CAGA12 (4A4) media: D10+0.75 ug/mL puromycin

Procedure:

On Day 0, cells were seeded for transfection. SW480/P6 (clone 1E7) cells were detached with trypsin and pellet (spin 5 min @ 200×g). Cell pellet was resuspended in D10 media and viable cells per ml were counted. Cells were seeded at 5.0×10⁶ cells/12 ml/100 mm tissue culture dish. For CAGA12 cells, cells were passaged at a density of 1.0 million per T75 flask, to be used for the assay on Day 3. Cultures were incubated at 37° C. and 5% CO₂.

On Day 1, integrin-expressing cells were transfected. Manufacturer's protocol for transfection with Lipofectamine® 3000 reagent was followed. Briefly, the following were diluted into OptiMEM™ I, for 125 μl per well: 7.5 μg DNA (presenting molecule)+7.5 μg DNA (proTGFβ1), 30 μl P3000, and Up to 125 μl with OptiMEM I. The well was mixed by pipetting DNA together, then OptiMEM was added. P3000 was added, and everything was mixed well by pipetting. A master mix of Lipofectamine3000 was made, to be added to DNA mixes: for the LTBP1 assay: 15 μl Lipofectamine3000, up to 125 μl in OptiMEM I, per well; for the LTBP3 assay: 45 ul Lipofectamine3000, up to 125 μl in OptiMEM I, per well. Diluted Lipofectamine3000 was added to DNA, mixed well by pipetting, and incubated at room temp for 15 min. After the incubation, the solution was mixed a few times by pipetting, and then 250 μl of DNA:Lipofectamine3000 (2×125 μl) per dis h was added dropwise. Each dish was gently swirled to mix and the dish was returned to the tissue culture incubator for ˜24 hrs.

On Days 1-2, the assay plates were coated with human fibronectin. Specifically, lyophilized fibronectin was diluted to 1 mg/ml in ultra-pure distilled water(sterile). 1 mg/ml stock solution was diluted to 19.2 μg/ml in PBS (sterile). Added 50 μl/well to assay plate (high binding) and incubated O/N in tissue culture incubator (37° C. and 5% CO2). Final concentration was 3.0 μg/cm².

On Day 2, transfected cells were plated for assay and inhibitor addition. First, the fibronectin coating was washed by adding 200 μl/well PBS to the fibronectin solution already in the assay plate. Removed wash manually with multichannel pipette. Wash was repeated for two washes total. The plate was allowed to dry at room temperature with lid off prior to cell addition. The cells were then plated by detaching with trypsin and pellet (spin 5 min @ 200×g.). The pellet was resuspended in assay media and viable cells were counted per ml. For the LTBP1 assay cells were diluted to 0.10×10⁶ cells/ml and seed 50 μl per well (5,000 cells per well). For the LTBP3 assay, cells were diluted to 0.05×10⁶ cells/ml and seed 50 μl per well (2,500 cells per well). To prepare functional antibody dilutions, antibodies were pre-diluted to a consistent working concentration in vehicle. Stock antibodies were serially diluted in vehicle (PBS is optimal, avoid sodium citrate buffer). Each point of serial dilution was diluted into assay media for a 4× final concentration of antibody. Added 25 μl per well of 4× antibody and incubated cultures at 37° C. and 5% CO₂ for ˜24 hours.

On Day 3, the TGFβ reporter cells were added. CAGA12 (clone 4A4) cells for the assay were detached with trypsin and pellet(spin 5 min @ 200×g.). The pellet was resuspended in assay media and count viable cells per ml. Cells were diluted to 0.4×10⁶ cell/ml and seed 50 μl per well (20,000 cells per well). Cells were returned to incubator.

On Day 4, the assay was read (16-20 hours after antibody and/or reporter cell addition). Bright-Glo reagent and test plate were allowed to come to room temperature before reading. Read settings on BioTekSynergy H1 were set using TMLC_std protocol—this method has an auto-gain setting. Selected positive control wells for autoscale (high). 100 μL of Bright-Glo reagent was added per well. Incubated for 2 min with shaking, at room temperature, protected plate from light. The plate was read on BioTek Synergy H1.

FIG. 1 depicts inhibition of LTBP1-proTGFβ1 activation in the LN229 assay. FIG. 2 depicts inhibition of LTBP3-proTGFβ1 complex activation in LN229 cells.

Assay II. Activation of Latent TGFβ1 Presented on the Cell Surface

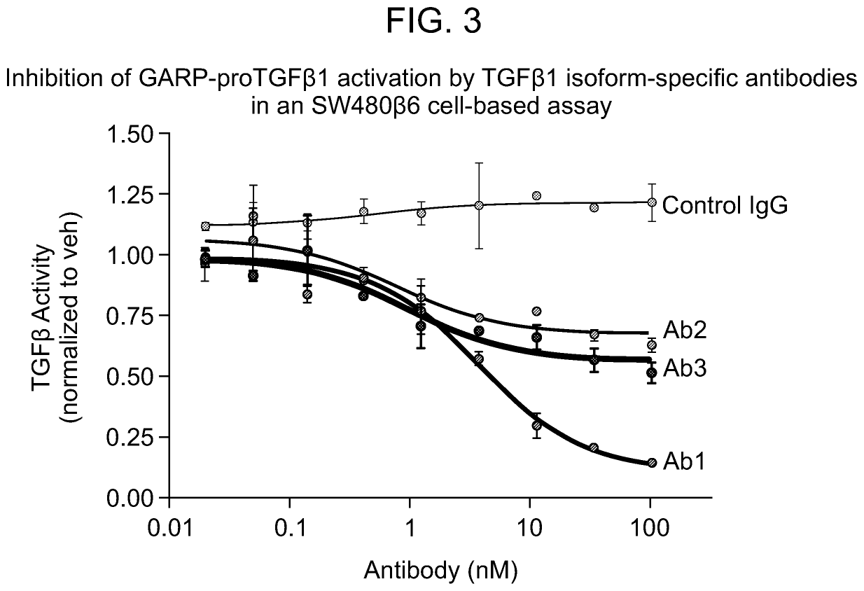

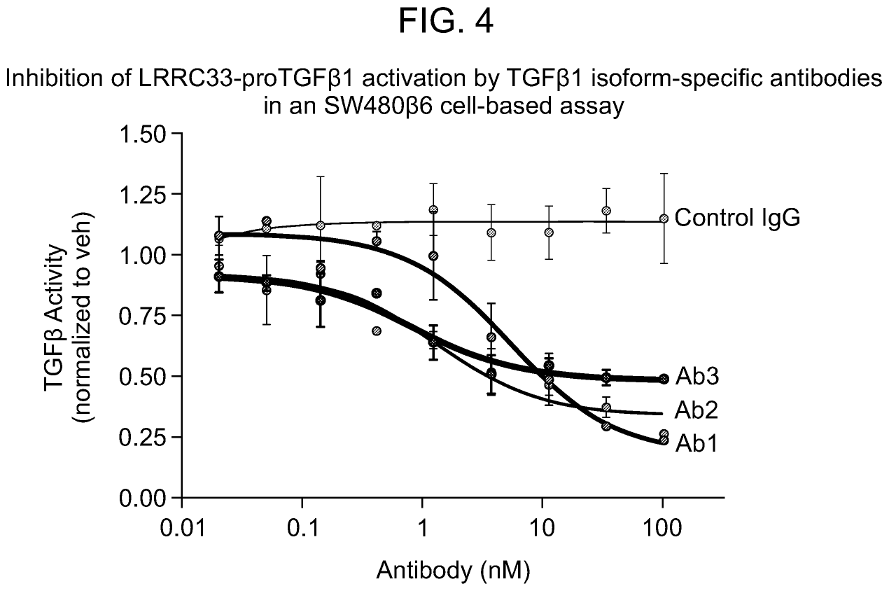

For the assay depicted in FIG. 3 and FIG. 4, the following protocol was developed. This assay, or “direct-transfection” protocol, is optimal for cell-surface presented TGFβ1 (GARP or LRRC33 presenter) activation by integrin cells.

Materials:
MvLu1-CAGA12 cells (Clone 4A4)
SW480/β6 cells (Clone 1E7) (αV subunit is endogenously expressed at high levels; P6 subunit is stably overexpressed)
LN229 cell line (high levels of endogenous αVβ8 integrin)
Costar white walled TC treated 96 well assay plate #3903
Greiner Bio-One High Binding white ρclear 96 well assay plate #655094
Human Fibronectin (Corning #354008) P200 multichannel pipet
P20, P200, and P1000 pipets with sterile filter tips for each
Sterile microfuge tubes and rack
Sterile reagent reservoirs
0.4% trypan blue
2 mL, 5 mL, 10 mL, and 25 mL sterile pipets
Tissue culture treated 100 mm or 150 mm plates
70% Ethanol
Opti-MEM reduced serum media (Life Tech #31985-070)
Lipofectamine 3000 (Life Tech #L3000015)
Bright-Glo luciferase assay reagent(Promega #E2620)
0.25% Tryspin+0.53 mM EDTA
proTGFβ1 expression plasmid, human
LTBP1 S expression plasmid, human
LTBP3 expression plasmid, human
LRRC32 (GARP) expression plasmid, human
LRRC33 expression plasmid, human

Equipment:
BioTek Synergy H1 plate reader
Tissue culture hood
Bench top centrifuge
CO₂ incubator 37° C. 5% CO₂
37° C. water/bead bath
Platform shaker
Microscope
Hemocytometer/countess

Definitions:
CAGA12 4A4 cells: Derivative of MvLu1 cells (Mink Lung Epithelial Cells), stably transfected with CAGA12 synthetic promoter, driving luciferase gene expression
DMEM-0.1% BSA: Assay media; base media is DMEM (Gibco Cat #11995-065), media also contains BSA diluted to 0.1% w/v, penicillin/streptinomycin, and 4 mM glutamine
D10:DMEM 10% FBS, P/S, 4 mM glutamine, 1% NEAA, 1× GlutaMAX (GibcoCat #35050061)
SW480/β6 Media: D10+1000 ug/mL G-418
CAGA12 (4A4) media: D10+0.75 ug/mL puromycin

Methods:

On Day 0, integrin expressing cells were seeded for transfection. Cells were detached with trypsin and pelleted (spin 5 min @ 200×g). Cell pellet was resuspended in D10 media and count viable cells per ml. Cells were diluted to 0.1×10⁶ cells/ml and seeded 100 μl per well (10,000 cells per well) in an assay plate. For CAGA12 cells, passaged at a density of 1.5 million per T75 flask, to be used for the assay on Day 2. Cultures were incubated at 37° C. and 5% CO₂.

On Day 1, cells were transfected. The manufacturer's protocol was followed for transfection with Lipofectamine 3000 reagent. Briefly, the following was diluted into OptiMEM I, for 5 μl per well: 0.1 μg DNA (presenting molecule)+0.1 μg DNA (proTGFβ1), 0.4 μl P3000, and up to 5 μl with OptiMEM I. The well was mixed by pipetting DNA together, then add OptiMEM. Add P3000 and mix everything well by pipetting. A master mix was made with Lipofectamine3000, to be added to DNA mixes: 0.2 μl Lipofectamine3000, up to 5 μl in OptiMEM I, per well. Diluted Lipofectamine3000 was added to DNA, mixed well by pipetting, and incubated at room temp for 15 min. After the incubation, the solution was mixed a few times by pipetting, and then 10 ul per well of DNA:Lipofectamine3000 (2×5 μl) was added. The cell plate was returned to the tissue culture incubator for ˜24 hrs.

On Day 2, the antibody and TGFβ reporter cells were added. In order to prepare functional antibody dilutions, stock antibody in vehicle (PBS is optimal) was serially diluted. Then each point was diluted into assay media for 2× final concentration of antibody. After preparing antibodies, the cell plate was wished twice with assay media, by aspirating (vacuum aspirator) followed by the addition of 100 ul per well assay media. After second was h, the assay media was replaced with 50 ul per well of 2× antibody. The cell plate was returned to the incubator for-15-20 min.

In order to prepare the CAGA12 (clone 4A4) cells for the assay, the cells were detached with trypsin and pelleted (spin 5 min @ 200×g.). The pellet was resuspended in assay media and viable cells per ml were counted. Cells were diluted to 0.3×10⁶ cells/ml and seeded 50 μl per well (15,000 cells per well). Cells were returned to incubator.

On Day 3, the assay was read about 16-20 hours after the antibody and/or reporter cell addition. Bright-Glo reagent and test plate were allowed to come to room temperature before reading. The read settings on BioTek Synergy H1 were set to use TMLC_std protocol—this method has an auto-gain setting. Positive control wells were set for autoscale (high). 100 μL of Bright-Glo reagent was added per well. Incubated for 2 min with shaking, at room temperature, protected plate from light. The plate was read on BioTekSynergy H1.

FIG. 3 depicts inhibition of GARP pro-TGFβ1 activation in the SW480β6 assay. FIG. 4 shows inhibition of LRRC33-pro-TGFβ1 activation in the SW480β6 assay.

To calculated IC50 values, raw luminescence values were normalized against wells treated with PBS only (vehicle). Normalized values were plotted using PRISM® software, and values were calculated using 3-point non-linear regression.

Table 15 shows calculated IC50 values for isoform-specific antibodies. Reported IC50 values are averaged from duplicate wells of a single dose-response experiment; dose response curves and IC50 values are representative of multiple independent experiments.

TABLE 15
IC50s for isoform-specific antibodies against proTGFβ1-presenting
molecule complexes as measured by an in vitro potency assay.

IC50 (nM)

| | hLTBP1- | hLTBP3- | hGARP- | hLRRC33- |
|---|---|---|---|---|
| Ab Ref. | proTGFβ1 | proTGFβ1 | proTGFβ1 | proTGFβ1 |
| Ab3 | 24.29 | 10.42 | 0.981 | 0.8578 |
| Ab11 | 2.954 | 1.562 | 0.7902 | 1.268 |
| Ab12 | 3.794 | 1.555 | 1.077 | 2.434 |
| Ab2 | 3.394 | 0.8673 | 0.7758 | 1.375 |
| Ab19 | 1.656 | 1.599 | 1.34 | 0.4014 |
| Ab20 | 1.851 | 1.184 | 0.3872 | 1.354 |
| Ab35 | 6.246 | 2.707 | 3.715 | 5.37 |
| Ab1 | 14.47 | 2.707 | 3.715 | 5.37 |

### Example 4. Inhibition of Acute Fibrosis by Anti-proTGFβ1 Antibodies in the Unilateral Ureteral Obstruction (UUO) Model of Acute Kidney Fibrosis

Inhibition of acute fibrosis by anti-TGFβ1 antibodies was tested in the unilateral ureteral obstruction (UUO) model of acute kidney fibrosis. In this model, fibrosis is induced in male mice by permanent surgical ligation of the left ureter on study day 0. Sham-treated mice, which underwent surgery but did not have their ureters obstructed, were included as a healthy control in these experiments.

Control or test antibodies (Ab3, Ab2) were administered to mice by intraperitoneal (i.p.) injection on study days −1 and 4. Kidneys were collected at the end of study, on day 5 after surgery, and RNA was harvested from these tissues. The degree of fibrosis induction was subsequently assessed by quantitative polymerase chain reaction (qPCR) for a panel of fibrosis-associated genes, including Collagen I (Col1a1), Collagen III (Col3a1), Fibronectin 1 (Fn1), Lysyl Oxidase (Lox), Lysyl Oxidase-like 2 (Lox/2), Smooth muscle actin (Acta2), Matrix metalloprotease (Mmp2), and Integrin alpha 11 (Itga11) (Rolfe, Irvine, Grobbelaar, & Linge, 2007) (Tamaki et al., 1994) (Bansal et al., 2017)(Leaf& Duffield, 2016).

Effect of Ab2 or Ab3 Treatment on Collagen Gene Expression

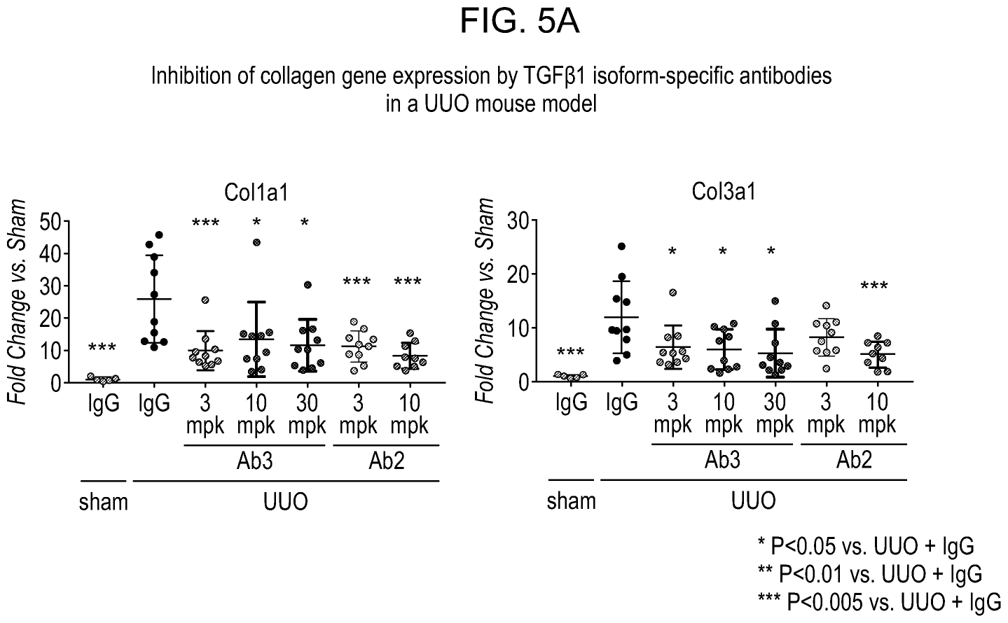

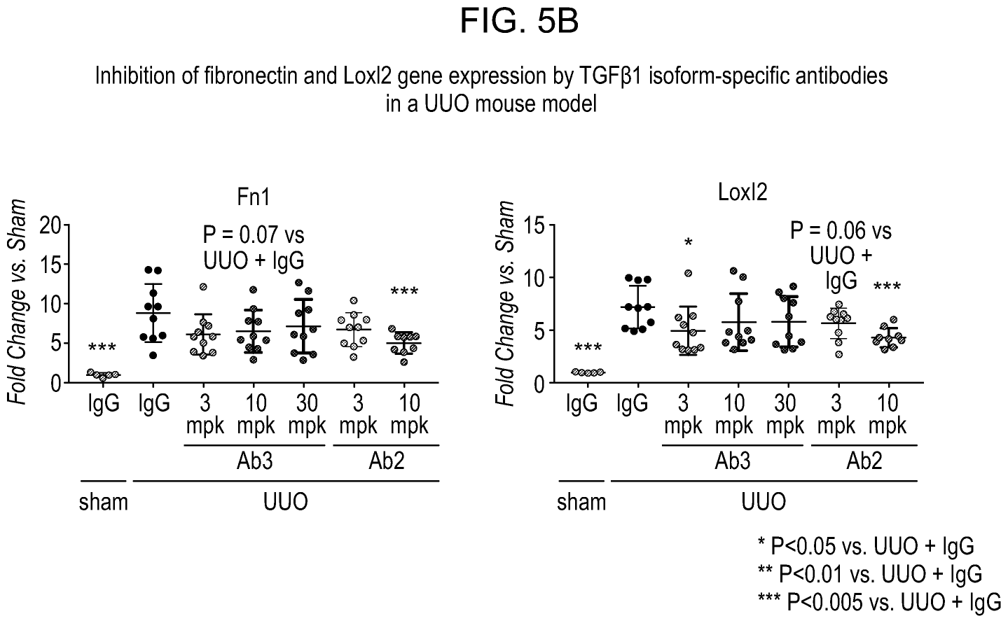

Col1a1 and Col3a1 are key drivers of fibrosis. As shown in FIG. 5A, Col1a1 is induced 10- to 40-fold in obstructed kidneys and Col3a1 is upregulated 5- to 25-fold (P<0.005, compare sham+IgG treated mice to UUO+IgG group). UUO mice treated with 3, 10, or 30 mg/kg/wk of Ab3 show reduced expression of both collagen genes compared to the UUO+IgG (P<0.05). Treatment with 10 mg/kg/wk of Ab2 also suppressed fibrotic gene induction by UUO (P<0.005 compared to UUO+IgG). Samples from the 3 mg/kg/wk Ab2 group also show a trend towards reduced Col3a1 expression, though this effect does not reach statistical significance. Taken together, these data suggest that TGFβ1 inhibition with either Ab2 or Ab3 potently ameliorates the collagen induction associated with UUO.

Effect of Ab2 or Ab3 Treatment on Fibronectin and Lysyl Oxidase-Like 2 Gene Expression

Fn1 and Lox/2 encode proteins that play roles in deposition and stiffness of extracellular matrix in fibrosis. As shown in FIG. 5B, both genes are upregulated in samples from the UUO+IgG group (P<0.005 vs. Sham+IgG), though the fold increase in gene expression for both genes, but particularly for Lox/2, is smaller than for the Collagen genes. In samples treated with 3, 10, or 30 mg/kg/wk of Ab3, a trend towards reduced Fn1 and Lox/2 (vs. UUO+IgG) was noted, but this treatment effect is only statistically significant for Lox/2 expression, and only at the 3 mg/kg/wk dose (Fn1 at the 10 mg/kg/wk dose is approaching statistical significance, with P=0.07). Treatment with 10 mg/kg/wk Ab2, however, leads to a highly significant (P<0.005 vs UUO+IgG) inhibition of both Fn1 and Lox/2. A lower, 3 mg/kg/wk dose of Ab2 leads to a reduction of both Fn1 and, particularly, Lox/2 (P=0.06), though suppression of neither gene is statistically significant.

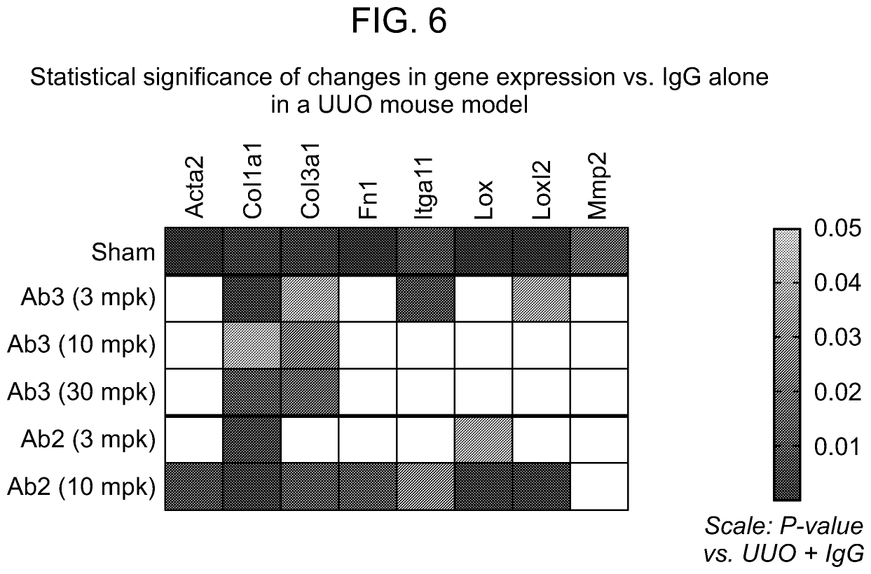

FIG. 6 summarizes the statistical significance of changes in gene expression (vs. UUO+IgG) after treatment in the UUO model. Ab3 showed reduction in Col1a1 and Col3a1 at all doses tested. Statistically significant changes were also observed in Itga11 and Lox/2 (both levels were reduced relative to UUO+IgG), but only in the 3 mg/kg/wk dose. In contrast, all genes examined except Mmp2 showed a statistically significant change in expression (all levels reduced relative to UUO+IgG) after treatment with 10 mg/kg/wk Ab2. Col1a1 and Lox expression were also reduced at the 3 mg/kg/wk dose.

### Example 5: Inhibition of TGFβ Signaling by Anti-proTGFβ1 Antibodies in a Genetic Model of Alport Syndrome

The murine Col4a3−/− model is an established genetic model of autosomal recessive Alport syndrome. Alport mice lack a functional collagen 4A3 gene (Col4A3−/−) and therefore cannot form normal type IV collagen trimers, which require α3, α4, and α5 chains. Col4a3−/− mice develop fibrosis in the kidney consistent with renal fibrosis in human patients, including interstitial fibrosis and tubular atrophy, and Col4a3−/− mice develop end-stage renal disease (ESRD) between 10 and 30 week of age, depending on the genetic background of the mouse. The structural and functional manifestation of renal pathology in Col4a3−/− mice, combined with the progression to ESRD make Col4a3−/− mice an ideal model to understand kidney fibrosis. Previous reports point to the importance of the TGFβ signaling pathway in this process, and treatment with either αvρ6 integrin, a known activator of TGFβ, or with a TGFβ ligand trap has been reported to prevent renal fibrosis and inflammation in Alport mice (Hahm et al. (2007) The American Journal of Pathology, 170(1): 110-125).

Ab3 and Ab2, which are isoform-specific, inhibitors of TGFβ1 activation, were tested for their ability to inhibitor mitigate renal fibrosis in Alport mice as follows.

F1 offspring from Col4a3+/− males on a 129/Sv genetic background crossed to Col4a3+/− females on a C57/Bl6 genetic were employed for the study. These mice typically exhibit proteinuria by 4-5 weeks old and typically progress to ESRD by 14-15 weeks old, providing a good therapeutic window for testing efficacy of treatment.

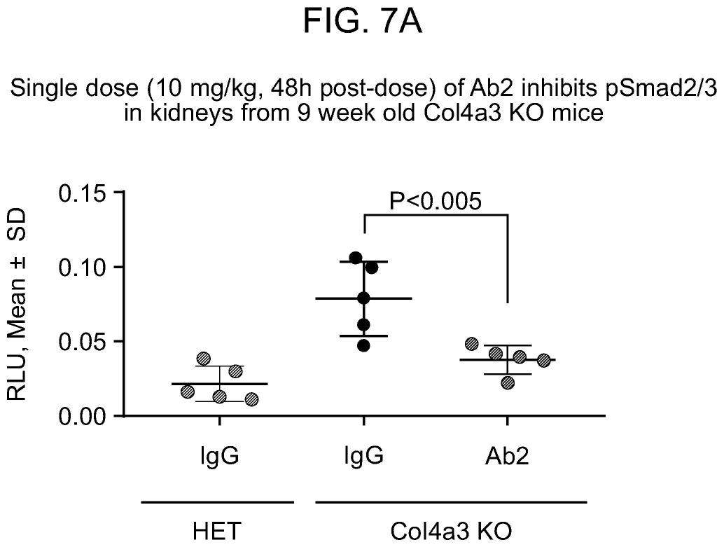

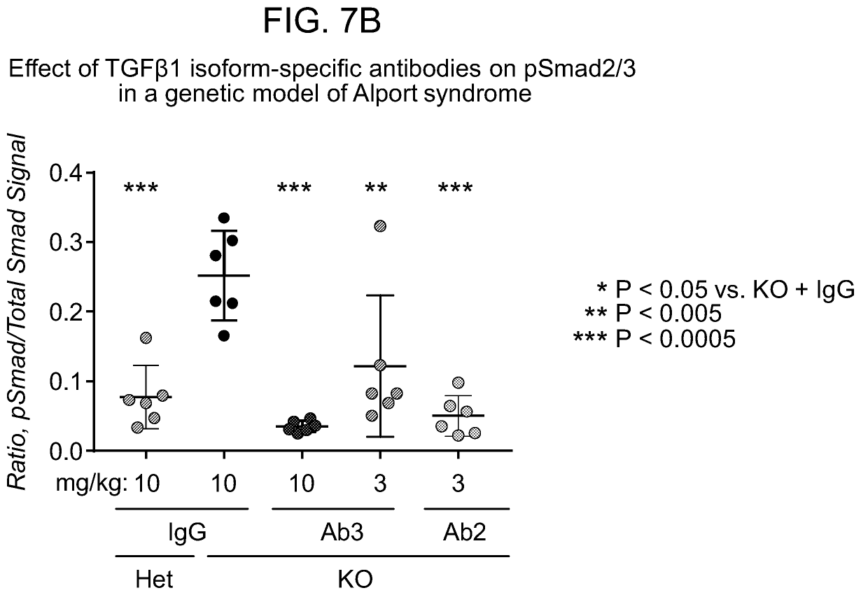

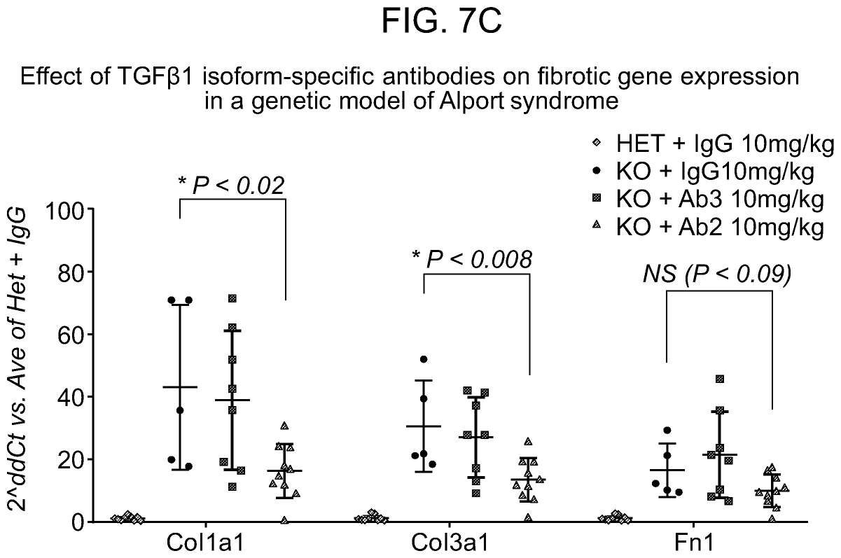

It is well documented that TGFβ receptor activation leads to a downstream signaling cascade of intra cellular events, including phosphorylation of Smad2/3. Therefore, the ability of Ab2 antibody treatment to inhibit TGFβ signaling may be assessed in kidney lysate samples by measuring relative phosphorylation levels of Smad2/3 as assayed by ELISA (Cell Signaling Technologies) according to the manufacturer's instructions. Accordingly, 9 week old Col4a3−/− mice were dosed with 10 mg/kg Ab2 intraperitoneally (i.p.) 48 hours prior to animal sacrifice and tissue collection. FIG. 7A provides a graph showing relative ratios of phosphorylated vs. total (phosphorylated and unphospohrylated) Smad2/3. A single dose of Ab2 was sufficient to significantly inhibit pSmad2/3 signaling in whole kidney lysates from 9 week old Col4a3−/− mice, demonstrating efficient target engagement by Ab2 in this model.

In a separate study, Col4a3−/− mice were treated with Ab2 or Ab3 beginning six weeks afterbirth. Mice were dosed i.p. twice weekly with either 5 mg/kg or 1.5 mg/kg Ab3 or with 1.5 mg/kg Ab2 for a test duration of six weeks. An IgG was used as negative control in both heterozygous (Col4a3+/−; Het) and knock out (Col4a3−/−; KO) mice. Following six weeks of antibody treatment (12 weeks after birth), animals were sacrificed, and the kidneys were collected for analyses.

FIG. 7B shows relative ratios of phosphorylated vs. total Smad2/3 in whole kidney lysates prepared from samples of animals treated with Ab3 or Ab2. Treatment with either antibody showed a significant reduction in relative phosphorylation of Smad2/3, as compared to IgG treated Col4a3−/− mice. The average Smad ratios in the treated KO mice were equivalent to those of heterozygous control.

To assess the functional effect of TGFβ inhibition, a third study was carried out in the Col4a3−/− model of kidney fibrosis. In this case, mice were treated i.p. twice a week with a 5 mg/kg dose of Ab3 or Ab2, beginning six weeks after birth and continuing until mice were sacrificed at 14 weeks old. Treatment effect was assessed by gene expression, which was evaluated by quantitative polymerase chain reaction (qPCR) using cDNA generated from the kidneys of mice in this study. FIG. 7C shows expression of genes encoding three proteins commonly associated with fibrosis, Collagen 1 (Col1a1), Collagen 3 (Col3a1), and Fibronectin 1 (Fn1). In Col4a3−/− kidneys from animals treated with IgG control antibody, expression of all three genes is significantly upregulated relative to that in heterozygous Col4a3+/− mice. Treatment with Ab2 significantly reduced the expression of Col1a1 and Col3a1 in Col4a3−/− mice. Expression of Fn1 showed a similar trend in Ab2-treated Col4a3−/− mice, though this effect was not statistically significant (P<0.09).

### Example 6: Inhibition of TGFβ Signaling by Anti-proTGFβ1 Antibodies in Choline-Deficient High Fat Diet (CDHFD)Model of Mouse NASH

The Choline deficient high fat diet (CDHFD) is an established dietary model of Non-Alcoholic steatohepatitis (NASH). In this model, male C57BL/6J mice are fed a choline deficient, 0.1% Methionine, high fat diet for 12 weeks. Three to six weeks after the start of the CDHFD, expression of α-sma protein (a marker for activation of hepatic stellate cells) increases and the development of hepatic fibrosis is accompanied by an increase in the hydroxyproline content accompanied by a rise in intrahepatic collagen synthesis and deposition (Matsumoto et. al, Int J Exp Pathol. 2013 April; 94(2):93-103).

Ab3 and Ab2 were tested for their ability to inhibit and/or reduce the extent of liver fibrosis in mice on CDHFD as follows.

Animals in the test cohorts were on the CDHFD for the duration of the study. A separate cohort of mice were administered a regular chow diet as a study control. Antibodies Ab3 and Ab2) were administered to mice by intraperitoneal (i.p.) injection beginning at 4 weeks post start of the CDHFD. Antibody test concentrations were as follows: 15 mg/kg, 5 mg/kg or 1.5 mg/kg twice a week (i.e., 30, 10, or 3 mg/kg/week) for a test duration of 8 weeks (i.e., weeks 4 thru 12). An IgG isotype antibody was used as a negative control at 15 mg/kg twice weekly (30 mg/kg/week). Following 8 weeks of dosing, animals were sacrificed and livers collected for analyses.

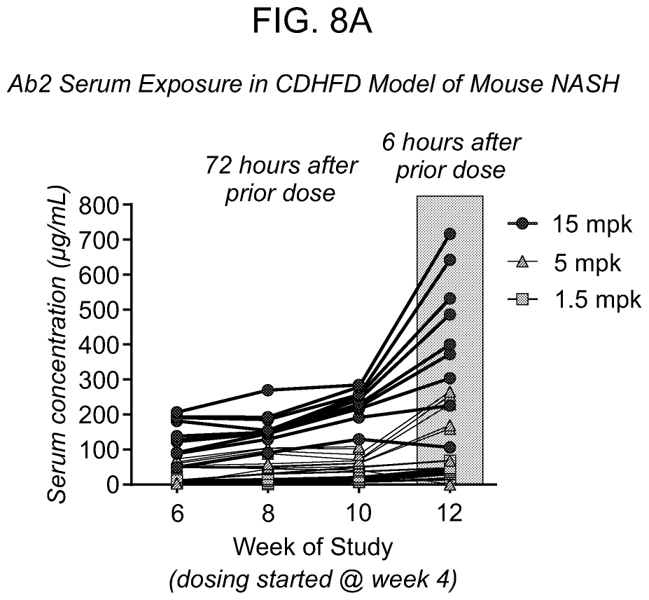

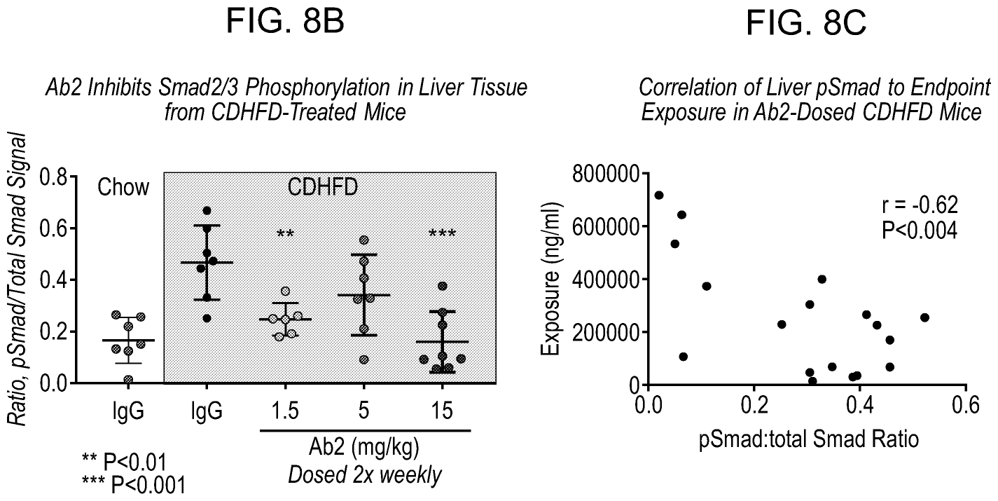

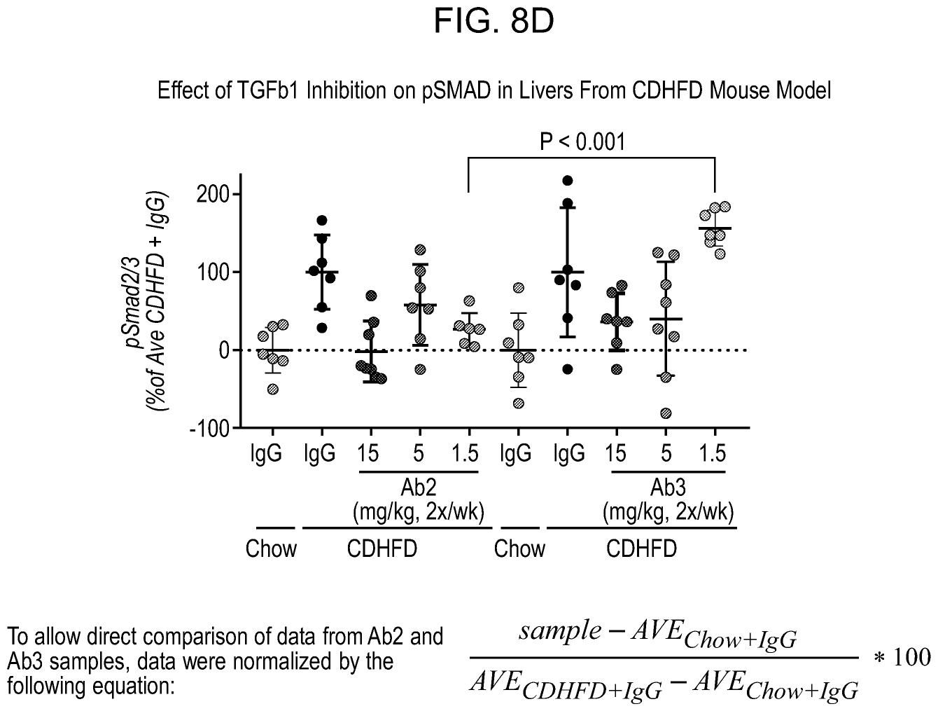

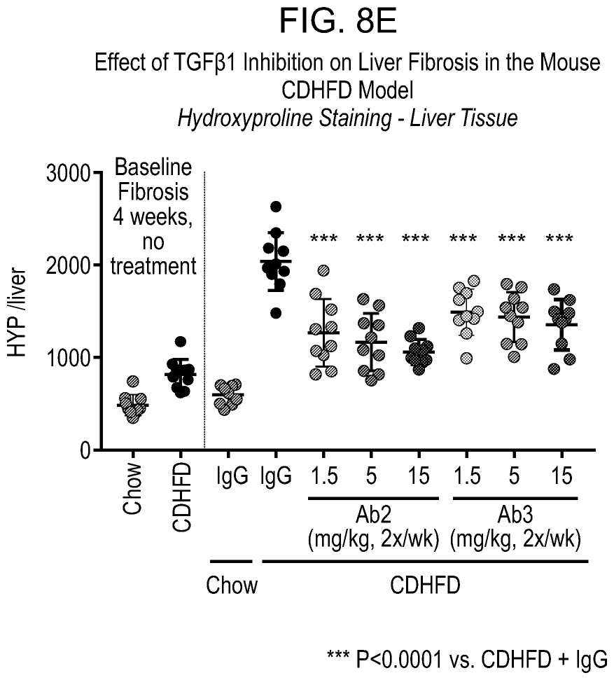

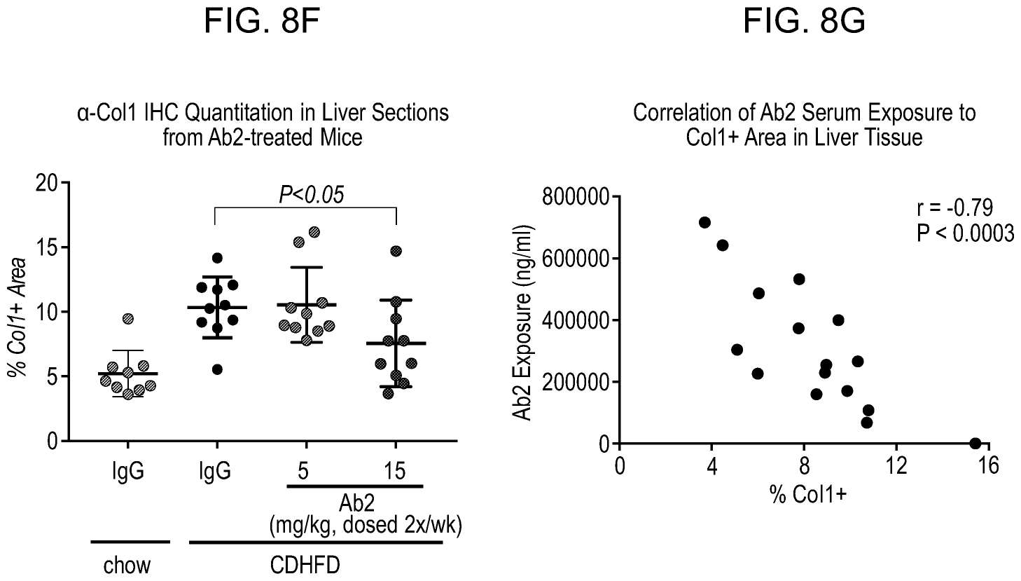

Serum measurements were also taken to determine the extent of antibody exposure during the study. Serum samples were collected 72 hours after Ab2 dosing at weeks 6, 8, and 10. At week 12, animals were given their final Ab2 injection and serum was taken 6 hrs post injection and at the time of sacrifice. FIG. 8A shows the serum levels of Ab2 during the duration of the study, indicating that we have quantifiable levels of Ab2 exposure in serum throughout the study. The two highest test doses of Ab2 at 30 and 10 mg/kg/week provide the best coverage throughout the study despite any presumed effect by the rise of anti-drug antibodies throughout the 8 weeks of dosing (van Brummelen et. al. The Oncologist. 2016 July; 21:1-9)

TGFβ1 receptor engagement leads to intracellular signaling events including the phosphorylation of Smad2 and Smad3. Accordingly, Ab3 and Ab2 were tested for their ability to inhibit Smad2/3 in liver lysates from CDHFD-treated mice by ELISA (Cell Signaling Technologies) according to manufacturer's protocol. As shown in FIG. 8B, Ab2 significantly reduced the relative ratio of phosphorylated Smad2/3 compared to IgG control. FIG. 8C demonstrates a inverse correlation between serum levels of Ab2 and phosphorylated Smad2/3. FIG. 8D shows Ab2 significantly reduces phosphorylation of Smad2/3 as compared to Ab3 and IgG treated mice on CDHFD (see, e.g., 1.5 mg/kg dose).

Hydroxyproline is a signature amino acid for fibrillar collagens and comprises approximately 13.5% of the protein. Fibrosis that occurs in the liver has the capacity to develop into chronic hepatitis, liver sclerosis, liver cancer, pulmonary fibrosis and glomerulonephritis (Qui et al. 2014 Mol. Med. Reports 2014 10; 1157-1163). Hydroxyproline acts as an important diagnostic indicator of the severity of fibrosis. As shown in FIG. 8E, animals undergoing treatment with Ab3 and Ab2 demonstrated a marked reduction in collagen deposition when compared to the control CDHFD animals administered IgG control Ab, indicating a positive treatment affect.

To assess the levels of type 1 collagen deposition in livers from CDHFD-treated mice, immunohistochemistry (IHC) protocols were developed for anti-mouse type I collagen antibody (rabbit polyclonal; Abcam; ab21286) using the Leica Bond RX staining system. Mouse liver was collected and fixed in 10% neutral buffered formalin (NBF) for 24 hours at room temperature. Fixed livers were then trimmed into 3-5 mm cross sections and stored in 70% ethanol until processed for paraffin infiltration and embedding. Paraffin embedded livers were sectioned at 4 μm and mounted on slides for IHC staining. Type 1 collagen primary antibody was used at a 5 μg/mL final concentration along with a matched isotype primary control (rabbit monoclonal IgG; Cell Signaling Technologies; 3900S). Epitope retrieval was performed with a pH 6 citrate buffer incubated at 100° C. for 20 minutes. Slides were washed in a tris-based buffer, incubated with a peroxide blocking reagent, then washed 3× in buffer, and incubated with a protein blocking reagent for 20 minutes before a 30 minute primary antibody incubation. Slides were again washed with buffer before incubation with a Leica HRP-polymer conjugate for 8 minutes. Slides were then washed 2× in buffer and 1× in deionized (DI) water before incubated with a Leica diaminobenzidine (DAB) chromogen for 10 minutes. Slides were then washed 3× with DI water before counterstaining with hematoxylin for 5 minutes. After final wash steps, stained IHC slides were then dehydrated and coverslipped using a xylene based mounting media.

Stained slides were sent to HistoTox Labs for imaging with an Aperio AT2 whole slide scanner and collagen staining was analyzed using Visiopharm image analysis software. Regions of interest (ROI) were selected by digitally tracing the tissue perimeter, marking each region for analysis. Tissue regions exhibiting folds or tears were excluded from analysis. Total collagen positive area was measured using an intensity-based thresholding algorithm which classified each pixel of the image as high positive, medium positive, low positive, or negative staining area. Total high, medium, and low pixel areas were summed together as a single positive value and divided by the total analyzed area of the tissue, resulting in the percentage of collagen positive area in the tissue. As shown in FIG. 8F, animals undergoing 15 mg/kg treatment with Ab2 had less total collagen I positive area than control CDHFD animals. In FIG. 8G, Ab2 treated animals showed an inverse correlation between their end-of-study exposure levels and their collagen IHC positive area; animals with high exposure showed much less collagen contently IHC, indicating lower levels of fibrosis in the liver.

### Example 7: Inhibition of TGFβ Signaling by Anti-proTGFβ1 Antibodies in a CCL₄ Model of Liver Fibrosis

Carbon tetrachloride (CCl₄) treatment of mice is a well-characterized model of liver fibrosis. In this model, Balb/C mice were dosed intraperitoneally (i.p.) twice weekly with 2.5 μl of a 20% CCl₄ solution in corn oil per gram bodyweight. This CCl₄ dosing was maintained throughout the duration of the study(6 weeks). After two weeks of CCl₄ dosing, dosing with test articles started. Mice were dosed i.p. with either twice weekly doses of 15 mg/kg, 5 mg/kg or 1.5 mg/kg of Ab2 or Ab3 twice a week (i.e., 30, 10, or 3 mg/kg/week), with twice weekly 5 mg/kg (10 mg/kg/week) doses of the pan-TGFβ antibody 1D11, or with twice weekly doses of 15 mg/kg (30 mg/kg/week) of a control IgG antibody. After 6 weeks of CCl₄ dosing (4 weeks of antibody treatment), mice were sacrificed and liver tissue was collected.

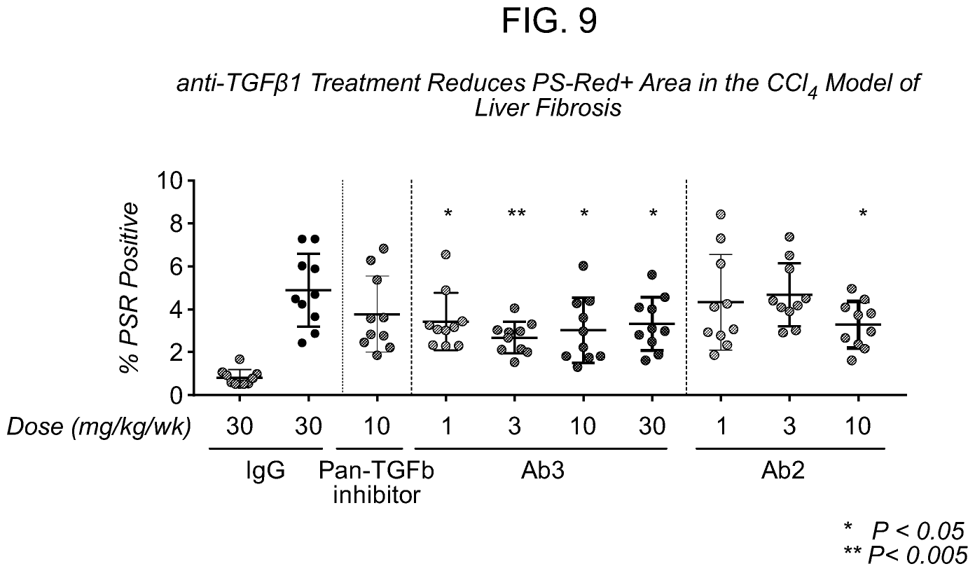

The hydroxyproline assay (see description above) was used to measure fibrosis in liver lysates from this study. FIG. 9 shows the results of this assay, in which treatment with all doses of Ab3 or with the high (15 mg/kg) dose of Ab2 showed a statistically significant reduction in liver hydroxyproline. Mice treated the lower doses of Ab2 (5 or 1.5 mg/kg) showed a trend towards reduced liver hydroxyproline, but these differences are not statistically significant due to experimental variation in this model. The treatment effect of both TGFβ1-specific antibodies was similar to that of the pan-TGFβ inhibitor 1D11.

### Example 8. Determination of Binding Regions for Ab3 and Ab2 by HDX-MS

Hydrogen/Deuterium exchange mass spectrometry (HDX-MS) is a widely used technique for exploring protein conformation in solution. HDX-MS methodology is described, for example, in Wei et al., Drug Discov Today. 2014 January; 19(1): 95-102; Engen J R. Anal Chem. 2009 Oct. 1; 81(19):7870-5, incorporated by reference in its entirety herein. Briefly, HDX-MS relies on the exchange of the protein backbone amide hydrogens with deuterium in solution. The backbone amide hydrogens involved in weak hydrogen bonds or located at the surface of the protein may exchange rapidly while those buried in the interior or those involved in stabilizing hydrogen bonds exchange more slowly. Based on the mass difference between hydrogen (1 Da) and deuterium (2 Da), which can be detected for example by mass spectrometry, the H-D exchange can be monitored overtime, Thus, by measuring HDX rates of backbone amide hydrogens, one can obtain information on protein dynamics, conformation and protein-protein interaction such as antibody-antigen-binding.

To gain insights for purposes of epitope mapping, HDX-MS was carried out to determine the binding regions of proTGFβ1 involved in Ab3-antigen-binding (e.g., where in the proTGFβ complex Ab3 was binding). In HDX-MS, the regions of an antigen that are tightly bound by an antibody are protected from proton exchange, due to protein-protein interaction, while regions that are exposed to solvent can readily undergo proton exchange. Based on this, binding regions within the antigen, which are protected by the specific binding of the antibody, were identified.

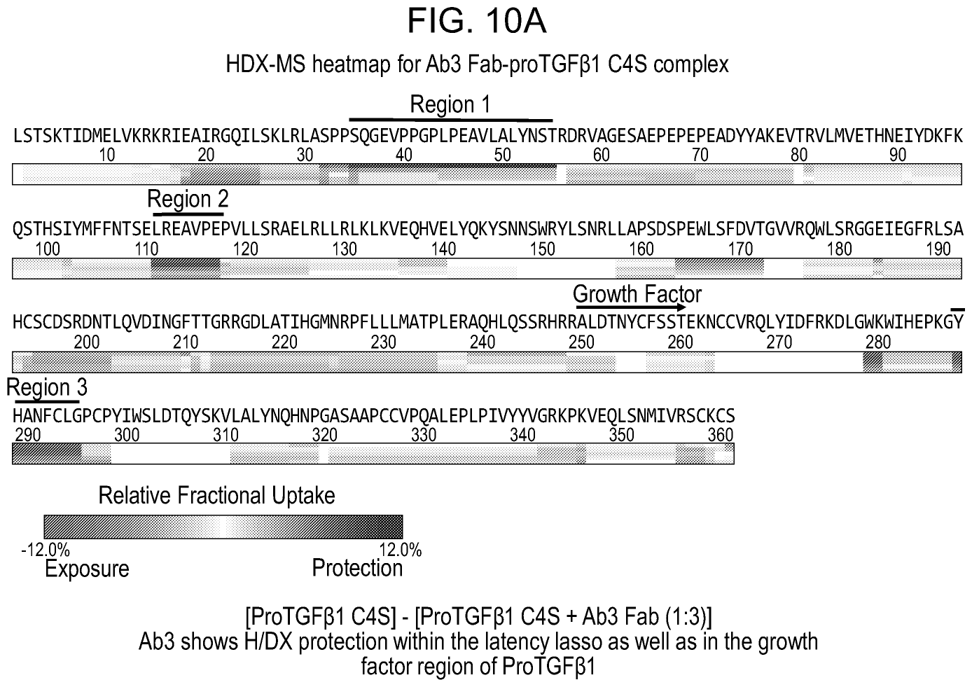

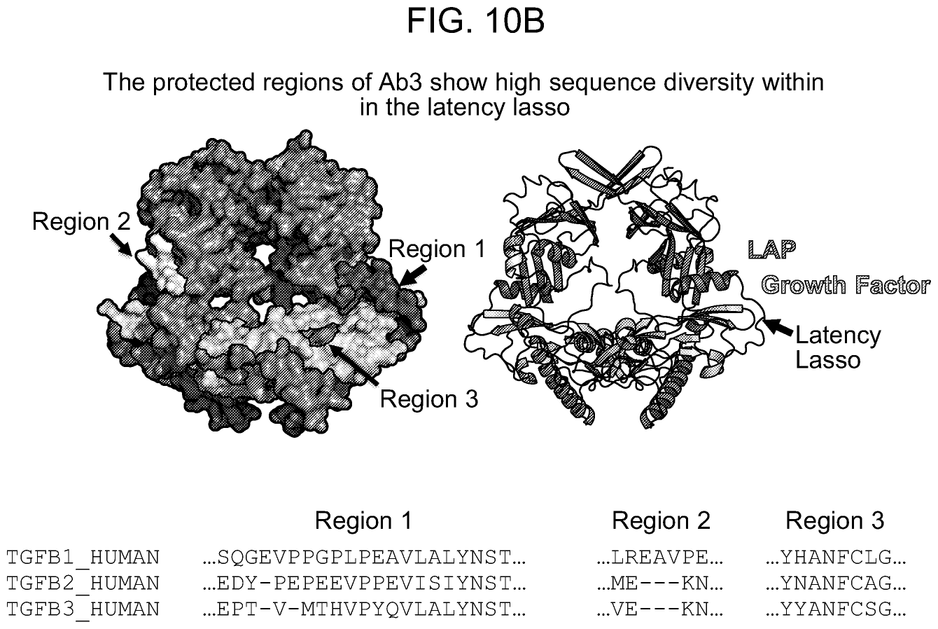

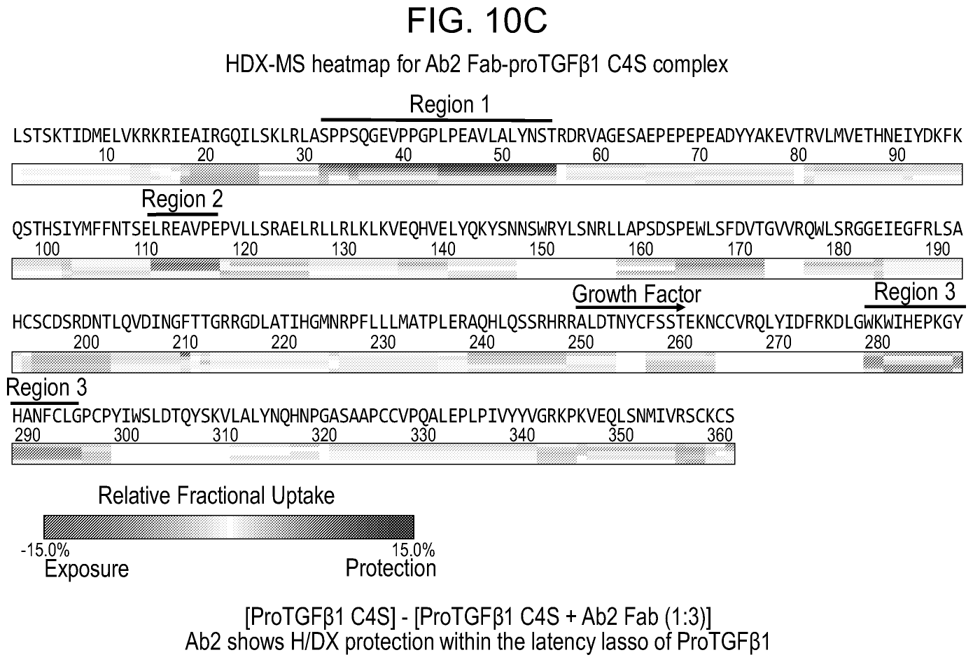

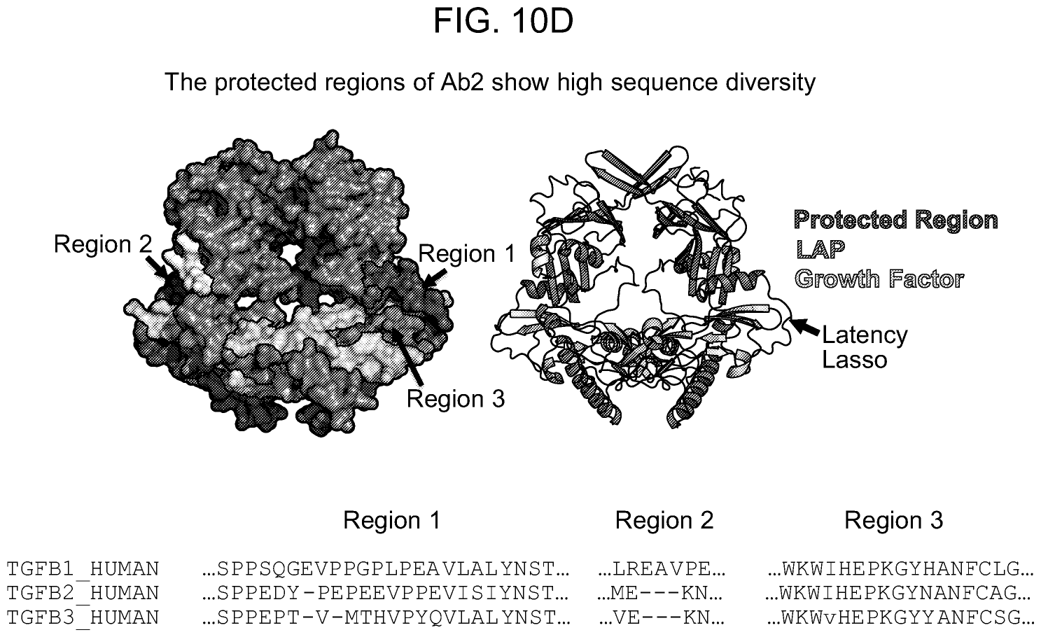

A modified proTGFβ1 complex (proTGFβ1 C4S) in which the cysteine residue at position 4 of the pro-domain has been substituted with a serine residue (described in WO 2014/182676) was used to assess Ab3 binding by HDX-MS. It is known that the cysteines from a proTGFβ1 homodimer are involved in forming covalent bonds with a presenting molecule, such as an LTBP, GARP and LRRC33.A 1:3 molar ratio of proTGFβ1 to Ab3 Fab was used in the experiment. The result is summarized in FIG. 10A. As shown there are at least three regions (labeled regions 1, 2 and 3 in FIG. 10A) in proTGFβ1 where H/D exchange was significantly protected upon binding to Ab3 Fab suggesting that these regions or portions thereof may represent binding region(s) of Ab3 to proTGFβ1. Region 1 (SQGEVPPGPLPEAVLALYNST; SEQ ID NO: 258) resulted in the highest H/D exchange among all peptic peptides resolved in proTGFβ1 and is mapped onto the latency lasso within the prodomain of proTGFβ1. The other two regions that showed significant H/D exchange protections are region 2 (LREAVPE; SEQ ID NO: 259) and region 3 (YHANFCLG; SEQ ID NO: 260) and are mapped onto the α3 helix within the prodomain and part of the growth factor of proTGFβ1, respectively. Amino acid sequence alignment of regions 1 and 2 comparing all three proTGFβ isoforms showed significant sequence diversity in these regions (see FIG. 101B) and thus provides a structural argument for the specificity of Ab3 to proTGFβ1 over the other isoforms.

To gain further structural insights into binding mechanism of Ab2 to proTGFβ1, we performed HDX-MS on proTGFβ1 C4S with Ab2 Fab using a 1:3 molar ratio of proTGFβ1 to Ab2 Fab (see FIG. 10C). As shown in FIG. 10C, there are at least three regions in proTGFβ1 that showed significant H/D exchange protection suggesting that these regions or portions thereof may represent the binding region(s) of Ab2 in proTGFβ1. Region 1 (SPPSQGEVPPGPLPEAVLALYNST; SEQ ID NO: 261) showed the highest H/D exchange among all peptic peptides resolved in proTGFβ1 and is mapped onto the latency lasso within the prodomain of proTGFβ1. Regions 2 (LREAVPE; SEQ ID NO: 259) and 3 (WKWIHEPKGYHANFCLG; SEQ ID NO: 262) are mapped onto the a3 helix within the prodomain and part of the growth factor of proTGFβ1, respectively. Amino acid sequence alignment of regions 1 and 2 comparing all proTGFβ isoforms -1. -2, and -3 showed sequence diversity in these regions (see FIG. 10D) and may provide the structural argument for the specificity of Ab2 to proTGFβ1 over the other isoforms

### Example 9: Determination of proTGFβ1 Binding Regions for Ab2 by X-Ray Crystallography

The epitope of Ab2 in proTGFβ1 was further elucidated by solving the crystal structure of the ternary complex of human proTGFβ1:Ab2-Fab:AbX-Fab. In this work, an uncleavable human proTGFβ1 C4S/R249A variant was used that spans residues 30-390 based on the full-length sequence of human proTGFβ1 (Uniprot ID P01137). The numbering system that will be used for the rest of this document will designate position 1 as the first amino acid residue after the removal of the signal sequence for proTGFβ isoforms. The C4S mutation renders proTGFβ1 not capable of covalent crosslinking to any known presenting molecules, e.g. LTBPs and GARP, while the R249A renders proTGFβ1 resistant to proprotein convertase/furin protease cleavage, thus maintaining proTGFβ1 in its uncleaved, proprotein form. ProTGFβ1 C4S/R249A was co-expressed with C-terminally 6×-His tagged Ab2-Fab in kifunensine-treated expi293 mammalian expression system to reduce N-linked glycosylation events. The proTGFβ1 C4S/R249A:Ab2-Fab was affinity purified, treated with endoglycosidase H and purified by gel filtration. The proTGFβ1 C4S/R249A:Ab2-Fab complex was then incubated with AbX-Fab to generate the ternary complex. The AbX Fab in this context was used as an auxiliary protein, which acts as a crystallization chaperone to increase the probability of obtaining protein crystals.

Crystallization experiments were performed at room temperature in a sitting drop format. Crystals suitable for X-ray analysis were obtained using 18% polyethylene glycol 6000, 0.2 M MgCl₂, 0.1 M sodium acetate buffer, pH 5.0. The crystals were cryoprotected by soaking in the mother liquor supplemented with 25% glycerol and flashed frozen in liquid nitrogen. X-ray diffraction data were collected at the SER-CAT beamline 22-ID at the Advanced Photon Source (APS) at the Argonne National Laboratory (ANL) using a Dectris Eiger 16M detector. The diffraction images were processed with X-ray Detector Software (XDS) in space group C2221 with an asymmetric unit containing one half of a 2:2:2 complex. The structure of the ternary complex was solved by molecular replacement using Phaser. Ab2-Fab was modeled using Fab structure of the same human germlines IGKV1-39/IGHV3-23 (PDB ID 5I19). The AbX-Fab was modeled using the Fab structure of human germlines IGHV1/IGKV3-11 (PDB ID 5I16). The core of the proTGFβ1 monomer was modeled using the previously reported crystal structure of proTGFβ1 (PDB ID 5VQP). The structure was manually rebuilt with Coot and refined using Refmac5 to a final resolution of 3.4 Å with R_{work} and R_{free} values 21% and 29%, respectively.

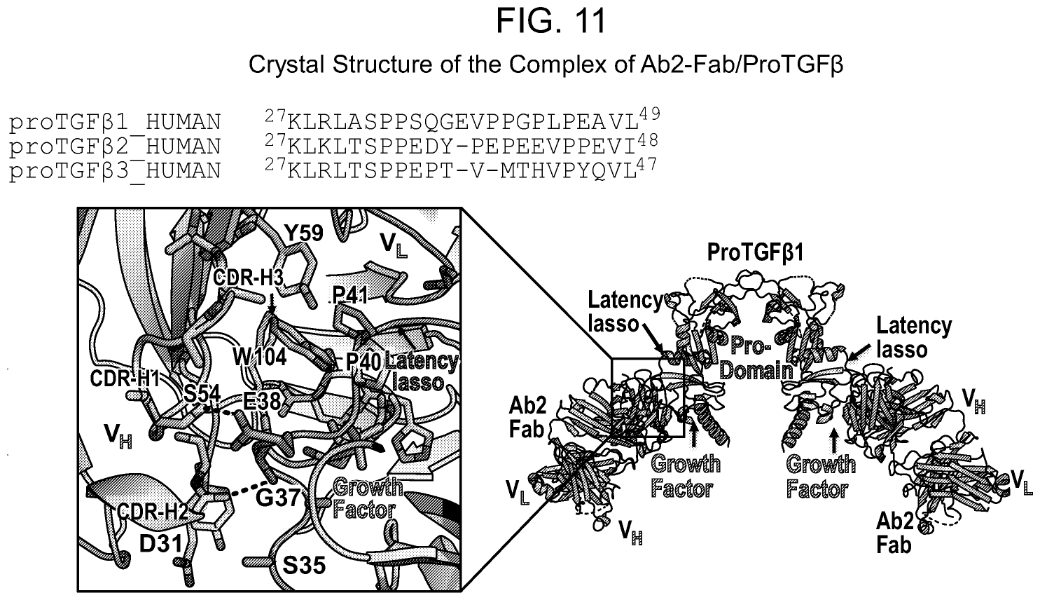

In the present crystal form, proTGFβ1 exists as a homodimer that sits on the crystallographic two-fold axis so that the asymmetric unit contains one proTGFβ1 monomer and two Fabs, while the entire complex has a 2:2:2 stoichiometry (FIG. 11). Ab2-Fab and AbX-Fab do not compete for binding proTGFβ1. Consistent with this fact, their binding epitopes on proTGFβ1 are spatially distant. Ab2-Fab binds a linear epitope formed by residues 35-43 with in the proline-rich loop of proTGFβ1 within the latency lasso. Pro40 and Pro41 of the proTGFβ1 are particularly important for binding as they are engaged in the pi stacking interactions with Trp104 and Tyr59 in the H-CDR3 and H-CDR1, respectively, of the Ab2-Fab. There are also relevant interactions in the growth factor residues (R274, K280, H283) with the heavy (S101, G102 H103) and light (Y49) chain residues of Ab2 Fab. Finally, sequence alignment of the epitope region in the latency lasso across TGFβ family showed significant diversity, which affords the exquisite specificity of Ab2 over other relevant members in the TGFβ family, e.g. TGFβ2, TGFβ3. The epitope and paratope for the interaction between proTGFβ1 and Ab2-Fab are listed in Table 16.

TABLE 16
Epitope and paratope for the interactions between proTGFβ1 and
Ab2-Fab. Letters h and l denote heavy and light chains, respectively.

| ProTGFβ1 | Ab2 |
|---|---|
| (Epitope) | (Paratope) |
| S35 | D31h |
| G37 | D31h, A33h |
| E38 | S54h |
| V39 | G102h |
| P40 | W104h, Y59h |
| P41 | W104h, Y59h |
| G42 | T91l, Y92l, Y32l |
| P43 | Y92l, Y32l |
| R274 | Y49l |
| K280 | S101h, H103h |
| H283 | G102h |

### Example 10. Ab3 and Ab2 Exhibit Reduced Toxicity as Compared to the ALK5 Kinase Inhibitor LY2109761 and a Pan-TGF/3 Antibody

To evaluate the potential in vivo toxicity of Ab3 and Ab2, as compared to the small molecule TGF-β type I receptor (ALK5) kinase inhibitor LY2109761 and to a pan-TGFβ antibody (hIgG4; neutralizing all three TGFβ growth factors), toxicity studies were performed in rats. The selection of rat as the species for this toxicology study was based on the previous reports that rats are more sensitive to TGFβ inhibition as compared to mice. Similar toxicities observed in rats have been also observed in other mammalian species, such as dogs, non-human primates, as well as humans.

Briefly, female Fischer 344 rats (FIGS. 12A and 12B) or Sprague Dawley (SD) rats were administered with either Ab3 at 3 mg/kg (1 group, n=5), at 10 mg/kg (1 group, n=5), at 30 mg/kg (1 group, n=5), or at 100 mg/kg (1 group, n=5); Ab2 at 10 mg/kg (1 group, n=5), at 30 mg/kg (1 group, n=5), or at 100 mg/kg (1 group, n=5); pan-TGFβ antibody at 3 mg/kg (1 group, n=5), at 10 mg/kg (1 group, n=5), at 30 mg/kg (1 group, n=5), or at 100 mg/kg (1 group, n=5); LY2109761 at 200 mg/kg (1 group, n=5) or 300 mg/kg (1 group, n=5); or PBS (pH 7.4) vehicle control (1 group, n=5).

Animals receiving pan-TGFβ antibody were dosed once intravenously (at day 1) at a volume of 10 mL/kg and sacrificed at day 8 and necropsies performed. Animals receiving either Ab3 or Ab2 were dosed once weekly for 4 weeks at a volume of 10 mL/kg. Animals receiving LY2109761 were dosed by oral gavage once daily for five or seven days. Animals were sacrificed and necropsies performed.

As shown in FIG. 12A and Table 17, animals administered pan-TGFβ antibody exhibited similar toxicities to those described in animals administered LY2109761 as described in PCT/US2018/012601, which is incorporated herein by reference in its entirety. Specifically, animals administered 3 mg/kg of the pan-TGFβ antibody exhibited heart valve findings (e.g., valvulopathy) similar to those described in the animals administered LY2109761. Animals administered 30 mg/kg of the pan-TGFβ antibody exhibited atrium findings similar to those described in animals administered LY2109761. Animals administered 100 mg/kg of the pan-TGFβ antibody exhibited myocardium findings similar to those described in animals administered LY2109761, and animals administered 30 mg/kg of pan-TGFβ antibody had hemorrhage in the myocardium. One animal administered 100 mg/kg of the pan-TGFβ antibody had moderate intramural necrosis with hemorrhage in a coronary artery, which was associated with slight perivascular mixed inflammatory cell infiltrates. Bone findings in animals administered the pan-TGFβ antibody and LY2109761 consisted of macroscopic abnormally shaped sternum and microscopic increased thickness of the hypertrophic zone in the endplate of the sternum and physis of the femur and tibia. Subsequent studies in a different rat strain (SD rats) with LY2109761 and pan-TGFβ as shown in FIG. 12C also demonstrated similar toxicities.

TABLE 17
Microscopic Heart Findings in Animals
Receiving the Pan-TGFβ Antibody

| | | Pan-TGFβ Antibody | | | |
|---|---|---|---|---|---|
| Heart | Dose Level (mg/kg/day) | 0 | 3 | 30 | 100 |
| Heart valves | | | | | |
| Valvulopathy | Minimal | 0 | 2 | 0 | 0 |
| | Slight | 0 | 2 | 4 | 5 |
| | Moderate | 0 | 0 | 1 | 0 |
| Atrium | | | | | |
| Infiltrate, mixed cell | Minimal | 0 | 0 | 1 | 2 |
| | Slight | 0 | 0 | 1 | 1 |
| Hyperplasia, | Minimal | 0 | 0 | 3 | 1 |
| endothelium | | | | | |
| Hemorrhage | Minimal | 0 | 0 | 1 | 0 |
| Myocardium | | | | | |
| Degeneration/ | Slight | 0 | 0 | 0 | 2 |
| necrosis | | | | | |
| Hemorrhage | Minimal | 0 | 0 | 2 | 1 |
| | Slight | 0 | 0 | 1 | 1 |
| Infiltrate, mixed cell, | Slight | 0 | 0 | 0 | 1 |
| base | | | | | |
| Coronary artery | | | | | |
| Necrosis with | Moderate | 0 | 0 | 0 | 1 |
| hemorrhage | | | | | |
| Infiltrate, mixed cell, | Slight | 0 | 0 | 0 | 1 |
| perivascular | | | | | |

However, unlike pan-TGFβ antibody or LY2109761-treated animals, animals administered Ab3 or Ab2 for 4 weeks showed no observable test article-related toxcity in either rat strain, and the NOAEL for both Ab3 and Ab2 was 100 mg/kg weekly dose, which was the highest dose tested (FIGS. 12B and 12D).

In summary, animals treated with Ab3 or Ab2 at all doses tested (10 mg/kg, 30 mg/kg or 100 mg/kg) over a period of 4 weeks exhibited no toxic effects over background in any of the following parameters: myocardium degeneration or necrosis, atrium hemorrhage, myocardium hemorrhage, valve hemorrhage, valve endothelium hyperplasia, valve stroma hyperplasia, mixed inflammatory cell infiltrates in heart valves, mineralization, necrosis with hemorrhage in coronary artery, necrosis with inflammation in aortic root, necrosis or inflammatory cell infiltrate in cardiomyocyte, and valvulopathy. The NOAEL for both Ab3 (in Fischer rats and in SD rats) and Ab2 (in SD rats) was a weekly dose of 100 mg/kg, the highest dose tested. Thus, treatment with isoform-specific inhibitors of TGFβ1 activation surprisingly resulted in significantly improved safety profiles, e.g., reduced mortality, reduced cardiotoxcity, and reduced bone findings as compared to pan-TGFβ inhibition.

### Example 11: Bioinformatic Analysis of Relative Expressions of TGFβ1, TGFβ2 and TGFβ3

To evaluate the expression of TGFβ isoforms in cancerous tumors, gene expression (RNAseq) data from publically available datasets was examined. Using a publically available online interface tool (Fire browse) to examine expression of TGFβ isoforms in The Cancer Genome Atlas (TCGA), the differential expression of RNA encoding TGFβ isoforms in both normal and cancerous tissue were first examined. All tumor RNAseq datasets in the TCGA database for which there were normal tissue comparators were selected, and expression of the TGFB1, TGFB2, and TGFB3 genes was examined (FIG. 13A). Data from the Firebrowse interface are represented as log 2 of reads per kilobase million (RPKM).

These data suggest that in most tumor types (gray), TGFB1 is the most abundantly expressed transcript of the TGFβ isoforms, with log 2(RPKM) values generally in the range of 4-6, vs. 0-2 for TGFB2 and 2-4 for TGFB3. We also note that in several tumor types, the average level of both TGFB1 and TGFB3 expression are elevated relative to normal comparator samples (black), suggesting that increased expression of these TGFβ isoforms may be associated with cancerous cells. Because of the potential role of TGFβ signaling in suppressing the host immune system in the cancer microenvironment, we were interested to note that TGFB1 transcripts were elevated in cancer types for which anti-PD1 or anti-PDL1 therapies are approved—these indications are labeled in gray on FIG. 13A.

Note that while RPKM >1 is generally considered to be the minimum value associated with biologically relevant gene expression (Hebenstreit et al., 2011; Wagner et al., 2013), however for subsequent analyses, more stringent cutoffs of RPKM (or of the related measure FPKM (see Conesa et al, 2016)) >10 or >30 to avoid false positives were used. For comparison, all three of those thresholds are indicated on FIG. 13A.

The large interquartile ranges in FIG. 13A indicate significant variability in TGFβ isoform expression among individual patients. To identify cancers whereat least a subset of the patient population have tumors that differentially express the TGFβ1 isoform, RNAseq data from individual tumor samples in the TCGA dataset was analyzed, calculating the number of fragments per kilobase million (FPKM). RPKM and FPKM are roughly equivalent, though FPKM corrects for double-counting reads at opposite ends of the same transcript (Conesa et al., 2016). Tumor samples were scored as positive for TGFβ1, TGFβ2, or TGFβ3 expression if the FPKM value the transcript was >30 and the fraction of patients (expressed as %) of each cancer type that expressed each TGFβ isoform were calculated (FIG. 13B).

As shown in FIG. 13B, a majority of tumor types in the TGCA dataset show a significant percentage of individual samples that are TGFβ1 positive, with some cancer types, including acute myeloid leukemia, diffuse large B-cell lymphoma, and head and neck squamous cell carcinoma, expressing TGFβ1 in more than 80% of all tumor samples. Consistent with the data in FIG. 13A, fewer cancer types are positive for TGFβ2 or TGFβ3, though several cancers show an equal or greater percentage of tumor samples that are TGFβ3 positive, including breast invasive carcinoma, mesothelioma, and sarcoma. These data suggest that cancer types may be stratified for TGFβ isoform expression, and that such stratification may be useful in identifying patients who are candidates for treatment with TGFβ isoform-specific inhibitors.

To further investigate this hypothesis, the log 2(FPKM) RNAseq data from a subset of individual tumor samples was plotted in a heat map (FIG. 13C), setting the color threshold to reflect FPKM>30 as a minimum transcript level to be scored TGFβ isoform-positive.

Each sample is represented as a single row in the heat map, and samples are arranged by level of TGFβ1 expression (highest expression levels at top). Consistent with the analysis in FIG. 13B, a significant number of samples in each cancer type are positive for TGFB1 expression. However, this representation also highlights the fact that many tumors express solely TGFB1 transcripts, particularly in the esophageal carcinoma, bladder urothelial, lung adenocarcinoma, and cutaneous melanoma cancer types. Interestingly, such TGFB1 skewing is nota feature of all cancers, as samples from breast invasive carcinoma show a much larger number of samples that are TGFB3-positive than are TGFB1 positive. Nonetheless, this analysis indicates that the P1 isoform is the predominant, and in most cases, the only, TGFβ family member present in tumors from a large number of cancer patients. Taken together with data suggesting that TGFβ signaling plays a significant role in immunosuppression in the cancer microenvironment, these findings also point to the utility of TGFβ1-specific inhibition in treatment of these tumors.

To identify mouse models in which to test the efficacy of TGFβ1-specific inhibition as a cancer therapeutic, TGFβ isoform expression in RNAseq data from a variety of cell lines used in mouse syngeneic tumor models was analyzed. For this analysis, two representations of the data were generated. First, similar to the data in FIG. 3, we generated a heat map of the log 2(FPKM) values for tumors derived from each cell line (FIG. 13D, left). Because this analysis was used to identify syngeneic models expressing high TGFB1 that are TGFB2 and TGFB3 negative, we were primarily concerned with avoiding false negatives, and we set our “positive” threshold at FPKM>1, well below that in the representations in FIGS. 13B and 13C.

As the data representation in FIG. 13D (left) makes clear, a number of syngeneic tumors commonly, including MC-38, 4T-1, and EMT6, express significant levels of both TGFβ1 and TGFβ3. In contrast, the A20 and EL4 models express TGFβ1 almost exclusively, and the S91 and P815 tumors show a strong bias for TGFB1 expression.

To further evaluate the differential expression of TGFB1 vs TGFB2 and/or TGFB3, the minΔTGFB1 was calculated, defined as the smaller value of log 2(FPKM_{TGFB1})−log 2(FPKM_{TGFB2}) or log 2(FPKM_{TGFB1})−log 2(FPKM_{TGFB3}). The minΔTGFB1 for each model is shown as a heat map in FIG. 13D (right), and underscores the conclusion from FIG. 13D (left) that syngeneic tumors from the A20, EL4, S91, and/or P815 cell lines may represent excellent models in which to test the efficacy of TGFβ1-specific inhibitors.

### Example 12. Effects of Ab3 and Ab2 in Combination with Anti-PD-1 Antibody on Tumor Progression in the MBT2 Syngeneic Bladder Cancer Mouse Model

To evaluate the effects of Ab3 and Ab2 in combination with an anti-PD-1 antibody to decrease bladder carcinoma tumor progression, the MBT2 syngeneic bladder cancer mouse model was used. This is a very aggressive and fast-growing tumor model, and is very difficult to overcome tumor progression with drug treatment.

Tumor Cell Culture

MBT-2 is a poorly differentiated murine bladder cancer cell line derived from a transplantable N-[4-(5-nitro-2-furyl)-2-thiazolyl] formamide-induced bladder cancer in a female C3H/He mouse. The cells were cultured in Roswell Park Memorial Institute (RPMI)-1600 medium with 10% fetal bovine serum and 100 μg/ml streptomycin in a 5% CO2 atmosphere at 37° C. The culture medium was replaced every other day, and subculture was performed when the cellular confluence reached 90%. Cells were harvested from subconfluent cultures by trypsinization and were washed in serum-free medium. Single cell suspensions with >90% cell viability were determined by Trypan blue exclusion. The cells were resuspended in phosphate-buffered saline (PBS) before injection.

In Vivo Implantation and Tumor Growth

The MBT2 cells used for implantation were harvested during log phase growth and resuspended in phosphate buffered saline (PBS). On the day of tumor implant, each test mouse was injected subcutaneously in the flank with 5×10⁵ cells (0.1 mL cell suspension), and tumor growth was monitored. When tumors reached an average between 40-80 mm³a mice were randomized into groups of 15. Tumors were measured in two dimensions using calipers, and volume was calculated using the formula:

Tumor Volume (mm³)=w²×l/2

where w=width and l=length, in mm, of the tumor. Tumor weight may be estimated with the assumption that 1 mg is equivalent to 1 mm³ of tumor volume.

Treatment

Briefly, mice (n=15) bearing subcutaneous MBT2 tumors (40 to 80 mm³) on Day 1 were administered intraperitoneally (i.p.) once a week for 29 days Ab3 at 10 mg/kg in a dosing volume of 10 mL/kg, Ab3 at 30 mg/kg in a dosing volume of 10 mL/kg, Ab2 at 3 mg/kg in a dosing volume of 10 mL/kg or Ab2 at 10 mg/kg in a dosing volume of 10 mL/kg. Rat anti mouse PD-1 antibody (RMP1-14-rIgG2a, BioXCel) was administered i.p. twice a week at 10 mg/kg in a dosing volume of 10 mL/kg for 29 days.

Group 1 received anti-PD-1 antibody only. Group 2 received Ab3 (10 mg/kg) in combination with anti-PD-1 antibody. Group 3 received Ab3 (30 mg/kg) in combination with anti-PD-1 antibody. Group 4 received Ab2 (3 mg/kg) in combination with anti-PD-1 antibody. Group 5 received Ab2 (10 mg/kg) in combination with anti-PD-1 antibody. Treatment with isotype control was used as a control group (data not shown).

Endpoint and Tumor Growth Delay (TGD) Analysis

Tumors were measured using calipers twice per week, and each animal was euthanized when its tumor reached the endpoint volume of 1,200 mm³. Mice that exited the study for tumor volume endpoint were documented as euthanized for tumor progression (TP), with the date of euthanasia.

Partial response (PR)due to treatment is defined as the tumor volume was 50% or less of its Day 1 volume for three consecutive measurements during the course of the study and equal to or greater than 13.5 mm3 for one or more of these three measurements. In a complete response (CR) the tumor volume was less than 13.5 mm3 for three consecutive measurements during the course of the study. Anti-PD1/Ab3 at 10 mg/kg had 0 PR and 4 CR at end of study. Anti-PD1/Ab3 at 30 mg/kg had 1 PR and 1 CR at end of study. Anti-PD1/Ab2 at 3 mg/kg had 1 CR and no other responses at 10 mg/kg. Percent tumor growth delay (% TGD) is defined as the increase in the median time to endpoint in a treatment group compared to the untreated control, expressed as a percentage of the median time to endpoint (TTE) of the control:

T=median TTE for treatment

C=median TTE for control

% TGD=((T−C)/C)*100

Anti-PD1/Ab2% TGD at 3 mg/kg was 11.4 and at 10 mg/kg was 13.6. Anti-PD1/Ab3 at 10 mg/kg had 191% TGD and at 30 mg/kg was 196.

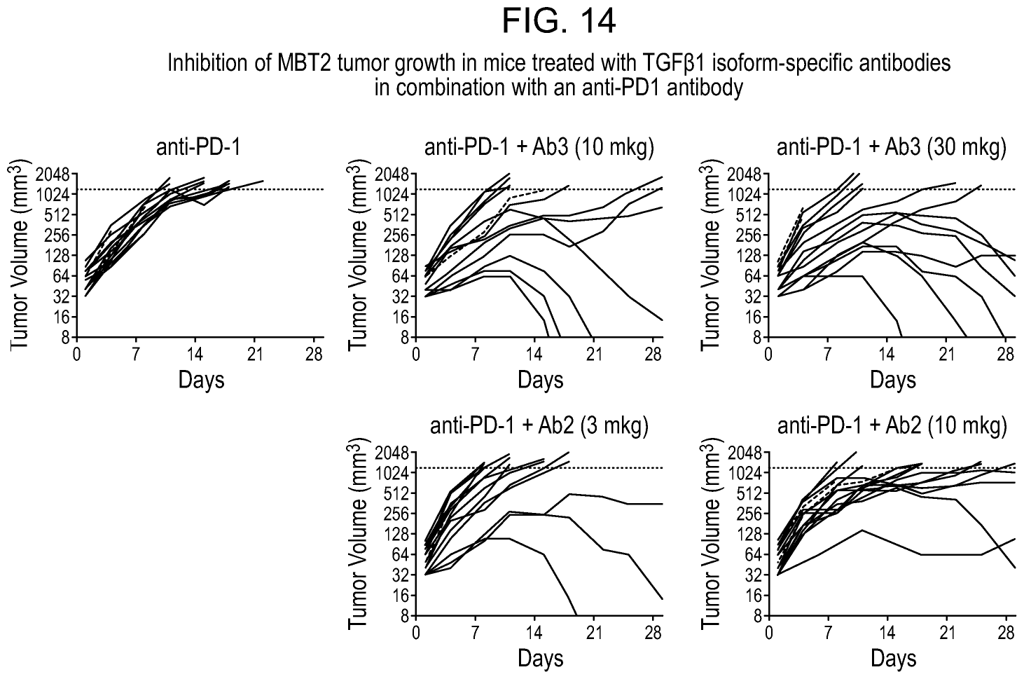

As shown in FIG. 14, administration of Ab3, at both the 10 mg/kg and 30 mg/kg doses, in combination with anti-PD-1, and Ab2 at both the 3 mg/kg and 10 mg/kg doses, in combination with anti-PD-1, delayed tumor growth as compared to PD-1 alone. As shown in FIG. 14, most of the mice treated with PD-1 alone reached a tumor volume of 1024 mm³ (as indicated by the dotted line) between about day 8 and day 20, whereas mice treated with Ab3 at 10 mg/kg, Ab3 at 30 mg/kg, Ab2 at 3 mg/kg, or Ab2 at 10 mg/kg took up to about 28 days to reach a tumor volume of 1024 mm³.

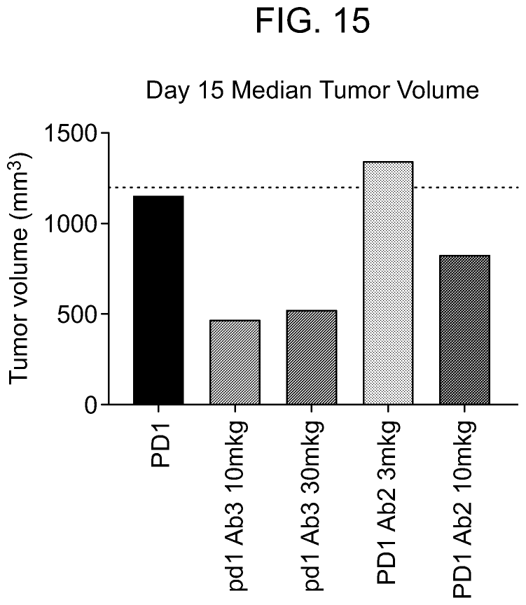

FIG. 15 is a graph that shows the median tumor volume (mm3) at day 15 in mice administered Ab3 at 30 mg/kg or 10 mg/kg or Ab2 at 3 mg/kg or 10 mg/kg, in combination with anti-PD1. As shown in FIG. 15, the median tumor volume at day 15 in mice treated with Ab3 at 10 mg/kg, Ab3 at 30 mg/kg, or Ab2 at 10 mg/kg was about 700 mm³ or less, while the median tumor volume at day 15 in mice treated with Ab2 at 3 mg/kg or PD-1 alone was 1000 mm³ or more.

### Example 13. Effects of Ab3 and Ab2 in Combination with Anti-PD-1 Antibody on Tumor Progression in the CloudmanS91 Melanoma Cancer Mouse Model

To evaluate the effects of Ab3 and Ab2 in combination with an anti-PD-1 antibody to decrease melanoma tumor progression, the CloudmanS91 mouse melanoma model was used.

Tumor Cell Culture

Clone M3 [Cloudman S91 melanoma] (ATCC® CCL-53.1™) cells were obtained from the American Type Culture Collection (ATCC), and were maintained at CR Discovery Services as exponentially growing suspension cultures in Kaighn's modified Ham's F12 Medium supplemented with 2.5% fetal bovine serum, 15% horse serum, 2 mM glutamine, 100 units/mL penicillin G sodium, 100 μg/mL streptomycin sulfate and 25 μg/mL gentamicin. The tumor cells were grown in tissue culture flasks in a humidified incubator at 37° C., in an atmosphere of 5% CO₂ and 95% air.

In Vivo Implantation and Tumor Growth

On the day of tumor implant, each female DBA/2 test mouse was injected subcutaneously in the flank with 5×10⁶ CloudmanS91 cells in 50% matrigel, and tumor growth was monitored. When tumors reached a volume of 125-175 mm³ mice were randomized into groups of 12 with identical mean tumor volumes and dosing began. Tumors were measured in two dimensions using calipers, and volume was calculated using the formula:

Tumor Volume (mm³)=w²×l/2

where w=width and l=length, in mm, of the tumor. Tumor weight may be estimated with the assumption that 1 mg is equivalent to 1 mm³ of tumor volume.

Treatment

Briefly, mice (n=12) bearing subcutaneous CloudmanS91 tumors (125-175 mm³) on Day 1 were administered intraperitoneally (i.p.) once a week for 60 days Ab3 at 10 mg/kg in a dosing volume of 10 mL/kg; Ab3 at 30 mg/kg in a dosing volume of 10 mL/kg; Ab2 at 10 mg/kg in a dosing volume of 10 mL/kg; or Ab2 at 30 mg/kg in a dosing volume of 10 mL/kg. Rat anti mouse PD-1 antibody (RMP1-14-rIgG2a, BioXCel) was administered i.p. twice a week at 10 mg/kg in a dosing volume of 10 mL/kg for 60 days.

Group 1 received anti-PD-1 antibody only. Group 2 received Ab3 (10 mg/kg) in combination with anti-PD-1 antibody. Group 3 received Ab3 (30 mg/kg) in combination with anti-PD-1 antibody. Group 4 received Ab2 (10 mg/kg) in combination with anti-PD-1 antibody. Group 5 received Ab2 (30 mg/kg) in combination with anti-PD-1 antibody. An untreated control was used, data not shown.

Endpoint and Tumor Growth Delay (TGD) Analysis

Tumors were measured using calipers twice per week, and each animal was euthanized when its tumor reached the endpoint volume of 2,000 mm³ or at the end of the study (Day 60), whichever happened earlier. Mice that exited the study for tumor volume endpoint were documented as euthanized for tumor progression (TP), with the date of euthanasia. The time to endpoint (TTE) for analysis was calculated for each mouse according to the methods described in WO 2018/129329.

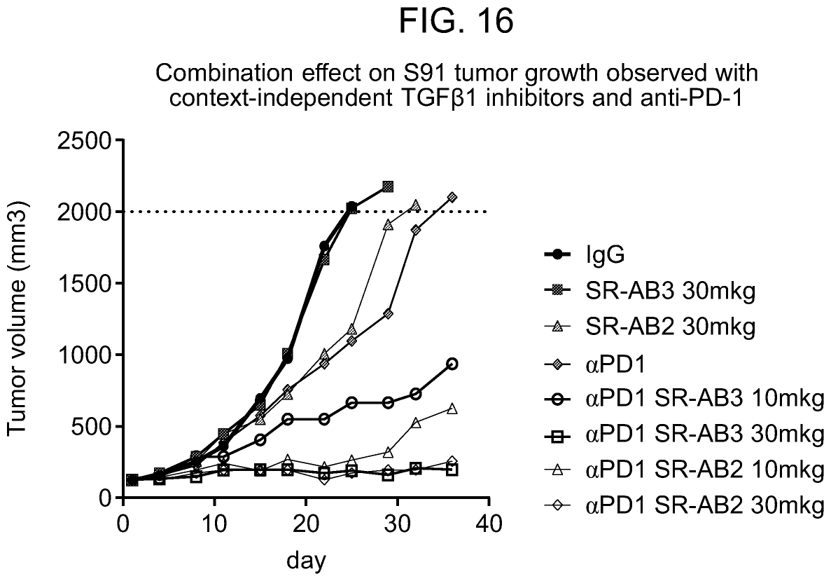

FIG. 16 shows the median tumor progression after treatment with Ab3 or Ab2 in combination with anti-PD-1 antibody. As shown in FIG. 16, administration of Ab3 or Ab2 at 10 mg/kg and 30 mg/kg, in combination with anti-PD1, delayed tumor growth. FIGS. 17A and 17B provide representative immunohistochemistry sections of CloudmanS91 tumor model, stained with a CD8+ cell marker. FIGS. 18A and 18B provide representative immunohistochemistry sections of CloudmanS91 tumor, stained with the macrophage marker, F4/80. As shown in FIGS. 17B and 18B, treatment with an anti-PD-1 inhibitor and a isoform-specific TGFb1 inhibitor increased CD8+ T cell infiltration and macrophage infiltration in S91 tumors.

### Example 14: Effect of pH on Ab2 Binding to proTGFb1 C4S

The FortéBio Octet Red384 was utilized to test pH-sensitivity of Ab2 (human IgG4) and a reference antibody as control (“R1”) (human IgG4), which is also an isoform-selective inhibitor of TGFβ1 that binds the latent complex in a pH-insensitive (pH-independent) matter. The antibodies were tested against sensor-immobilized human proTGFβ1 C4S, which contains a mutation in the prodomain to facilitate proTGFβ1 expression without disulfide linkage to a presentation molecule. Polystyrene 96-well black half area plates (Greiner Bio-One) and amine reactive second-generation (AR2G) biosensors (ForteBio) were utilized for this experiment.

The amine reactive second-generation (AR2G) reagent kit (FortéBio part no. 18-5095) was utilized according to the manufacturer's specifications for the assessment of pH sensitivity of Ab2 and R1 binding to human proTGFβ1 C4S. AR2G biosensors were first allowed to hydrate in water offline for at least 10 minutes before the initiation of the experiment. Upon initiation of the experiment, AR2G tips were equilibrated in water for 1 minute. Then, the tips were moved into an activation solution for 5 minutes. The activation solution consists of 18 parts water, 1 part 400 mM EDC (1-Ethyl-3-[3-dimethylaminopropyl] carbodiimide hydrochloride), and 1 part 200 mM s-NHS (N-hydroxysulfosuccinimide). The activation solution is prepared immediately before use in the experiment. After tip activation, the tips were loaded for 3 minutes with a 10 ug/mL solution of human proTGFβ1 C4S in 10 mM sodium acetate buffer pH 5. After loading, the tips were quenched in ethanolamine pH 8.5 for 15 minutes. The tips were then baseline/blocked with a 20 minute incubation in 1× enhanced kinetics buffer. 1× enhanced kinetics buffer (1×EKB) is 1× kinetics buffer (ForteBio part no 18-1105 diluted from 10× with PBS) with the addition of 2% BSA (Sigma A3059-100G), 0.5 M NaCl (FisherS671-10), and 0.09% tween-20 (P7949-500M). Tips were then allowed to associate in a 10 ug/mL solution of Ab2 or R1 in 1×EKB (pH 7.3) for 10 minutes before the final 10 minute dissociation step. The dissociation was performed in 1×KB at pH 7 and pH 5.

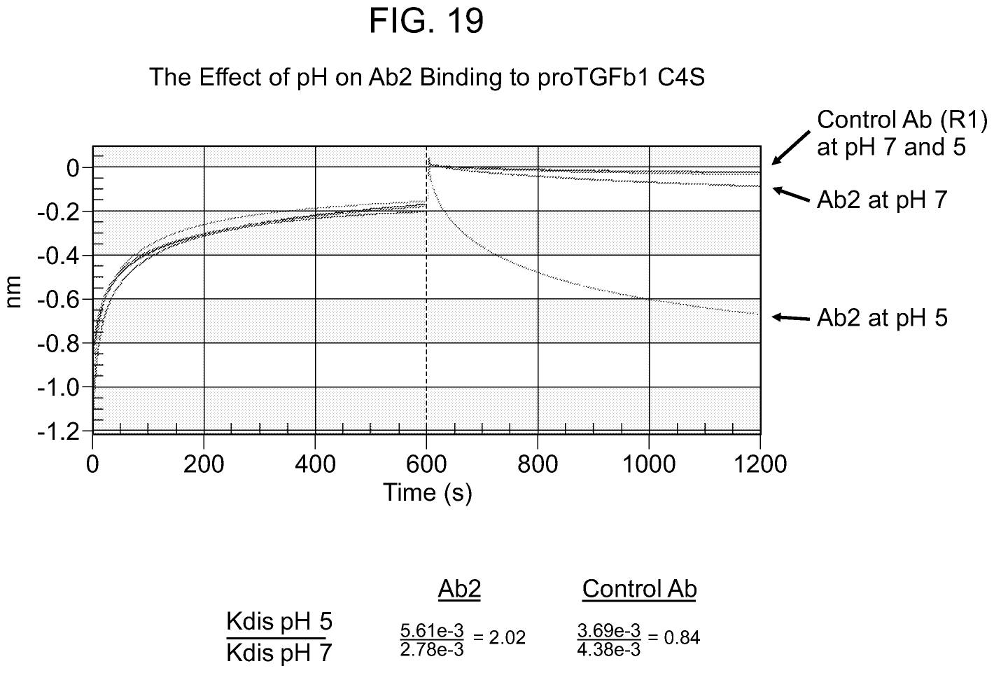

FIG. 19 shows the binding kinetics of Ab2 and R1 at different pHs. R1 showed minimal dissociation at pH 7 or pH 5 (K_{dis}=4.38 e⁻³ and K_{dis}=3.69 e⁻³, respectively). Ab2 showed some dissociate at pH 7 (K_{dis}=2.78 e⁻³). However, Ab2 showed a dramatic dissociation at pH 5 (K_{dis}=5.61 e⁻³), indicating that Ab2 is pH-sensitive.

The various features and embodiments of the present invention, referred to in individual sections above apply, as appropriate, to other sections, mutatis mutandis. Consequently, features specified in one section may be combined with features specified in other sections, as appropriate.

### LIST OF CERTAIN EMBODIMENTS

Non-limiting embodiments of the present disclosure are listed below:

1. An isolated monoclonal antibody or antigen-binding fragment thereof that specifically binds a proTGFβ1 complex wherein the antibody or the fragment comprises H-CDR1, H-CDR2, H-CDR3, L-CDR1, L-CDR2, and L-CDR3, wherein:
i) the H-CDR1 has an amino acid sequence represented by FTF(X₁)(X₂)(X₃)AM(X₄), wherein X₁ is A or S; X₂ is N, D, S, or A; X₃ is Y or F; and/or X₄ is S, T, or V (SEQ ID NO: 252);
ii) the H-CDR2 has an amino acid sequence represented by (X₁)IS(X₂)(X₃)(X₄)(X₅)(X₆)(X₇)Y(X₃)ADSVKG, wherein optionally, X₁ is S or A; X₂ is G or S; X₃ is S, T, or F; X₄ is G or A; X₅ is G, A, F, or S; X₆ is A, H, T, S, or V; X₇ is T or I; and/or, X₈ is Y or F (SEQ ID NO: 253);
iii) the H-CDR3 has an amino acid sequence represented by A(X₁)VSS(X₂)(X₃)WD(X₄)D(X₅), wherein optionally, X₁ is R or T; X₂ is G or Y; X₃ is H or L; X₄ is F, Y, or L; and/or X₅ is Y or E (SEQ ID NO: 254);
iv) the L-CDR1 has an amino acid sequence represented by (X₁)ASQ(X₂)IS(X₃)(X₄)LN, wherein optionally, X₁ is R or Q; X₂ is S or D; X₃ is S or N; and/or X₄ is F, Y or S (SEQ ID NO: 255);
v) the L-CDR2 has an amino acid sequence represented by (X₁)AS(X₂)L(X₃)(X₄), wherein optionally, X₁ is D or A; X₂ is S or N; X₃ is Q or E; and/or X₄ is S or T (SEQ ID NO: 256); and,
vi) the L-CDR3 has an amino acid sequence represented by QQ(X₁)(X₂)(X₃)(X₄)P(X₅)T, wherein optionally, X₁ is S, A, T, or V; X₂ is F, Y or P; X₃ is S, N, T, or D; X₄ is A, L, V, or P; and/or X₅ is F or L (SEQ ID NO: 257).
   2. The isolated monoclonal antibody or antigen-binding fragment thereof, according to embodiment 1, wherein:
i) the H-CDR1 comprises a D at position X₂;
ii) the H-CDR2 comprises an S at position X₃;
iii) the H-CDR3 comprises a G at position X₂ is a G and an H at position X₃;
iv) the L-CDR2 immediately follows a Y; and,
vi) the L-CDR3 comprises a Tat position X₁ and a Y at position X₂.
   3. An isolated monoclonal antibody or antigen-binding fragment thereof that specifically binds a proTGFβ1 complex wherein the antibody or the fragment comprises H-CDR1, H-CDR2, H-CDR3, L-CDR1, L-CDR2, and L-CDR3, wherein:
i) the H-CDR1 has an amino acid sequence represented by FTF(X₁)D(X₃)AM(X₄), wherein X₁ is A or S; X₃ is Y or F; and/or X₄ is S, T, or V (SEQ ID NO: 276);
ii) the H-CDR2 has an amino acid sequence represented by (X₁)IS(X₂)S(X₄)(X₅)(X₆)(X₇)Y(X₈)ADSVKG, wherein optionally, X₁ is S or A; X₂ is G or S; X₄ is G or A; X₅ is G, A, F, or S; X₆ is A, H, T, S, or V; X₇ is T or I; and/or, X₈ is Y or F (SEQ ID NO: 277);
iii) the H-CDR3 has an amino acid sequence represented by A(X₁)VSSGHWD(X₄)D(X₅), wherein optionally, X₁ is R or T; X₄ is F, Y, or L; and/or X₅ is Y or E (SEQ ID NO: 278);
iv) the L-CDR1 has an amino acid sequence represented by (X₁)ASQ(X₂)IS(X₃)(X₄)LN, wherein optionally, X₁ is R or Q; X₂ is S or D; X₃ is S or N; and/or X₄ is F, Y or S (SEQ ID NO: 255);
v) the L-CDR2 has an amino acid sequence represented by Y(X₁)AS(X₂)L(X₃)(X₄), wherein optionally, X₁ is D or A; X₂ is S or N; X₃ is Q or E; and/or X₄ is S or T (SEQ ID NO: 279); and,
vi) the L-CDR3 has an amino acid sequence represented by QQTY(X₃)(X₄)P(X₅)T, wherein optionally, X₃ is S, N, T, or D; X₄ is A, L, V, or P; and/or X₅ is F or L (SEQ ID NO: 280).
   4. The antibody or the fragment according to any one of the preceding embodiments, wherein the antibody comprises:
i) an H-CDR1 comprising an amino acid sequence as set forth in SEQ ID NO: 2;
ii) an H-CDR2 comprising an amino acid sequence as set forth in SEQ ID NO: 4;
iii) an H-CDR3 comprising an amino acid sequence as set forth in SEQ ID NO: 6;
iv) an L-CDR1 comprising an amino acid sequence as set forth in SEQ ID NO: 8;
v) an L-CDR2 comprising an amino acid sequence as set forth in SEQ ID NO: 10; and,
vi) an L-CDR3 comprising an amino acid sequence as set forth in SEQ ID NO: 12; optionally, wherein each CDR has at least 85% sequence identity with the corresponding CDR.
   5. The antibody or the fragment according to any one of the preceding embodiments, wherein the antibody comprises:
i) an H-CDR1 comprising an amino acid sequence as set forth in SEQ ID NO: 108;
ii) an H-CDR2 comprising an amino acid sequence as set forth in SEQ ID NO: 121;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   6. An isolated monoclonal antibody or antigen-binding fragment thereof that specifically binds a proTGFβ1 complex, wherein the antibody or the fragment comprises a variable heavy chain sequence at least 95% identical to SEQ ID NO: 13, and a variable light chain sequence at least 95% identical to SEQ ID NO: 15.
   7. An isolated monoclonal antibody or antigen-binding fragment thereof that specifically binds a proTGFβ1 complex, wherein the antibody or the fragment comprises a variable heavy chain sequence at least 90% identical to SEQ ID NO: 13, and a variable light chain sequence at least 90% identical to SEQ ID NO: 15,
wherein:
i) the variable heavy chain comprises amino acid residues D31, A33, S54, Y59, S101, G102, H103, and W104, as set forth in SEQ ID NO: 13, and;
ii) the variable light chain comprises amino acid residues Y32, Y49, T91, and Y92, as set forth in SEQ ID NO: 15.
   8. The antibody or the fragment according to embodiment 7, wherein the variable heavy chain sequence is at least 95% identical to SEQ ID NO: 13, and a variable light chain sequence is at least 95% identical to SEQ ID NO: 15.
   9. The antibody or the fragment according to any one of the preceding embodiments, wherein the antibody comprises a heavy chain comprising an amino acid sequence as set forth in SEQ ID NO: 17, and a light chain comprising an amino acid sequence as set forth in SEQ ID NO: 19.
   10. An antibody or antigen-binding fragment thereof that specifically binds a proTGFβ1 complex, wherein the antibody comprises:
i) an H-CDR1 comprising an amino acid sequence as set forth in SEQ ID NO: 108;
ii) an H-CDR2 comprising an amino acid sequence as set forth in SEQ ID NO: 109;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 112; and,
vi) an L-CDR3 comprising an amino acid sequence as set forth in SEQ ID NO: 113; optionally, wherein each CDR has at least 85% sequence identity with the corresponding CDR.
   11. An antibody or antigen-binding fragment thereof that specifically binds a proTGFβ1 complex, wherein the antibody comprises a variable heavy chain sequence at least 95% identical to SEQ ID NO: 210, and a variable light chain sequence at least 95% identical to SEQ ID NO: 211.
   12. An antibody or antigen-binding fragment thereof that specifically binds a proTGFβ1 complex, wherein the antibody comprises:
i) an H-CDR1 comprising an amino acid sequence as set forth in SEQ ID NO: 114;
ii) an H-CDR2 comprising an amino acid sequence as set forth in SEQ ID NO: 115;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 112; and,
vi) an L-CDR3 comprising an amino acid sequence as set forth in SEQ ID NO: 113; optionally, wherein each CDR has at least 85% sequence identity with the corresponding CDR.
   13. An antibody or antigen-binding fragment thereof that specifically binds a proTGFβ1 complex, wherein the antibody comprises a variable heavy chain sequence at least 95% identical to SEQ ID NO: 212, and a variable light chain sequence at least 95% identical to SEQ ID NO: 211.
   14. An antibody or antigen-binding fragment thereof that specifically binds a proTGFβ1 complex, wherein the antibody comprises:
i) an H-CDR1 comprising an amino acid sequence as set forth in SEQ ID NO: 120;
ii) an H-CDR2 comprising an amino acid sequence as set forth in SEQ ID NO: 121;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 112; and,
vi) an L-CDR3 comprising an amino acid sequence as set forth in SEQ ID NO: 113; optionally, wherein each CDR has at least 85% sequence identity with the corresponding CDR.
   15. An antibody or antigen-binding fragment thereof that specifically binds a proTGFβ1 complex, wherein the antibody comprises a variable heavy chain sequence at least 95% identical to SEQ ID NO: 214, and a variable light chain sequence at least 95% identical to SEQ ID NO: 211.
   16. An antibody or antigen-binding fragment thereof that specifically binds a proTGFβ1 complex, wherein the antibody comprises:
i) an H-CDR1 comprising an amino acid sequence as set forth in SEQ ID NO: 126;
ii) an H-CDR2 comprising an amino acid sequence as set forth in SEQ ID NO: 127;
iii) an H-CDR3 comprising an amino acid sequence as set forth in SEQ ID NO: 110;
iv) an L-CDR1 comprising an amino acid sequence as set forth in SEQ ID NO: 129;
v) an L-CDR2 comprising an amino acid sequence as set forth in SEQ ID NO: 130; and,
vi) an L-CDR3 comprising an amino acid sequence as set forth in SEQ ID NO: 131; optionally, wherein each CDR has at least 85% sequence identity with the corresponding CDR.
   17. An antibody or antigen-binding fragment thereof that specifically binds a proTGFβ1 complex, wherein the antibody comprises a variable heavy chain sequence at least 95% identical to SEQ ID NO: 216, and a variable light chain sequence at least 95% identical to SEQ ID NO: 217.
   18. An antibody or antigen-binding fragment thereof that specifically binds a proTGFβ1 complex, wherein the antibody comprises:
i) an H-CDR1 comprising an amino acid sequence as set forth in SEQ ID NO: 126;
ii) an H-CDR2 comprising an amino acid sequence as set forth in SEQ ID NO: 127;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   19. An antibody or antigen-binding fragment thereof that specifically binds a proTGFβ1 complex, wherein the antibody comprises a variable heavy chain sequence at least 95% identical to SEQ ID NO: 216, and a variable light chain sequence at least 95% identical to SEQ ID NO: 15.
   20. An antibody or antigen-binding fragment thereof that specifically binds a proTGFβ1 complex, wherein the antibody comprises:
i) an H-CDR1 comprising an amino acid sequence as set forth in SEQ ID NO: 114;
ii) an H-CDR2 comprising an amino acid sequence as set forth in SEQ ID NO: 115;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   21. An antibody or antigen-binding fragment thereof that specifically binds a proTGFβ1 complex, wherein the antibody comprises a variable heavy chain sequence at least 95% identical to SEQ ID NO: 212, and a variable light chain sequence at least 95% identical to SEQ ID NO: 15.
   22. An antibody or antigen-binding fragment thereof that specifically binds a proTGFβ1 complex, wherein the antibody comprises:
i) an H-CDR1 comprising an amino acid sequence as set forth in SEQ ID NO: 108;
ii) an H-CDR2 comprising an amino acid sequence as set forth in SEQ ID NO: 145;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 148; and,
vi) an L-CDR3 comprising an amino acid sequence as set forth in SEQ ID NO: 149; optionally, wherein each CDR has at least 85% sequence identity with the corresponding CDR.
   23. An antibody or antigen-binding fragment thereof that specifically binds a proTGFβ1 complex, wherein the antibody comprises a variable heavy chain sequence at least 95% identical to SEQ ID NO: 222, and a variable light chain sequence at least 95% identical to SEQ ID NO: 223.
   24. An antibody or antigen-binding fragment thereof that specifically binds a proTGFβ1 complex, wherein the antibody comprises:
i) an H-CDR1 comprising an amino acid sequence as set forth in SEQ ID NO: 150;
ii) an H-CDR2 comprising an amino acid sequence as set forth in SEQ ID NO: 151;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 148; and,
vi) an L-CDR3 comprising an amino acid sequence as set forth in SEQ ID NO: 149; optionally, wherein each CDR has at least 85% sequence identity with the corresponding CDR.
   25. An antibody or antigen-binding fragment thereof that specifically binds a proTGFβ1 complex, wherein the antibody comprises a variable heavy chain sequence at least 95% identical to SEQ ID NO: 224, and a variable light chain sequence at least 95% identical to SEQ ID NO: 223.
   26. An antibody or antigen-binding fragment thereof that specifically binds a proTGFβ1 complex, wherein the antibody comprises:
i) an H-CDR1 comprising an amino acid sequence as set forth in SEQ ID NO: 114;
ii) an H-CDR2 comprising an amino acid sequence as set forth in SEQ ID NO: 115;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   27. An antibody or antigen-binding fragment thereof that specifically binds a proTGFβ1 complex, wherein the antibody comprises a variable heavy chain sequence at least 95% identical to SEQ ID NO: 226, and a variable light chain sequence at least 95% identical to SEQ ID NO: 15.
   28. An antibody or antigen-binding fragment thereof that specifically binds a proTGFβ1 complex, wherein the antibody comprises:
i) an H-CDR1 comprising an amino acid sequence as set forth in SEQ ID NO: 168;
ii) an H-CDR2 comprising an amino acid sequence as set forth in SEQ ID NO: 115;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   29. An antibody or antigen-binding fragment thereof that specifically binds a proTGFβ1 complex, wherein the antibody comprises a variable heavy chain sequence at least 95% identical to SEQ ID NO: 228, and a variable light chain sequence at least 95% identical to SEQ ID NO: 15.
   30. An antibody or antigen-binding fragment thereof that specifically binds a proTGFβ1 complex, wherein the antibody comprises:
i) an H-CDR1 comprising an amino acid sequence as set forth in SEQ ID NO: 120;
ii) an H-CDR2 comprising an amino acid sequence as set forth in SEQ ID NO: 121;
iii) an H-CDR3 comprising an amino acid sequence as set forth in SEQ ID NO: 110;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   31. An antibody or antigen-binding fragment thereof that specifically binds a proTGFβ1 complex, wherein the antibody comprises a variable heavy chain sequence at least 95% identical to SEQ ID NO: 230, and a variable light chain sequence at least 95% identical to SEQ ID NO: 15.
   32. An antibody or antigen-binding fragment thereof that specifically binds a proTGFβ1 complex, wherein the antibody comprises:
i) an H-CDR1 comprising an amino acid sequence as set forth in SEQ ID NO: 114;
ii) an H-CDR2 comprising an amino acid sequence as set forth in SEQ ID NO: 121;
iii) an H-CDR3 comprising an amino acid sequence as set forth in SEQ ID NO: 182;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   33. An antibody or antigen-binding fragment thereof that specifically binds a proTGFβ1 complex, wherein the antibody comprises a variable heavy chain sequence at least 95% identical to SEQ ID NO: 232, and a variable light chain sequence at least 95% identical to SEQ ID NO: 15.
   34. An antibody or antigen-binding fragment thereof that specifically binds a proTGFβ1 complex, wherein the antibody comprises:
i) an H-CDR1 comprising an amino acid sequence as set forth in SEQ ID NO: 120;
ii) an H-CDR2 comprising an amino acid sequence as set forth in SEQ ID NO: 121;
iii) an H-CDR3 comprising an amino acid sequence as set forth in SEQ ID NO: 188;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   35. An antibody or antigen-binding fragment thereof that specifically binds a proTGFβ1 complex, wherein the antibody comprises a variable heavy chain sequence at least 95% identical to SEQ ID NO: 234, and a variable light chain sequence at least 95% identical to SEQ ID NO: 15.
   36. An antibody or antigen-binding fragment thereof that specifically binds a proTGFβ1 complex, wherein the antibody comprises:
i) an H-CDR1 comprising an amino acid sequence as set forth in SEQ ID NO: 150;
ii) an H-CDR2 comprising an amino acid sequence as set forth in SEQ ID NO: 193;
iii) an H-CDR3 comprising an amino acid sequence as set forth in SEQ ID NO: 194;
iv) an L-CDR1 comprising an amino acid sequence as set forth in SEQ ID NO: 111;
v) an L-CDR2 comprising an amino acid sequence as set forth in SEQ ID NO: 136; and,
vi) an L-CDR3 comprising an amino acid sequence as set forth in SEQ ID NO: 12; optionally, wherein each CDR has at least 85% sequence identity with the corresponding CDR.
   37. An antibody or antigen-binding fragment thereof that specifically binds a proTGFβ1 complex, wherein the antibody comprises a variable heavy chain sequence at least 95% identical to SEQ ID NO: 236, and a variable light chain sequence at least 95% identical to SEQ ID NO: 15.
   38. An antibody or antigen-binding fragment thereof that specifically binds a proTGFβ1 complex, wherein the antibody comprises:
i) an H-CDR1 comprising an amino acid sequence as set forth in SEQ ID NO: 126;
ii) an H-CDR2 comprising an amino acid sequence as set forth in SEQ ID NO: 127;
iii) an H-CDR3 comprising an amino acid sequence as set forth in SEQ ID NO: 200;
iv) an L-CDR1 comprising an amino acid sequence as set forth in SEQ ID NO: 111;
v) an L-CDR2 comprising an amino acid sequence as set forth in SEQ ID NO: 112; and,
vi) an L-CDR3 comprising an amino acid sequence as set forth in SEQ ID NO: 113; optionally, wherein each CDR has at least 85% sequence identity with the corresponding CDR.
   39. An antibody or antigen-binding fragment thereof that specifically binds a proTGFβ1 complex, wherein the antibody comprises a variable heavy chain sequence as set forth in SEQ ID NO: 238, and a variable light chain sequence as set forth in SEQ ID NO: 211.
   40. An antibody or antigen-binding fragment thereof that specifically binds a proTGFβ1 complex, wherein the antibody comprises:
i) an H-CDR1 comprising an amino acid sequence as set forth in SEQ ID NO: 126;
ii) an H-CDR2 comprising an amino acid sequence as set forth in SEQ ID NO: 127;
iii) an H-CDR3 comprising an amino acid sequence as set forth in SEQ ID NO: 206;
iv) an L-CDR1 comprising an amino acid sequence as set forth in SEQ ID NO: 111;
v) an L-CDR2 comprising an amino acid sequence as set forth in SEQ ID NO: 112; and,
vi) an L-CDR3 comprising an amino acid sequence as set forth in SEQ ID NO: 113.
   41. An antibody or antigen-binding fragment thereof that specifically binds a proTGFβ1 complex, wherein the antibody comprises a variable heavy chain sequence having the sequence set forth in SEQ ID NO: 240, and a variable light chain sequence having the sequence set forth in SEQ ID NO: 211.
   42. An antibody or antigen-binding fragment thereof that specifically binds a proTGFβ1 complex, wherein the antibody comprises a variable heavy chain sequence at least 95% identical to SEQ ID NO: 216, and a variable light chain sequence at least 95% identical to SEQ ID NO: 243.
   43. An isolated monoclonal antibody or antigen-binding fragment thereof that specifically binds a proTGFβ1 complex, wherein the antibody comprises two or more of the following CDRs:
i) an H-CDR1 comprising an amino acid sequence as set forth in SEQ ID NO: 250, optionally with 1 amino acid change;
ii) an H-CDR2 comprising an amino acid sequence as set forth in SEQ ID NO: 251, optionally with 1 amino acid change;
iii) an H-CDR3 comprising an amino acid sequence as set forth in SEQ ID NO: 110, optionally with 1 amino acid change;
iv) an L-CDR1 comprising an amino acid sequence as set forth in SEQ ID NO: 111, optionally with 1 amino acid change;
v) an L-CDR2 comprising an amino acid sequence as set forth in SEQ ID NO: 136, optionally with 1 amino acid change; and,
vi) an L-CDR3 comprising an amino acid sequence as set forth in SEQ ID NO: 12, optionally with 1 amino acid change.
with the proviso that the antibody comprises at least one CDR that is selected from the group consisting of the H-CDR1, H-CDR2, L-CDR2 and L-CDR3 listed above.
   44. An isolated monoclonal antibody or antigen-binding fragment thereof that specifically binds a proTGFβ1 complex, wherein the antibody comprises:
i) an H-CDR1 comprising an amino acid sequence as set forth in SEQ ID NO: 250, optionally with 1 or 2 amino acid changes;
ii) an H-CDR2 comprising an amino acid sequence as set forth in SEQ ID NO: 251, optionally with 1 amino acid change;
iii) an H-CDR3 comprising an amino acid sequence as set forth in SEQ ID NO: 110, optionally with 1 amino acid change;
iv) an L-CDR1 comprising an amino acid sequence as set forth in SEQ ID NO: 111, optionally with 1 amino acid change;
v) an L-CDR2 comprising an amino acid sequence as set forth in SEQ ID NO: 136, optionally with 1 amino acid change; and,
vi) an L-CDR3 comprising an amino acid sequence as set forth in SEQ ID NO: 12, optionally with 1 or 2 amino acid changes.
   45. An isolated monoclonal antibody or antigen-binding fragment thereof that specifically binds a proTGFβ1 complex and inhibits TGFβ1 activation, wherein the antibody or the fragment binds each of the hLTBP1-proTGFβ1 and hLTBP3-proTGFβ1 complexes with KD of ≤1 nM, and binds each of the hGARP-proTGFβ1 and hLRRC33-proTGFβ1 complexes with KD >1 nM, as measured by solution equilibrium titration.
   46. An isolated monoclonal antibody or antigen-binding fragment thereof that specifically binds a proTGFβ1 complex and inhibits TGFβ1 activation, wherein the antibody or the fragment binds each of the hLTBP1-proTGFβ1 and hLTBP3-proTGFβ1 complexes with KD of ≤1 nM as measured by solution equilibrium titration, and, wherein the average affinity for hLTBP1-proTGFβ1 and hLTBP3-proTGFβ1 complexes is at least five-fold greater than the average affinity for hGARP-proTGFβ1 and hLRRC33-proTGFβ1 complexes.
   47. An isolated monoclonal antibody or antigen-binding fragment thereof that specifically binds each of: hLTBP1-proTGFβ1, hLTBP3-proTGFβ1, hGARP-proTGFβ1 and hLRRC33-proTGFβ1 complexes with KD of ≤10 nM (preferably ≤5 nM); and, wherein the antibody has greater than five-fold matrix/LTBP-bias in relative affinities between at least one of the matrix-associated complexes (e.g., hLTBP1-proTGFβ1 and/or hLTBP3-proTGFβ1) and at least one of the cell-associated complexes (e.g., hGARP-proTGFβ1 and/or hLRRC33-proTGFβ1).
   48. The antibody or fragment of any one of the preceding embodiments, wherein the antibody or fragment binds at least a portion of the prodomain of the proTGFβ1.
   49. The antibody or fragment of embodiments 48, wherein the portion of the prodomain comprises one or more amino acid residues of Latency Lasso.
   50. The antibody or fragment of any one of the preceding embodiments, wherein the antibody or fragment binds a combinatorial epitope that comprises one or more amino acid residues of the Latency Lasso (within the prodomain) and one or more amino acid residues of the growth factor domain of the proTGFβ1.
   51. The antibody or antigen-binding fragment thereof according to any one of the preceding embodiments, wherein the antibody makes contact with the following amino acid residues on a proTGFβ1 complex S35, G37, E38, V39, P40, P41, G42, P43, R274, K280, and H283, as set forth in SEQ ID NO: 24.
   52. The antibody or the fragment according to any one of the preceding embodiments, wherein the antibody or the fragment inhibits release of mature growth factor from each of the following proTGFβ1 complexes with an IC₅₀ of ≤10 nM against a hLTBP1-TGFβ1 complex and a hLTBP3-TGFβ1 complex as measured by a cell-based potency assay.
   53. The antibody or the fragment according to any one of the preceding embodiments, wherein the antibody or the fragment inhibits release of mature growth factor from each of the following proTGFβ1 complexes with an IC₅₀ of ≤5 nM against a hLTBP1-TGFβ1 complex and a hLTBP3-TGFβ1 complex as measured by a cell-based potency assay.
   54. The antibody or the fragment according to any one of the preceding embodiments, wherein measured KD values for GARP-proTGFβ1 complex are about 5-20-fold greater than measured K_{D} values to human LRRC33-proTGFβ1, human LTBP1-proTGFβ1 and human LTBP3-proTGFβ1 complexes.
   55. The antibody or the fragment according to any one of the preceding embodiments, wherein the antibody or the fragment wherein the measured KD values fora human GARP-proTGFβ1 complex is 1 nM or greater, while measured KD values for each of the other three complexes (human LRRC33-proTGFβ1, human LTBP1-proTGFβ1 and human LTBP3-proTGFβ1) is in a sub-nanomolar range (˜0.1-0.9 nM).
   56. The antibody or the fragment of any of the preceding embodiments, wherein the antibody inhibits release of mature TGFβ1 growth factor from each of the proTGFβ1 complexes but not from proTGFβ2 or proTGFβ3 complexes.
   57. The antibody or the fragment according to any one of the preceding embodiments, wherein the antibody or fragment is a human IgG1 or IgG4 subtype.
   58. The antibody or the fragment according to any one of the preceding embodiments, wherein the antibody or fragment comprises a Serto Pro mutation that produces an IgG1-like hinge.
   59. The antibody or the fragment according to any one of the preceding embodiments, wherein the antibody or fragment comprises a hinge region comprising the amino acid sequence as set forth in SEQ ID NO: 54.
   60. The antibody or the fragment according to any one of the preceding embodiments, wherein the antibody or fragment cross-reacts with a murine counterpart.
   61. The antibody or the fragment according to any one of the preceding embodiments, wherein the antibody or fragment cross-reacts with human, cynomolgus, mouse, and rat counterparts.
   62. The antibody or the fragment according to any one of the preceding embodiments, wherein the antibody or the fragment cross-reacts with human and mouse forms of each of the following proTGFβ1 complexes:
a) a LTBP1-proTGFβ1 complex;
b) a LTBP3-proTGFβ1 complex;
c) a GARP-proTGFβ1 complex and,
d) a LRRC33-proTGFβ1 complex.
   63. The antibody or the fragment according to any one of the preceding embodiments, wherein the antibody or the fragment is a pH-sensitive antibody characterized in that the antibody or the fragment has a lower affinity to the antigen at pH 5 than at pH 7, as measured by biolayer interferometry.
   64. The antibody or the fragment according to any one of the preceding embodiments, wherein the antibody or the fragment specifically binds a proTGFβ1 C4S protein and has a K_{dis} (a.k.a. K_{off}) of ≥5×10⁻³ s⁻¹ (e.g., ≥5.1×10⁻³, 5.2×10⁻³, ≥5.3×10⁻³, ≥5.4×10⁻³, ≥5.5×10⁻³, ≥5.6×10⁻³, ≥5.7×10⁻³, ≥5.8×10⁻³, ≥5.9×10⁻³, or ≥6.0×10⁻³) at pH 5, as measured by a suitable affinity assay (e.g., biolayer interferometry, and/or surface plasmon resonance).
   65. The antibody or the fragment according to any one of the preceding embodiments, wherein the antibody or the fragment specifically binds a GARP-TGFβ1 complex, a LTBP1-TGFβ1 complex, a LTBP3-TGFβ1 complex and a LRRC33-TGFβ1 complex and has a K_{dis} (a.k.a. K_{off}) of ≥5×10⁻³ s⁻¹ (e.g., ≥5.1×10⁻³, ≥5.2×10⁻³, ≥5.3×10⁻³, ≥5.4×10⁻³, ≥5.5×10⁻³, ≥5.6×10⁻³, ≥5.7×10⁻³, ≥5.8×10⁻³, ≥5.9×10⁻³, or ≥6.0×10⁻³) at pH 5 for one or more of the complexes, as measured by a suitable affinity assay (e.g., biolayer interferometry, and/or surface plasmon resonance).
   66. The antibody or the fragment according to any one of the preceding embodiments, wherein the antibody or fragment has a K_{dis}≥5.6×10⁻³ at pH 5.
   67. The antibody or the fragment according to any one of the preceding embodiments, wherein the antibody or fragment has a pH 5 K_{dis} to pH 7 K_{dis} ratio (i.e., K_{dis} at pH 5:K_{dis} at pH7) of ≥1.5 (e.g., ≥1.6, ≥1.7, ≥1.8, ≥1.9, or ≥2.0), as measured by a suitable affinity assay (e.g., biolayer interferometry, and/or surface plasmon resonance).
   68. The antibody or the fragment according to any one of the preceding embodiments, wherein the antibody or fragment has a K_{dis} ratio of ≥2.0, as measured by biolayer interferometry.
   69. The antibody or the fragment according to any one of the preceding embodiments, wherein the antibody or fragment has a maximally tolerated dose (MTD) of >100 mg/kg when dosed weekly to a patient for at least 4 weeks.
   70. A composition comprising the antibody or the fragment according to any one of the preceding embodiments, and a pharmaceutically acceptable excipient.
   71. A containing means comprising the composition of embodiment 66, wherein optionally the containing means is a vial or a syringe, wherein optionally the containing means contains a single dosage unit or multiple dosage units.
   72. A kit comprising the composition of embodiment 70 or 71.
   73. Use of the antibody or the fragment according to any one of the preceding embodiments in the manufacture of a medicament for the treatment of a TGFβ1-related disorder in a subject.
   74. The antibody or the fragment according to any one of the preceding embodiments, for the treatment of a TGFβ1-related disorder in a subject.
   75. The antibody or the fragment for use according to embodiment 69, wherein the TGFβ1-related disorder is fibrotic disorder or cancer.
   76. The antibody or the fragment for use according to embodiment 75, wherein the fibrotic disorder is an organ fibrosis, wherein optionally the organ fibrosis is selected from the group consisting of: kidney fibrosis, liver fibrosis, lung fibrosis, cardiac fibrosis, pancreatic fibrosis, skin fibrosis, scleroderma, muscle fibrosis, uterine fibrosis, and endometriosis and wherein further optionally the organ fibrosis is an advanced organ fibrosis.
   77. The antibody or the fragment for use according to embodiment 73, for the treatment of a disease or disorders involving dysregulation of the extracellular matrix.
   78. The antibody or the fragment for use according to embodiment 73, wherein the disease or disorder comprises a fibrotic disorder, a disease involving endothelial-to-mesenchymal transition (EndMT), a disease involving epithelial-to-mesenchymal transition (EMT), or a disease involving proteases.
   79. The antibody or fragment for use according to embodiment 76, wherein the subject has nonalcoholic steatohepatitis (NASH) or non-alcoholic fatty liver disease (NAFLD).
   80. The antibody or the fragment for use according to embodiment 76, wherein the lung fibrosis is idiopathic pulmonary fibrosis (IPF).
   81. The antibody or the fragment for use according to embodiment 76, wherein the subject has chronic kidney disease (CKD).
   82. The antibody or fragment for use according to embodiment 74, wherein the TGFβ1-related disorder is a fibrotic disorder comprising chronic inflammation, wherein optionally the disorder is a muscular dystrophy, multiple sclerosis (MS), or Cystic Fibrosis (CF).
   83. The antibody or fragment for use according to embodiment 82, wherein the muscular dystrophy is Duchenne muscular dystrophy (DMD).
   84. The antibody or fragment for use according to embodiment 82, wherein the MS comprises perivascular fibrosis.
   85. The antibody or fragment for use according to embodiment 73, wherein the TGFβ1-related disorder is a myeloproliferative disorder.
   86. The antibody or fragment for use according to embodiment 85, wherein the myeloproliferative disorder is myelofibrosis.
   87. The antibody or fragment for use according to embodiment 73, wherein the TGFβ1-related disorder is cancer.
   88. The antibody or fragment for use according to embodiment 87, wherein the cancer is characterized by acquired resistance or innate resistance.
   89. The antibody or fragment for use according to any one of embodiments 87 and 88, wherein the cancer comprises a solid tumor.
   90. The antibody or fragment for use according to embodiment 89, wherein the solid tumor is poorly responsive to a cancer therapy, wherein optionally the cancer therapy is a checkpoint inhibitor therapy.
   91. The antibody or fragment for use according to embodiments 89-90, wherein the tumor is characterized by immune exclusion.
   92. The antibody or fragment for use according to embodiments 89-91, wherein the tumor comprises macrophage infiltration.
   93. The antibody or fragment for use according to embodiment 89-92, wherein the tumor comprises stroma enriched with CAFs.
   94. The antibody or fragment for use according to embodiments 87-93, wherein the subject is receiving or is a candidate for receiving a cancer therapy selected from the group consisting of: chemotherapy, radiation therapy, CAR-T, cancer vaccine, checkpoint inhibitor therapy.
   95. The antibody or fragment for use according to embodiment 87-94, wherein the subject is not a candidate for undergoing surgical resection of the tumor.
   96. The antibody or fragment for use according to any one of embodiments 1-69 for use in the enhancement of host immunity in a human subject,
wherein the subject has cancer, and
wherein the immune responses comprise anti-cancer immunity.
   97. The antibody or fragment for use according to embodiment 96 wherein the enhancement of host immunity includes reducing immune-exclusion from a tumor or promoting immune cell infiltrates into a tumor.
   98. The antibody or fragment for use according to embodiments 96 and 97, wherein the subject is receiving or a candidate for receiving an engineered immune cell therapy.
   99. The antibody or fragment for use according to any one of embodiments 96-98, wherein the subject is receiving or is a candidate for receiving a cancer vaccine.
   100. The antibody or fragment for use according to any one of embodiments 85-99, wherein the subject is receiving or is a candidate for receiving an immune checkpoint inhibitor therapy, wherein optionally the subject is poorly responsive to the immune checkpoint inhibitor therapy.
   101. The antibody or fragment for use according to any one of embodiment 73-100 wherein the subject is further treated with a second therapy, wherein optionally the second therapy comprises a TGFβ3 inhibitor.
   102. The antibody or fragment for use according to embodiment 101, wherein the antibody or fragment, and the TGFβ3 inhibitor, are administered to the subject simultaneously, serially, or separately.
   103. The antibody or fragment for use according to embodiment 102, wherein the antibody or fragment, and the TGFβ3 inhibitor, are formulated into a single formulation, or into separate formulations.
   104. The antibody or fragment for use according to embodiment 73-103, wherein the subject has TGFβ1-positive and TGFβ3-positive cancer or fibrotic tissue.
   105. The antibody or fragment for use according to any one of embodiments 103 or 104, wherein the subject is, or has been determined to be, partially responsive to the TGFβ1 inhibitor therapy.
   106. The antibody or fragment for use according to any one of embodiments 73-105, wherein the antibody or fragment, alone or in combination with one or more additional therapeutics, block activation of macrophage-associated TGFb1.
   107. The antibody or fragment for use according to any one of embodiments 75-79, wherein the antibody or fragment, alone or in combination with one or more additional therapeutics, blocks hepatic stellate cell activation.
   108. The antibody or fragment for use according to any one of embodiments 75-79, wherein the antibody or fragment, alone or in combination with one or more additional therapeutics, reduces liver surface nodes (LSN) and/or liver stiffness.
   109. The antibody or fragment for use according to any one of embodiments 75-79, wherein the antibody or fragment, alone or in combination with one or more additional therapeutics, reduces hepatic fat as measured by MRI-PDFF.
   110. The antibody or fragment for use according to any one of embodiments 109, wherein the reduction of hepatic fat is at least ≥20%, e.g., ≥20%, ≥25%, ≥30%, ≥35%, ≥40%, ≥45%, or ≥50%, as compared to the hepatic fat prior to treatment.
   111. The antibody or fragment for use according to any one of embodiments 75-79, wherein the antibody or fragment alone or in combination with one or more additional therapeutics reduces serum ALT and/or GGT by at least 20%, e.g., ≥20%, ≥25%, ≥30%, ≥35%, ≥40%, ≥45%, or ≥50%, as compared to the ALT and/or GGT prior to treatment.
   112. The antibody or fragment for use according to any one of embodiments 75-79, wherein the antibody or fragment alone or in combination with one or more additional therapeutics reduces bile acid synthesis.
   113. The antibody or fragment for use according to any one of embodiments 75-79, wherein the antibody or fragment, alone or in combination with one or more additional therapeutics, reduces intrahepatic triglyceride content to ≤5.5% of liver volume.
   114. The antibody or fragment for use according to any one of embodiments 73-113, wherein the antibody or fragment has at least a 3-fold, 6-fold, and more preferably 10-fold therapeutic window.
   115. The antibody or fragment for use according to any one of embodiments 73-114, wherein the antibody or fragment is therapeutically effective at 0.1 mg/kg to about 30 mg/kg when administered weekly to a patient.
   116. The antibody or fragment for use according to embodiment 115, wherein the antibody or fragment is therapeutically effective at 3 mg/kg to about 30 mg/kg when administered weekly to a patient.
   117. A method for treating a subject having a TGFβ1-related disease or condition, comprising the following steps:
i) selecting a patient diagnosed with a TGFb1-related disease or condition; and,
ii) administering to the patient the antibody or the fragment according to anyone of embodiments 1-69 in an amount effective to treat the disease or condition.
   118. The method of embodiment 117, wherein the TGFb1-related disease or condition is fibrosis (e.g., organ fibrosis).
   119. The method of embodiment 118, wherein the fibrosis is liver fibrosis, lung fibrosis, kidney fibrosis, or cardiac fibrosis.
   120. The method of any one embodiments 117-119, wherein the selection step (i) comprises detection of immune cells or one or more markers thereof.
   121. The method of anyone of claims 117-120, wherein the immune cell marker is selected from the group consisting of: CD8, CD3, CD4, CD11b, CD163, CD68, CD14, CD34, CD25, CD47.
   122. The method of claim 117 or 121, wherein the immune cells are selected from the group consisting of: cytotoxic T lymphocytes, regulatory T cells, MDSCs, tumor-associated macrophages, NK cells, dendritic cells, and neutrophils.
   123. The method of anyone of embodiments 117-119, wherein the selection step (i) comprises detection of fibrosis markers (e.g., PAI-1, ACTA2, CCL2, Col1a1, Col3a1, FN-1, CTGF, and/or TGFβ1).
   124. The method of anyone embodiments 117-119, wherein the selection step (i) comprises detection of myofibroblasts cells, or one or more markers thereof.
   125. The method of any one embodiments 117-124, wherein the selection step (i) comprises detection of hepatic steatosis, hepatic triglycerides, immune cells, and/or myofibroblasts.
   126. The method of any one of embodiments 120-125, wherein the detection comprises a biopsy analysis, serum marker analysis, and/or in vivo imaging.
   127. The method of embodiment 126, wherein the in vivo imaging comprises ultrasound, ultrasound elastography, CT scan, MRI, PET-SPECT, and/or optical fluorescence/bioluminescence.
   128. The method of embodiment 126, wherein the in vivo imaging comprises FibroScan (TE), pSWE, 2D-3D SWE, and/or MRE.
   129. The method of embodiment 126, wherein the in vivo imaging comprises ultrasonography, CAP, MRI-PDFF, and/or MRS.
   130. The method of any one of embodiments 126-129, wherein the in vivo imaging comprises director indirect labeling of immune cells or antibody that binds a cell-surface marker of immune cells.
   131. The method of any one of claims 126-129, wherein the in vivo imaging comprises the use of a tracer.
   132. The method of claim 131, wherein the tracer is a radioisotope.
   133. The method of claim 132, wherein the radioisotope is a positron-emitting isotope.
   134. The method of claim 133, wherein the radioisotope is selected from the group consisting of: ¹⁸F, ¹¹C, ¹³N, ¹⁵O, ⁶Ga, ¹⁷⁷Lu, and ⁸⁹Zr.
   135. The method of any one of claims 126-134, wherein the in vivo imaging comprise the use of labeled antibodies in immune-PET.
   136. The method of any one of embodiments 117-135, wherein the treatment reduces triglycerides, steatosis, liver surface nodules, inflammation, and/or macrophages, in the diseased tissue.
   137. The method of any one of embodiments 117-136, wherein the treatment reduces intrahepatic triglyceride content to ≤5.5% of liver volume.
   138. The method of any one of embodiments 117-137, wherein the treatment reduces MDSCs in the diseased tissue.
   139. The method of any one of embodiments 117-138, wherein the treatment reduces macrophages in the diseased tissue.
   140. The method of anyone of embodiments 117-139, wherein the treatment blocks activation of macrophage-associated TGFb1.
   141. The method of anyone of embodiments 117-140, wherein the treatment blocks hepatic stellate cell activation.
   142. The method of anyone of embodiments 117-141, wherein the treatment reduces liver surface nodes (LSN) and/or liver stiffness.
   143. The method of any one of embodiments 117-142, wherein the treatment reduces hepatic fat as measured by MRI-PDFF.
   144. The method of any one of embodiments 117-143, wherein the treatment reduces of hepatic fat is at least 20%, e.g., ≥20%, ≥25%, ≥30%, ≥35%, ≥40%, ≥45%, or ≥50%, as compared to the hepatic fat prior to treatment.
   145. The method of any one of embodiments 117-144, wherein the treatment reduces serum ALT and/or GGT by at least 20%, e.g., ≥20%, ≥25%, ≥30%, ≥35%, ≥40%, ≥45%, or ≥50%, as compared to the ALT and/or GGT prior to treatment.
   146. The method of any one of embodiments 117-145, wherein the treatment reduces bile acid synthesis.
   147. The method of any one of embodiments 117-146, wherein the treatment reduces intrahepatic triglyceride content to ≤5.5% of liver volume.
   148. The method of any one of embodiments 117-147, wherein the effective amount is from 0.1 mg/kg to 30 mg/kg, optionally 3 mg/kg to 30 mg/kg.
   149. The method of any one of embodiments 126-148, wherein the in vivo imaging is performed for monitoring a therapeutic response to the TGFβ1 inhibition therapy in the subject.
   150. A method for treating cancer, the method comprising steps of:
i) selecting a patient diagnosed with cancer comprising a solid tumor, wherein the solid tumor is or is suspected to be an immune excluded tumor;
ii) administering to the patient the antibody or the fragment according to anyone of embodiments 1-65 in an amount effective to treat the cancer, wherein the patient has received, or is a candidate for receiving a cancer therapy selected from the group consisting of: immune checkpoint inhibition therapies, chemotherapies, radiation therapies, engineered immune cell therapies, and cancer vaccine therapies.
   151. The method of claim 150, wherein the immune checkpoint inhibitor is a PD-1 inhibitor or a PD-L1 inhibitor.
   152. The method of claim 151, wherein the selection step (i) comprises detection of immune cells or one or more markers thereof.
   153. The method of claim 152, wherein the detection comprises a tumor biopsy analysis, serum marker analysis, and/or in vivo imaging.
   154. The method of claim 152 or 153, wherein the immune cells are selected from the group consisting of: cytotoxic T lymphocytes, regulatory T cells, MDSCs, tumor-associated macrophages, NK cells, dendritic cells, and neutrophils.
   155. The method of anyone of claims 153-154, wherein the immune cell marker is selected from the group consisting of: CD8, CD3, CD4, CD11b, CD163, CD68, CD14, CD34, CD25, CD47.
   156. The method of claim 153, wherein the in vivo imaging comprises T cell tracking.
   157. The method of claim 153 or 156, wherein the in vivo imaging comprises the use of PET-SPECT, MRI and/or optical fluorescence/bioluminescence.
   158. The method of claim 156 or 157, wherein the in vivo imaging comprises director indirect labeling of immune cells or antibody that binds a cell-surface marker of immune cells.
   159. The method of any one of embodiments 153-158, wherein the in vivo imaging comprises the use of a tracer.
   160. The method of claim 159, wherein the tracer is a radioisotope.
   161. The method of claim 160, wherein the radioisotope is a positron-emitting isotope.
   162. The method of claim 161, wherein the radioisotope is selected from the group consisting of: ¹⁸F, ¹¹C, ¹³N, ¹⁵O, ⁶⁸Ga, ¹⁷⁷Lu, and ⁸⁹Zr.
   163. The method of any one of claims 153-162, wherein the in vivo imaging comprise the use of labeled antibodies in immune-PET.
   164. The method of any one of claims 153-163, wherein the in vivo imaging is performed for monitoring a therapeutic response to the TGFβ1 inhibition therapy in the subject.
   165. The method of claim 164, wherein the therapeutic response comprises conversion of an immune excluded tumor into an inflamed tumor.
   166. The method of embodiment 125, wherein the selected patient has an intrahepatic triglyceride content of >5.5% of liver volume, optionally wherein the intrahepatic triglyceride content is >10% of liver volume.
   167. A method of manufacturing the antibody or the fragment according to any one of embodiments 1-44, the method comprising steps of:
i) providing an antigen that comprises a proTGFβ1 complex, optionally comprising at least two of: LTBP1, LTBP3, GARP, LRRC33 or a fragment thereof,
ii) selecting fora pool of antibodies or fragments for ability to bind the antigen of step (i);
iii) optionally removing antibodies or fragments from the pool that show undesirable binding profiles;
iv) selecting fora pool of antibodies or fragments selected from step(s) (ii) and/or(iii) for ability to inhibit TGFβ1;
v) optionally generating a fully human or humanized antibody or fragment of an antibody, antibodies or fragments selected from step (iv) so as to provide a human or humanized inhibitor;
vi) carrying out in vitro binding assay to determine affinities for LTBP1-proTGFβ1, LTBP3-proTGFβ1, GARP-proTGFβ1 and LRRC33-proTGFβ1,
optionally, vii) carrying out functional assay to determine or confirm activity of the inhibitor towards TGFβ1, TGFβ2 and/or TGFβ3.
   168. The method of embodiment 167, further comprising a step of evaluating a candidate antibody or a fragment thereof in an in vivo efficacy study and in vivo toxicology study in a preclinical animal model, thereby determining effective amounts shown to be both efficacious and safe or tolerable.
   169. The method of embodiment 167 or 168, further comprising a step of formulating into a pharmaceutical composition.
   170. The composition according to embodiment 169 for therapeutic use in the treatment of fibrosis in a human subject.
   171. The composition according to embodiment 169 for therapeutic use in the treatment of myelofibrosis in a human subject.
   172. The composition according to embodiment 169 for therapeutic use in the treatment of cancer in a human subject.
   173. The antibody or fragment of any one of embodiments 1-44, wherein the antibody or fragment protects at least a portion of an amino acid sequence of proTGFβ1 comprising SPPSQGEVPPGPLPEAVLALYNST (SEQ ID NO: 261) from solvent exposure as measured by HDX-MS.
   174. The antibody or fragment of any one of embodiments 1-44 and 173, wherein the antibody or fragment protects at least a portion of an amino acid sequence of proTGFβ1 comprising LREAVPE (SEQ ID NO: 259) from solvent exposure as measured by HDX-MS.
   175. The antibody or fragment of any one of embodiments 1-44, 173, and 174, wherein the antibody or fragment protects at least a portion of an amino acid sequence of proTGFβ1 comprising WKWIHEPKGYHANFCLG (SEQ ID NO: 262) from solvent exposure as measured by HDX-MS.

Those skilled in the art will recognize, or be able to ascertain using no more than routine experimentation, many equivalents to the specific embodiments of the invention described herein. Such equivalents are intended to be encompassed by the following claims.

1. An isolated monoclonal antibody or an antigen-binding fragment thereof that specifically binds a proTGFβ1 complex, wherein the antibody or the antigen-binding fragment comprises an H-CDR1, an H-CDR2, an H-CDR3, an L-CDR1, an L-CDR2, and an L-CDR3, wherein:
i) the H-CDR1 has an amino acid sequence represented by FTF(X₁)(X₂)(X₃)AM(X₄), wherein X₁ is A or S; X₂ is N, D, S, or A; X₃ is Y or F; and/or X₄ is S, T, or V (SEQ ID NO: 252);
ii) the H-CDR2 has an amino acid sequence represented by (X₁)IS(X₂)(X₃)(X₄)(X₅)(X₆)(X₇)Y(X₈)ADSVKG, wherein optionally, X₁ is S or A; X₂ is G or S; X₃ is S, T, or F; X₄ is G or A; X₅ is G, A, F, or S; X₆ is A, H, T, S, or V; X₇ is T or I; and/or, Xₐ is Y or F (SEQ ID NO: 253);
iii) the H-CDR3 has an amino acid sequence represented by A(X₁)VSS(X₂)(X₃)WD(X₄)D(X₅), wherein optionally, X₁ is R or T; X₂ is G or Y; X₃ is H or L; X₄ is F, Y, or L; and/or X₅ is Y or E (SEQ ID NO: 254);
iv) the L-CDR1 has an amino acid sequence represented by (X₁)ASQ(X₂)IS(X₃)(X₄)LN, wherein optionally, X₁ is R or Q; X₂ is S or D; X₃ is S or N; and/or X₄ is Y or S (SEQ ID NO: 255);
v) the L-CDR2 has an amino acid sequence represented by (X₁)AS(X₂)L(X₃)(X₄), wherein optionally, X₁ is D or A; X₂ is S or N; X₃ is Q or E; and/or X₄ is S or T (SEQ ID NO: 256); and,
vi) the L-CDR3 has an amino acid sequence represented by QQ(X₁)(X₂)(X₃)(X₄)P(X₅)T, wherein optionally, X₁ is S, A, T, or V; X₂ is F, Y or P; X₃ is S, N, T, or D; X₄ is A, L, V, or P; and/or X₅ is F or L (SEQ ID NO: 257).

2. (canceled)

3. (canceled)

4. (canceled)

5. An isolated monoclonal antibody or an antigen-binding fragment thereof that specifically binds a proTGFβ1 complex, wherein the isolated monoclonal antibody or the antigen-binding fragment thereof comprises a heavy chain variable domain that is at least 95% identical to the amino acid sequence set forth in SEQ ID NO: 13, and a light chain variable domain that is at least 95% identical to the amino acid sequence set forth in SEQ ID NO: 15.

6. The isolated monoclonal antibody or the antigen-binding fragment thereof according to claim 5, wherein:
i) the heavy chain variable domain comprises amino acid residues D31, A33, S54, Y59, S101, G102, H103, and W104, as set forth in SEQ ID NO: 13, and
ii) the light chain variable domain comprises amino acid residues Y32, Y49, T91, and Y92, as set forth in SEQ ID NO: 15.

7. The isolated monoclonal antibody or the antigen-binding fragment thereof according to claim 1, wherein the antibody is a human IgG4 or IgG1 subtype.

8. The isolated monoclonal antibody or the antigen-binding fragment thereof according to claim 1, wherein the antibody or the antigen-binding fragment inhibits release of mature TGFβ1 growth factor from each of the proTGFβ1 complexes but not from proTGFβ2 or proTGFβ3 complexes.

9. The isolated monoclonal antibody or the antigen-binding fragment thereof according to claim 1, wherein the antibody or the antigen-binding fragment inhibits release of mature growth factor from each of the following proTGFβ1 complexes with an IC₅₀ of ≤5 nM as measured by a cell-based potency assay:
a) a human LTBP1-proTGFβ1 complex;
b) a human LTBP3-proTGFβ1 complex;
c) a human GARP-proTGFβ1 complex; and,
d) a human LRRC33-proTGFβ1 complex.

10. A composition comprising the isolated monoclonal antibody or the antigen-binding fragment according to claim 1, and a pharmaceutically acceptable excipient.

11. A method of treating a fibrotic disorder in a subject, the method comprising administering the isolated monoclonal antibody or the antigen-binding fragment thereof according to claim 1.

12. (canceled)

13. (canceled)

14. The method of claim 11, wherein the fibrotic disorder is an organ fibrosis.

15. The method of claim 20, wherein the lung fibrosis is idiopathic pulmonary fibrosis (IPF).

16. The method of claim 14, wherein the subject has chronic kidney disease (CKD).

17. The method of claim 14, wherein the subject has nonalcoholic steatohepatitis (NASH) or non-alcoholic fatty liver disease (NAFLD).

18. The method of claim 11, wherein the fibrotic disorder is a fibrotic disorder comprising chronic inflammation.

19. The method of claim 11, wherein the subject is further treated with a second therapy, wherein the second therapy comprises a TGFβ3 inhibitor.

20. The method of claim 14, wherein the organ fibrosis is selected from the group consisting of a kidney fibrosis, a liver fibrosis, a lung fibrosis, cardiac fibrosis, a pancreatic fibrosis, a skin fibrosis, scleroderma, a muscle fibrosis, a uterine fibrosis, and endometriosis.

21. The method of claim 14, wherein the organ fibrosis is an advanced organ fibrosis.

22. The method of claim 18, wherein the fibrotic disorder comprising chronic inflammation is a muscular dystrophy, multiple sclerosis (MS), or Cystic Fibrosis (CF).

23. The isolated monoclonal antibody or an antigen-binding fragment thereof, of claim 5, wherein the antibody or the antigen-binding fragment thereof, is pH sensitive.


SEQUENCE LISTING (295 sequences)

SEQ ID NO: 1 - skipped

SEQ ID NO: 2 - protein, 9 aa, Artificial Sequence
  source: /note=“Description of Artificial Sequence: Synthetic peptide”
GFTFADYAM

SEQ ID NO: 3 - skipped

SEQ ID NO: 4 - protein, 7 aa, Artificial Sequence
  source: /note=“Description of Artificial Sequence: Synthetic peptide”
ISGSGAA

SEQ ID NO: 5 - skipped

SEQ ID NO: 6 - protein, 13 aa, Artificial Sequence
  source: /note=“Description of Artificial Sequence: Synthetic peptide”
CARVSSGHWDFDY

SEQ ID NO: 7 - skipped

SEQ ID NO: 8 - protein, 6 aa, Artificial Sequence
  source: /note=“Description of Artificial Sequence: Synthetic peptide”
QSISSY

SEQ ID NO: 9 - skipped

SEQ ID NO: 10 - protein, 3 aa, Artificial Sequence
  source: /note=“Description of Artificial Sequence: Synthetic peptide”
AAS

SEQ ID NO: 11 - skipped

SEQ ID NO: 12 - protein, 9 aa, Artificial Sequence
  source: /note=“Description of Artificial Sequence: Synthetic peptide”
  source: /note=“See specification as filed for detailed description of substitutions and preferred embodiments”
QQTYTVPLT

SEQ ID NO: 13 - protein, 119 aa, Artificial Sequence
  source: /note=“Description of Artificial Sequence: Synthetic polypeptide”
EVQLLESGGGLVQPGGSLRLSCAASGFTFADYAMTWVRQAPGKGLEWVSAISGSGAATYFADSVKGRFTISRDNSKNTLYLQMNSLRAEDTAVYYCARVSSGHWDFDYWGQGTLVTVSS

SEQ ID NO: 14 - skipped

SEQ ID NO: 15 - protein, 107 aa, Artificial Sequence
  source: /note=“Description of Artificial Sequence: Synthetic polypeptide”
DIQLTQSPSSLSASVGDRVTITCRASQSISSYLNWYQQKPGKAPKLLIYAASNLQSGVPSRFSGSGSGTDFTLTISSLQPEDFATYYCQQTYTVPLTFGGGTKVEIK

SEQ ID NO: 16 - skipped

SEQ ID NO: 17 - protein, 445 aa, Artificial Sequence
  source: /note=“Description of Artificial Sequence: Synthetic polypeptide”
EVQLLESGGGLVQPGGSLRLSCAASGFTFADYAMTWVRQAPGKGLEWVSAISGSGAATYFADSVKGRFTISRDNSKNTLYLQMNSLRAEDTAVYYCARVSSGHWDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - skipped

SEQ ID NO: 19 - protein, 214 aa, Artificial Sequence
  source: /note=“Description of Artificial Sequence: Synthetic polypeptide”
DIQLTQSPSSLSASVGDRVTITCRASQSISSYLNWYQQKPGKAPKLLIYAASNLQSGVPSRFSGSGSGTDFTLTISSLQPEDFATYYCQQTYTVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20-23 - skipped (4 entries)

SEQ ID NO: 24 - protein, 361 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 25 - protein, 361 aa, Artificial Sequence
  source: /note=“Description of Artificial Sequence: Synthetic polypeptide”
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - protein, 360 aa, Artificial Sequence
  source: /note=“Description of Artificial Sequence: Synthetic polypeptide”
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 27 - protein, 360 aa, Artificial Sequence
  source: /note=“Description of Artificial Sequence: Synthetic polypeptide”
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 28 - protein, 395 aa, Homo sapiens
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 29 - protein, 395 aa, Artificial Sequence
  source: /note=“Description of Artificial Sequence: Synthetic polypeptide”
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 30 - protein, 394 aa, Artificial Sequence
  source: /note=“Description of Artificial Sequence: Synthetic polypeptide”
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 31 - protein, 394 aa, Artificial Sequence
  source: /note=“Description of Artificial Sequence: Synthetic polypeptide”
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 32 - protein, 392 aa, Homo sapiens
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 33 - protein, 392 aa, Artificial Sequence
  source: /note=“Description of Artificial Sequence: Synthetic polypeptide”
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 34 - protein, 391 aa, Artificial Sequence
  source: /note=“Description of Artificial Sequence: Synthetic polypeptide”
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 35 - protein, 391 aa, Artificial Sequence
  source: /note=“Description of Artificial Sequence: Synthetic polypeptide”
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 36 - protein, 361 aa, Mus sp.
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 37 - protein, 361 aa, Macaca sp.
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 38 - protein, 249 aa, Mus sp.
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 39 - protein, 249 aa, Macaca sp.
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRR

SEQ ID NO: 40 - protein, 360 aa, Mus sp.
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHGALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 41 - protein, 361 aa, Mus sp.
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 42 - protein, 361 aa, Macaca sp.
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 43 - protein, 360 aa, Macaca sp.
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 44 - protein, 1260 aa, Macaca sp.
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRAGALSTGALPPLAPEGDSVASKHAIYAVQVIADPPGPGEGPPAQHAAFLVPLGPGQISAEVQAPPPVVNVRVHHPPEASVQVHRIESSNAEGAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPADGTAAFKEICPAGKGYHILTSHQTLTIQGESDFSLFLHPDGPPKPQQLPESPSQAPPPEDTEEERGVTTDSPVSEERSVQQSHPTATTSPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCVDVDECEAGDVCDNGICTNTPGSFQCQCLSGYHLSRDRSHCEDIDECDFPAACIGGDCINTNGSYRCLCPQGHRLVGGRKCQDIDECTQDPGLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGAEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPQAGPALTFDDCCCRQGRGWGAQCRPCPPRGAGSQCPTSQSESNSFWDTSPLLLGKPRRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 45 - protein, 1228 aa, Mus sp.
GPAGERGTGGGGALARERFKVVFAPVICKRTCLKGQCRDSCQQGSNMTLIGENGHSTDTLTGSAFRVVVCPLPCMNGGQCSSRNQCLCPPDFTGRFCQVPAAGTGAGTGSSGPGLARTGAMSTGPLPPLAPEGESVASKHAIYAVQVIADPPGPGEGPPAQHAAFLVPLGPGQISAEVQAPPPVVNVRVHHPPEASVQVHRIEGPNAEGPASSQHLLPHPKPPHPRPPTQKPLGRCFQDTLPKQPCGSNPLPGLTKQEDCCGSIGTAWGQSKCHKCPQLQYTGVQKPVPVRGEVGADCPQGYKRLNSTHCQDINECAMPGNVCHGDCLNNPGSYRCVCPPGHSLGPLAAQCIADKPEEKSLCFRLVSTEHQCQHPLTTRLTRQLCCCSVGKAWGARCQRCPADGTAAFKEICPGKGYHILTSHQTLTIQGESDFSLFLHPDGPPKPQQLPESPSRAPPLEDTEEERGVTMDPPVSEERSVQQSHPTTTTSPPRPYPELISRPSPPTFHRFLPDLPPSRSAVEIAPTQVTETDECRLNQNICGHGQCVPGPSDYSCHCNAGYRSHPQHRYCVDVNECEAEPCGPGKGICMNTGGSYNCHCNRGYRLHVGAGGRSCVDLNECAKPHLCGDGGFCINFPGHYKCNCYPGYRLKASRPPICEDIDECRDPSTCPDGKCENKPGSFKCIACQPGYRSQGGGACRDVNECSEGTPCSPGWCENLPGSYRCTCAQYEPAQDGLSCIDVDECEAGKVCQDGICTNTPGSFQCQCLSGYHLSRDRSRCEDIDECDFPAACIGGDCINTNGSYRCLCPLGHRLVGGRKCKKDIDECSQDPGLCLPHACENLQGSYVCVCDEGFTLTQDQHGCEEVEQPHHKKECYLNFDDTVFCDSVLATNVTQQECCCSLGAGWGDHCEIYPCPVYSSAEFHSLVPDGKRLHSGQQHCELCIPAHRDIDECILFGAEICKEGKCVNTQPGYECYCKQGFYYDGNLLECVDVDECLDESNCRNGVCENTRGGYRCACTPPAEYSPAQAQCLIPERWSTPQRDVKCAGASEERTACVWGPWAGPALTFDDCCCRQPRLGTQCRPCPPRGTGSQCPTSQSESNSFWDTSPLLLGKSPRDEDSSEEDSDECRCVSGRCVPRPGGAVCECPGGFQLDASRARCVDIDECRELNQRGLLCKSERCVNTSGSFRCVCKAGFTRSRPHGPACLSAAADDAAIAHTSVIDHRGYFH

SEQ ID NO: 46 - protein, 1373 aa, Macaca sp.
NHTGRIKVVFTPSICKVTCTKGSCQNSCEKGNTTTLISENGHAADTLTATNFRVVLCHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYKFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCTNGDCSNLEGSYMCSCHKGYTRTPDHKHCKDIDECQQGNLCVNGQCKNTEGSFRCTCGQGYQLSAAKDQCEDIDECQHHHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVEQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPMKDSDDYAQLCNIPVTGRRQPYGRDALVDFSEQYAPEADPYFIQDRFLNSFEELQAEECGILNGCENGRCVRVQEGYTCDCFDGYHLDTAKMTCVDVNECDELNNRMSLCKNAKCINTEGSYKCLCLPGYVPSDKPNYCTPLNTALNLEKDSDLE

SEQ ID NO: 47 - protein, 1374 aa, Mus sp.
NHTGRIKVVFTPSICKVTCTKGNCQNSCQKGNTTTLISENGHAADTLTATNFRVVICHLPCMNGGQCSSRDKCQCPPNFTGKLCQIPVLGASMPKLYQHAQQQGKALGSHVIHSTHTLPLTMTSQQGVKVKFPPNIVNIHVKHPPEASVQIHQVSRIDSPGGQKVKEAQPGQSQVSYQGLPVQKTQTVHSTYSHQQLIPHVYPVAAKTQLGRCFQETIGSQCGKALPGLSKQEDCCGTVGTSWGFNKCQKCPKKQSYHGYTQMMECLQGYKRVNNTFCQDINECQLQGVCPNGECLNTMGSYRCSCKMGFGPDPTFSSCVPDPPVISEEKGPCYRLVSPGRHCMHPLSVHLTKQICCCSVGKAWGPHCEKCPLPGTAAFKEICPGGMGYTVSGVHRRRPIHQHIGKEAVYVKPKNTQPVAKSTHPPPLPAKEEPVEALTSSWEHGPRGAEPEVVTAPPEKEIPSLDQEKTRLEPGQPQLSPGVSTIHLHPQFPVVVEKTSPPVPVEVAPEASTSSASQVIAPTQVTEINECTVNPDICGAGHCINLPVRYTCICYEGYKFSEQLRKCVDIDECAQVRHLCSQGRCENTEGSFLCVCPAGFMASEEGTNCIDVDECLRPDMCRDGRCINTAGAFRCEYCDSGYRMSRRGYCEDIDECLKPSTCPEEQCVNTPGSYQCVPCTEGFRGWNGQCLDVDECLQPKVCTNGSCTNLEGSYMCSCHRGYSPTPDHRHCQDIDECQQGNLCMNGQCRNTDGSFRCTCGQGYQLSAAKDQCEDIDECEHHHLCSHGQCRNTEGSFQCVCNQGYRASVLGDHCEDINECLEDSSVCQGGDCINTAGSYDCTCPDGFQLNDNKGCQDINECAQPGLCGSHGECLNTQGSFHCVCEQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRVTEDSGVDRQPREEKKECYYNLNDASLCDNVLAPNVTKQECCCTSGAGWGDNCEIFPCPVQGTAEFTEMCPRGKGLVPAGESSYDTGGENYKDADECLLFGEEICKNGYCLNTQPGYECYCKQGTYYDPVKLQCFDMDECQDPNSCIDGQCVNTEGSYNCFCTHPMVLDASEKRCVQPTESNEQIEETDVYQDLCWEHLSEEYVCSRPLVGKQTTYTECCCLYGEAWGMQCALCPMKDSDDYAQLCNIPVTGRRRPYGRDALVDFSEQYGPETDPYFIQDRFLNSFEELQAEECGILNGCENGRCVRVQEGYTCDCFDGYHLDMAKMTCVDVNECSELNNRMSLCKNAKCINTEGSYKCLCLPGYIPSDKPNYCTPLNSALNLDKESDLE

SEQ ID NO: 48 - protein, 646 aa, Mus sp.
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVNLILLLSFTLVSAIVLTTLATICFLRRQKLSQQYKA

SEQ ID NO: 49 - protein, 611 aa, Mus sp.
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVN

SEQ ID NO: 50 - protein, 1375 aa, Unknown
  source: /note=“Description of Unknown: LTBP1S sequence”
NHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGVGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 51 - protein, 1260 aa, Unknown
  source: /note=“Description of Unknown: LTBP3 sequence”
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRTGALSTGALPPLAPEGDSVASKHAIYAVQVIADPPGPGEGPPAQHAAFLVPLGPGQISAEVQAPPPVVNVRVHHPPEASVQVHRIESSNAESAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPTDGTAAFKEICPAGKGYHILTSHQTLTIQGESDFSLFLHPDGPPKPQQLPESPSQAPPPEDTEEERGVTTDSPVSEERSVQQSHPTATTTPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCLDVDECEAGDVCDNGICSNTPGSFQCQCLSGYHLSRDRSHCEDIDECDFPAACIGGDCINTNGSYRCLCPQGHRLVGGRKCQDIDECSQDPSLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGSEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPLAGPALTFDDCCCRQGRGWGAQCRPCPPRGAGSHCPTSQSESNSFWDTSPLLLGKPPRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 52 - protein, 645 aa, Unknown
  source: /note=“Description of Unknown: GARP sequence”
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 53 - protein, 610 aa, Unknown
  source: /note=“Description of Unknown: sGARP sequence”
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNIN

SEQ ID NO: 54 - protein, 5 aa, Artificial Sequence
  source: /note=“Description of Artificial Sequence: Synthetic peptide”
CPPCP

SEQ ID NO: 55 - protein, 13 aa, Artificial Sequence
  source: /note=“Description of Artificial Sequence: Synthetic peptide”
ASTKGPSVFPLAP

SEQ ID NO: 56 - protein, 6 aa, Artificial Sequence
  source: /note=“Description of Artificial Sequence: Synthetic peptide”
ASTKGP

SEQ ID NO: 57 - protein, 12 aa, Artificial Sequence
  source: /note=“Description of Artificial Sequence: Synthetic peptide”
TVAAPSVFIFPP

SEQ ID NO: 58 - protein, 5 aa, Artificial Sequence
  source: /note=“Description of Artificial Sequence: Synthetic peptide”
TVAAP

SEQ ID NO: 59 - protein, 16 aa, Artificial Sequence
  source: /note=“Description of Artificial Sequence: Synthetic peptide”
AKTTPKLEEGEFSEAR

SEQ ID NO: 60 - protein, 17 aa, Artificial Sequence
  source: /note=“Description of Artificial Sequence: Synthetic peptide”
AKTTPKLEEGEFSEARV

SEQ ID NO: 61 - protein, 9 aa, Artificial Sequence
  source: /note=“Description of Artificial Sequence: Synthetic peptide”
AKTTPKLGG

SEQ ID NO: 62 - protein, 10 aa, Artificial Sequence
  source: /note=“Description of Artificial Sequence: Synthetic peptide”
SAKTTPKLGG

SEQ ID NO: 63 - protein, 6 aa, Artificial Sequence
  source: /note=“Description of Artificial Sequence: Synthetic peptide”
SAKTTP

SEQ ID NO: 64 - protein, 6 aa, Artificial Sequence
  source: /note=“Description of Artificial Sequence: Synthetic peptide”
RADAAP

SEQ ID NO: 65 - protein, 9 aa, Artificial Sequence
  source: /note=“Description of Artificial Sequence: Synthetic peptide”
RADAAPTVS

SEQ ID NO: 66 - protein, 12 aa, Artificial Sequence
  source: /note=“Description of Artificial Sequence: Synthetic peptide”
RADAAAAGGPGS

SEQ ID NO: 67 - protein, 27 aa, Artificial Sequence
  source: /note=“Description of Artificial Sequence: Synthetic peptide”
RADAAAAGGGGSGGGGSGGGGSGGGGS

SEQ ID NO: 68 - protein, 18 aa, Artificial Sequence
  source: /note=“Description of Artificial Sequence: Synthetic peptide”
SAKTTPKLEEGEFSEARV

SEQ ID NO: 69 - protein, 5 aa, Artificial Sequence
  source: /note=“Description of Artificial Sequence: Synthetic peptide”
ADAAP

SEQ ID NO: 70 - protein, 12 aa, Artificial Sequence
  source: /note=“Description of Artificial Sequence: Synthetic peptide”
ADAAPTVSIFPP

SEQ ID NO: 71 - protein, 6 aa, Artificial Sequence
  source: /note=“Description of Artificial Sequence: Synthetic peptide”
QPKAAP

SEQ ID NO: 72 - protein, 13 aa, Artificial Sequence
  source: /note=“Description of Artificial Sequence: Synthetic peptide”
QPKAAPSVTLFPP

SEQ ID NO: 73 - protein, 6 aa, Artificial Sequence
  source: /note=“Description of Artificial Sequence: Synthetic peptide”
AKTTPP

SEQ ID NO: 74 - protein, 13 aa, Artificial Sequence
  source: /note=“Description of Artificial Sequence: Synthetic peptide”
AKTTPPSVTPLAP

SEQ ID NO: 75 - protein, 6 aa, Artificial Sequence
  source: /note=“Description of Artificial Sequence: Synthetic peptide”
AKTTAP

SEQ ID NO: 76 - protein, 13 aa, Artificial Sequence
  source: /note=“Description of Artificial Sequence: Synthetic peptide”
AKTTAPSVYPLAP

SEQ ID NO: 77 - protein, 15 aa, Artificial Sequence
  source: /note=“Description of Artificial Sequence: Synthetic peptide”
GGGGSGGGGSGGGGS

SEQ ID NO: 78 - protein, 15 aa, Artificial Sequence
  source: /note=“Description of Artificial Sequence: Synthetic peptide”
GENKVEYAPALMALS

SEQ ID NO: 79 - protein, 15 aa, Artificial Sequence
  source: /note=“Description of Artificial Sequence: Synthetic peptide”
GPAKELTPLKEAKVS

SEQ ID NO: 80 - protein, 15 aa, Artificial Sequence
  source: /note=“Description of Artificial Sequence: Synthetic peptide”
GHEAAAVMQVQYPAS

SEQ ID NO: 81 - protein, 24 aa, Artificial Sequence
  source: /note=“Description of Artificial Sequence: Synthetic peptide”
TVAAPSVFIFPPTVAAPSVFIFPP

SEQ ID NO: 82 - protein, 26 aa, Artificial Sequence
  source: /note=“Description of Artificial Sequence: Synthetic peptide”
ASTKGPSVFPLAPASTKGPSVFPLAP

SEQ ID NO: 83 - protein, 692 aa, Homo sapiens
MELLPLWLCLGFHFLTVGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLYLVLILPSCLTLLVACTVIVLTFKKPLLQVIKSRCHWSSVY

SEQ ID NO: 84 - protein, 660 aa, Artificial Sequence
  source: /note=“Description of Artificial Sequence: Synthetic polypeptide”
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLHHHHHH

SEQ ID NO: 85-100 - skipped (16 entries)

SEQ ID NO: 101 - protein, 689 aa, Artificial Sequence
  source: /note=“Description of Artificial Sequence: Synthetic polypeptide”
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLIIILTFILVSAILLTTLAACCCVRRQKFNQQYKA

SEQ ID NO: 102-107 - skipped (6 entries)

SEQ ID NO: 108 - protein, 9 aa, Artificial Sequence
  source: /note=“Description of Artificial Sequence: Synthetic peptide”
FTFADYAMT

SEQ ID NO: 109 - protein, 17 aa, Artificial Sequence
  source: /note=“Description of Artificial Sequence: Synthetic peptide”
AISGTGAHTYYADSVKG

SEQ ID NO: 110 - protein, 12 aa, Artificial Sequence
  source: /note=“Description of Artificial Sequence: Synthetic peptide”
  source: /note=“See specification as filed for detailed description of substitutions and preferred embodiments”
ARVSSGHWDFDY

SEQ ID NO: 111 - protein, 11 aa, Artificial Sequence
  source: /note=“Description of Artificial Sequence: Synthetic peptide”
  source: /note=“See specification as filed for detailed description of substitutions and preferred embodiments”
RASQSISSYLN

SEQ ID NO: 112 - protein, 7 aa, Artificial Sequence
  source: /note=“Description of Artificial Sequence: Synthetic peptide”
DASSLQS

SEQ ID NO: 113 - protein, 9 aa, Artificial Sequence
  source: /note=“Description of Artificial Sequence: Synthetic peptide”
QQSYSAPFT

SEQ ID NO: 114 - protein, 9 aa, Artificial Sequence
  source: /note=“Description of Artificial Sequence: Synthetic peptide”
FTFSDYAMV

SEQ ID NO: 115 - protein, 17 aa, Artificial Sequence
  source: /note=“Description of Artificial Sequence: Synthetic peptide”
AISGSGFTTYYADSVKG

SEQ ID NO: 116-119 - skipped (4 entries)

SEQ ID NO: 120 - protein, 9 aa, Artificial Sequence
  source: /note=“Description of Artificial Sequence: Synthetic peptide”
FTFSSFAMT

SEQ ID NO: 121 - protein, 17 aa, Artificial Sequence
  source: /note=“Description of Artificial Sequence: Synthetic peptide”
AISGSGAATYFADSVKG

SEQ ID NO: 122-125 - skipped (4 entries)

SEQ ID NO: 126 - protein, 9 aa, Artificial Sequence
  source: /note=“Description of Artificial Sequence: Synthetic peptide”
FTFRNYAMS

SEQ ID NO: 127 - protein, 17 aa, Artificial Sequence
  source: /note=“Description of Artificial Sequence: Synthetic peptide”
SISGSGGATYYADSVKG

SEQ ID NO: 128 - skipped

SEQ ID NO: 129 - protein, 11 aa, Artificial Sequence
  source: /note=“Description of Artificial Sequence: Synthetic peptide”
QASQDISNSLN

SEQ ID NO: 130 - protein, 7 aa, Artificial Sequence
  source: /note=“Description of Artificial Sequence: Synthetic peptide”
DASNLET

SEQ ID NO: 131 - protein, 9 aa, Artificial Sequence
  source: /note=“Description of Artificial Sequence: Synthetic peptide”
QQAPNLPFT

SEQ ID NO: 132-135 - skipped (4 entries)

SEQ ID NO: 136 - protein, 7 aa, Artificial Sequence
  source: /note=“Description of Artificial Sequence: Synthetic peptide”
  source: /note=“See specification as filed for detailed description of substitutions and preferred embodiments”
AASNLQS

SEQ ID NO: 137-144 - skipped (8 entries)

SEQ ID NO: 145 - protein, 17 aa, Artificial Sequence
  source: /note=“Description of Artificial Sequence: Synthetic peptide”
AISSFASAIYYADSVKG

SEQ ID NO: 146-147 - skipped (2 entries)

SEQ ID NO: 148 - protein, 7 aa, Artificial Sequence
  source: /note=“Description of Artificial Sequence: Synthetic peptide”
AASSLQS

SEQ ID NO: 149 - protein, 9 aa, Artificial Sequence
  source: /note=“Description of Artificial Sequence: Synthetic peptide”
QQVYDPPLT

SEQ ID NO: 150 - protein, 9 aa, Artificial Sequence
  source: /note=“Description of Artificial Sequence: Synthetic peptide”
FTFSAYAMT

SEQ ID NO: 151 - protein, 17 aa, Artificial Sequence
  source: /note=“Description of Artificial Sequence: Synthetic peptide”
AISGTGGSTYYADSVKG

SEQ ID NO: 152-167 - skipped (16 entries)

SEQ ID NO: 168 - protein, 9 aa, Artificial Sequence
  source: /note=“Description of Artificial Sequence: Synthetic peptide”
FTFSNYAMS

SEQ ID NO: 169-181 - skipped (13 entries)

SEQ ID NO: 182 - protein, 12 aa, Artificial Sequence
  source: /note=“Description of Artificial Sequence: Synthetic peptide”
ATVSSGHWDFDY

SEQ ID NO: 183-187 - skipped (5 entries)

SEQ ID NO: 188 - protein, 12 aa, Artificial Sequence
  source: /note=“Description of Artificial Sequence: Synthetic peptide”
ARVSSGHWDFDE

SEQ ID NO: 189-192 - skipped (4 entries)

SEQ ID NO: 193 - protein, 17 aa, Artificial Sequence
  source: /note=“Description of Artificial Sequence: Synthetic peptide”
AISGSGGVTYYADSVKG

SEQ ID NO: 194 - protein, 12 aa, Artificial Sequence
  source: /note=“Description of Artificial Sequence: Synthetic peptide”
ARVSSGHWDYDY

SEQ ID NO: 195-199 - skipped (5 entries)

SEQ ID NO: 200 - protein, 12 aa, Artificial Sequence
  source: /note=“Description of Artificial Sequence: Synthetic peptide”
ARVSSYLWDFDY

SEQ ID NO: 201-205 - skipped (5 entries)

SEQ ID NO: 206 - protein, 12 aa, Artificial Sequence
  source: /note=“Description of Artificial Sequence: Synthetic peptide”
ARVSSGHWDLDY

SEQ ID NO: 207-209 - skipped (3 entries)

SEQ ID NO: 210 - protein, 119 aa, Artificial Sequence
  source: /note=“Description of Artificial Sequence: Synthetic polypeptide”
EVQLLESGGGLVQPGGSLRLSCAASGFTFADYAMTWVRQAPGKGLEWVSAISGTGAHTYYADSVKGRFTISRDNSKNTLYLQMNSLRAEDTAVYYCARVSSGHWDFDYWGQGTLVTVSS

SEQ ID NO: 211 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYDASSLQSGVPSRFSGSGSGTDFTLTISSLQPEDFATYYCQQSYSAPFTFGQGTKVEIK

SEQ ID NO: 212 - protein, 119 aa, Artificial Sequence
  source: /note=“Description of Artificial Sequence: Synthetic polypeptide”
QVQLVESGGGVVQPGRSLRLSCAASGFTFSDYAMVWVRQAPGKGLEWVSAISGSGFTTYYADSVKGRFTISRDNSKNTLYLQMNSLRAEDTAVYYCARVSSGHWDFDYWGQGTLVTVSS

SEQ ID NO: 213 - skipped

SEQ ID NO: 214 - protein, 119 aa, Artificial Sequence
  source: /note=“Description of Artificial Sequence: Synthetic polypeptide”
QVQLVESGGGVVQPGGSLRLSCAASGFTFSSFAMTWVRQAPGKGLEWVSAISGSGAATYFADSVKGRFTISRDNSKNTLYLQMNSLRAEDTAVYYCARVSSGHWDFDYWGQGTLVTVSS

SEQ ID NO: 215 - skipped

SEQ ID NO: 216 - protein, 119 aa, Artificial Sequence
  source: /note=“Description of Artificial Sequence: Synthetic polypeptide”
EVQLLESGGGLVQPGGSLRLSCAASGFTFRNYAMSWVRQAPGKGLEWVSSISGSGGATYYADSVKGRFTISRDNSKNTLYLQMNSLRAEDTAVYYCARVSSGHWDFDYWGQGTLVTVSS

SEQ ID NO: 217 - protein, 107 aa, Artificial Sequence
  source: /note=“Description of Artificial Sequence: Synthetic polypeptide”
DIQMTQSPSSLSASVGDRVTITCQASQDISNSLNWYQQKPGKAPKLLIYDASNLETGVPSRFSGSRSGTDFTFTISSLQPEDIATYYCQQAPNLPFTFGGGTKVEIK

SEQ ID NO: 218-221 - skipped (4 entries)

SEQ ID NO: 222 - protein, 119 aa, Artificial Sequence
  source: /note=“Description of Artificial Sequence: Synthetic polypeptide”
EVQLLESGGGLVQPGGSLRLSCAASGFTFADYAMTWVRQAPGKGLEWVSAISSFASAIYYADSVKGRFTISRDNSKNTLYLQMNSLRAEDTAVYYCARVSSGHWDFDYWGQGTLVTVSS

SEQ ID NO: 223 - protein, 107 aa, Artificial Sequence
  source: /note=“Description of Artificial Sequence: Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQVYDPPLTFGGGTKVEIK

SEQ ID NO: 224 - protein, 119 aa, Artificial Sequence
  source: /note=“Description of Artificial Sequence: Synthetic polypeptide”
EVQLLESGGGLVQPGGSLRLSCAASGFTFSAYAMTWVRQAPGKGLEWVSAISGTGGSTYYADSVKGRFTISRDNSKNTLYLQMNSLRAEDTAVYYCARVSSGHWDFDYWGQGTLVTVSS

SEQ ID NO: 225 - skipped

SEQ ID NO: 226 - protein, 119 aa, Artificial Sequence
  source: /note=“Description of Artificial Sequence: Synthetic polypeptide”
EVQLVESGGGVVQPGRSLRLSCAASGFTFSDYAMVWVRQAPGKGLEWVSAISGSGFTTYYADSVKGRFTISRDNSKNTLYLQMNSLRAEDTAVYYCARVSSGHWDFDYWGQGTLVTVSS

SEQ ID NO: 227 - skipped

SEQ ID NO: 228 - protein, 119 aa, Artificial Sequence
  source: /note=“Description of Artificial Sequence: Synthetic polypeptide”
EVQLLESGGGLVQPGGSLRLSCAASGFTFSNYAMSWVRQAPGKGLEWVSAISGSGFTTYYADSVKGRFTISRDNSKNTLYLQMNSLRAEDTAVYYCARVSSGHWDFDYWGQGTLVTVSS

SEQ ID NO: 229 - skipped

SEQ ID NO: 230 - protein, 119 aa, Artificial Sequence
  source: /note=“Description of Artificial Sequence: Synthetic polypeptide”
EVQLLESGGGVVQPGGSLRLSCAASGFTFSSFAMTWVRQAPGKGLEWVSAISGSGAATYFADSVKGRFTISRDNSKNTLYLQMNSLRAEDTAVYYCARVSSGHWDFDYWGQGTLVTVSS

SEQ ID NO: 231 - skipped

SEQ ID NO: 232 - protein, 119 aa, Artificial Sequence
  source: /note=“Description of Artificial Sequence: Synthetic polypeptide”
QVQLVESGGGVVQPGRSLRLSCAASGFTFSDYAMVWVRQAPGKGLEWVSAISGSGAATYFADSVKGRFTISRDNSKNTLYLQMNSLRAEDTAVYYCATVSSGHWDFDYWGQGTLVTVSS

SEQ ID NO: 233 - skipped

SEQ ID NO: 234 - protein, 119 aa, Artificial Sequence
  source: /note=“Description of Artificial Sequence: Synthetic polypeptide”
QVQLVESGGGVVQPGRSLRLSCAASGFTFSSFAMTWVRQAPGKGLEWVSAISGSGAATYFADSVKGRFTISRDNSKNTLYLQMNSLRAEDTAVYYCARVSSGHWDFDEWGQGTLVTVSS

SEQ ID NO: 235 - skipped

SEQ ID NO: 236 - protein, 119 aa, Artificial Sequence
  source: /note=“Description of Artificial Sequence: Synthetic polypeptide”
EVQLLESGGGLVQPGGSLRLSCAASGFTFSAYAMTWVRQAPGKGLEWVSAISGSGGVTYYADSVKGRFTISRDNSKNTLYLQMNSLRAEDTAVYYCARVSSGHWDYDYWGQGTLVTVSS

SEQ ID NO: 237 - skipped

SEQ ID NO: 238 - protein, 119 aa, Artificial Sequence
  source: /note=“Description of Artificial Sequence: Synthetic polypeptide”
EVQLLESGGGLVQPGGSLRLSCAASGFTFRNYAMSWVRQAPGKGLEWVSSISGSGGATYYADSVKGRFTISRDNSKNTLYLQMNSLRAEDTAVYYCARVSSYLWDFDYWGQGTLVTVSS

SEQ ID NO: 239 - skipped

SEQ ID NO: 240 - protein, 119 aa, Artificial Sequence
  source: /note=“Description of Artificial Sequence: Synthetic polypeptide”
EVQLLESGGGLVQPGGSLRLSCAASGFTFRNYAMSWVRQAPGKGLEWVSSISGSGGATYYADSVKGRFTISRDNSKNTLYLQMNSLRAEDTAVYYCARVSSGHWDLDYWGQGTLVTVSS

SEQ ID NO: 241-242 - skipped (2 entries)

SEQ ID NO: 243 - protein, 107 aa, Artificial Sequence
  source: /note=“Description of Artificial Sequence: Synthetic polypeptide”
DIQLTQSPSSLSASVGDRVTITCQASQDISNFLNWYQQKPGKAPKLLIYDASNLETGVPSRFSGSGSGTDFTFTISSLQPEDIATYYCQQVFNPPLTFGGGTKVEIK

SEQ ID NO: 244-249 - skipped (6 entries)

SEQ ID NO: 250 - protein, 9 aa, Artificial Sequence
  source: /note=“Description of Artificial Sequence: Synthetic peptide”
  source: /note=“See specification as filed for detailed description of substitutions and preferred embodiments”
FTFSDYAMT

SEQ ID NO: 251 - protein, 17 aa, Artificial Sequence
  source: /note=“Description of Artificial Sequence: Synthetic peptide”
  source: /note=“See specification as filed for detailed description of substitutions and preferred embodiments”
AISGSGAATYYADSVKG

SEQ ID NO: 252 - protein, 9 aa, Artificial Sequence
  source: /note=“Description of Artificial Sequence: Synthetic peptide”
  VARIANT (4)..(4): /replace=“Ser”
  VARIANT (5)..(5): /replace=“Asp” or “Ser” or “Ala”
  VARIANT (6)..(6): /replace=“Phe”
  VARIANT (9)..(9): /replace=“Thr” or “Val”
  SITE (1)..(9): /note=“Variant residues given in the sequence have no preference with respect to those in the annotations for variant positions”
FTFANYAMS

SEQ ID NO: 253 - protein, 17 aa, Artificial Sequence
  source: /note=“Description of Artificial Sequence: Synthetic peptide”
  VARIANT (1)..(1): /replace=“Ala”
  VARIANT (4)..(4): /replace=“Ser”
  VARIANT (5)..(5): /replace=“Thr” or “Phe”
  VARIANT (6)..(6): /replace=“Ala”
  VARIANT (7)..(7): /replace=“Ala” or “Phe” or “Ser”
  VARIANT (8)..(8): /replace=“His” or “Thr” or “Ser” or “Val”
  VARIANT (9)..(9): /replace=“Ile”
  VARIANT (11)..(11): /replace=“Phe”
  SITE (1)..(17): /note=“Variant residues given in the sequence have no preference with respect to those in the annotations for variant positions”
SISGSGGATYYADSVKG

SEQ ID NO: 254 - protein, 12 aa, Artificial Sequence
  source: /note=“Description of Artificial Sequence: Synthetic peptide”
  VARIANT (2)..(2): /replace=“Thr”
  VARIANT (6)..(6): /replace=“Tyr”
  VARIANT (7)..(7): /replace=“Leu”
  VARIANT (10)..(10): /replace=“Tyr” or “Leu”
  VARIANT (12)..(12): /replace=“Glu”
  SITE (1)..(12): /note=“Variant residues given in the sequence have no preference with respect to those in the annotations for variant positions”
ARVSSGHWDFDY

SEQ ID NO: 255 - protein, 11 aa, Artificial Sequence
  source: /note=“Description of Artificial Sequence: Synthetic peptide”
  VARIANT (1)..(1): /replace=“Gln”
  VARIANT (5)..(5): /replace=“Asp”
  VARIANT (8)..(8): /replace=“Asn”
  VARIANT (9)..(9): /replace=“Tyr” or “Ser”
  SITE (1)..(11): /note=“Variant residues given in the sequence have no preference with respect to those in the annotations for variant positions”
RASQSISSFLN

SEQ ID NO: 256 - protein, 7 aa, Artificial Sequence
  source: /note=“Description of Artificial Sequence: Synthetic peptide”
  VARIANT (1)..(1): /replace=“Ala”
  VARIANT (4)..(4): /replace=“Asn”
  VARIANT (6)..(6): /replace=“Glu”
  VARIANT (7)..(7): /replace=“Thr”
  SITE (1)..(7): /note=“Variant residues given in the sequence have no preference with respect to those in the annotations for variant positions”
DASSLQS

SEQ ID NO: 257 - protein, 9 aa, Artificial Sequence
  source: /note=“Description of Artificial Sequence: Synthetic peptide”
  VARIANT (3)..(3): /replace=“Ala” or “Thr” or “Val”
  VARIANT (4)..(4): /replace=“Tyr” or “Pro”
  VARIANT (5)..(5): /replace=“Asn” or “Thr” or “Asp”
  VARIANT (6)..(6): /replace=“Leu” or “Val” or “Pro”
  VARIANT (8)..(8): /replace=“Leu”
  SITE (1)..(9): /note=“Variant residues given in the sequence have no preference with respect to those in the annotations for variant positions”
QQSFSAPFT

SEQ ID NO: 258 - protein, 21 aa, Homo sapiens
SQGEVPPGPLPEAVLALYNST

SEQ ID NO: 259 - protein, 7 aa, Homo sapiens
LREAVPE

SEQ ID NO: 260 - protein, 8 aa, Homo sapiens
YHANFCLG

SEQ ID NO: 261 - protein, 24 aa, Homo sapiens
SPPSQGEVPPGPLPEAVLALYNST

SEQ ID NO: 262 - protein, 17 aa, Homo sapiens
WKWIHEPKGYHANFCLG

SEQ ID NO: 263 - protein, 249 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 264 - protein, 45 aa, Homo sapiens
LSTCKTIDMELVKRKRIEAIRGQILSKLRLASPPSQGEVPPGPLP

SEQ ID NO: 265 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 266 - protein, 4 aa, Homo sapiens
RHRR

SEQ ID NO: 267 - protein, 204 aa, Homo sapiens
EAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 268 - protein, 29 aa, Homo sapiens
CVRQLYIDFRKDLGWKWIHEPKGYHANFC

SEQ ID NO: 269 - protein, 35 aa, Homo sapiens
CVPQALEPLPIVYYVGRKPKVEQLSNMIVRSCKCS

SEQ ID NO: 270 - protein, 15 aa, Homo sapiens
LASPPSQGEVPPGPL

SEQ ID NO: 271 - protein, 27 aa, Homo sapiens
LASPPSQGEVPPGPLPEAVLALYNSTR

SEQ ID NO: 272 - protein, 29 aa, Homo sapiens
LSTCKTIDMELVKRKRIEAIRGQILSKLR

SEQ ID NO: 273 - protein, 10 aa, Homo sapiens
AVLALYNSTR

SEQ ID NO: 274 - protein, 20 aa, Homo sapiens
NGFTTGRRGDLATIHGMNRP

SEQ ID NO: 275 - protein, 13 aa, Homo sapiens
CSCDSRDNTLQVD

SEQ ID NO: 276 - protein, 9 aa, Artificial Sequence
  source: /note=“Description of Artificial Sequence: Synthetic peptide”
  VARIANT (4)..(4): /replace=“Ser”
  VARIANT (6)..(6): /replace=“Phe”
  VARIANT (9)..(9): /replace=“Thr” or “Val”
  SITE (1)..(9): /note=“Variant residues given in the sequence have no preference with respect to those in the annotations for variant positions”
FTFADYAMS

SEQ ID NO: 277 - protein, 17 aa, Artificial Sequence
  source: /note=“Description of Artificial Sequence: Synthetic peptide”
  VARIANT (1)..(1): /replace=“Ala”
  VARIANT (4)..(4): /replace=“Ser”
  VARIANT (6)..(6): /replace=“Ala”
  VARIANT (7)..(7): /replace=“Ala” or “Phe” or “Ser”
  VARIANT (8)..(8): /replace=“His” or “Thr” or “Ser” or “Val”
  VARIANT (9)..(9): /replace=“Ile”
  VARIANT (11)..(11): /replace=“Phe”
  SITE (1)..(17): /note=“Variant residues given in the sequence have no preference with respect to those in the annotations for variant positions”
SISGSGGATYYADSVKG

SEQ ID NO: 278 - protein, 12 aa, Artificial Sequence
  source: /note=“Description of Artificial Sequence: Synthetic peptide”
  VARIANT (2)..(2): /replace=“Thr”
  VARIANT (10)..(10): /replace=“Tyr” or “Leu”
  VARIANT (12)..(12): /replace=“Glu”
  SITE (1)..(12): /note=“Variant residues given in the sequence have no preference with respect to those in the annotations for variant positions”
ARVSSGHWDFDY

SEQ ID NO: 279 - protein, 8 aa, Artificial Sequence
  source: /note=“Description of Artificial Sequence: Synthetic peptide”
  VARIANT (2)..(2): /replace=“Ala”
  VARIANT (5)..(5): /replace=“Asn”
  VARIANT (7)..(7): /replace=“Glu”
  VARIANT (8)..(8): /replace=“Thr”
  SITE (1)..(8): /note=“Variant residues given in the sequence have no preference with respect to those in the annotations for variant positions”
YDASSLQS

SEQ ID NO: 280 - protein, 9 aa, Artificial Sequence
  source: /note=“Description of Artificial Sequence: Synthetic peptide”
  VARIANT (5)..(5): /replace=“Asn” or “Thr” or “Asp”
  VARIANT (6)..(6): /replace=“Leu” or “Val” or “Pro”
  VARIANT (8)..(8): /replace=“Leu”
  SITE (1)..(9): /note=“Variant residues given in the sequence have no preference with respect to those in the annotations for variant positions”
QQTYSAPFT

SEQ ID NO: 281 - protein, 20 aa, Homo sapiens
EDYPEPEEVPPEVISIYNST

SEQ ID NO: 282 - protein, 4 aa, Homo sapiens
MEKN

SEQ ID NO: 283 - protein, 8 aa, Homo sapiens
YNANFCAG

SEQ ID NO: 284 - protein, 19 aa, Homo sapiens
EPTVMTHVPYQVLALYNST

SEQ ID NO: 285 - protein, 4 aa, Homo sapiens
VEKN

SEQ ID NO: 286 - protein, 8 aa, Homo sapiens
YYANFCSG

SEQ ID NO: 287 - protein, 23 aa, Homo sapiens
SPPEDYPEPEEVPPEVISIYNST

SEQ ID NO: 288 - protein, 17 aa, Homo sapiens
WKWIHEPKGYNANFCAG

SEQ ID NO: 289 - protein, 22 aa, Homo sapiens
SPPEPTVMTHVPYQVLALYNST

SEQ ID NO: 290 - protein, 17 aa, Homo sapiens
WKWVHEPKGYYANFCSG

SEQ ID NO: 291 - protein, 23 aa, Homo sapiens
KLRLASPPSQGEVPPGPLPEAVL

SEQ ID NO: 292 - protein, 22 aa, Homo sapiens
KLKLTSPPEDYPEPEEVPPEVI

SEQ ID NO: 293 - protein, 21 aa, Homo sapiens
KLRLTSPPEPTVMTHVPYQVL

SEQ ID NO: 294 - protein, 6 aa, Unknown
  source: /note=“Description of Unknown: TGFbeta sequence”
LSKLRL

SEQ ID NO: 295 - protein, 6 aa, Artificial Sequence
  source: /note=“Description of Artificial Sequence: Synthetic 6xHis tag”
HHHHHH